(19) United States
(12) Patent Application Publication
(10) Pub. No.: US 2021/0155691 A1
(43) Pub. Date: May 27, 2021

(54) METHODS OF PREVENTING OR TREATING NON-HEMATOPOIETIC SLAMF7 POSITIVE AND SLAMF7 NEGATIVE CANCERS

(71) Applicant: ADAERATA, LIMITED PARTNERSHIP, Montreal (CA)

(72) Inventors: André VEILLETTE, Montréal (CA); Jun CHEN, Montréal (CA)

(21) Appl. No.: 17/047,130

(22) PCT Filed: Apr. 15, 2019
(86) PCT No.: PCT/CA2019/050457

§ 371 Date: Oct. 13, 2020

Related U.S. Application Data
(60) Provisional application No. 62/658,243, filed on Apr. 16, 2018.

(51) Int. Cl. C07K 16/28; A61K 39/395; A61K 45/06; A61P 35/00; G01N 33/574

(52) U.S. Cl. CPC C07K 16/2803; A61K 39/3955; A61K 45/06; A61K 2039/545; G01N 33/57492; C07K 2317/52; C07K 2317/24; A61P 35/00

(57) ABSTRACT

A method for the prevention and/or treatment of a neoplastic disease comprising a solid tumor in a subject in need thereof, said method comprising administering an effective amount of a signal regulatory protein alpha (SIRPalpha)-cluster of differentiation 47 (CD47) checkpoint inhibitor or a composition comprising the inhibitor, and a pharmaceutically acceptable carrier, to a subject having solid tumor cells expressing signaling lymphocytic activation molecule family member 7 (SLAMF7) and CD47.

### CROSS REFERENCE TO RELATED APPLICATIONS

This application is a PCT application filed on Apr. 15, 2019 and published in English under PCT Article 21(2), which itself claims benefit of U.S. provisional application Ser. No. 62/658,243, filed on Apr. 16, 2018. All documents above are incorporated herein in their entirety by reference.

### STATEMENT REGARDING FEDERALLY SPONSORED RESEARCH OR DEVELOPMENT

N.A.

### FIELD OF THE DISCLOSURE

The present disclosure relates to methods of preventing or treating non-hematopoietic SLAMF7 positive and SLAMF7 negative cancers. More specifically, the present disclosure is concerned with such methods and with methods of selecting treatment in view of SLAMF7 presence or absence on tumor cells.

### REFERENCE TO SEQUENCE LISTING

Pursuant to 37 C.F.R. 1.821(c), a sequence listing is submitted herewith as an ASCII compliant text file named sequence listing 12810-678_5T25, that was created on Apr. 15, 2019 and having a size of 62 kilobytes. The content of the aforementioned file named sequence listing 12810-678_5T25 is hereby incorporated by reference in its entirety.

### BACKGROUND OF THE DISCLOSURE

Cancer cells elude antitumor immunity through multiple mechanisms, including up-regulated expression of ligands for inhibitory immune checkpoint receptors¹⁻⁴. Phagocytosis by macrophages plays a critical role in cancer control⁵⁻⁸. Therapeutic blockade of signal regulatory protein alpha (SIRPalpha), an inhibitory receptor on macrophages, or of its ligand cluster of differentiation 47 (CD47) expressed on tumor cells, improves tumor cell elimination in vitro and in vivo⁵⁻⁸, suggesting that blockade of the SIRPalpha-CD47 checkpoint could be useful to treat human cancer⁹⁻¹².

Neoplastic Disease

The transformation of a normal cell into a malignant cell results, among other things, in the uncontrolled proliferation of the progeny cells, which exhibit immature, undifferentiated morphology, exaggerated survival and pro-angiogenic properties. Once a tumor has formed, cancer cells can leave the original tumor site and migrate to other parts of the body via the bloodstream and/or the lymphatic system by a process called metastasis. In this way, the disease may spread from one organ or part to another non-contiguous organ or part.

The increased number of cancer cases reported around the world is a major concern. Currently there is only a handful of treatments available for specific types of cancer and these treatments provide only limited efficacy and are often associated with toxicity. In addition, one of the biggest concerns of all cancer treatments is the development of chemotherapy resistance.

All steps of cancer progression as well as the development of drug resistance arise as a result of the acquisition of a series of fixed DNA sequence abnormalities, mutations, many of which ultimately confer a growth advantage upon the cells in which they have occurred. Some mutations lead, for example, to the overexpression or constitutive activation of oncogenes not normally expressed by normal mature cells.

Tumor Profiling

Although the understanding of the molecular pathogenesis of cancer has advanced in the last two decades, risk assessment continues to be solely based on a few clinical parameters. Many studies conducted in recent years support the concept that the prognostic assessment of cancer should routinely include the investigation of molecular biomarkers. Also, because side effects of many treatments are severe, there is a need for targeted therapy. In cancer therapy, the quest for better treatment modalities includes better stratification of patients into populations of likely responders to a proposed therapy using small molecules capable of inhibiting hyperactive pathways without adverse effects. In addition, supplementing conventional diagnostics with molecular information should help to identify patients with pre-malignant lesions, patients at risk of developing drug resistance, patients with aggressive tumors for whom maximal therapy is appropriate and others who might survive with less toxic adjuvant therapy of reduced intensity (and thus suffer from fewer, less severe side-effects). Therefore, the development of robust and sensitive assays based on biomarkers linked to appropriate chemotherapeutic agents is certainly a need in cancer.

More specifically, there is a need for alternative targeted anti-neoplastic preventions and/or treatments adapted to specific tumor characteristics.

The present description refers to a number of documents, the content of which is herein incorporated by reference in their entirety.

### SUMMARY OF THE DISCLOSURE

The present inventors found that macrophages were much more efficient at phagocytosis of SLAMF7 positive tumor cells, compared to SLAMF7 negative tumor cells, in response to SIRPalpha-CD47 blockade. More particularly, using a mouse lacking the SLAM (Signaling lymphocytic activation molecule) family of homotypic hematopoietic cell-specific receptors¹³⁻¹⁵, the inventors determined that phagocytosis of tumor cells during SIRPalpha-CD47 blockade was strictly dependent on SLAM family receptors in vitro and in vivo. In both mouse and human cells, this function required a single SLAM family member, SLAM family member 7 (SLAMF7) (also named CRACC, CS1, CD319), expressed on macrophages and tumor cell targets. In contrast to most SLAM receptors functions¹³⁻¹⁵, SLAMF7-mediated phagocytosis was independent of SAP adaptors. Instead, it depended on the ability of SLAMF7 to interact with integrin Mac-1¹⁶⁻¹⁸, and utilize signals involving immunoreceptor tyrosine-based activation motifs (ITAMs)^{19,20}. The inventors also showed that the SLAMF7-mediated phagocytosis was Fc receptor independent. These findings elucidate the mechanism by which macrophages engulf and destroy certain tumor cells. They also reveal a novel SAP adaptor-independent function for a SLAM receptor. These findings show that patients with tumors expressing SLAMF7 are more likely to respond to SIRPalpha-CD47 checkpoint blockade therapy and that non-Fc receptor binding SIRPalpha-CD47 checkpoint inhibitors are effective against such tumors. These findings also show that patients with tumors not expressing SLAMF7 are not likely to respond to SIRPalpha-CD47 checkpoint blockade therapy but that they are more likely to respond to a therapy using a SLAMF7 inhibitor which would result in activation of T cells (or T lymphocytes) (SLAMF7 inhibits T cell activation)⁴¹, along eventually with another agent that activate T cells.

More specifically, in accordance with the present disclosure, there are provided the following items:

1. A method for the prevention and/or treatment of a neoplastic disease comprising a solid tumor in a subject in need thereof, said method comprising administering an effective amount of (i) a signal regulatory protein alpha (SIRPalpha)-cluster of differentiation 47 (CD47) checkpoint inhibitor; or (ii) a composition comprising the inhibitor, and a pharmaceutically acceptable carrier; to a subject having solid tumor cells expressing signaling lymphocytic activation molecule family member 7 (SLAMF7) and CD47.

2. The method of item 1, wherein the solid tumor is a bile duct, breast, colorectal, esophagus, glioma, liver, non-small cell lung, melanoma, ovary, pancreas, soft tissue, stomach, upper aerodigestive or urinary tract tumor.

3. The method of item 1, wherein the solid tumor is a glioma, liver, non-small cell lung, melanoma, upper aerodigestive or urinary tract tumor.

4. The method of item 1, wherein the solid tumor is a non-small cell lung tumor or a melanoma.

5. The method of any one of items 1 to 4, further comprising detecting (i) SLAMF7 expression and/or activity; (ii) CD47 expression and/or activity; or (iii) a combination of at (i) and (ii) in the tumor cells.

6. The method of any one of items 1 to 5, wherein the SIRPalpha-CD47 checkpoint inhibitor is a non-Fc receptor binding inhibitor.

7. The method of any one of items 1 to 6, wherein the SIRPalpha-CD47 checkpoint inhibitor is an antibody or antibody fragment that specifically binds to CD47 and/or an antibody or an antibody fragment that specifically binds to SIRPalpha.

8. The method of any one of items 1 to 7, wherein the SIRPalpha-CD47 checkpoint inhibitor is a non-Fc receptor binding antibody fragment.

9. The method of any one of items 1 to 8, further comprising administering at least one further therapeutic agent to the subject.

10. The method of item 9, wherein the at least one further therapeutic agent comprises a SLAMF7 agonist.

11. The method of item 10, wherein the SLAMF7 agonist is elotuzumab.

12. A method for stratifying a subject having a neoplastic disease comprising a solid tumor comprising detecting signaling lymphocytic activation molecule family member 7 (SLAMF7) expression and/or activity in the subject's tumor cells, wherein said detecting enables the stratification of the subject, preferably wherein when SLAMF7 expression and/or activity is detected the subject's tumor cells, the subject is included in a clinical trial for a SIRPalpha-CD47 checkpoint inhibitor.

13. The method of item 12, wherein when SLAMF7 expression and/or activity is detected, the method further comprises administering an effective amount of (i) a signal regulatory protein alpha (SIRPalpha)-cluster of differentiation 47 (CD47) checkpoint inhibitor; or (ii) a composition comprising the inhibitor, and a pharmaceutically acceptable carrier, to the subject.

14. The method of item 13, wherein the SIRPalpha-CD47 checkpoint inhibitor is a non-Fc receptor binding inhibitor.

15. The method of item 13 or 14, wherein the SIRPalpha-CD47 checkpoint inhibitor is an antibody or an antibody fragment that specifically binds to CD47 and/or an antibody or an antibody fragment that specifically binds to SIRPalpha.

16. The method of any one of items 13 to 15, wherein the SIRPalpha-CD47 checkpoint inhibitor is a non-Fc receptor binding antibody fragment.

17. The method of any one of items 13 to 16, further comprising administering at least one further therapeutic agent to the subject.

18. The method of item 17, wherein the at least one further therapeutic agent comprises a SLAMF7 agonist.

19. The method of item 18, wherein the SLAMF7 agonist is elotuzumab.

20. The method of item 12, wherein when SLAMF7 expression and/or activity is not detected, the method further comprises administering (a) an effective amount of (i) a SLAMF7 inhibitor; (ii) an SIRPalpha-CD47 checkpoint inhibitor and of an Fc receptor-binding antibody or fragment thereof targeting an antigen expressed at the surface of the subject's tumor cells; or (iii) a combination of (i) and (ii); or (b) a composition comprising (a), and a pharmaceutically acceptable carrier, to the subject.

21. The method of item 20, further comprising administering at least one further therapeutic agent to the subject.

22. The method of item 21, wherein the at least one further therapeutic agent comprises another agent that activates T cells.

23. A kit for preventing and/or treating a neoplastic disease comprising a solid tumor in a subject, comprising (a) a signal regulatory protein alpha (SIRPalpha)-cluster of differentiation 47 (CD47) checkpoint inhibitor; and (b) (i) a pharmaceutically acceptable carrier; (ii) at least one further therapeutic agent; or (iii) a combination of (i) and (ii).

24. The kit of item 23, wherein the SIRPalpha-CD47 checkpoint inhibitor is a non-Fc receptor binding inhibitor.

25. The kit of item 23 or 24, wherein the SIRPalpha-CD47 checkpoint inhibitor is an antibody or an antibody fragment that specifically binds to CD47 and/or an antibody or an antibody fragment that specifically binds to SIRPalpha.

26. The kit of any one of items 23 to 25, wherein the SIRPalpha-CD47 checkpoint inhibitor is a non-Fc receptor binding antibody fragment.

27. The kit of any one of items 23 to 26, wherein the at least one further therapeutic agent comprises a SLAMF7 agonist.

28. The kit of item 27, wherein the SLAMF7 agonist is elotuzumab.

29. A method for the prevention and/or treatment of a neoplastic disease comprising a solid tumor in a subject in need thereof, said method comprising administering an effective amount of (i) a signaling lymphocytic activation molecule family member 7 (SLAMF7) inhibitor; or (ii) a composition comprising the inhibitor, and a pharmaceutically acceptable carrier; to a subject having solid tumor cells that do not express signaling lymphocytic activation molecule family member 7 (SLAMF7).

30. The method of item 29, wherein the solid tumor is a bile duct, breast, colorectal, esophagus, glioma, liver, non-small cell lung, melanoma, ovary, pancreas, soft tissue, stomach, upper aerodigestive or urinary tract tumor.

31. The method of item 29, wherein the solid tumor is a glioma, liver, non-small cell lung, melanoma, upper aerodigestive or urinary tract tumor.

32. The method of item 29, wherein the solid tumor is a non-small cell lung tumor or a melanoma.

33. The method of item 29 or 32, further comprising determining SLAMF7 expression and/or activity in the tumor cells.

34. The method of any one of items 29 to 32, further comprising administering at least one further therapeutic agent to the subject.

35. The method of item 34, wherein the at least one further therapeutic agent comprises another agent that activates T cells.

36. A kit for preventing and/or treating a neoplastic disease comprising a solid tumor in a subject, comprising (a) a signaling lymphocytic activation molecule family member 7 (SLAMF7) inhibitor; and (b) (i) a pharmaceutically acceptable carrier; (ii) at least one further therapeutic agent; or (iii) a combination of (i) and (ii).

37. The kit of item 36, wherein the at least one further therapeutic agent comprises another agent that activates T cells.

38. A method for the prevention and/or treatment of a neoplastic disease comprising a solid tumor in a subject in need thereof, said method comprising administering (i) an effective amount of (a) a signaling lymphocytic activation molecule family member 7 (SLAMF7) protein or nucleic acid; or (b) a composition comprising the protein or nucleic acid, and a pharmaceutically acceptable carrier; and (ii) an effective amount of (a) a signal regulatory protein alpha (SIRPalpha)-cluster of differentiation 47 (CD47) checkpoint inhibitor; or (b) a composition comprising the SIRPalpha-CD47 checkpoint inhibitor, and a pharmaceutically acceptable carrier, to a subject having solid tumor cells that do not express signaling lymphocytic activation molecule family member 7 (SLAMF7).

39. The method of item 38, wherein the administrations of (i) and (ii) are performed sequentially.

40. A kit for stratifying a subject having a neoplastic disease comprising a solid tumor, comprising (a) a signaling lymphocytic activation molecule family member 7 (SLAMF7) ligand; and (b) (i) a cluster of differentiation 47 (CD47) ligand; (ii) signal regulatory protein alpha (SIRPalpha) ligand; or (iii) a combination of (i) and (ii).

41. The kit of item 40, wherein (i) the SLAMF7 ligand is an antibody that specifically binds to SLAMF7; (ii) the CD47 ligand is an antibody that specifically binds to CD47; (iii) the SIRPalpha ligand is an antibody that specifically binds to SIRPalpha; or (iv) any combination of at least two of (i) to (iii).

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIGS. 1A-B. Generation of SLAMF7 KO and SLAMF1 KO mice. (FIG. 1A) The relevant segment of the wild-type Slamf7 locus, including exon 2 that contains the initiating ATG (arrowhead), is depicted at the top. Below is the targeting plasmid used to create the SLAMF7 KO mouse. The middle fragment contains a neo-thymidine kinase (tk) cassette bordered by frt sites and a 1.0 kb-genomic fragment bearing exon 2 of Slamf7, flanked by loxP sites. The targeted allele is depicted underneath. After expression of Cre recombinase, the neo-tk cassette and exon 2 were removed to generate the Slamf7^{-/-} allele. (FIG. 1B) The relevant segment of the wild-type Slamf1 locus, including exon 1 that contains the initiating ATG (arrowhead), is depicted at the top. The targeting construct is shown below. The construct allows disruption and introduction of a stop codon (TGA) in exon 1. The middle fragment contains the neo cassette, which is bordered by frt sites and one loxP site. The targeted allele containing the neo cassette is depicted below. The neo-deleted allele, which was generated by transient expression of the Flpe recombinase, is shown at the bottom.

FIGS. 2A-L. Macrophages phagocytose a subset of hematopoietic cells. (FIG. 2A) Phagocytosis assay. MU: macrophage; Ctl: control. (top panel) Bone marrow-derived macrophages (BMDMs) from wild-type mice were seeded on coverslips and incubated with CFSE-labeled L1210 cells (B cell lymphocytic leukemia), in the presence of blocking anti-CD47 antibody or control IgG. After 2 hours, cells were extensively washed and phagocytosis of L1210 (green) was assessed by fluorescence microscopy. Phagocytosed target cells are shown by arrows. Representative fields are shown. Mφs, macrophages. Scale bar, 50 μm. (lower panel) (FIG. 2B) The experiment was the same as in FIG. 2A, except that BMDMs were labeled with Cell Trace Violet (CTV), and phagocytosis was assessed by confocal microscopy. Representative macrophages (red) without or with phagocytosed targets (green) are depicted. In one case (bottom right panel), one L1210 cell (green), shown by arrow, is non-phagocytosed. Scale bar, 5 μm. (FIG. 2C) The experiment was the same as in FIG. 2A, except that Tac (CD25)-positive CFSE-labeled L1210 cells were used as targets and phagocytosis was assessed by flow cytometry. After several washes, non-captured L1210 cells were excluded by gating out CD25-positive cells. Phagocytosis was assessed by analysing CFSE fluorescence in F4/80-positive BMDMs. Top panel shows results obtained with non-authenticated cells. Lower panel shows results obtained with authenticated cells (representative of n=4). (FIG. 2D) The experiment was the same as in FIG. 2A, except that L1210 was loaded with pHrodo™ Green, a pH-sensitive dye that is non-fluorescent at neutral pH but becomes green fluorescent in acidic environments such as phagolysosomes. Phagocytosis was assessed using flow cytometry by measuring pHrodo™ Green fluorescence in gated F4/80-positive macrophages. (FIG. 2E) The experiment was the same as in FIG. 2A, except that various other mouse hematopoietic cells were used as targets: CB17-3A8 (Abl-transformed B cell leukemia), SP2/0 (multiple myeloma), P815 (mastocytoma) and WEHI-3B (myelomonocytic leukemia). Bars represent the average percentage of BMDMs showing phagocytosis of targets from at least three independent experiments for each cell type. Error bars represent standard deviations. Top panel shows results obtained with non-authenticated cells. Lower panel shows results obtained with authenticated cells. (FIG. 2F) Same as FIG. 2E except that F(ab′)₂ fragments of Ab were used instead of intact Ab. (FIG. 2G) The experiment was the same as in FIG. 2E, except that thioglycolate-elicited peritoneal macrophages were used for phagocytosis. Top panel with non-authenticated cells. Lower panel with authenticated cells. (FIG. 2H) The experiment was the same as in FIG. 2E, except that IFN-gamma-treated BMDMs were used for phagocytosis. (FIG. 2I) The experiment was the same as in FIG. 2E, except that activated CD4⁺ T cells from wild-type mice were used as targets. (FIG. 2J) Cell death (in the absence of added macrophages) was examined by staining with annexin V and propidium iodide (PI), and flow cytometry. (FIG. 2K) Same as FIG. 2J, except that cell proliferation was studied by CFSE dilution and flow cytometry. MFI: mean fluorescence intensity. (FIG. 2L) Same as FIG. 2J, except that Ca²⁺ fluxes were analyzed using the Ca²⁺ indicator dye Indo-1, and flow cytometry. Ionomycin served as positive control. Time of addition of stimuli is shown by arrow. (FIG. 2M) Same as FIG. 2J, except that protein tyrosine phosphorylation was detected by anti-phosphotyrosine (P.tyr) immunoblotting. (FIG. 2N) The experiment was the same as in FIG. 2E, except that various other mouse tumor cell lines were used as targets: MEL (erythroleukemia), BI-141 (T cell hybridoma), EL-4 (T cell lymphoma), RMA-S (T cell lymphoma), YAC-1 (thymoma), B16 (melanoma), CMT-93 (rectal carcinoma) and L929 (immortalized fibroblast). Top panel with non-authenticated cells. Lower panel with authenticated cells. (FIG. 2O) Expression of CD47 (dotted lines) in authenticated cells; m: mouse; h: human. Filled curves: isotype controls. (FIG. 2P) CD47-deficient variants of L1210 were generated by CRISPR-Cas-mediated gene editing, using two distinct guide RNA sequences (#1 and #2). Expression of CD47 in parental and CD47-K0 L1210 cells was analyzed by flow cytometry (left; dotted lines). Filled curves represent staining with isotype control antibody. Phagocytosis assay is shown on the right. Parental L1210 cells treated with anti-CD47 Ab or control IgG are also depicted, as controls. (FIG. 2Q) The experiment was the same as in FIG. 2E, except that mouse BMDMs were incubated with human hematopoietic and non-hematopoietic cell lines as targets: Raji (B cell lymphoma), Daudi (B cell lymphoma), Colo205 (colon carcinoma), SW480 (colon carcinoma) and SW620 (colon carcinoma). Given that the anti-human CD47 MAb is of mouse origin, F(ab′)2 fragments of antibodies were utilized. Top panel with non-authenticated cells. Lower panel with authenticated cells. FIG. 2R) The experiment was the same as in FIG. 2Q, except that BMDMs pretreated with LPS were used. (FIG. 2S) The experiment was the same as in FIG. 2E, except that normal mouse hematopoietic target cells were used. Authenticated cells. n.s., not significant; *: p<0.05; **: p<0.01; ***p<0.001 (two-tailed Student's t-tests). In first set of experiments, Representative of n=6 (FIG. 2A, P (left panel)), n=2 (FIGS. 2B, F (lower panel), G, L, M, N (upper panel), Q (upper panel)), n=4 (FIGS. 2C, Q (lower panel), R (upper panel) or n=3 (FIGS. 2D, E, F (upper panel), H, I, J, K, P (right panel), S). In FIG. 2E lower panel: results pooled from 5 (L1210, P815, WEHI-3), 3 (CB17-3A8) or 4 (SP2/0)). In FIG. 2N, lower panel: 3 (MEL, BI-141, BW5147.3) or 4 (EL-4, RMA-S, YAC-1, B16, CMT-93, L929). In FIG. 2O: pooled from a total of 8 (L1210), 6 (P815), 7 (WEHI-3) or 5 (CB17-3A8, SP2/0). In graphs other than histograms, each symbol represents one mouse. All data are means+/−s.e.m.

FIGS. 3A-W. SLAM receptors are required for phagocytosis of hematopoietic target cells in vitro and in vivo. (FIG. 3A) Expression of LRP-1 in BMDMs generated from LRP-1-deficient mice (Lrp1^{fl/fl};Lys2-Cre) and mice expressing Lys2-Cre alone (as control (Ctl)) was verified by immunoblot. (FIG. 3B) phagocytosis of L1210 or P815 LRP-1-deficient mice (Lrp1^{fl/fl};Lys2-Cre) and mice expressing Lys2-Cre alone (as control (Ctl)), in the presence of control IgG or anti-CD47, was determined as detailed for FIG. 2E. Top panel with non-authenticated cells. Lower panel with authenticated cells. (FIGS. 3C-A to 3C-H). BMDMs from WT or SFR KO mice were analyzed by flow cytometry using antibodies directed against various cell surface markers, including SLAM receptors (dotted lines). Filled curves represent staining with isotype control antibody. (FIG. 3D) The experiment was the same as in FIG. 2E, except that BMDMs from wild-type (WT) or SFR KO mice were analyzed. Middle panel with non-authenticated cells. Lower panel with authenticated cells. (FIG. 3E) The experiment was the same as in FIG. 3D, except that peritoneal macrophages were studied. Top panel with non-authenticated cells. Lower panel with authenticated cells. (FIG. 3F) The experiment was the same as in FIG. 2K, except that BMDMs from WT or SFR KO mice were used. (FIG. 3G) Same as in FIG. 2E, except that human targets Raji and Daudi were used Given that the anti-human CD47 MAb is of mouse origin, F(ab′)₂ fragments of antibodies were utilized. (FIG. 3H) The experiment was the same as in FIG. 2E except that used F(ab′)₂ fragments of Ab instead of intact Ab and used SFR KO macrophages. (FIGS. 3I-A to 3I-D) Phagocytosis of L1210 cells by WT or SFR KO BMDMs was analyzed as detailed for FIGS. 2C-D, using a flow cytometry-based assay (FIGS. 3I-A and 31-B) or the pHrodo™-based assay (FIGS. 3I-C and 3I-D). Representative experiments are depicted in FIGS. 3I-A and 3I-C, whereas graphic representations of the results from multiple independent experiments are shown in FIGS. 3I-B and 3I-D. Bars represent the average percentage of BMDMs showing phagocytosis of targets from at least three independent experiments for each cell type. Error bars represent standard deviations. Top panel in in FIGS. 3I-B and 3I-D with non-authenticated cells. Lower panel with authenticated cells. (FIG. 3J) The ability of BMDMs from WT or SFR KO mice to phagocytose parental or CD47 KO L1210 cells was analyzed, as detailed for FIG. 2J. Top panel with non-authenticated cells. Lower panel with authenticated cells. (FIG. 3K) Expression of CD47 (dotted lines) on parental and CD47 KO L1210 cells. Filled curves: isotype controls. (FIG. 3L) The ability of WT (left) or SFR KO (right) BMDMs to phagocytose WT or SFR KO activated CD4⁺ T cells was tested, as explained for FIG. 2I. (FIGS. 3M-A and 3M-B) The ability of BMDMs from WT or SFR KO mice to phagocytose IgG-containing immune complexes (I.C.), GFP-expressing E. coli or IgG-opsonized sheep red blood cells (sRBCs) was examined by flow cytometry (dotted lines), as detailed in Example 1. BMDMs in the absence of phagocytosis are shown as filled curves. Phagocytosis of apoptotic thymocytes was also analyzed, using a microscopy-based assay as detailed in Example 1. Bars in FIG. 3M-B represent the average percentage of BMDMs showing phagocytosis for each cell type. Error bars represent standard deviations. Top panel of FIG. 3M-B with non-authenticated cells. Lower panel of FIG. 3M-B with authenticated cells. (FIG. 3N) The ability of BMDMs from WT or SFR KO mice to phagocytose RBCs from WT or CD47 KO mice (mRBCs) was analyzed by microscopy, as detailed for FIG. 2E. Top panel with non-authenticated cells. Lower panel with authenticated cells. (FIG. 3O) The ability of BMDMs from WT or SFR KO mice to phagocytose IgG-opsonized L1210 cells was analyzed, in the presence of anti-CD47 or control IgG, as detailed for FIG. 1E and specified in Example 1. Bars represent the average percentage of BMDMs showing phagocytosis of targets. Error bars represent standard deviations. Top panel with non-authenticated cells. Lower panel with authenticated cells. (FIG. 3P) WT or SFR KO mice (n=6 per group) were injected with thioglycolate (TG) intra-peritoneally (I.P.) on day (D) 0. On D4, they were injected I.P. with CFSE-labeled L1210 cells, in the presence of control IgG or anti-CD47. On 05, cells were recovered from the peritoneal cavity by lavage and the number of remaining L1210 cells was determined as detailed in Example 1. Each symbol represents a different mouse. Mean values are depicted with horizontal bars. Error bars represent standard deviations. (FIG. 3Q) This analysis is from the experiment depicted in FIG. 3P. After peritoneal lavage, cells were analyzed by flow cytometry, in the presence of a fixed number of fluorescent beads to allow quantitation of total cell numbers. Beads are boxed in R1, while L1210 cells (labeled with CFSE) are boxed in R2. (FIG. 3R) This analysis is from the experiment depicted in FIG. 3P. This experiment is the same as the one depicted in FIG. 3M. Numbers of peritoneal macrophages at the time of the peritoneal lavage were determined by flow cytometry. Bars represent the average numbers of peritoneal macrophages under each condition. Error bars represent standard deviations. Top panel with non-authenticated cells. Lower panel with authenticated cells. (FIG. 3S) Schematic representation of the experiment shown TG, thioglycolate (upper panel). The experiment was the same as in FIG. 3P, except that only WT mice (n=2) were analyzed. Moreover, mice were injected with liposomes containing clodronate or phosphate-buffered saline (PBS) at D-1 and D3. Bars represent the average numbers of remaining L1210 cells in each group. Top panel with non-authenticated cells. Lower panel with authenticated cells. (FIG. 3T) This analysis is from the experiment shown in FIG. 3S. It was performed as detailed for FIG. 3Q. (FIG. 3U) This analysis is from the experiment shown in FIG. 3O. Numbers of peritoneal macrophages at the moment of the peritoneal lavage were determined by flow cytometry. It was performed as detailed for FIG. 3R. Top panel with non-authenticated cells. Lower panel with authenticated cells. (FIG. 3V) Growth of L1210 injected sub-cutaneously in RAG-1 or RAG-1 SFR dKO mice. Bars represent the average numbers of peritoneal macrophages under each condition. Error bars represent standard deviations. (FIG. 3W) Tumors from experiment depicted in FIG. 3V were dissected, weighted, measured and analyzed by flow cytometry. Two RAG-1 KO mice treated with anti-CD47 (mice 9 and 10) showed no clinically detectable tumor when alive. However, upon dissection, small nodules with no detectable weight on the scale were present. These nodules were processed and analyzed as for the other tumors. L1210 were GFP⁺; macrophages were Ly6G⁻CD11b⁺NK1.1⁻; neutrophils were Ly6G⁻CD11b⁺NK1.1⁻; and NK cells were Ly6G⁻CD11b⁺NK1.1⁺, n.s., not significant; *: p<0.05; **: p<0.01; ***p<0.001 (two-tailed Student's t-tests). In FIG. 3A: Flow cytometry profiles are representative of 5 (L1210, P815, CB17-3A8, WEHI-3, SP2/0, activated CD4⁺ T cells, Raji, Daudi), 3 (MEL, BI-141, EL-4, RMA-S, YAC-1, BW5147.3, 816, CMT-93, L929, thymocytes, resting CD4⁺ T cells, resting B cells, activated B cells) and 2 (SW480, SW620, Colo205) experiments Representative of n=1-3 (lack of LRP-1-encoding gene confirmed by immunoblot for 1 experiment and by genotyping for 3 experiments (data not shown)) (FIG. 3A), n=3 (FIGS. 3B, C, E (upper panel), F, H, J (upper and lower panels), K, L, M (upper panel), 0 (upper and lower panels)), n=6 (FIGS. 3D (middle panel), P (upper panel), Q (upper and lower panels), R (upper panel)), n=5 (FIG. 3G) n=3-4 (FIG. 3I (upper and lower panels) or n=2 (FIG. 3N (upper panel), S (upper and lower panels), T, U (upper and lower panels)) mice in independent experiments. In FIG. 3D (lower panel), results pooled from a total of 8 (L1210), 6 (P815), 7 (WEHI-3) or 5 (CB17-3A8, SP2/0). In FIG. 3E (lower panels), 3 (left) and 2 (right). In FIG. 3I, results pooled from a total of 4 (top) and 3 (bottom). In FIG. 3P (lower panel), 6 mice from 5 independent experiments. In FIG. 3R (lower panel), 6 mice analyzed in 5 independent experiments. For FIG. 3V, 11 mice from 2 of 4 independent experiments bar graphs represent mean volumes. For FIG. 3W, 11 mice in 2 of 4 independent experiments. In point form graphs, each symbol represents one mouse. All data are means+/−s.e.m.

FIGS. 4A-4H. Impact of SLAMF7 on phagocytosis. Within the SLAM family, SLAMF7 is necessary and sufficient for phagocytosis of hematopoietic target cells. (FIG. 4A) The experiment was the same as in FIG. 2E, except that BMDMs were from mice lacking individual SLAM family members, using L1210 as targets. Top panel with non-authenticated cells. Lower panel with authenticated cells. (FIGS. 4B-A to 4B-C) BMDMs from WT, SLAMF7 KO or SLAMF7 KO mice reconstituted with Slamf7 BAC transgene were analyzed by flow cytometry using antibodies directed against various cell surface markers, including SLAM receptors (dotted lines). Filled curves represent staining with isotype control antibody. (FIGS. 4C-A and 4C-B) The ability of BMDMs from WT of SLAMF7 KO mice to phagocytose the indicated targets, in the presence of anti-CD47 or control IgG, was tested as detailed in FIG. 2E. When human targets were used (Daudi, Raji), F(ab′)₂ fragments of anti-human CD47 or control IgG were used. In FIG. 4B-B, top panel with non-authenticated cells, and lower panel with authenticated cells. (FIGS. 4D-A and 4D-B) The ability of BMDMs from WT, or SFR KO mice to phagocytose IgG-containing immune complexes, GFP-expressing E. coli or IgG-opsonized L1210 cells (for the latter, in the presence of anti-CD47. or control IgG) was analyzed as detailed for FIGS. 3N, P. (FIG. 4E) The experiment was the same as in FIG. 2E, except that BMDMs (MΦs) from wild-type (WT) mice, SFR KO mice or SFR KO mice reconstituted with a BAC transgene solely containing the mouse Slamf7 gene were used. Top panel with non-authenticated cells. Lower panel with authenticated cells, (FIG. 4F) The experiment was the same as in FIG. 3Q, using BMDMs from SFR KO mice reconstituted with Slamf7 BAC transgene and L1210. (FIG. 4G) To generate BAC transgenic mice expressing SLAMF7, the C57BL/6 BAC clone RP23-145F9 was first truncated at the 3′ end to eliminate the Slamf1 gene. Then, a stop codon (denoted by X) was introduced in exon 2 of Slamf2, the gene coding for CD48, and a silent mutation (HindIII site; denoted by red vertical bar) was created in Slamf7 to allow screening of BAC transgenic mice. The transcriptional orientation of the Slam genes is depicted by arrows, while the relative positions of the genes in the clone are indicated by their distances from the 5′ end (in kilobases (kb)). (FIGS. 4H-A to 4H-E) BMDMs from WT, SFR KO or SFR KO-SLAMF7 BAC mice were analyzed by flow cytometry using antibodies directed against various cell surface markers, including SLAM receptors (dotted lines). Filled curves represent staining with isotype control antibody. n.s., not significant; *: p<0.05; **: p<0.01; ***p<0.001 (two-tailed Student's t-tests). Representative of n=3 (FIGS. 4B-A, 4B-B, C-A, D-A, D-B, E (upper and lower panels), F, G); n=3-5 (FIG. 4A (upper panel)). In FIG. 4A (lower panel), results pooled from a total of 8 (SLAMF7 KO) or 3 (all other KO mice). In FIG. 4C (lower panel), results pooled from a total of 4 mice studied in independent experiments. In point form graphs, each symbol represents one mouse. All data are means+/−s.e.m.

FIGS. 5A-L Impact of SLAMF7 on phagocytosis and enforced ectopic expression of SLAMF7 on MEL cells. (FIGS. 5A-A to 5A-C) Expression of SLAMF7 on the indicated mouse or human targets was determined by flow cytometry, using antibodies against mouse (m) or human (h) SLAMF7 (dotted lines). Filled curves represent staining with isotype control antibody. (FIG. 5B-A to 5B-F) Expression of various cell surface markers, including SFRs and their ligands (dotted lines); m: mouse; h: human. Filled curves: isotype controls. (FIGS. 5CA to 5C-P) Expression of various cell surface markers, including SFRs and their ligands (dotted lines); m: mouse; h: human. Filled curves: isotype controls. (FIG. 5D) MEL cells were transduced with retroviruses expressing GFP alone or in combination with mouse SLAMF7. Expression was SLAMF7 was analyzed by flow cytometry (dotted lines). Filled curves represent staining with isotype control antibody. Bars represent the average numbers of peritoneal macrophages under each condition. (FIG. 5E) WT, SFR KO or SLAMF7 KO BMDMs were tested for phagocytosis of MEL cells, ectopically expressing or not mouse SLAMF7, as detailed for FIG. 1E. (FIG. 5F) WT mice (n=5 per group) were tested in a peritoneal clearance assay, as detailed for FIG. 3Q, except that MEL cells expressing or not mouse SLAMF7 were used. Each symbol represents a different mouse. Mean values are depicted with horizontal bars. Error bars represent standard deviations. (FIG. 5G) This experiment is the same as the one shown in FIG. 3E. Numbers of peritoneal macrophages at the moment of the peritoneal lavage were determined by flow cytometry. Bars represent the average numbers of peritoneal macrophages under each condition. (FIGS. 5H-A and 5H-B) Phagocytosis of activated WT or SLAMF7 KO CD4⁺ T cells by WT MΦs. (FIG. 5I) Residual WT and SLAMF7 KO CD4⁺ T cells in blood of WT mice. Left: representative dot plot. (FIGS. 5JA to 5J-C) SFR KO BMDMs were transduced with retroviruses encoding green fluorescent protein (GFP) alone, or in combination with human (hSLAMF7) or mouse SLAMF7 (mSLAMF7). After sorting GFP-positive cells, phagocytosis of L1210 was assessed as detailed for FIG. 2E (right). Representative expression of SLAMF7 on sorted populations is depicted on the left (dotted lines). Filled curves represent staining with isotype control antibody. Top panel with non-authenticated cells. Lower panel with authenticated cells. (FIG. 5K) BMDMs from WT C57BL/6 mice or NRG mice were tested for phagocytosis of L1210 cells as detailed for FIG. 1E, except that rat anti-mSLAMF7 MAb 4G2 (or isotype control rat IgG) was added during the assay. Top panel with non-authenticated cells. Lower panel with authenticated cells. (FIG. 5L) Blood-derived human monocytes from healthy donors (n=3) were tested for phagocytosis of Raji cells in the presence of F(ab′)₂ fragments of anti-hCD47 or isotype control IgG, and in the additional presence of F(ab′)₂ fragments of mouse anti-hSLAMF7 162 or isotype control mouse IgG. Phagocytosis was assessed as detailed for FIG. 2E. Each symbol represents a different sample of blood-derived human monocytes. Mean values are depicted with horizontal bars. Error bars represent standard deviations. Top panel with non-authenticated cells. Lower panel with authenticated cells. n.s., not significant; *: p<0.05; **: p<0.01; ***p<0.001 (two-tailed Student's t-tests). Representative of n=3 (FIGS. 5D, E, H, J (upper panel), L (upper and lower panels)), n=4 (c), n=5 (FIGS. 5F, G), or n=2-3 (FIG. 5K (upper and lower panels)). In FIG. 5A, results pooled from a total of 8 (SLAMF7 KO) or 3 (all other KO mice). In FIG. 5B, flow cytometry profiles are representative of 3 (MEL, BI-141, EL-4, RMA-S, YAC-1, BW5147.3, B16, CMT-93, L929, thymocytes, resting CD4⁺ T cells, resting B cells, activated B cells) or 2 (SW480, SW620, Colo205). In FIG. 5C, flow cytometry profiles are representative of 4 independent experiments. In FIG. 5I, 6 mice in 3 experiments. In FIG. 5L (lower panel), results pooled from a total of 3 (C57BL16) or 2 (NRG) independent mice. In point form graphs F to L, each symbol represents one mouse or healthy donor. All data are means+/−s.e.m.

FIGS. 6A-H. SLAMF7− phagocytosis controls actin polarization and promotes independently of SAP adaptors and involves ITAM-dependent signaling pathways. (FIGS. 6A-A to 6A-C) Conjugate formation between BMDMs (WT or SFR KO; labeled with anti-F4/80 antibodies) and CFSE-labeled L1210 was studied for the indicated times at 37° C., in the presence of anti-CD47. Conjugates (boxed) were detected by flow cytometry (representative experiment left). The percentages of conjugate formation are indicated above the boxes. A statistical analysis of data from 3 independent experiments is shown in FIG. 6A-C. Bars represent the average number of conjugates. Error bars represent standard deviations. Top panel with non-authenticated cells. Lower panel with authenticated cells. (FIG. 6B) Conjugate formation (left) and phagocytosis (right) of L1210 by MΦs. (FIG. 6C) Actin polarization in MΦs incubated with L1210 detected by immunofluorescence. Cell Trace Violet-labeled BMDMs from WT or SFR KO mice were incubated with CFSE-labeled L1210 at 37° C., in the presence of anti-CD47. After 30 minutes, cells were fixed and stained with anti-actin mouse MAb AC-74 and Alexa Fluor 594-coupled goat anti-mouse IgG (top two panels), or Alexa Fluor 594-coupled goat anti-mouse IgG alone (bottom panel). Polarization of actin in conjugates between BMDMs and L1210 was studied by confocal microscopy, as detailed in Example 1. Examples of fully polarized and non-polarized conjugates are shown on the left top two panels. Arrows show polarization of actin. A quantitation of the data for 130 conjugates from 3 independent experiments is depicted on the right. Bars represent the average number of conjugates with fully polarized actin. Error bars represent standard deviations. Scale bar, 5 μm. Top panel with non-authenticated cells. Lower panel with authenticated cells. (FIGS. 6D-A to 6D-C) BMDMs from WT or SFR KO mice were transduced with retroviruses encoding GFP alone, or in combination with WT mSLAMF7 or a mSLAMF7 mutant in which the three intra-cytoplasmic tyrosines are mutated to phenylalanines (Y→F). After sorting GFP-positive cells, phagocytosis of L1210 was assessed as detailed for FIG. 2E (right). Expression of SLAMF7 on sorted populations determined by flow cytometry is depicted in FIGS. 6A-6B (dotted lines). Filled curves represent staining with isotype control antibody. In FIG. 6C, top panel with non-authenticated cells and lower panel with authenticated cells. (FIG. 6E) Phagocytosis of L1210 was assessed as detailed for FIG. 2E, using BMDMs from WT or EAT-2 KO mice. Top panel with non-authenticated cells. Lower panel with authenticated cells. (FIG. 6F) Phagocytosis of L1210 was assessed as detailed for FIG. 2E using WT or SFR KO BMDMs, except that assays were performed in the presence of pharmacological inhibitors of Src kinases (SU6656; 100 nM), Syk kinase (R406; 750 nM) or Btk family kinases (ibrutinib; 10 nM), or vehicle alone. These inhibitors had no deleterious impact on cell viability, as verified by staining cells with propidium iodide and annexin V (data not shown). Top panel with non-authenticated cells. Lower panel with authenticated cells. (FIGS. 6G-H) Phagocytosis of L1210 was assessed as detailed for FIG. 2E using BMDMs from WT, Syk KO or XID mice (right for 6F). Expression of Syk was verified by immunoblotting. Representative anti-Syk immunoblot is shown (left for 6F) Top panel with non-authenticated cells. Lower panel with authenticated cells. n.s., not significant; *: p<0.05; **: p<0.01; ***p<0.001 (two-tailed Student's (tests). Representative of n=4 (FIGS. 6A (upper panel), D (upper panel), G (upper and lower panels), H (upper and lower panels)), n=5 (FIG. 6D (lower panel) or n=3 (FIGS. C (upper and lower panels), E (upper and lower panels), F (upper and lower panels)). In FIG. 6A (lower panel), results pooled from a total of 3 mice studied in independent experiments. In FIG. 6B, results pooled from a total of 4 (left), 3 (right). In 6C, bars represent mean numbers of conjugates with fully polarized actin. In point form graphs, each symbol represents one mouse. All data are means+/−s.e.m.

FIGS. 7A-D. Impact of Syk and Btk kinases on phagocytosis and the function of SLAMF7 in phagocytosis requires the integrin Mac-1 and promotes actin polarization. (FIGS. 7A-A and 7A-B) BMDMs from WT or Syk KO mice were analyzed by flow cytometry using antibodies directed against various cell surface marker's, including SLAM receptors (dotted lines). Filled curves represent staining with isotype control antibody. (FIGS. 7B-A and 7B-B) The ability of BMDMs from WT or Syk KO mice to phagocytose GFP-expressing E. coli, IgG-opsonized L1210 cells (in the presence of anti-CD47 or control IgG) was analyzed as detailed for FIG. 3P or apoptotic thymocytes was analyzed as detailed for FIG. 4D. In FIG. 7B-B, top panel with non-authenticated cells and lower panel with authenticated cells. (FIGS. 7C-A to 7C-D) BMDMs from WT or XID mice were analyzed by flow cytometry using antibodies directed against various cell surface markers, including SLAM receptors (dotted lines). Filled curves represent staining with isotype control antibody. (FIGS. 7D-A and 7D-B) The ability of BMDMs from WT or XID mice to phagocytose GFP-expressing E. coli, IgG-opsonized L1210 cells (in the presence of anti-CD47 or control IgG) or apoptotic thymocytes was analyzed as detailed for FIG. 4D. FIG. 7D-A (left) with non-authenticated cells. FIG. 7D-B with authenticated cells. n.s., not significant; *: p<0.05; **: p<0.01; ***p<0.001 (two-tailed Student's t-tests). Representative of n=2 (FIGS. 7A, B (upper and lower panels), C, D (upper and lower panels)), Each symbol represents one mouse. All data are means+/−s.e.m.

FIGS. 8A-H. Impact of FcR gamma and DAP12 on phagocytosis. (FIG. 8A) Phagocytosis of L1210 was assessed as detailed for FIG. 2E using BMDMs from WT, and DAP12 KO mice. Expression DAP12 was verified by immunoblotting. (representative anti-DAP12 immunoblots are shown left). Top panel with non-authenticated cells. Lower panel with authenticated cells. (FIGS. 8B-A to 8B-D) BMDMs from WT or DAP12 KO mice were further analyzed by flow cytometry using antibodies directed against various cell surface markers, including SLAM receptors (dotted lines) (left), Filled curves represent staining with isotype control antibody. The ability of BMDMs from WT or DAP12 KO mice to phagocytose IgG-opsonized L1210 cells (in the presence of anti-CD47 or control IgG) was analyzed as detailed for FIG. 4D (right). FIG. 8B-D top panel with non-authenticated cells and lower panel with authenticated cells. (FIG. 8C) Phagocytosis of L1210 was assessed as detailed for FIG. 2E using BMDMs from WT and FcR gamma KO mice. Expression of FcR gamma was verified by immunoblotting. (representative anti-FcRgamma immunoblots are shown left). Top panel with non-authenticated cells, Lower panel with authenticated cells. (FIGS. 8D-A to 8D-F) BMDMs from WT or FcR gamma KO mice were analyzed by flow cytometry using antibodies directed against various cell surface markers, including SLAM receptors (dotted lines) (top). Filled curves represent staining with isotype control antibody. The ability of BMDMs from WT or FcR gamma mice to phagocytose IgG-opsonized L1210 cells (in the presence of anti-CD47 or control IgG was analyzed as detailed for FIG. 4D (bottom). In FIG. 8D-F, top panel with non-authenticated cells and lower panel with authenticated cells. (FIG. 8E) Phagocytosis of L1210 was assessed as detailed for FIG. 2E using BMDMs from WT and FcR gamma-DAP12 double KO mice. Top panel with non-authenticated cells. Lower panel with authenticated cells. (FIGS. 8F-A and 8F-B) The ability of BMDMs from WT or DAP12 KO mice to phagocytose GFP-expressing E. coli or apoptotic thymocytes was analyzed as detailed for FIG. 4D). (FIGS. 8G-A and 8G-B) The ability of BMDMs from WT or FcR gamma mice to phagocytose GFP-expressing E. coli or apoptotic thymocytes was analyzed as detailed for FIG. 4D). (FIGS. 8H-A to 8H-E) BMDMs from WT or dKO mice were analyzed by flow cytometry using antibodies directed against various cell surface markers, including SLAM receptors (dotted lines) (left). Filled curves represent staining with isotype control antibody. The ability of BMDMs from WT or dKO mice to phagocytose GFP-expressing E. co/i, IgG-opsonized L1210 cells (in the presence of anti-CD47 or control IgG) or apoptotic thymocytes was analyzed as detailed for FIG. 4D. in FIG. 8H-D non-authenticated cells and in FIG. 8H-E, authenticated cells. n.s., not significant; *: p<0.05; **p<0.01; ***p<0.001 ((two-tailed Student's t-tests). Representative of n=2 (FIGS. B (upper, middle and lower panels), D (upper and lower panels), F, G, H (upper and lower panels)). n=3 (FIGS. 8A (upper and lower panels), C (upper panel), E (upper and lower panels)), n=5 (FIG. 8C (lower panel) Each symbol represents one mouse. All data are means+/−s.e.m.

FIGS. 9A-G. SLAMF7-dependent phagocytosis requires ITAMs and Mac-1. (FIG. 9A) SLAMF7 was recovered by immunoprecipitation from Brij99-containing lysates of WT or SFR KO BMDMs. After several washes, proteins were eluted, digested with trypsin and identified by mass spectrometry. The GenInfo™ Identifier (gi) accession number and means of the normalized total ion counts (TICs) for each identified interactor polypeptide is shown. Duplicates were used for each genotype, and averages are shown for TIC values. Only receptors known to regulate macrophage activation are listed. Top panels with non-authenticated cells. Lower panel with authenticated cells. (FIG. 9B) The experiment was the same as FIG. 9A, except that CD11b was immunoprecipitated. Authenticated cells. (FIG. 9C) The experiment was the same as FIG. 9A, except the data for FcRs CD64 and CD16 are shown. Authenticated cells. (FIG. 9D) Lysates from the mouse macrophage cell line RAW264.7 expressing GFP alone or in combination with a FLAG-tagged version of mouse SLAMF7 (FLAG-SLAMF7) were immunoprecipitated with anti-FLAG. They were then probed by immunoblotting with anti-CD11b (Mac-1) or anti-SLAMF7. Total cell lysates were analyzed in parallel. (FIG. 9E) Co-localization of SLAMF7 and CD11b in RAW264.7 cells expressing GFP alone or with FLAG-SLAMF7 assessed by immunofluorescence. Semi-confluent RAW264.7 cells expressing GFP alone (bottom) or in combination with FLAG-SLAMF7 (top) were fixed and stained with antibodies against FLAG, CD11b (Mac-1) or CD18, as detailed in Example 1. Staining was detected by confocal microscopy. Scale bar, 5 μm. Top panels with non-authenticated cells. Lower panel with authenticated cells. (FIG. 9F) As in FIG. 9E, except that only RAW264.7 cells expressing FLAG-SLAMF7 were analyzed and that antibodies coupled to different fluorophores were used. Top panels with non-authenticated cells. Lower panel with authenticated cells. (FIGS. 9G-A and 9G-B) RAW264.7 derivatives expressing GFP alone or in combination with FLAG-SLAMF7 were analyzed by flow cytometry, using antibodies directed against FLAG and SLAMF7 (dotted lines). Filled curves represent staining with isotype control antibody. n.s., not significant; *: p<0.05; **: p<0.01; ***p<0.001. Representative of n=2 (FIG. 9A (upper panel)), n=3 (FIG. 9F (upper panel)), n=4 (FIGS. 9D, E (upper panel), G), n=6 (FIGS. 9B-C). In FIG. 9A (lower panel) results pooled from 2 experiments with a total of 5. In FIGS. 9F-G (lower panels), photographs are representative of 3 independent experiments. Each symbol represents one mouse. All data are means+/−s.e.m.

FIGS. 10A-F. SLAMF7-dependent phagocytosis requires ITAMs and Mac-1. (FIG. 10A) Phagocytosis of L1210 cells by WT macrophages was analyzed as detailed for FIG. 2E, in the presence of antibodies against integrins or control (Ctl) IgG. Top panels with non-authenticated cells. Lower panel with authenticated cells. (FIG. 10B) The experiment was as FIG. 2E, using WT or CD11 b KO BMDMs and L1210 cells as targets. Top panels with non-authenticated cells. Lower panel with authenticated cells. (FIGS. 10C-A to 10C-E) BMDMs from WT, CD11b KO or CD11a KO mice were analyzed by flow cytometry using antibodies directed against various cell surface markers, including SLAM receptors (dotted lines). Filled curves represent staining with isotype control antibody. (FIG. 10D) The ability of WT or CD11a KO BMDMs to phagocytose L1210 cells, in the presence of anti-CD47 or control IgG, was analyzed as detailed for FIG. 2E. Top panel with non-authenticated cells. Lower panel with authenticated cells. (FIG. 10E) The ability of BMDMs from WT or CD11 b KO mice to phagocytose L1210 cells opsonized with C3b, (left) or IgG (right), in the presence of anti-CD47 or control IgG, was analyzed as detailed for FIG. 4D. Top panels with non-authenticated cells. Lower panel with authenticated cells. (FIG. 10F) The ability of BMDMs from WT or SFR KO mice to phagocytose L1210 cells opsonized or not with C3bᵢ, in the presence of anti-CD47 or control IgG, was analyzed as detailed for FIG. 4D. Top panels with non-authenticated cells. Lower panel with authenticated cells. n.s., not significant; *: p<0.05; **p<0.01. ***p<0.001 (two-tailed Student's t-tests) Representative of n=3 (FIGS. 10A (lower panel), C, D (upper and lower panels), E (upper and lower panels), F (upper and lower panels)) or n=5 (FIGS. 10A (upper panel), B (upper and lower panels)). Each symbol represents one mouse. All data are means+/−s.e.m.

FIGS. 11A-D Gene expression analyses of SLAMF7 and CD47. Expression of SLAMF7 and CD47 RNA in human hematologic tumors. (FIG. 11A) RNA levels of SLAMF7 (top) and CD47 (bottom) in several types and sub-types of leukemia were analyzed, using data obtained from microarray experiments. Data for only one oligonucleotide probe are shown. However, similar findings were made with other SLAMF7 and CD47 probes (data not shown). Each symbol represents a different patient sample. Median expression for a given type or sub-type of malignancy is depicted by a horizontal line. For statistical analysis, Student's t-tests were performed comparing SLAMF7 expression in the combination of all AML and ALL, versus either MDS or CLL. AML, acute myelogenous leukemia; ALL, acute lymphoblastic leukemia; MDS, myelodysplastic syndrome; CML, chronic myelogenous leukemia; CLL, chronic lymphocytic leukemia. (FIG. 11B) Same as FIG. 11A, except that samples of multiple myeloma (MM) were analyzed. (FIG. 11C) Same as FIG. 11A, except that samples of AML and diffuse large B cell lymphoma (DLBCL) were studied. Moreover, RNA expression was quantitated by RNA sequencing. (FIG. 11D) Levels of SLAMF7 and CD47 RNAs for individual samples from selected tumor types, which displayed higher levels of SLAMF7 RNA, were analyzed in parallel using dot plots. ***p<0.001. n values, from left to right are: FIG. 11A MILE Study: 38, 41, 37, 28, 48, 352, 70, 237, 122, 13, 40, 36, 58, 174, 206, 76, 448; AML TOGA: 4, 20, 16, 91, 27, 6, 14, 1, 14, 3, 17, 3, 5, 7, 7, 6, 1, 2; FIG. 11B MM: 304; FIG. 11C TCGA AML: 173, TCGA DLBCL: 48; FIG. 11D 13, 206, 448, 20, 14, 17, 304, 173, 48.

FIGS. 12A-R. SLAM family receptors (SFRs), CD47, CD45 mRNA expression (microarray) in human hematopoietic and non-hematopoietic cell lines. (FIG. 12A) RNA levels of SLAMF7 in several types of human hematopoietic tumor cell lines were analyzed (AML, B cell ALL, T cell ALL, Leukemia other, CML, Lymphoma Burkitt, Lymphoma DLBCL, Lymphoma Hodgkin, Lymphoma other, multiple myeloma), using data obtained from a microarray experiment. (FIG. 12B) Same as FIG. 12A, except that RNA levels of CD47 were analyzed in the cell lines. (FIG. 12C) Same as FIG. 12A, except that RNA levels of PTPRC (CD45) were analyzed in the cell lines, (FIG. 12D) Same as FIG. 12A, except that RNA levels of SLAMF2 (CD48) were analyzed in the cell lines. (FIG. 12E) Same as FIG. 12A, except that RNA levels of SLAMF5 (CD84) were analyzed in the cell lines. (FIG. 12F) Same as FIG. 12A, except that RNA levels of SLAMF1 were analyzed in the cell lines. (FIG. 12G) Same as FIG. 12A, except that RNA levels of SLAMF4 (2B4) were analyzed in the cell lines. (FIG. 1211) Same as FIG. 12A, except that RNA levels of SLAMF3 (Ly-9) were analyzed in the cell lines. (FIG. 121) Same as FIG. 12A, except that RNA levels of SLAMF6 were analyzed in the cell lines. (FIG. 12J) RNA levels of SLAMF7 in several types of human non-hematopoietic tumor cell lines were analyzed (bile duct, breast, chondrosarcoma, colorectal, endometrium, esophagus, Ewings sarcoma, glioma, kidney, liver, lung non-small cell lung, small cell lung, medulloblastoma, mesothelioma, neuroblastoma, osteosarcoma, ovary, pancreas, prostate, soft tissue, stomach, thyroid, upper aerodigestive, urinary tract and other), using data obtained from a microarray experiment. (FIG. 12K) Same as FIG. 12A, except that RNA levels of CD47 were analyzed in the cell lines. (FIG. 12L) Same as FIG. 12A, except that RNA levels of PTPRC (CD45) were analyzed in the cell lines, (FIG. 12M) Same as FIG. 12A, except that RNA levels of SLAMF2 (CD48) were analyzed in the cell lines. (FIG. 12N) Same as FIG. 12A, except that RNA levels of SLAMF5 (CD84) were analyzed in the cell lines. (FIG. 120) Same as FIG. 12A, except that RNA levels of SLAMF1 were analyzed in the cell lines. (FIG. 12P) Same as FIG. 12A, except that RNA levels of SLAMF4 (2B4) were analyzed in the cell lines. (FIG. 12Q) Same as FIG. 12A, except that RNA levels of SLAMF3 (Ly-9) were analyzed in the cell lines. (FIG. 12R) Same as FIG. 12A, except that RNA levels of SLAMF6 were analyzed in the cell lines. Each symbol represents a different cell line. Median expression for a given malignancy is depicted by an horizontal line. AML, acute myelogenous leukemia; ALL, acute lymphoblastic leukemia; CML, chronic myelogenous leukemia; DLBCL, diffuse large B cell lymphoma. Number of tumor cell lines per tumor type is shown in parenthesis.

FIGS. 13A-T. Amino acid and nucleotide sequences of human SLAMF7 isoforms. FIGS. 13A-B present human SLAMF7 isoform a precursor amino acid sequence NP_067004.3 (SEQ ID NO: 1), and nucleotide sequence transcript variant 1 NM_021181.4 (SEQ ID NO: 2); FIGS. 13C-D present human SLAMF7 isoform b precursor amino acid sequence NP_001269517.1 (SEQ ID NO: 3), and nucleotide sequence transcript variant 2 NM_001282588.1 (SEQ ID NO: 4); FIGS. 13E-F present human SLAMF7 isoform c precursor amino acid sequence NP_001269518.1(SEQ ID NO: 5), and nucleotide sequence transcript variant 3 NM_001282589.1 (SEQ ID NO: 6); FIGS. 13G-H present human SLAMF7 isoform d precursor amino acid sequence NP_001269519 (SEQ ID NO: 7), and nucleotide sequence transcript variant 4 NM_001282590.1 (SEQ ID NO: 8); FIGS. 13I-J present human SLAMF7 isoform e precursor amino acid sequence NP_001269520.1 (SEQ ID NO: 9), and nucleotide sequence transcript variant 5 NM_001282591.1 (SEQ ID NO: 10); FIGS. 13K-L present human SLAMF7 isoform f precursor amino acid sequence NP_001269521.1 (SEQ ID NO: 11), and nucleotide sequence transcript variant 6 NM_001282592.1 (SEQ ID NO: 12); FIGS. 2M-N present human SLAMF7 isoform g precursor amino acid sequence NP_001269522.1 (SEQ ID NO: 13), and nucleotide sequence transcript variant 7 NM_001282593.1 (SEQ ID NO: 14); FIGS. 13O-P present human SLAMF7 isoform h precursor amino acid sequence NP_001269523.1 (SEQ ID NO: 15), and nucleotide sequence transcript variant 8 NM_001282594.1 (SEQ ID NO: 16); FIGS. 13Q-R present human SLAMF7 isoform i precursor amino acid sequence NP_001269524.1 (SEQ ID NO: 17), and nucleotide sequence transcript variant 9 NM_001282595.1 (SEQ ID NO: 18); FIGS. 13S-T present human SLAMF7 isoform J precursor amino acid sequence NP_001269525.1 (SEQ ID NO: 19), and nucleotide sequence transcript variant 10 NM_001282596.1 (SEQ ID NO: 20).

FIGS. 14A-C. Amino acid sequences of human CD47 (also called integrin associated protein) isoforms. FIG. 14A present human CD47 amino acid sequence CAA80977.1 (SEQ ID NO: 21); FIG. 14B present human CD47 isoform 1 amino acid sequence NP_001768.1 (SEQ ID NO: 22); and FIG. 14C present human CD47 isoform 2 amino acid sequence NP_942088.1 (SEQ ID NO: 23).

### DESCRIPTION OF ILLUSTRATIVE EMBODIMENTS

Genes and Proteins

SLAMF7

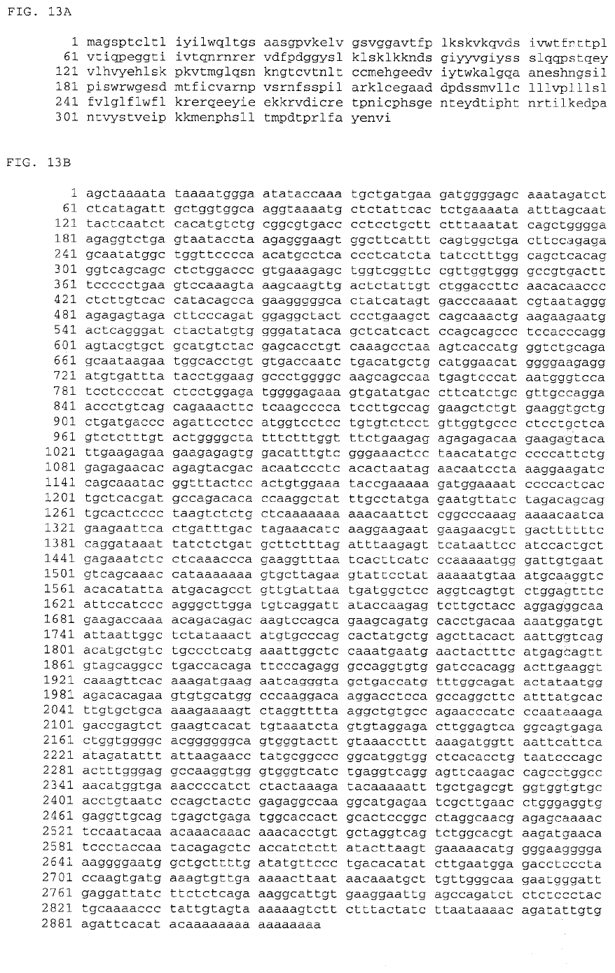

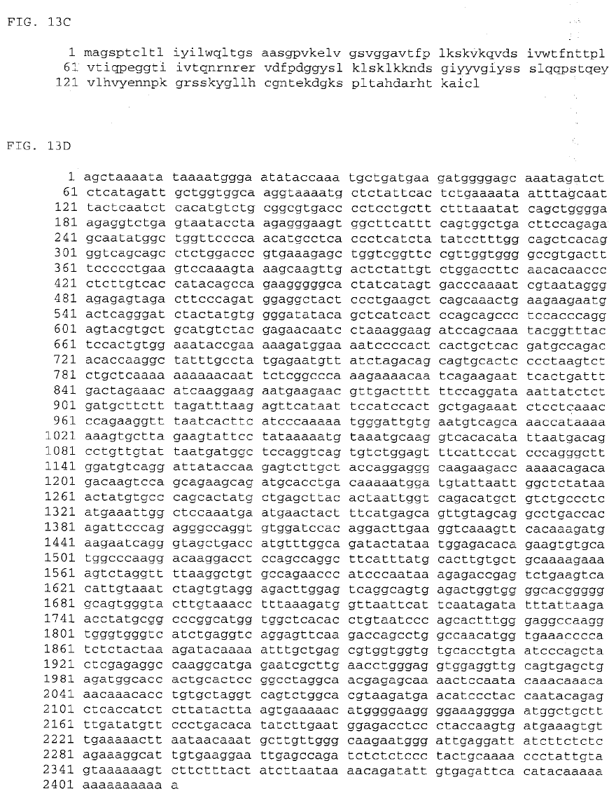

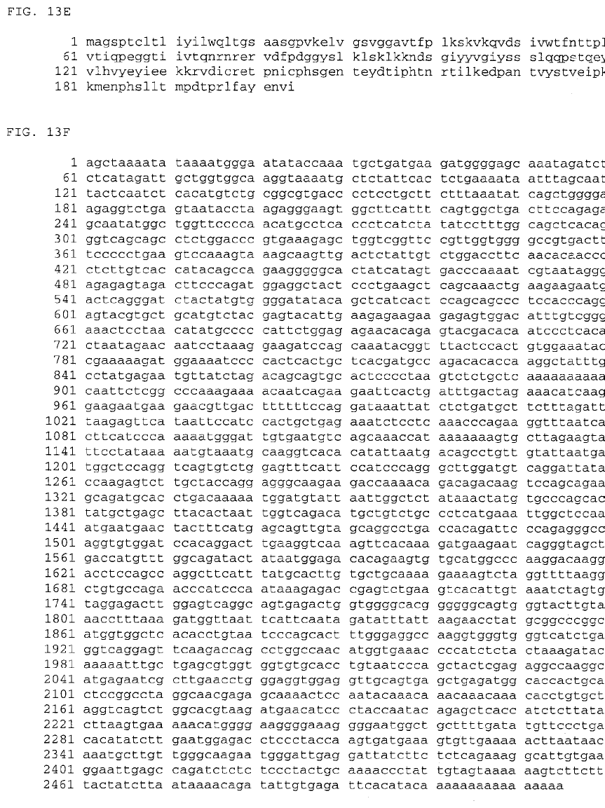

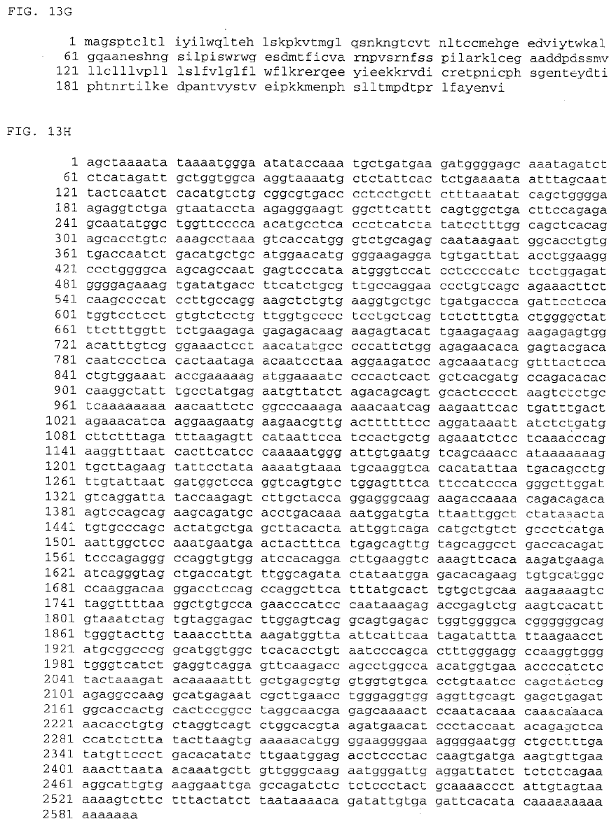

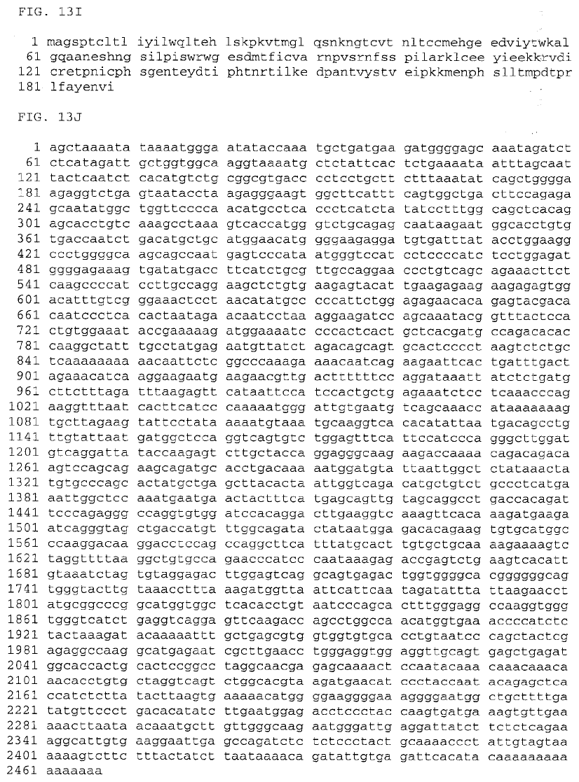

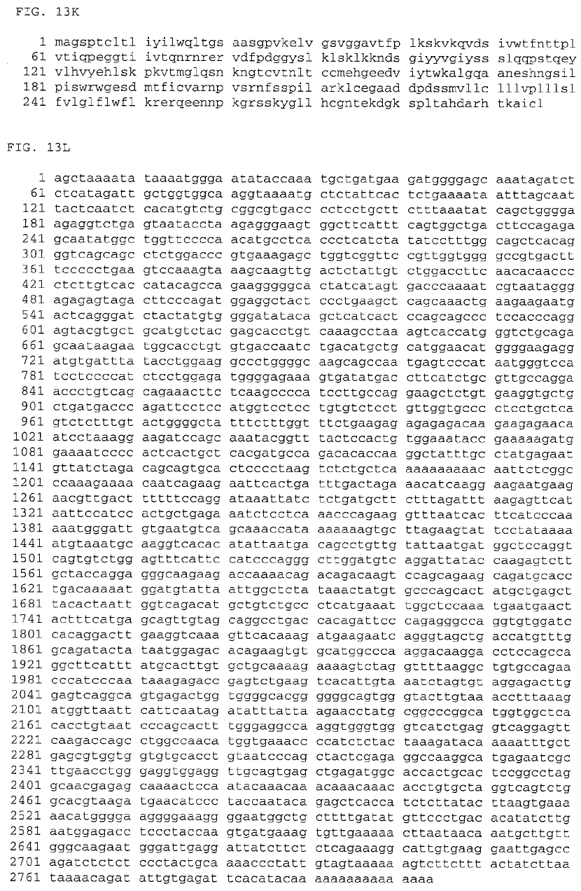

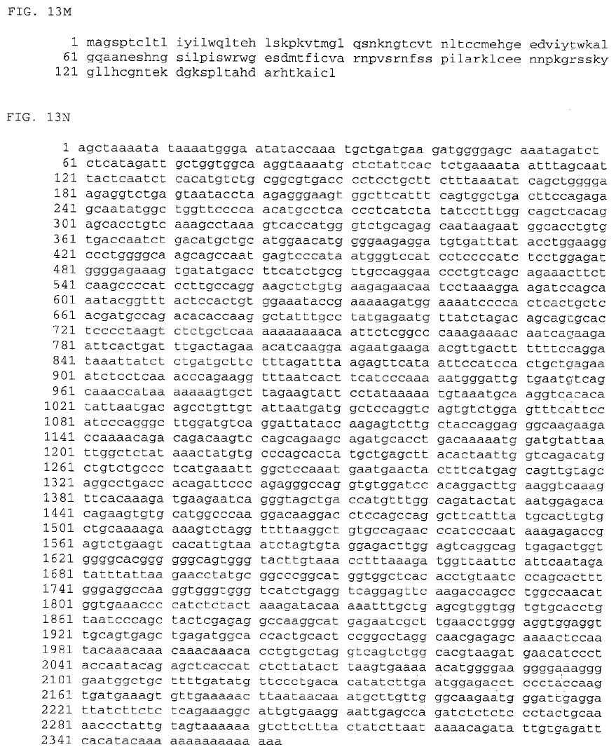

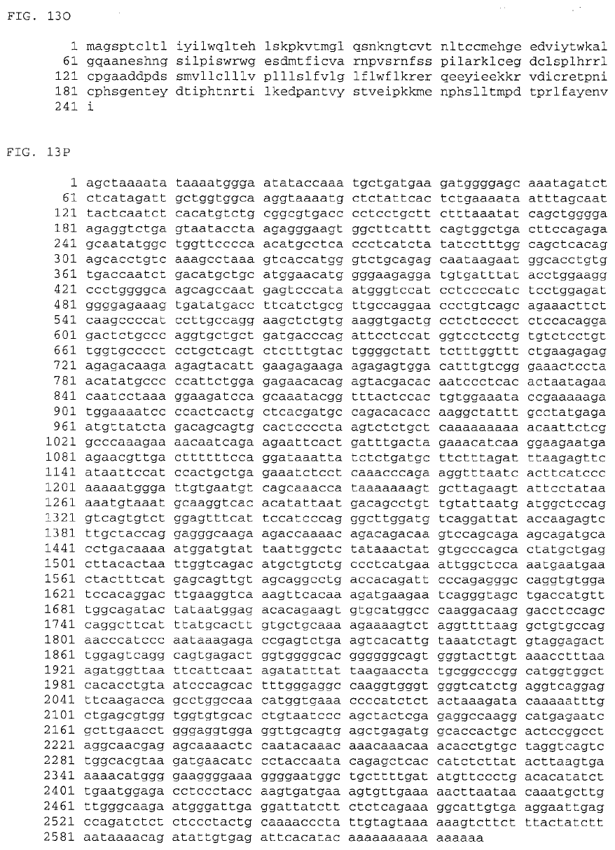

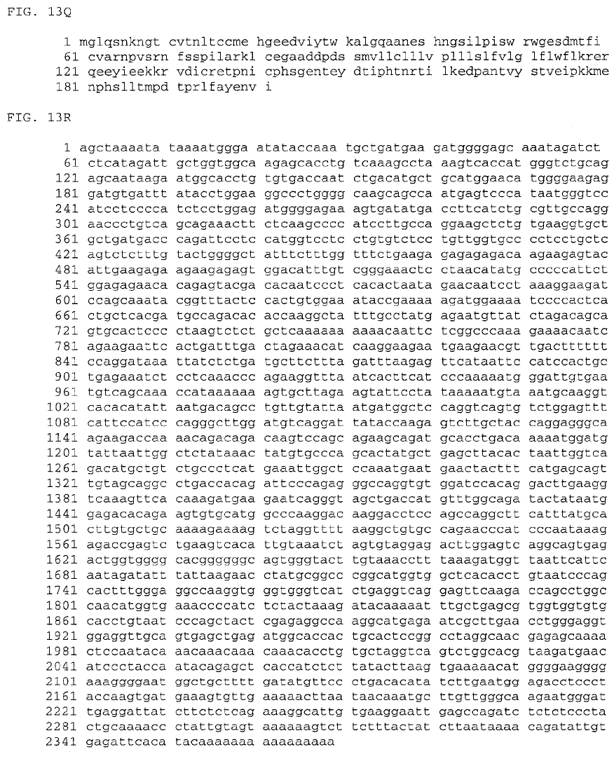

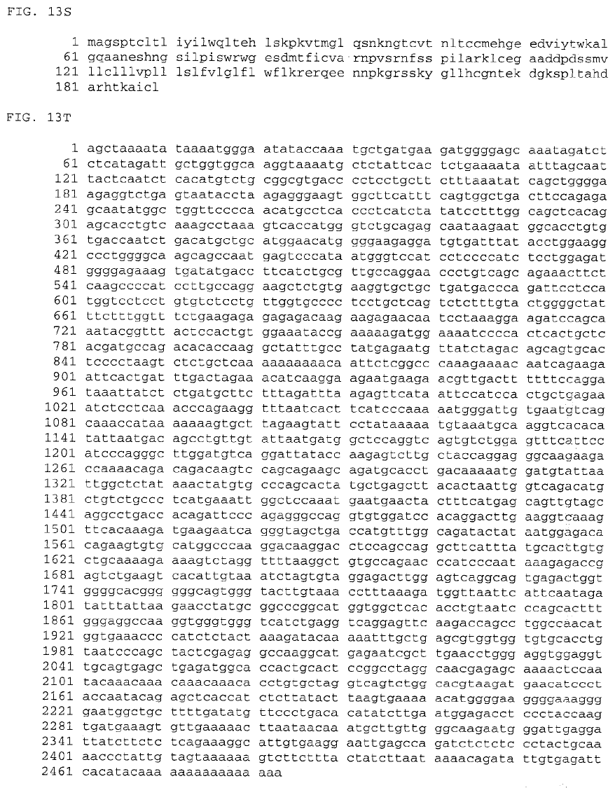

As used herein the terms “SLAMF7 gene” refers to nucleic acid (e.g., genomic DNA, cDNA, RNA) encoding the Signaling lymphocytic activation molecule family member 7 (SLAMF7). The description of the various aspects and embodiments of the disclosure is provided with reference to exemplary SLAMF7 nucleic acid sequences and amino acid sequence (e.g., as shown in FIGS. 13A-T). Such reference is meant to be exemplary only and the various aspects and embodiments of the disclosure are also directed to other SLAMF7 nucleic acids and polypeptides (also referred to SLAMF7 gene products), such as SLAMF7 nucleic acid or polypeptide mutants/variants, splice variants of SLAMF7 nucleic acids, SLAMF7 variants from species to species or subject to subject.

Consensuses derived from the alignments of certain SLAMF7 variants are also encompassed by the present disclosure. In specific embodiments of the consensus, each X in the consensus sequence is defined as being any amino acid, or absent when this position is absent in one or more of SLAMF7 Homo sapiens isoforms, variants or orthologues. In specific embodiment of the consensus, each X in the consensus sequences is defined as being any amino acid that constitutes a conserved or semi-conserved substitution of any of the amino acids in the corresponding position in the orthologues presented in the alignment, or absent when this position is absent in one or more of the orthologues presented in the alignment. Conservative substitutions are denoted by the symbol “:” and semi-conservative substitutions are denoted by the symbol “.”. In another embodiment, each X refers to any amino acid belonging to the same class as any of the amino acid residues in the corresponding position in the orthologues presented in the alignment, or absent when this position is absent in one or more of the orthologues presented in the alignment. In another embodiment, each X refers to any amino acid in the corresponding position of the orthologues presented in the alignment, or absent when this position is absent in one or more of the orthologues presented in the alignment. The Table below indicates which amino acid belongs to each amino acid class.

| Class | Name of the amino acids |
|---|---|
| Aliphatic | Glycine, Alanine, Valine, Leucine, |
| | Isoleucine |
| Hydroxyl or Sulfur/Selenium- | Serine, Cysteine, Selenocysteine, |
| containing | Threonine, Methionine |
| Cyclic | Proline |
| Aromatic | Phenylalanine, Tyrosine, |
| | Tryptophan |
| Basic | Histidine, Lysine, Arginine |
| Acidic and their Amide | Aspartate, Glutamate, Asparagine, |
| | Glutamine |

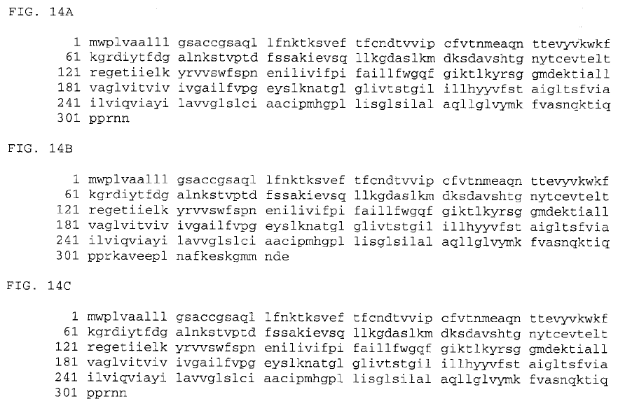

As used herein the terms “CD47 gene” refers to nucleic acid (e.g., genomic DNA, cDNA, RNA) encoding CD47. The description of the various aspects and embodiments of the disclosure is provided with reference to exemplary CD47 nucleic acid sequences and amino acid sequence (FIGS. 14A-D). Such reference is meant to be exemplary only and the various aspects and embodiments of the disclosure are also directed to other CD47 nucleic acids and polypeptides (also referred to CD47 gene products), such as CD47 nucleic acid or polypeptide mutants/variants, splice variants of CD47 nucleic acids, CD47 variants from species to species or subject to subject.

SIRPalpha

As used herein the terms “SIRPalpha gene” refers to nucleic acid (e.g., genomic DNA, cDNA, RNA) encoding SIRPalpha. The description of the various aspects and embodiments of the disclosure is provided with reference to exemplary SIRPalpha nucleic acid sequences and amino acid sequence. Such reference is meant to be exemplary only and the various aspects and embodiments of the disclosure are also directed to other SIRPalpha nucleic acids and polypeptides (also referred to SIRPalpha gene products), such as SIRPalpha nucleic acid or polypeptide mutants/variants, splice variants of SIRPalpha nucleic acids, SIRPalpha variants from species to species or subject to subject.

Protein Expression

As used herein the terms “SLAMF7 expression level” or “SLAMF7 expression”, or “CD47 expression level” or “CD47 expression”, refer to the measurement in a cell or a tissue of a SLAMF7 or CD47 gene product, respectively. SLAMF7 and CD47 expression levels could be evaluated at the polypeptide and/or nucleic acid levels (e.g., DNA or RNA) using any standard methods known in the art. The nucleic acid sequence of a nucleic acid molecule in a sample can be detected by any suitable method or technique of measuring or detecting gene sequence or expression. Such methods include, but are not limited to, polymerase chain reaction (PCR), reverse transcriptase-PCR (RT-PCR), in situ PCR, SAGE, quantitative PCR (q-PCR), in situ hybridization, Southern blot, Northern blot, sequence analysis, microarray analysis, detection of a reporter gene, or other DNA/RNA hybridization platforms, For RNA expression, preferred methods include, but are not limited to: extraction of cellular mRNA and Northern blotting using labeled probes that hybridize to transcripts encoding all or part of one or more of the genes of this disclosure; amplification of mRNA expressed from one or more of the genes of this disclosure using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this disclosure, arrayed on any of a variety of surfaces; in situ hybridization; and detection of a reporter gene.

In the context of this disclosure, “hybridization” means hydrogen bonding between complementary nucleoside or nucleotide bases. The terms “specifically hybridizable” and “complementary” are the terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed. Such conditions may comprise, for example, 400 mM NaCl, 40 mM PIPES pH 6.4, 1 mM EDTA, at 50 to 70° C. for 12 to 16 hours, followed by washing. The skilled person will be able to determine the set of conditions most appropriate for a test of complementarity of two sequences in accordance with the ultimate application of the hybridized nucleotides.

Methods to measure protein expression levels of selected genes of this disclosure, include, but are not limited to: Western blot, tissue microarray, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting (FACS), flow cytometry, and assays based on a property of the protein including but not limited to DNA binding, ligand binding, or interaction with other protein partners. In a further embodiment, the SLAMF7 and/or CD47 and/or SIRP expression level is measured by immunohistochemical staining, and the percentage and/or the intensity of immunostaining of immunoreactive cells in the sample is determined.

In an embodiment, the level of a SLAMF7 and/or CD47 and/or SIRP polypeptide is determined using an anti-SLAMF7 or an anti-CD47 antibody or an anti-SIRPalpha antibody. By “SLAMF7 antibody” and “anti-SLAMF7” or “CD47 antibody” and “anti-CD47” or “SIRPalpha antibody” and “anti-SIRPalpha”, in the present context is meant an antibody capable of detecting (i.e. binding to) a SLAMF7 protein or a SLAMF7 protein fragment or a CD47 protein or a CD47 protein fragment or a SIRPalpha protein or a SIRPalpha protein fragment, respectively.

Without being limited, SLAMF7 antibodies (which can be used for inhibiting the protein and/or for detection) include those listed in Table I below, CD47 antibodies include those listed in Table II below and SIRPalpha antibodies include those listed in Table III below.

TABLE I
Examples of commercially available SLAMF7 antibodies

| | Name/catalog | |
|---|---|---|
| Company | number | Type |
| Bristol-Myers Squibb | Elotuzumab | Humanized monoclonal |
| Company | | |
| Abcam | Ab95827 | Mouse monoclonal |
| | ab202840 | Rabbit polyclonal |
| Novus Biologicals | NBP2-12206 | Mouse monoclonal |
| Lifespan bioscience | LS-C125401-100 | Mouse monoclonal |
| Santa Cruz Biotechnology | sc-46517 | Goat polyclonal |
| | sc-46518 | Goat polyclonal |
| Cloud Clone Corp | PAK384Hu01 | Rabbit polyclonal |
| | MAK384Hu21 | Mouse monoclonal |
| | MAb 162 | |

TABLE II
Examples of CD47 antibodies (commercially
available or undergoing clinical trials)

| | Name/catalog | |
|---|---|---|
| Company | number | Type |
| Abcam | ab3283 | Mouse monoclonal |
| Santa Cruz Biotechnology | sc-12730 | Mouse monoclonal |
| Biolegend | CC2C6 | Mouse monoclonal |
| Ebioscience | Miap301 | Rat monoclonal against |
| | | mouse CD47 |
| | B6H12 | Mouse monoclonal against |
| | | human CD47 |
| | 2D3 | Mouse monoclonal against |
| | | human CD47 |
| Pharmingen | clone B6H12⁴⁴ | Anti-human |
| Stanford, Phase I clinical | Hu5F9-G4 | Anti-human monoclonal |
| study | | |
| Celgene/InhibRx | CC-90002 | Anti-human monoclonal |
| Novimmune SA | NI-1701 | Anti-human Anti-CD47 and |
| | | CD19 Bi-specific |
| | | monoclonal antibody |

Additional CD47 antibodies are listed in the following documents: WO2014093678 Therapeutic CD47 antibodies; US20140161805 Methods for manipulating phagocytosis mediated by CD47; US20140161825 Methods of treating acute myeloid leukemia by blocking CD47; US20120189625 Compositions and methods for treating hematological cancers targeting the SIRPalpha-CD47 interaction; US20120156724 Humanized anti-CD47 antibody; US20130142786 Humanized and chimeric monoclonal antibodies to CD47.

TABLE III
Examples of commercially available SIRPalpha antibodies

| | Name/catalog | |
|---|---|---|
| Company | number | Type |
| Abcam | ab8120 | Rabbit polyclonal |
| | ab53721 | Rabbit polyclonal |
| Ebioscience | P84 | Mouse monoclonal |
| | | against mouse SIRPa |
| | 15-414 | Mouse monoclonal |
| | | against human SIRPa |
| BD Bioscience | P84 | Mouse monoclonal |
| | | against mouse SIRPa |
| Santa Cruz | sc-376884 | mouse IgG₂ₐ |
| | sc-136067 | mouse IgG₁ |
| | sc-53115 | mouse lgG₂ₐ |
| | sc-17803 | mouse IgG₁ |
| | sc-53605 | mouse IgG₁ |
| | sc-376220 | mouse lgG_{2b} |
| | sc-373896 | mouse IgG₁ |
| | sc-55491 | mouse IgG |
| | sc-11374 | rabbit IgG |
| | sc-6921 | goat IgG |

Methods for normalizing the level of expression of a gene are well known in the art. For example, the expression level of a gene of the present disclosure can be normalized on the basis of the relative ratio of the mRNA level of this gene to the mRNA level of a housekeeping gene, or the relative ratio of the protein level of the protein encoded by this gene to the protein level of the housekeeping protein, so that variations in the sample extraction efficiency among cells or tissues are reduced in the evaluation of the gene expression level. A “housekeeping gene” is a gene the expression of which is substantially the same from sample to sample or from tissue to tissue, or one that is relatively refractory to change in response to external stimuli. A housekeeping gene can be any RNA molecule other than that encoded by the gene of interest that will allow normalization of sample RNA or any other marker that can be used to normalize for the amount of total RNA added to each reaction. For example, the GAPDH gene, the G6PD gene, the actin gene, ribosomal RNA, 36B4 RNA, PGK1, RPLP0, or the like, may be used as a housekeeping gene.

Methods for calibrating the level of expression of a gene are well known in the art. For example, the expression of a gene can be calibrated using reference samples, which are commercially available. Examples of reference samples include, but are not limited to: Stratagene™ QPCR Human Reference Total RNA, Clontech™ Universal Reference Total RNA, and XpressRef™ Universal Reference Total RNA.

In an embodiment, the above-mentioned method comprises determining the level of a SLAMF7 and/or CD47 and/or SIRP nucleic acid (e.g., nucleic acids as shown or encoding proteins as shown in in FIGS. 13A-T and FIGS. 14A-D) in the sample. In another embodiment, the above-mentioned method comprises determining the level of an SLAMF7 and/or CD47 polypeptide (e.g., polypeptides as shown in FIGS. 13 A-T and FIGS. 14A-D) and/or SIRPalpha polypeptide in the sample.

SLAMF7 Activity

As used herein the terms “SLAMF7 activity” and “SLAMF7 function” are used interchangeably and refer to detectable (direct or indirect) enzymatic, biochemical or cellular activity attributable to SLAMF7 (e.g., binding to SLAMF7 (e.g., on solid tumor cells), producing a macrophage ‘eat me signal’ (i.e. activating macrophage phagocytosis), stimulating cytoskeletal reorganization (e.g., promoting actin polarization towards target cells), binding to SAP adaptor EAT-2, co-localization of SLAMF7 with Mac-1 (CD11b and/or CD18). SLAMF7 activity could also be indirectly measured by evaluating the level of expression of SLAMF7, or a fragment thereof, in cells as well as in biological samples (e.g., tissue, organ, fluid).

CD47 Activity

As used herein the terms “CD47 activity” refer to detectable (direct or indirect) enzymatic, biochemical or cellular activity attributable to CD47 (e.g., interaction with/binding to SIRPalpha, modulating the functions of beta3 integrins, producing “don't eat me” signal (i.e. inhibiting macrophage phagocytosis)). CD47 activity could also be indirectly measured by evaluating the level of expression of CD47, or a fragment thereof, in cells as well as in biological samples (e.g., tissue, organ, fluid).

SIRPalpha Activity

As used herein the terms “SIRPalpha activity” refer to detectable (direct or indirect) enzymatic, biochemical or cellular activity attributable to SIRPalpha (e.g., interaction with/binding to CD47, producing “don't eat me signal” (i.e. inhibiting macrophage phagocytosis), binding to protein tyrosine phosphatase SHP-1). SIRPalpha activity could also be indirectly measured by evaluating the level of expression of SIRPalpha, or a fragment thereof, in cells as well as in biological samples (e.g., tissue, organ, fluid).

Modulation of SLAMF7 and/or CD47 and/or SIRPalpha Expression or Activity

The modulation of SLAMF7 and/or CD47 and/or SIRPalpha expression and/or activity could be achieved directly or indirectly by various mechanisms, which among others could act at the level of (i) transcription, for example by inhibiting any of these proteins promotor and thereby reducing their messenger RNA expression (e.g., by cytokine stimulation, etc.), (ii) translation, (iii) post-translational modifications, e.g., glycosylation, sulfation, phosphorylation, ubiquitination (e.g., polyubiquitinylation and proteasomal degradation), (iv) cellular localization (e.g., cytoplasmic versus nuclear localization), (v) protein-protein interaction. These regulatory processes occur through different molecular interactions that could be modulated using a variety of compounds or modulators.

In the context of the present disclosure, a “compound” is a molecule such as, without being so limited, an siRNA, antisense molecule, protein, peptide, small molecule, antibody, etc.

Immune Checkpoint

Tumors can use immune checkpoints, which can be stimulatory or inhibitory, to protect themselves from immune system attacks. Immune checkpoint therapy seeks to block inhibitory checkpoints to restore immune system function⁴⁵. A specific targeted by methods of the present disclosure is the interaction between CD47 and its ligand SIRP alpha. Binding of SIRP alpha to CD47, as SIRP alpha & CD47 immune checkpoint pathway, essentially sends a ‘don't eat me’ message to macrophages by initiating signaling to inhibit phagocytosis. Inhibitors to the SIRPalpha and CD47 interaction (antibodies to SIRPalpha or CD47, soluble forms of SIRPalpha, peptides, small molecules) may allow macrophage to phagocyte the tumor.

Another specific ligand-receptor interaction targeted by methods of the present disclosure is the interaction between the transmembrane programmed cell death 1 protein (PD-1) and its ligand, PD-1 ligand 1 (PD-L1). The binding of PD-L1 to PD1 on an immune cell surface, inhibits immune cell activity. PD-L1 possesses a key regulatory role on T cell activities⁴⁶. Antibodies that bind to either PD-1 or PD-L1 and therefore block the interaction may allow the T cells to attack the tumor.

Inhibitors

SLAMF7 Inhibitor

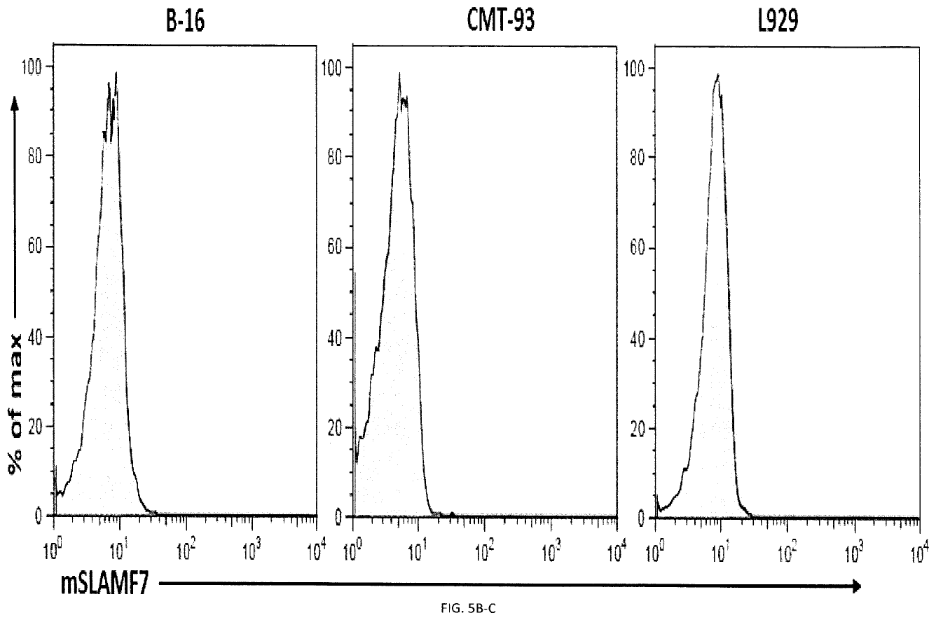

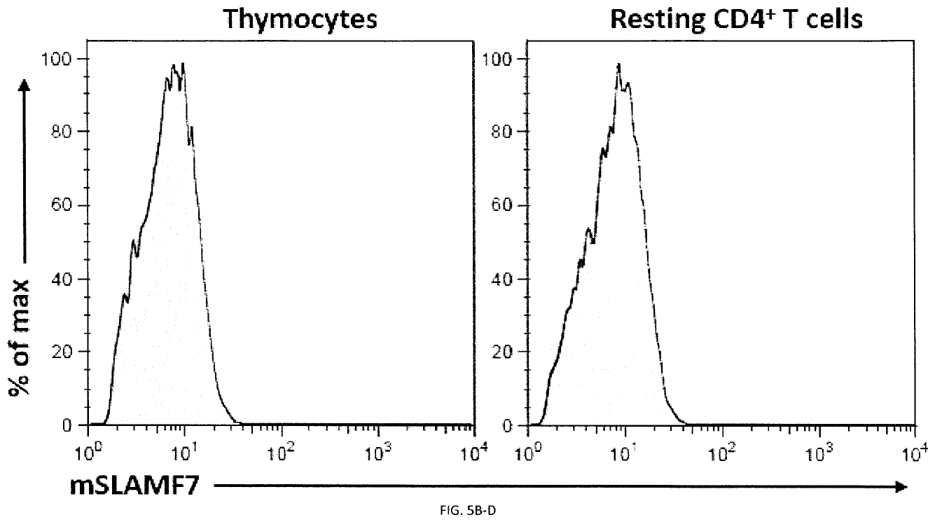

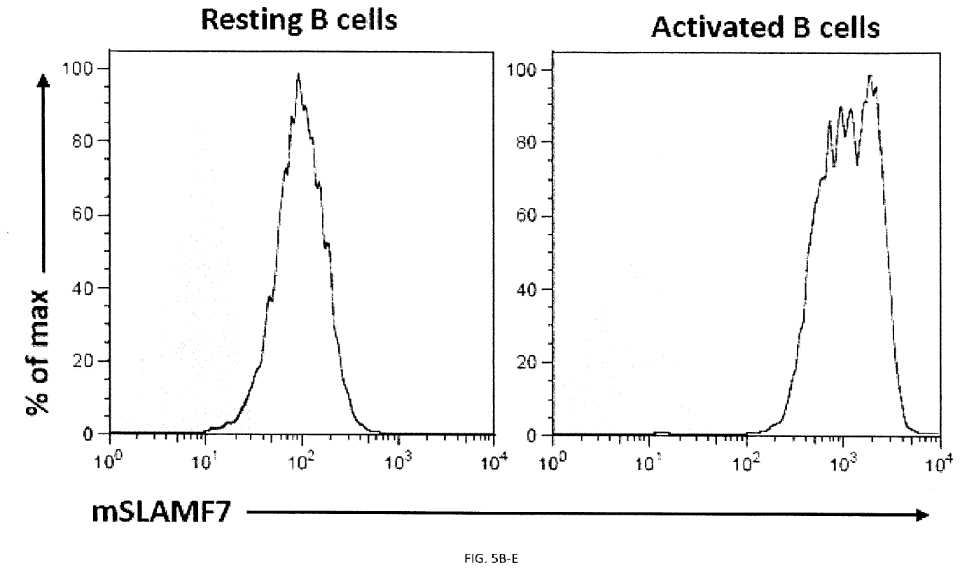

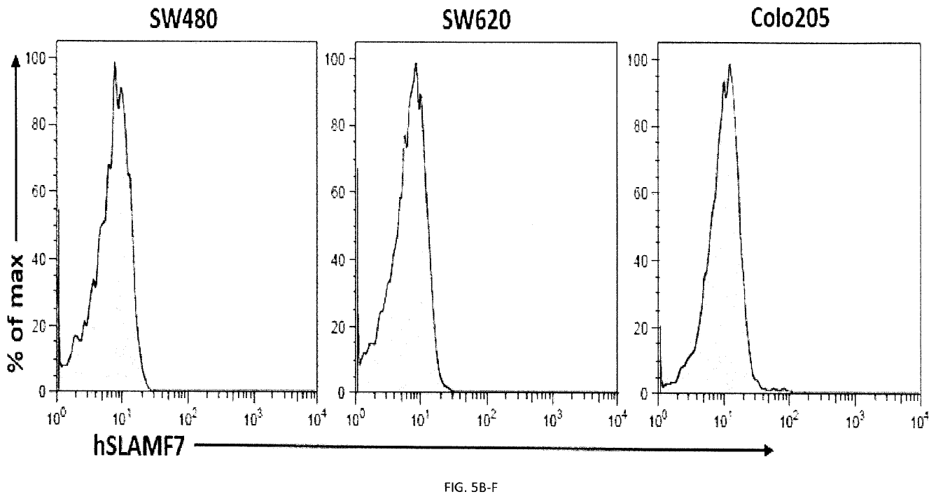

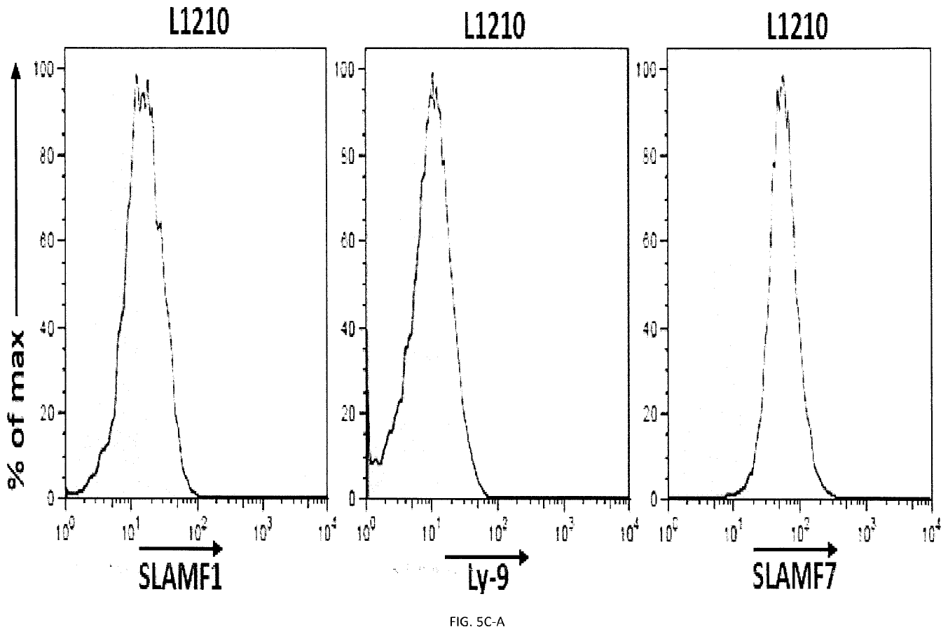

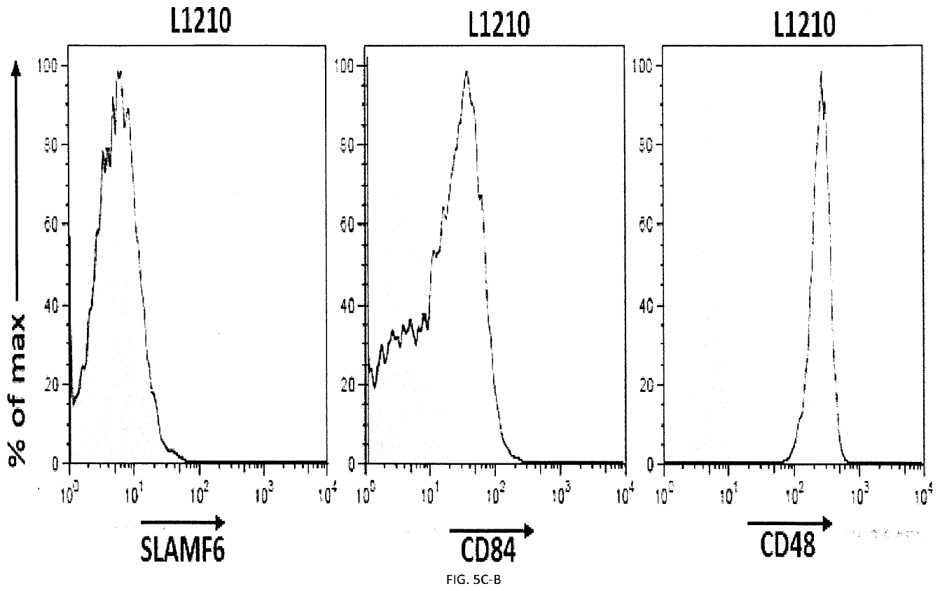

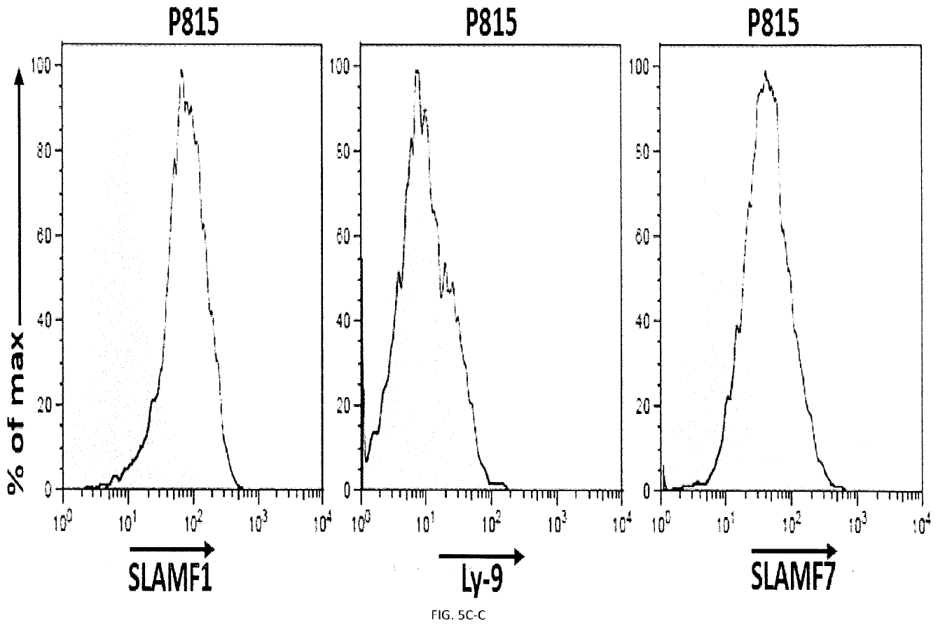

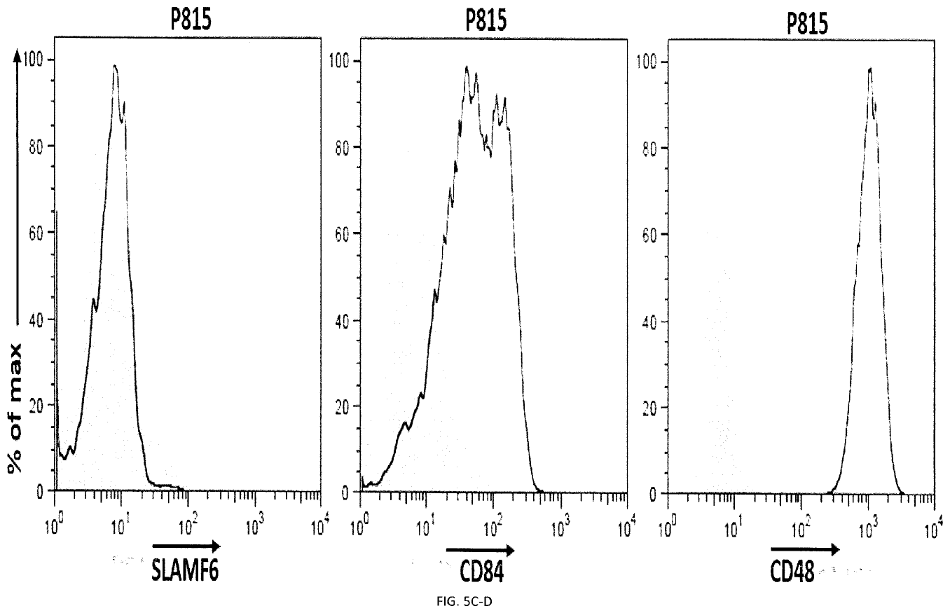

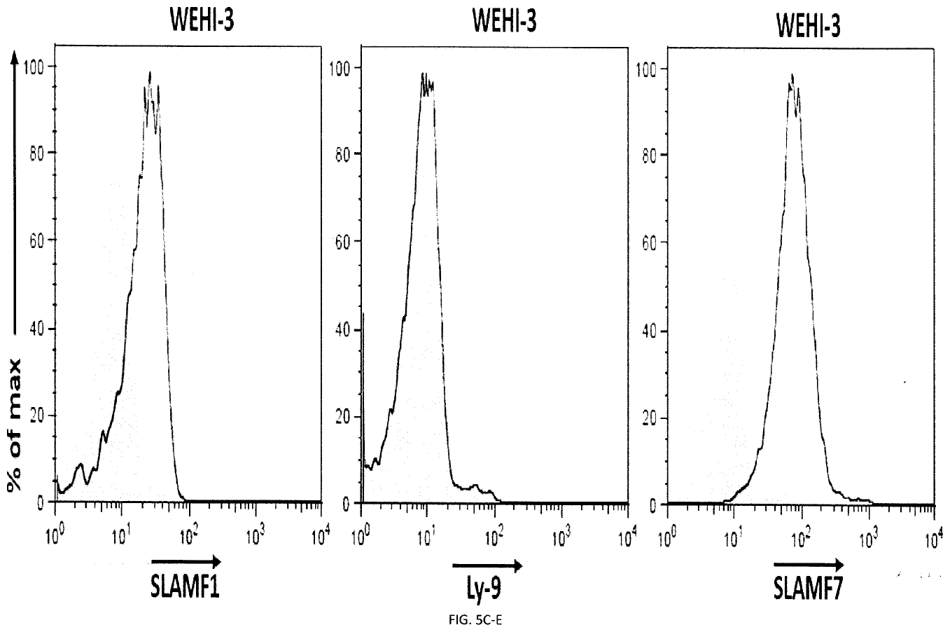

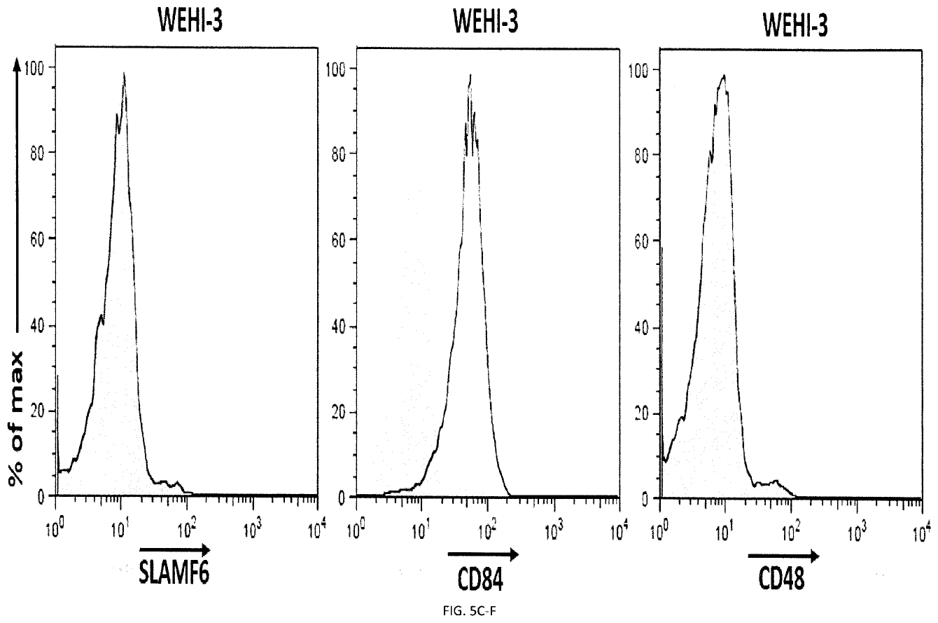

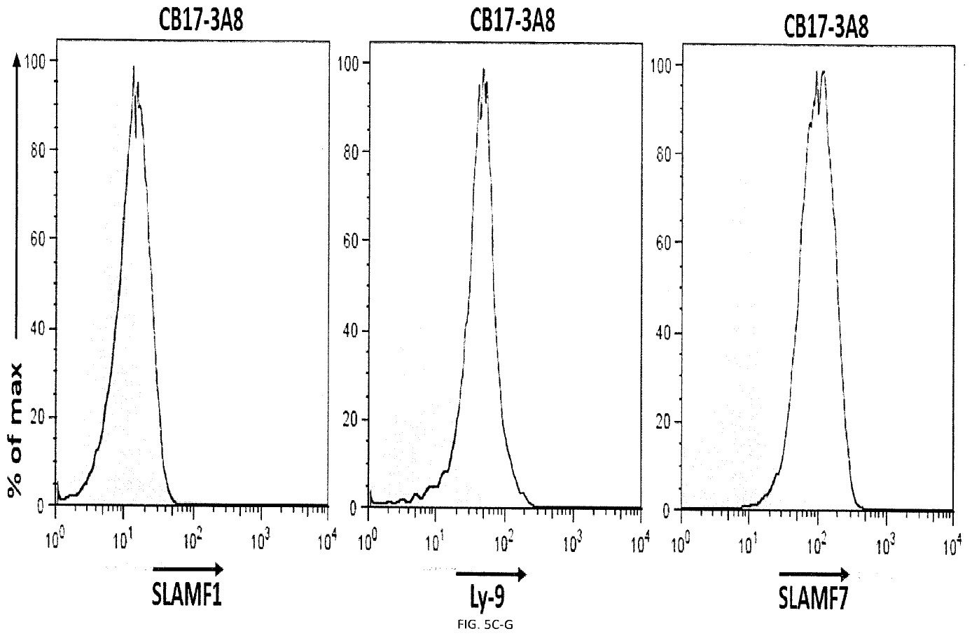

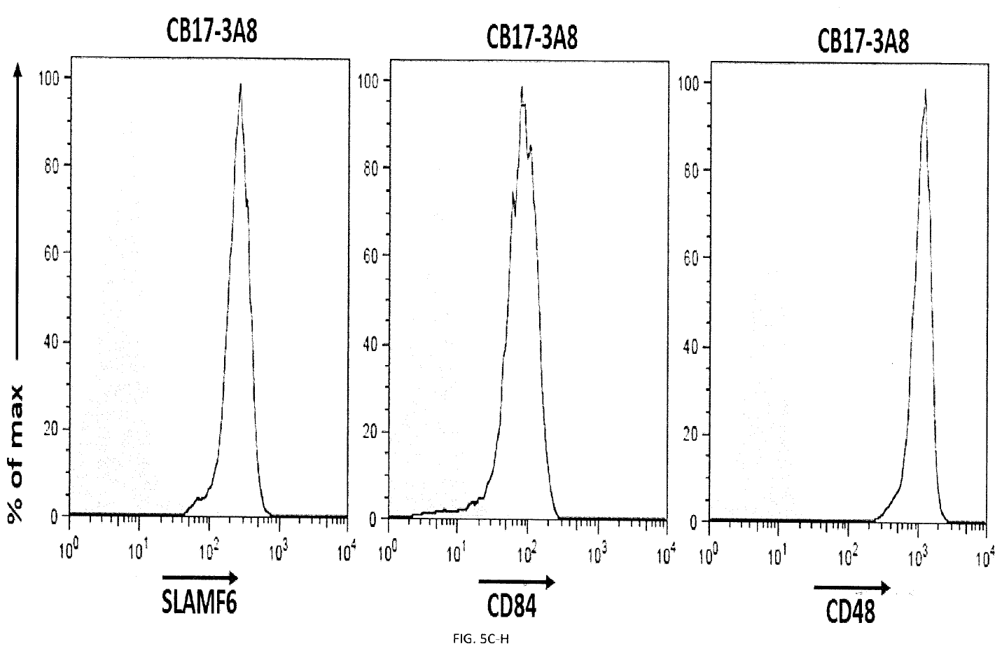

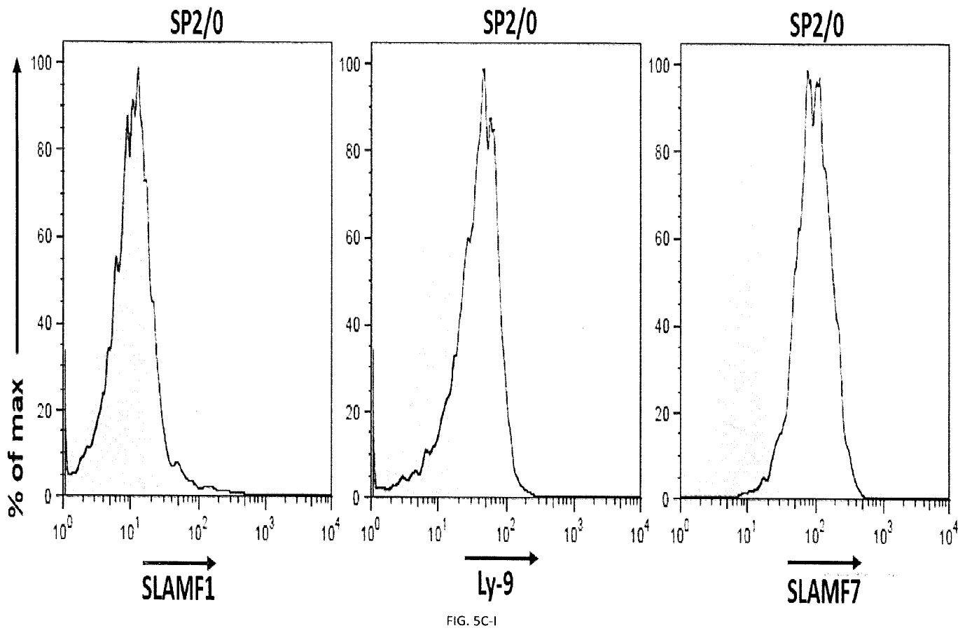

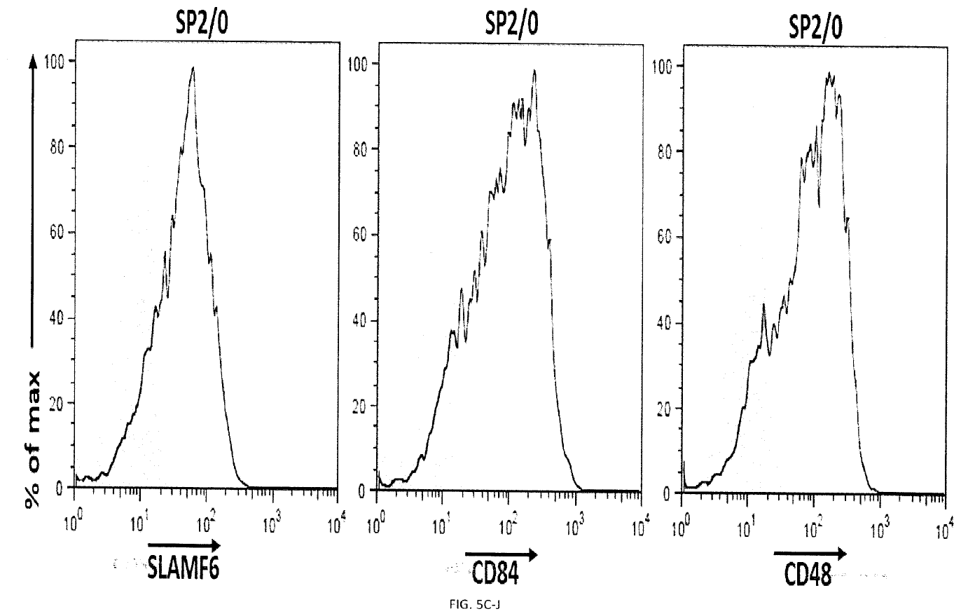

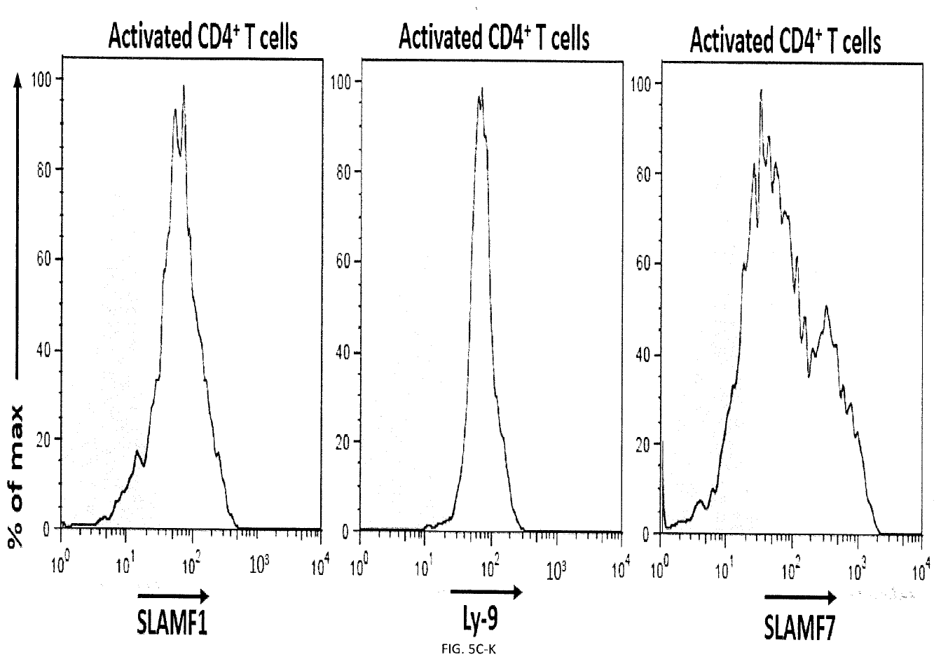

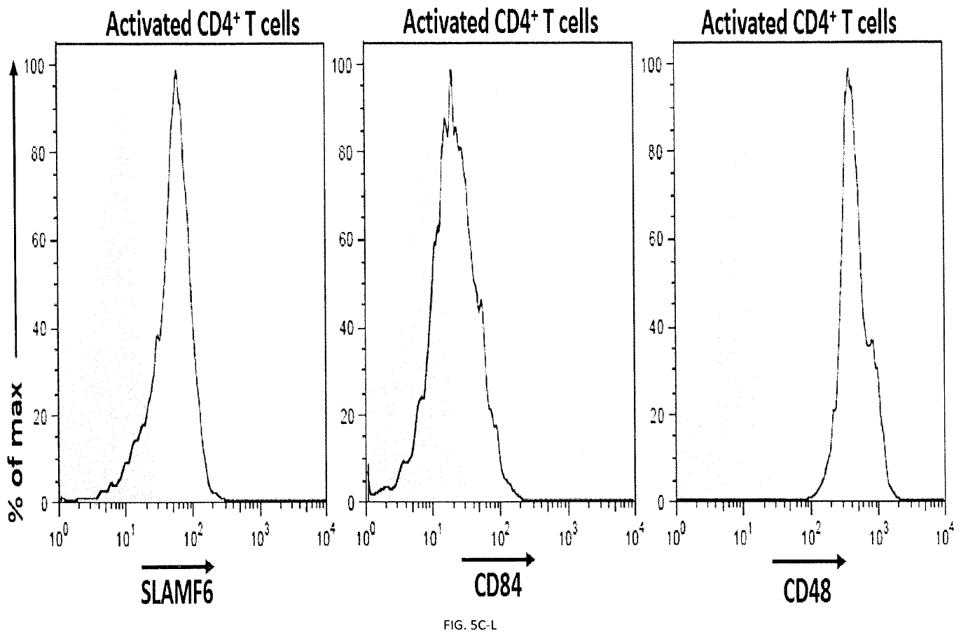

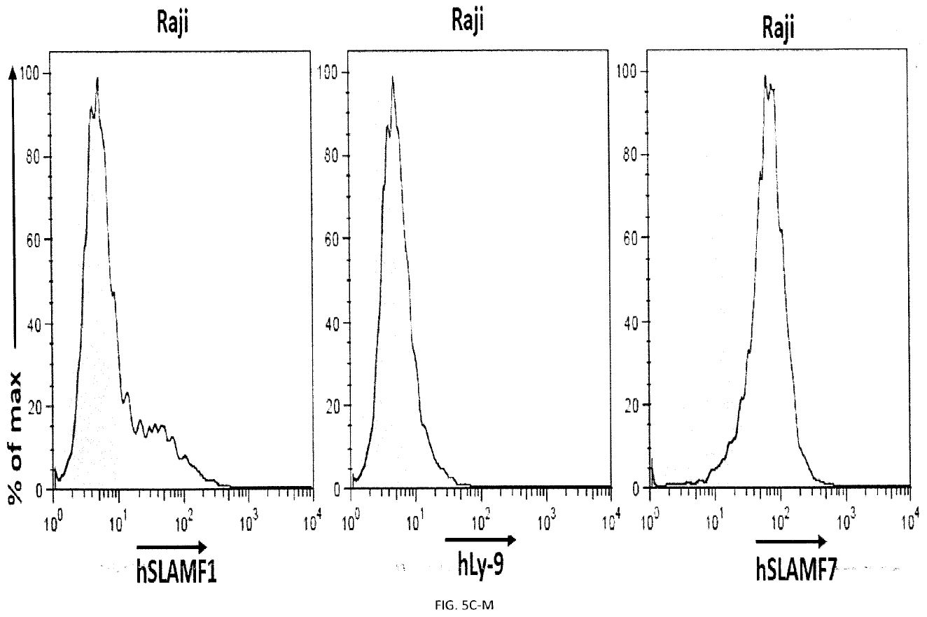

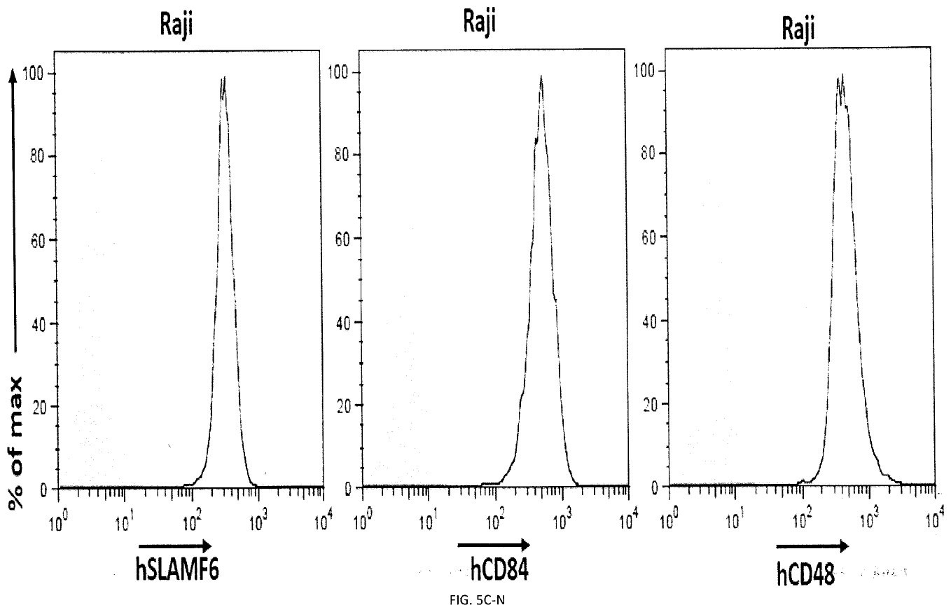

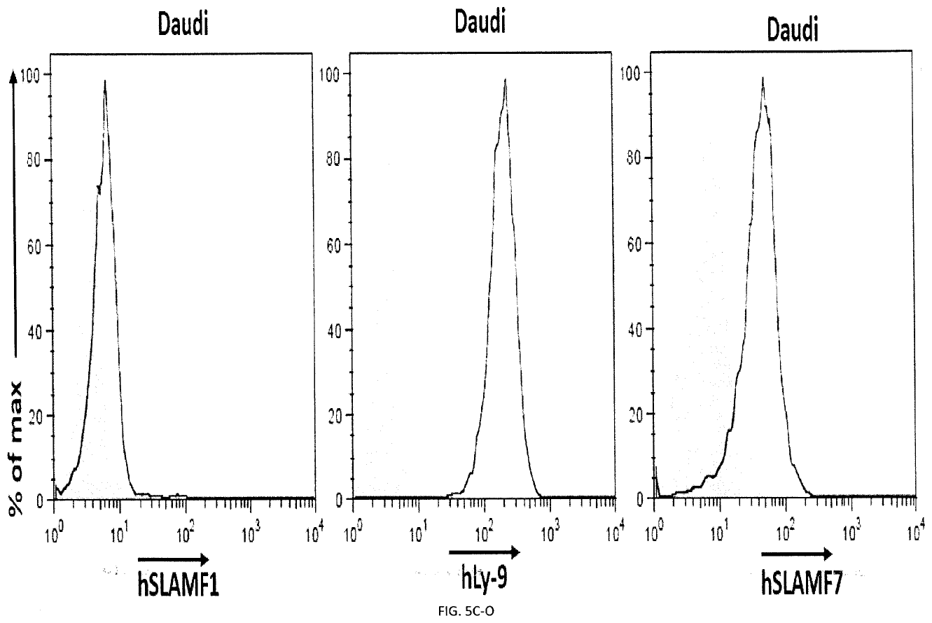

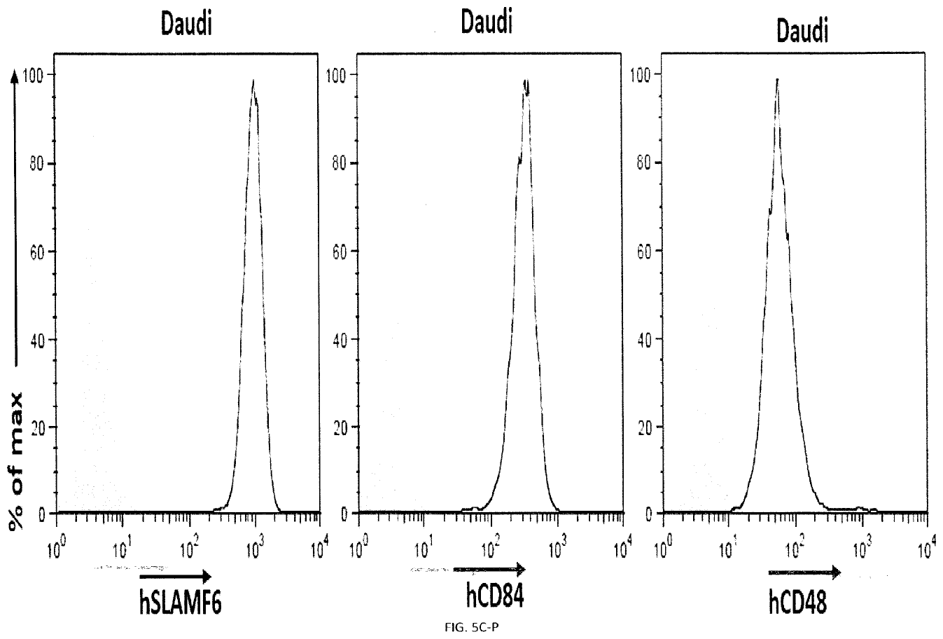

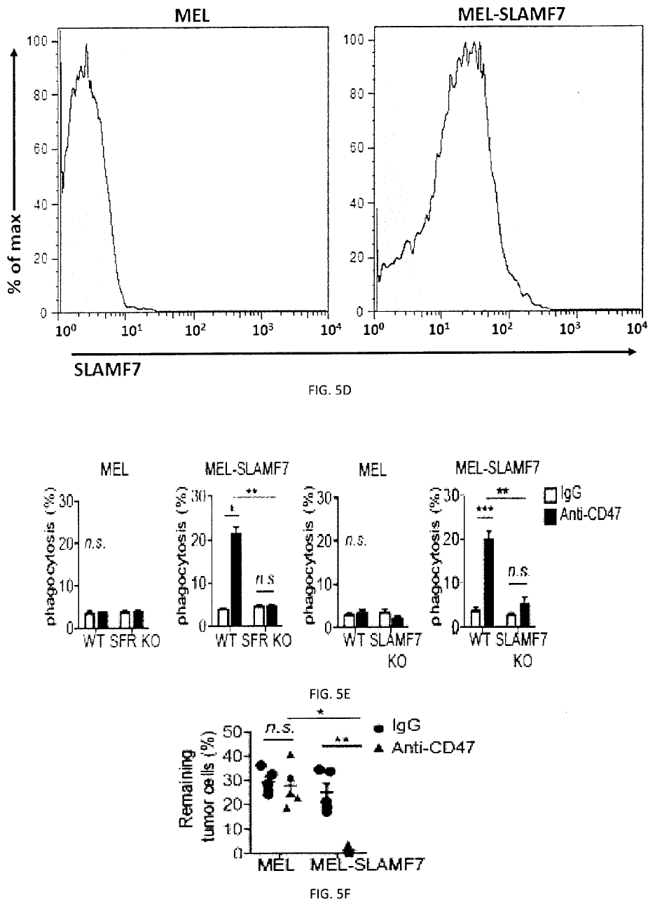

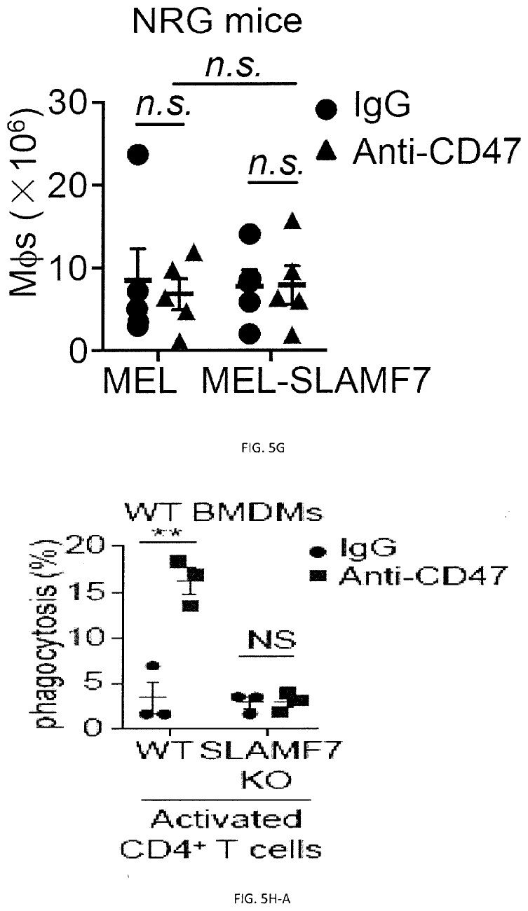

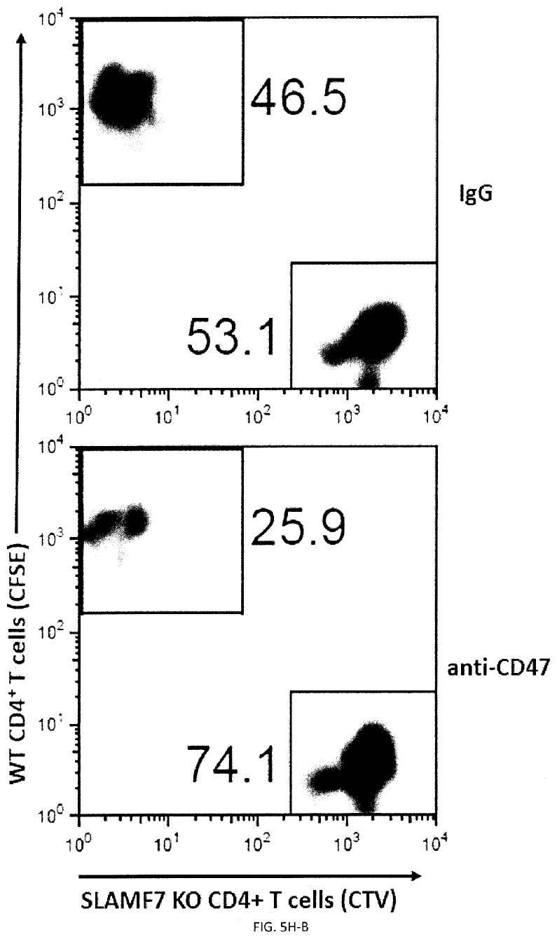

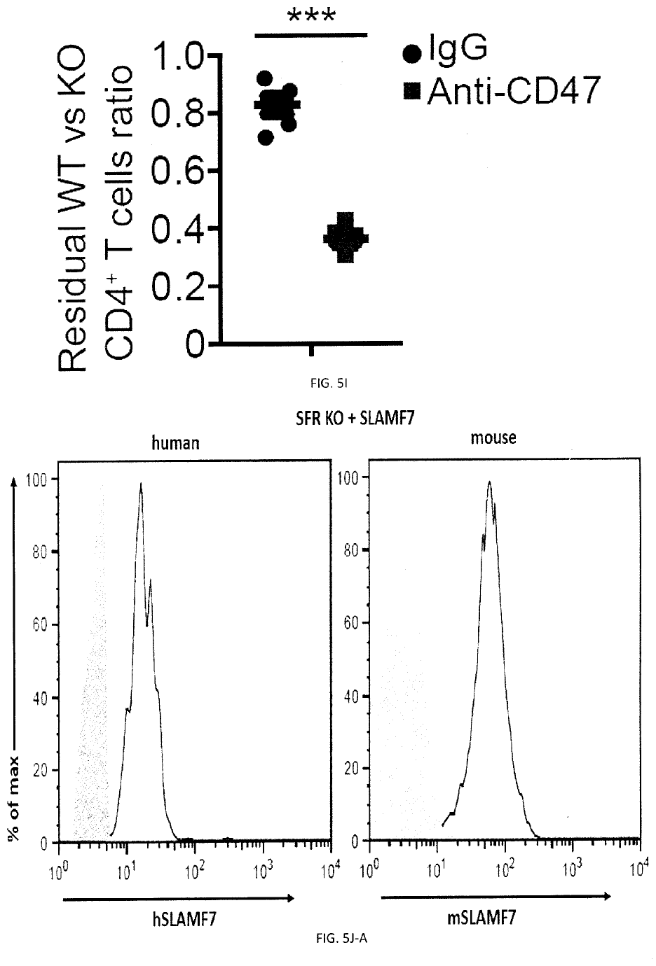

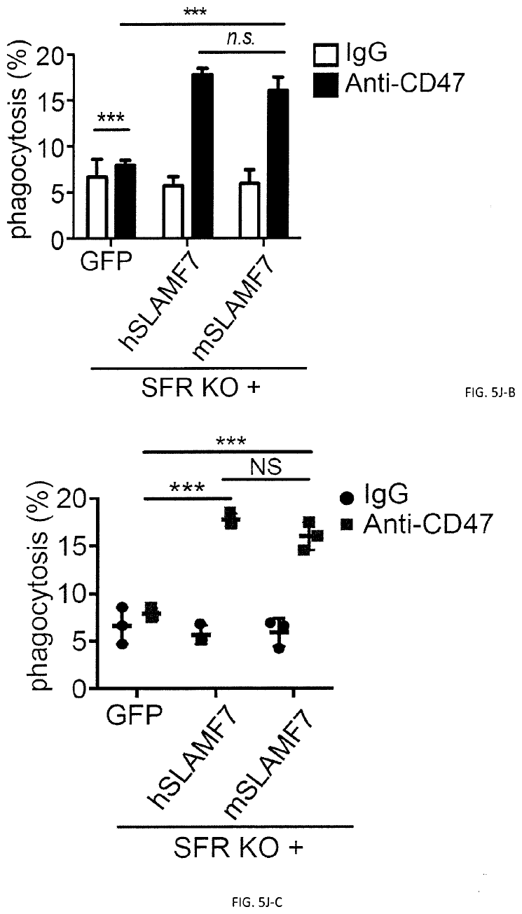

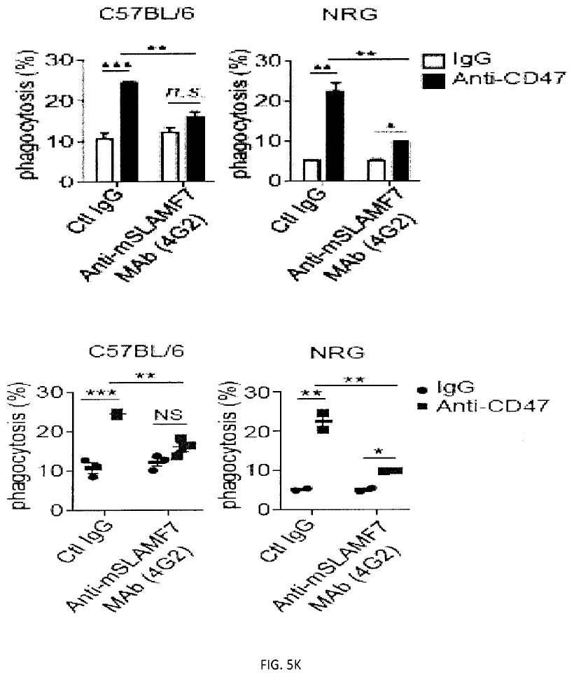

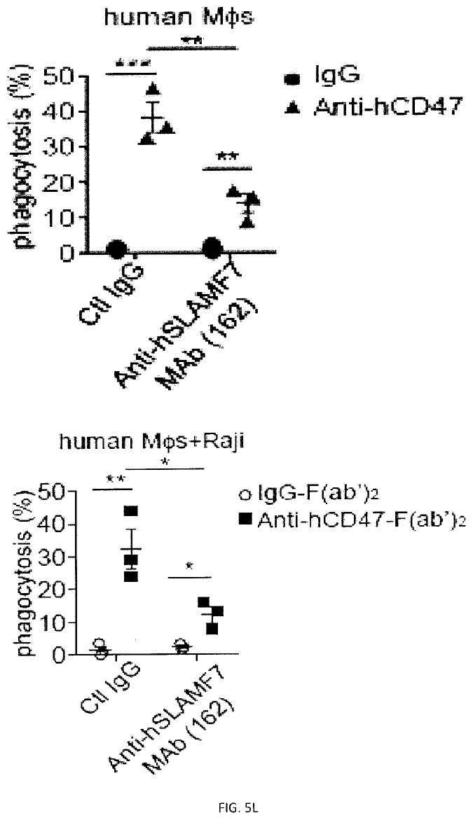

As used herein, “SLAMF7 inhibitor” refers to any compound or composition that directly or indirectly inhibits SLAMF7 expression and/or activity. Without being so limited, candidate compounds modulating the SLAMF7 expression and/or activity are tested using a variety of methods and assays. It includes molecules such as, without being so limited, siRNA, antisense molecule, protein, peptide, small molecule, antibody, etc. More particularly, it includes the anti-mouse SLAMF7 MAB4G2 which inhibits the SLAMF7 activity on phagocytosis by mouse macrophages (MABF917 (EMD Millipore)) (See instant FIG. 5K. The anti-human SLAMF7 MAB162 which inhibits the SLAMF7 activity on phagocytosis by human macrophages (see instant FIG. 5L). See also Table I above.

PD-1 Inhibitor

As used herein, “PD-1 inhibitor” refers to any compound or composition that directly or indirectly inhibits PD-1 expression and/or activity. Without being so limited, candidate compounds modulating the PD-1 expression and/or activity are tested using a variety of methods and assays. It includes molecules such as, without being so limited, siRNA, antisense molecule, protein, peptide, small molecule, antibody, etc. More particularly, it includes approved antibodies pembrolizumab (previously lambrolizumab), Nivolumab and pidilizumab, and other anti-PD-L1 antibodies currently in development including atezolizumab, avelumab and durvalumab; small proteins engineered to target PD-L1 such as Affimer™ biotherapeutic from Avacta Life Sciences. Approved inhibitors can be used for example to treat the following cancer types.

| | | |
|---|---|---|
| nivolumab | Hodgkin lymphoma | metastatic |
| | melanoma | metastatic |
| | non-small cell lung cancer | metastatic |
| | small cell lung cancer | metastatic |
| | hepatocellular carcinoma | metastatic |
| | renal cell carcinoma | metastatic |
| | ovarian cancer | metastatic |
| pembrolizumab | melanoma | metastatic |
| | non-small cell lung cancer | metastatic |
| | small cell lung cancer | metastatic |
| | head and neck cancer | metastatic |
| | urothelial cancer | metastatic |
| pidilizumab | melanoma | metastatic |

SIRPalpha-CD47 Checkpoint Inhibitor

As used herein, “SIRPalpha-CD47 checkpoint inhibitor” refers to any compound or composition that directly or indirectly inhibits SIRPalpha-CD47 checkpoint expression and/or activity. It includes SIRPalpha inhibitors and CD47 inhibitors listed herein as well as any other agent preventing the SIRPalpha-CD47 interaction or preventing SIRPalpha function and/or CD47 function. Without being so limited, candidate compounds modulating the SIRPalpha-CD47 checkpoint expression and/or activity are tested using a variety of methods and assays (e.g., the BPS bioscience assay kit 72044). It includes molecules such as, without being so limited, siRNA, antisense molecule, protein, peptide, small molecule, antibody, etc.

For example, an antibody or antibody fragment (e.g., anti-CD47 and anti-SIRPalpha) utilized in accordance with the present disclosure is in a format selected from, but not limited to, intact IgG, IgE and IgM, bi- or multi-specific antibodies (e.g., Zybodies™, etc.), single chain Fvs, polypeptide-Fc fusions, Fabs, cameloid antibodies, masked antibodies (e.g., Probodies™), Small Modular ImmunoPharmaceuticals (“SMIPs™), single chain or Tandem diabodies (TandAb™), VHHs, Anticalins™, Nanobodies (single domain antibodies), minibodies, BiTE™s, ankyrin repeat proteins or DARPINs™, Avimers™, a DART, a TCR-like antibody, Adnectins™, Affilins™, Trans-bodies™, Affibodies™, a TrimerX™, MicroProteins, Fynomers™, Centyrins™, and a Kalbitor™. In specific embodiments, antibodies of the present disclosure may lack a covalent modification (e.g., attachment of a glycan) that it would have if produced naturally. In specific embodiments, antibodies of the present disclosure may contain a covalent modification (e.g., attachment of a glycan, a payload, e.g., a detectable moiety, a therapeutic moiety, a catalytic moiety, etc., or other pendant group (e.g., poly-ethylene glycol, etc.). Antibodies of the present disclosure includes non-Fc receptor binding anti-CD47 and anti-SIRPalpha.

More particularly, it includes TTI-621 (SIRPaFc) from Trillium, an antibody-like fusion protein that blocks the inhibitory activity of CD47, a soluble form of SIRPa, a small molecule by Paradigm Shift Therapeutics, the anti-CD47 monoclonal antibody Hu5F9-G4, (Stanford, Phase I clinical study); the anti-CD47 monoclonal antibody CC-90002 (Celgene/InhibRx), the anti-CD47 and CD19 Bi-specific monoclonal antibody NI-1701 (Novimmune SA), anti-CD47-F(ab′)₂, anti-CD47 single domain antibody such as those described in Sockolosky⁵³, inhibitors listed in the following documents, which are incorporated herein by reference, WO2014093678 Therapeutic CD47 antibodies; US20140161805 Methods for manipulating phagocytosis mediated by CD47; US20140161825 Methods of treating acute myeloid leukemia by blocking CD47; US20120189625 Compositions and methods for treating hematological cancers targeting the SIRPalpha-CD47 interaction; US20120156724 Humanized anti-CD47 antibody; US20130142786 Humanized and chimeric monoclonal antibodies to CD47. See also Tables II and III above.

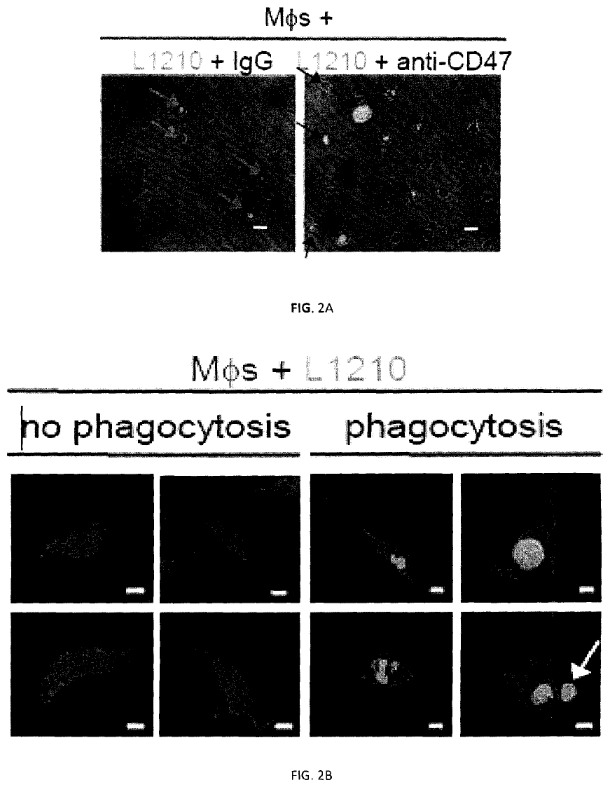

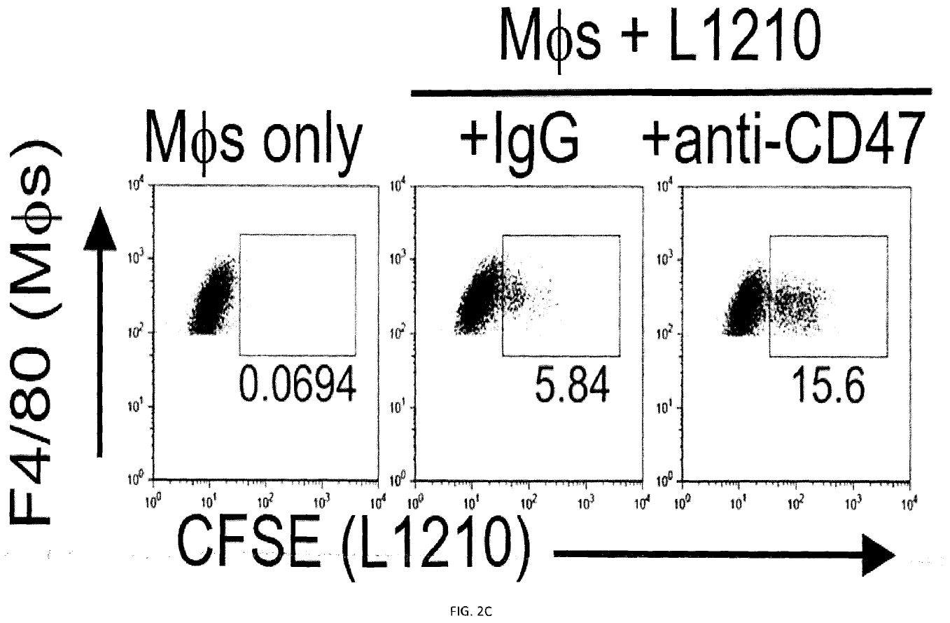

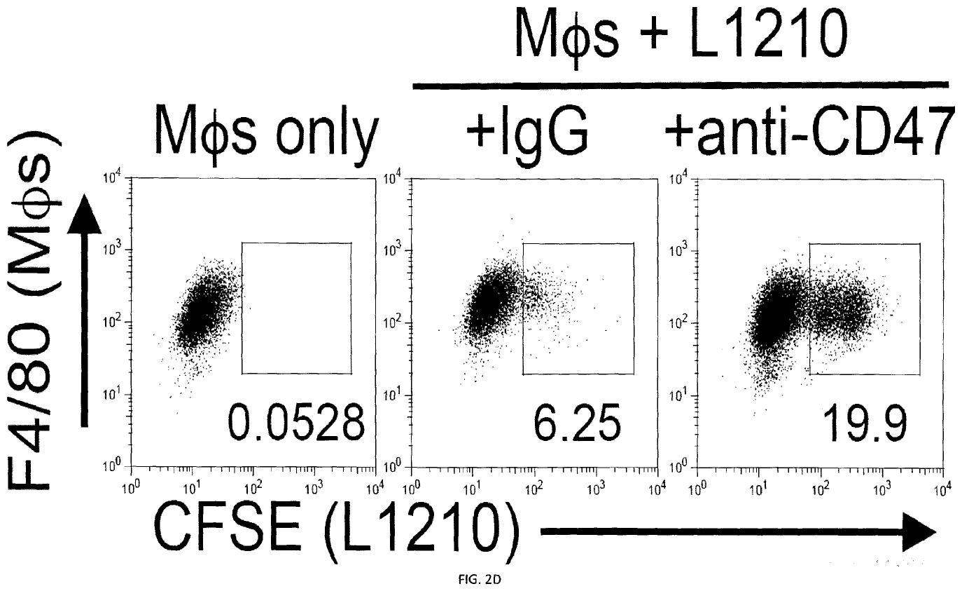

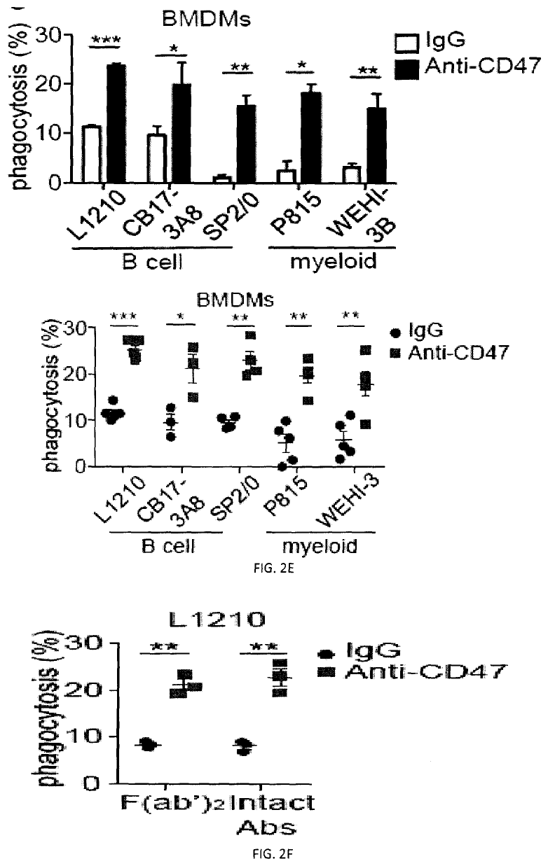

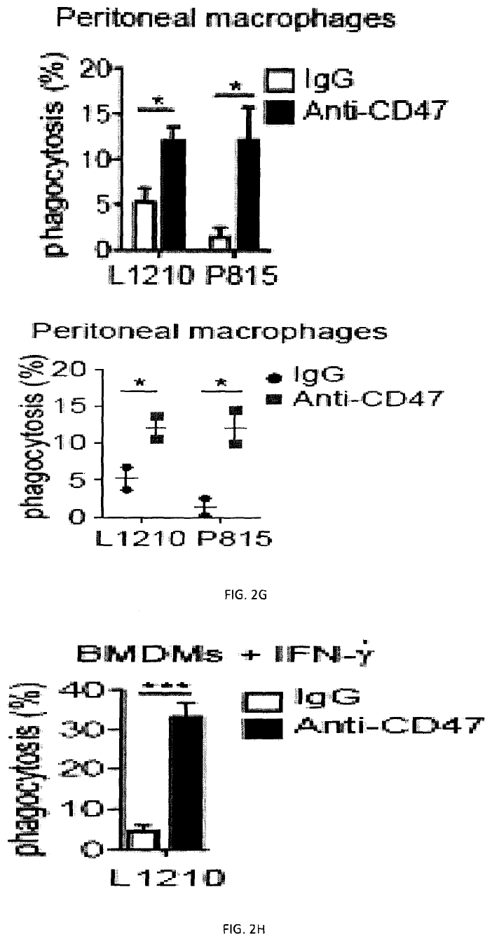

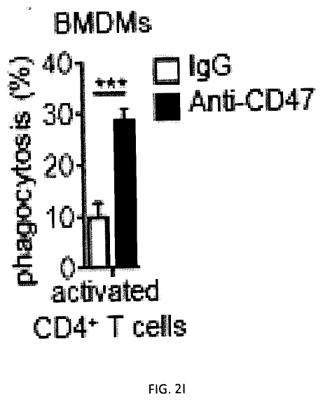

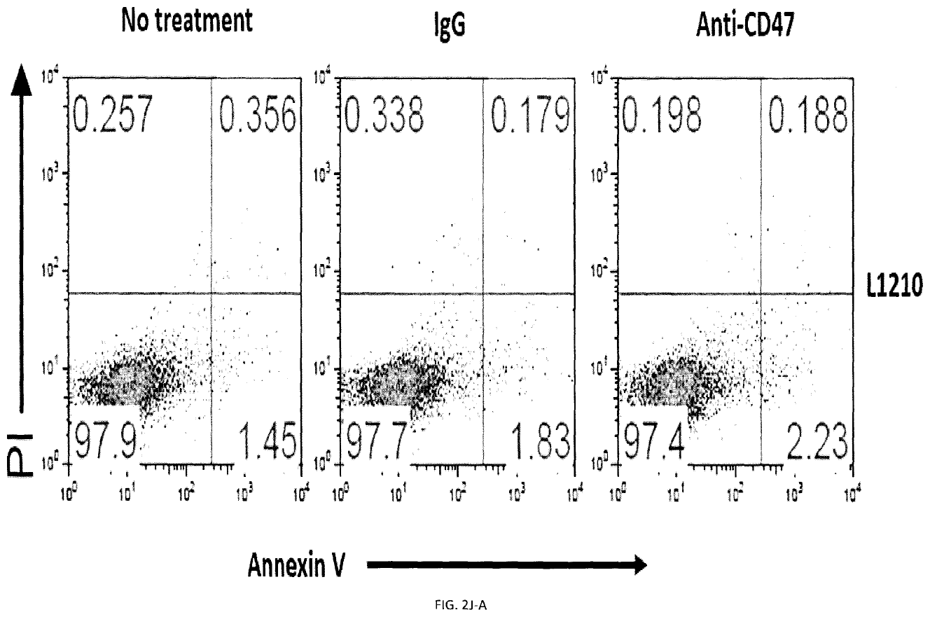

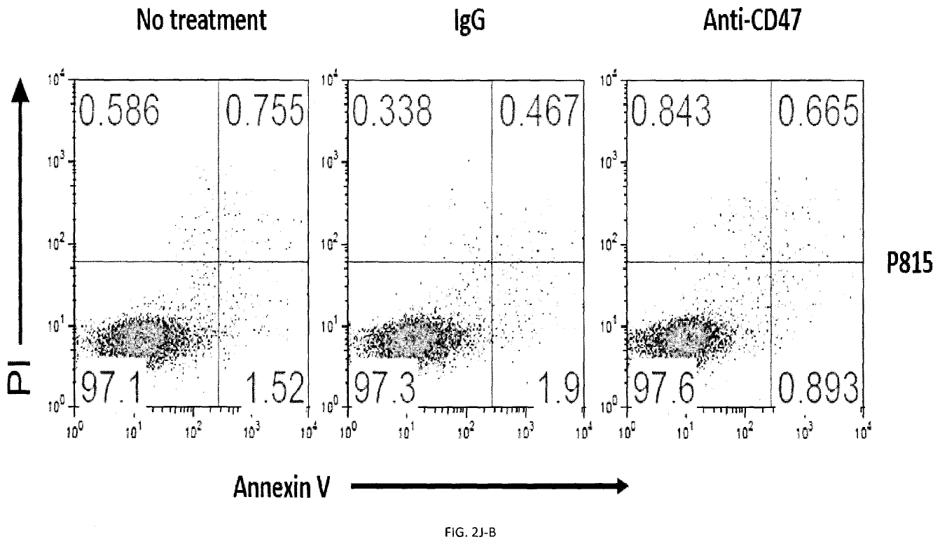

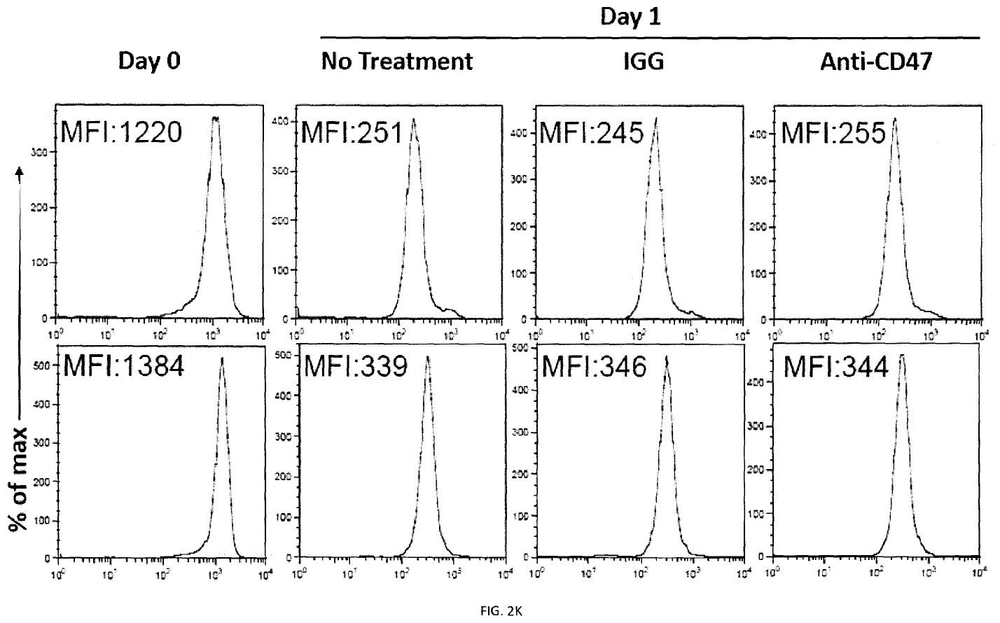

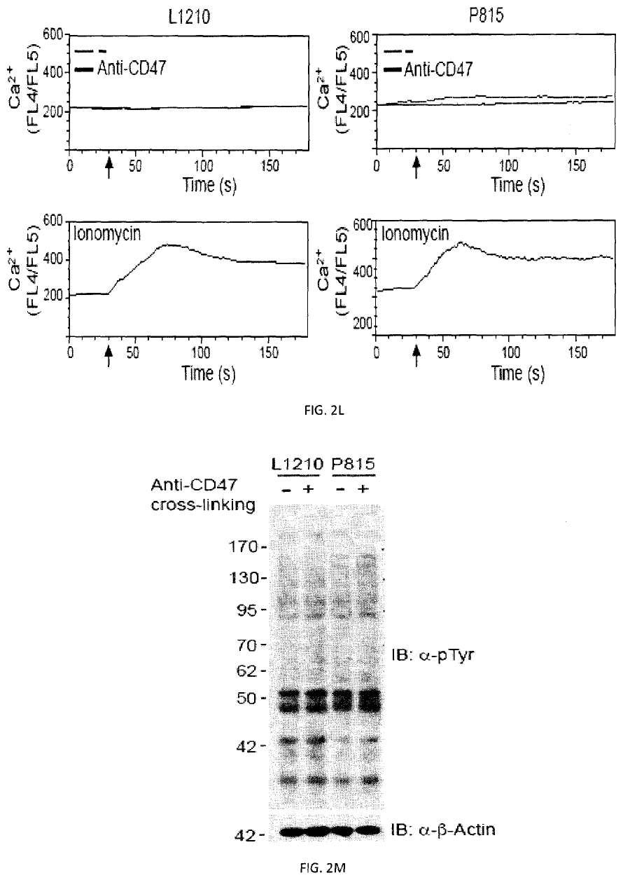

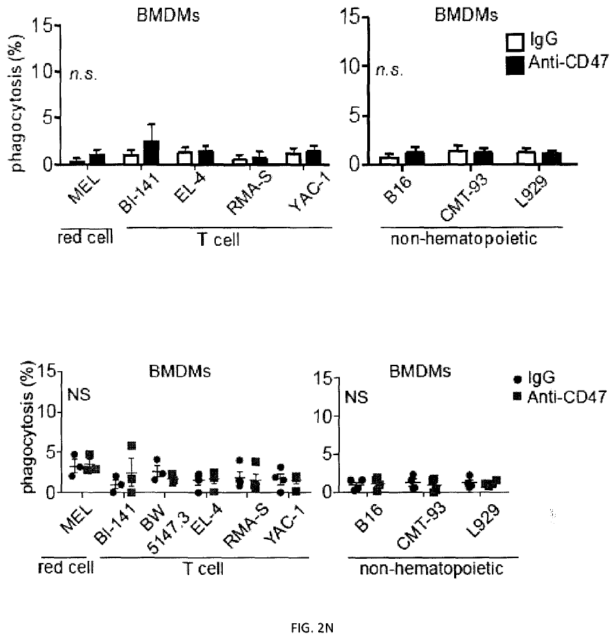

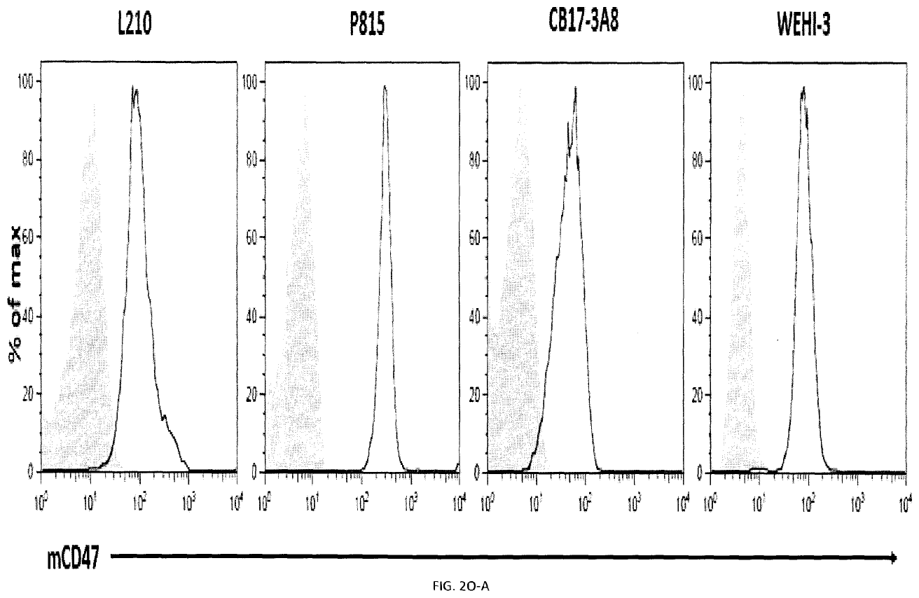

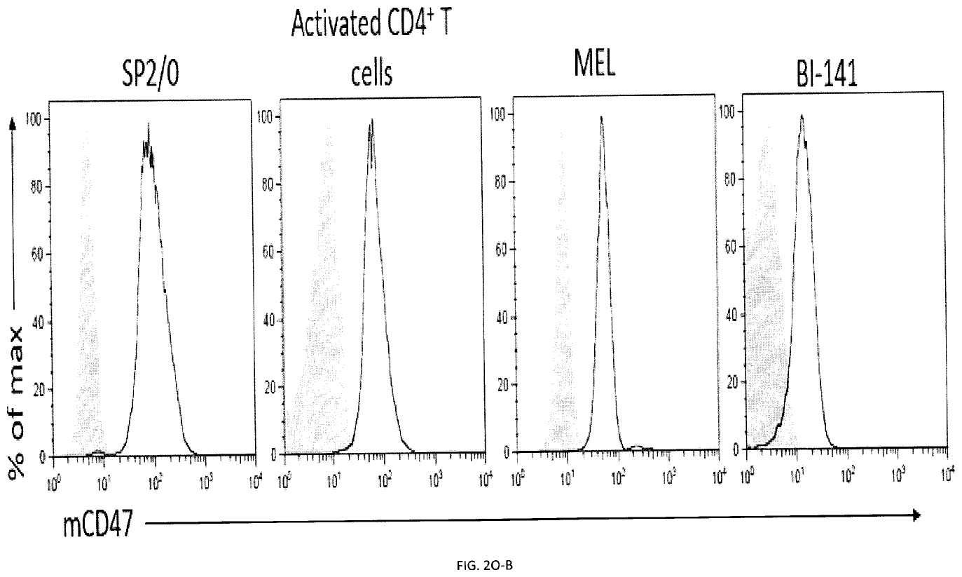

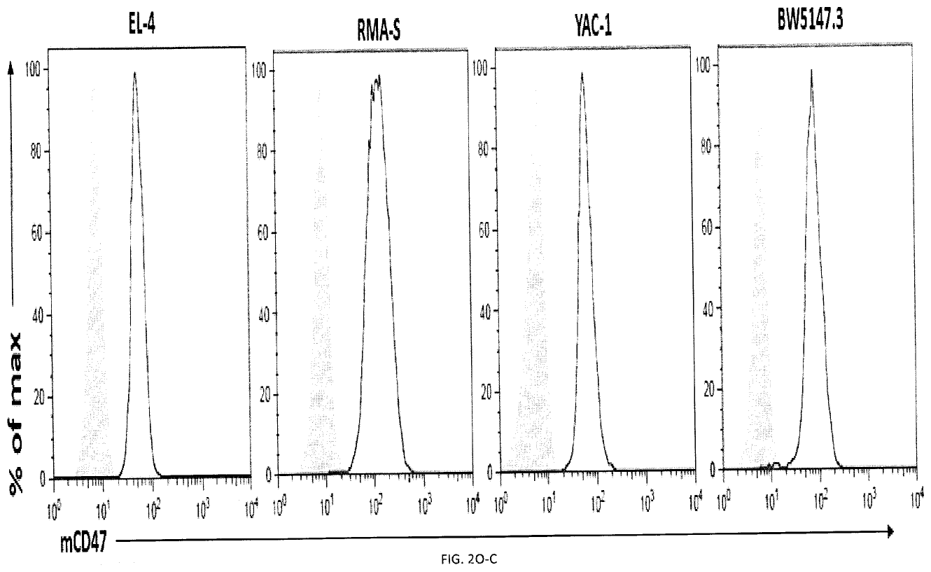

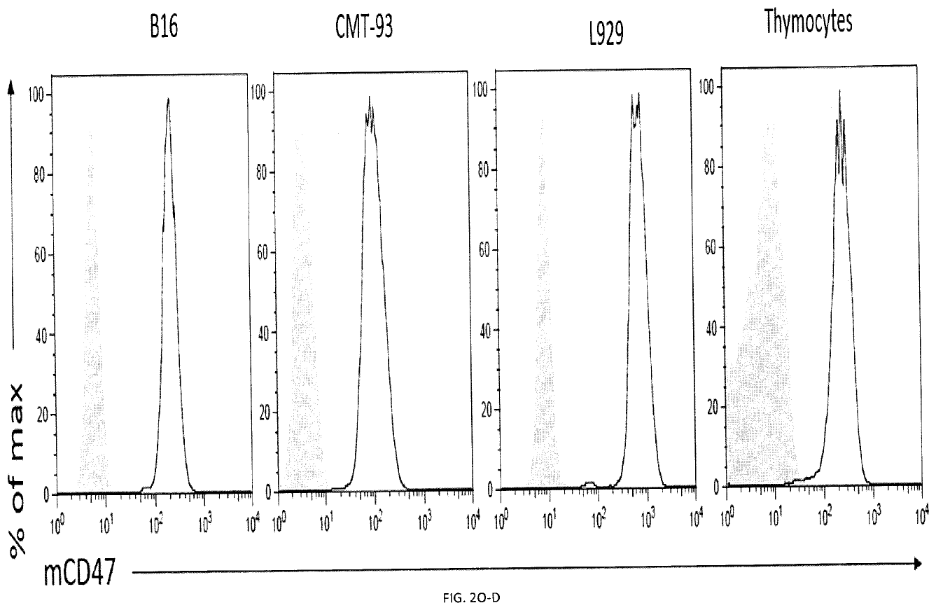

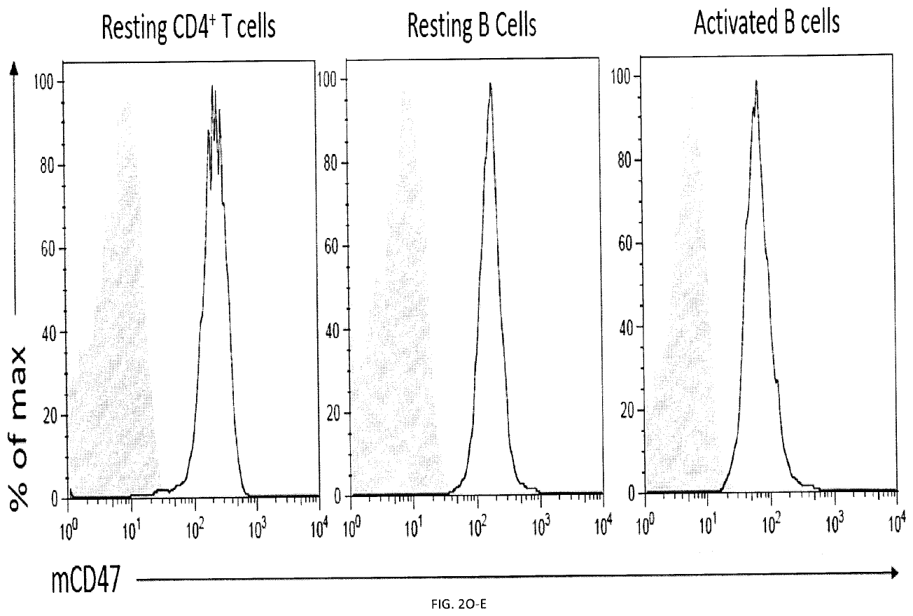

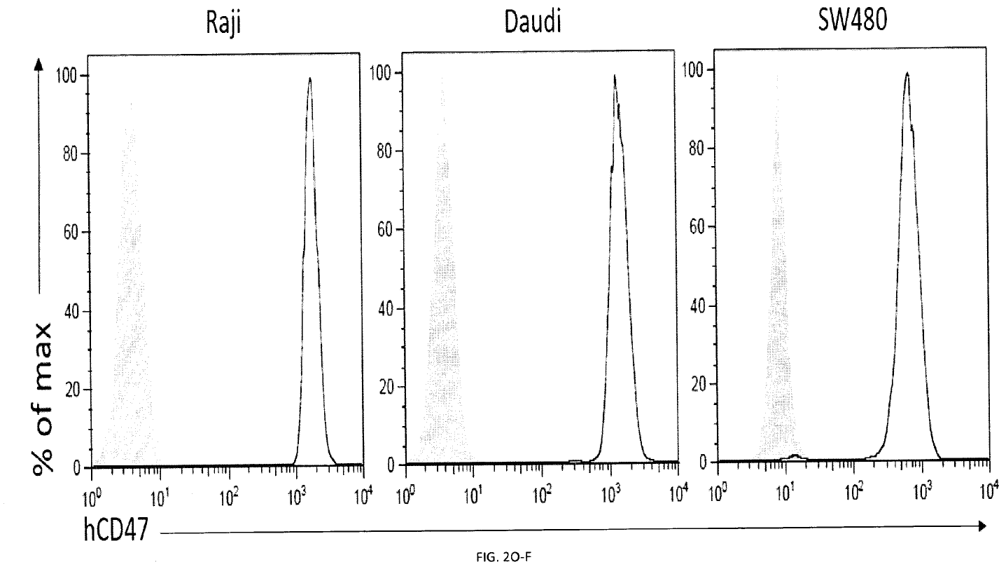

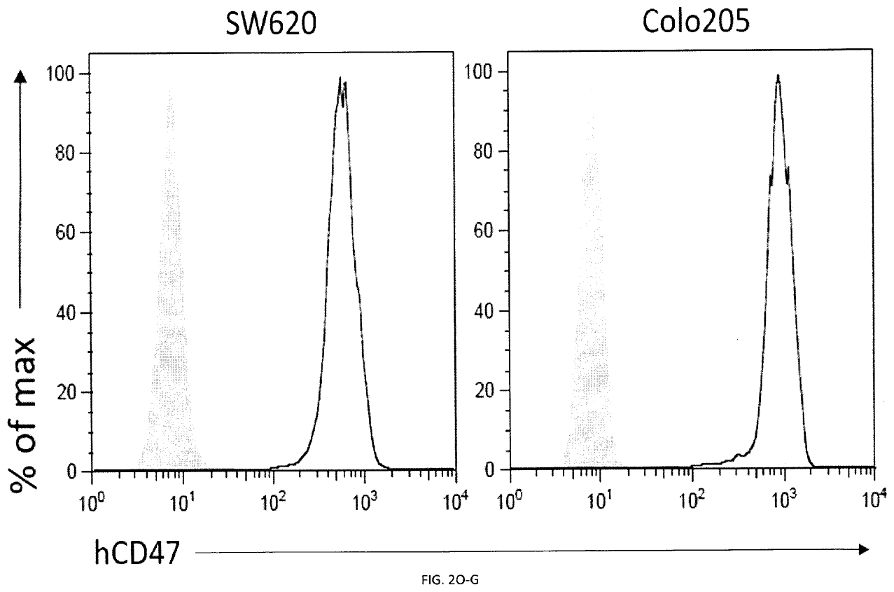

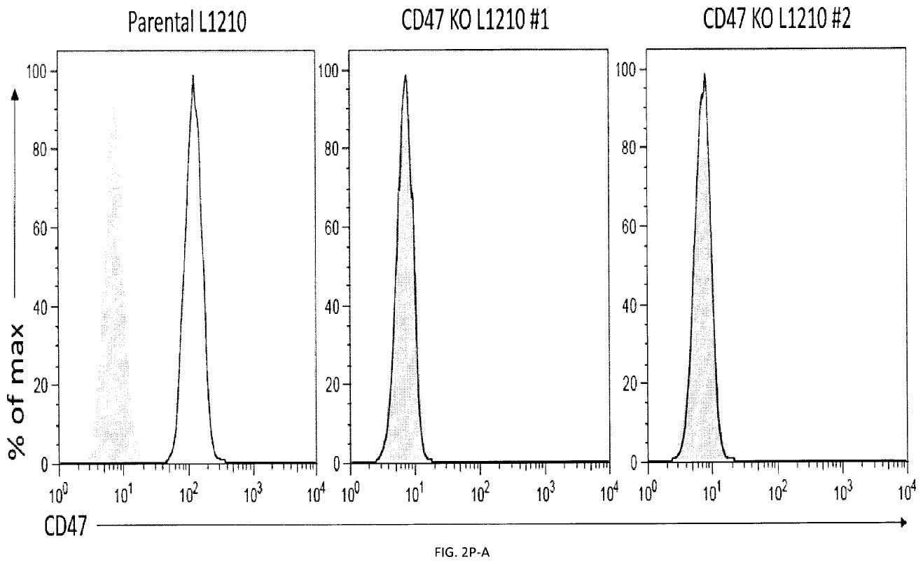

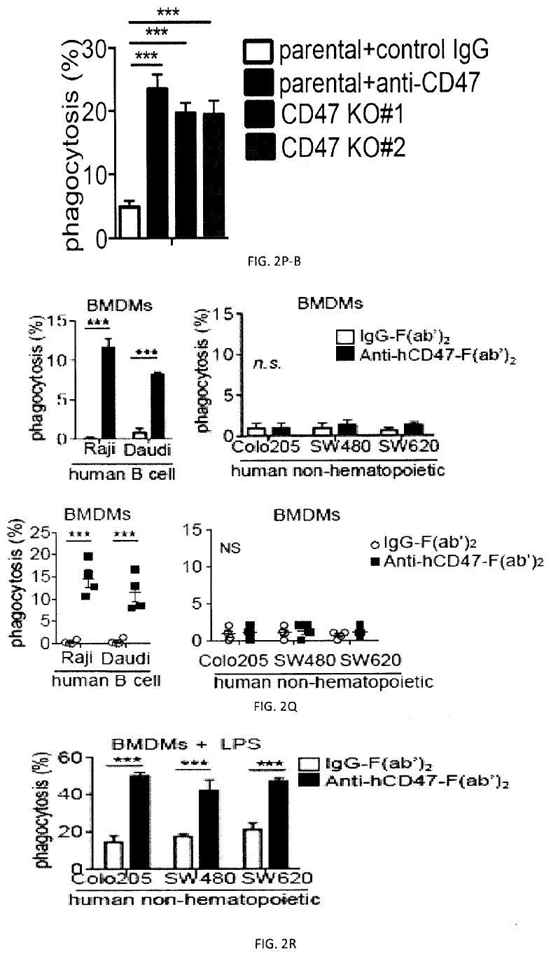

Since the inventors demonstrated that the phagocytosis of SLAMF7 positive tumors in the presence of an SIRPalpha-CD47 inhibitor is Fc receptor (FcR)-independent (See FIGS. 2F, 2Q-R, 3F, 3G, 3H, 4C-A, 4C-B, and 5L), particularly useful inhibitors to treat SLAMF7 positive tumors encompass non-Fc-receptor binding SIRPalpha-CD47 checkpoint inhibitors. Such inhibitors are advantageous in that they avoid potential toxicity that may be associated with Fc-binding inhibitors. For example, Fc containing SIRPalpha-CD47 checkpoint inhibitors may provoke Fc-receptor mediated phagocytosis of normal cells e.g., red blood cells, and, in turn, anemia.

As used herein the terms “non-Fc-receptor binding” refer to inhibitors without detectable or significant Fc-receptor binding. In a specific embodiment, it refers to an inhibitor that does not contain an Fc domain or contains an Fc domain that is non-functional i.e. that does not significantly or detectably bind to the Fc receptor. Such non-Fc-receptor binding inhibitors include small molecules, peptides/peptidomimetics, proteins that do not comprise an Fc domain (e.g., soluble form of SIRP), antibody fragments devoid Fc domain such as Fab, F(ab′)₂, single chain variable fragment (scFv), single monomeric variable antibody domain (also called nanobodies or single domain antibodies); and proteins or antibodies that comprise a non-functional Fc domain e.g., a Fc domain mutated to reduce Fc receptor binding (e.g., mutated at the lower hinge-C_{H}2 region).

CD47 Inhibitor

As used herein, “CD47 inhibitor” refer to any compound or composition that directly or indirectly inhibits CD47 expression and/or activity. Without being so limited, candidate compounds modulating the CD47 expression and/or activity are tested using a variety of methods and assays. It includes molecules such as, without being so limited, siRNA, antisense molecule, protein, peptide, small molecule, antibody, etc. More particularly, it includes TTI-621 from Trillium, an antibody-like fusion protein that blocks the inhibitory activity of CD47, Hu5F9-G4 (Stanford), CC-90002 (Celgene/InhibRx), NI-1701 (Novimmune SA), inhibitors listed in the following documents, which are incorporated herein by reference, WO2014093678 Therapeutic CD47 antibodies; US20140161805 Methods for manipulating phagocytosis mediated by CD47; US20140161825 Methods of treating acute myeloid leukemia by blocking CD47; US20120189625 Compositions and methods for treating hematological cancers targeting the SIRPalpha-CD47 interaction; US20120156724 Humanized anti-CD47 antibody; US20130142786 Humanized and chimeric monoclonal antibodies to CD47. More particularly, it includes SIRPalpha-CD47 checkpoint inhibitors as defined above including anti-CD47 antibodies or antibody fragments and non-Fc-receptor binding anti-CD47 inhibitors, and antibodies listed in Tables II above.

SIRPalpha Inhibitor

As used herein, “SIRPalpha inhibitor” refers to any compound or composition that directly or indirectly inhibits SIRPalpha expression and/or activity. Without being so limited, candidate compounds modulating the SIRPalpha expression and/or activity are tested using a variety of methods and assays. It includes molecules such as, without being so limited, siRNA, antisense molecule, protein, peptide, small molecule, antibody, etc. More particularly, it includes SIRPalpha-CD47 checkpoint inhibitors as defined above including anti-SIRPalpha antibodies or antibody fragments and non-Fc-receptor binding anti-SIRPalpha inhibitors, and antibodies listed in Table III above.

As used herein, “inhibition” or “decrease” of SLAMF7 and/or CD47 and/or SIRPalpha expression and/or activity refers to a reduction in SLAMF7 and/or CD47 and/or SIRPalpha expression level or activity level of at least 5% as compared to reference SLAMF7 and/or CD47 and/or SIRPalpha expression and/or activity (e.g., a measurement of SLAMF7 and/or CD47 and/or SIRPalpha expression and/or activity in the subject before treatment with an SLAMF7 and/or CD47 and/or SIRPalpha inhibitor). In an embodiment, the reduction in SLAMF7 and/or CD47 and/or SIRPalpha expression level or activity level is of at least 10% lower, in a further embodiment, at least 15% lower, in a further embodiment, at least 20% lower, in a further embodiment of at least 30%, in a further embodiment of at least 40%, in a further embodiment of at least 50% lower, in a further embodiment of at least 60% lower, in a further embodiment of at least 70% lower, in a further embodiment of at least 80%, in a further embodiment of at least 90%, in a further embodiment of 100% (complete inhibition).

Preferably, a SLAMF7 and/or CD47 and/or SIRPalpha inhibitor is a compound having a low level of cellular toxicity and acting in a reversible manner.

SLAMF7 Agonists

As used herein, “SLAMF7 agonist” refers to any compound or composition that directly or indirectly increases SLAMF7's expression and/or activity. It includes molecules such as, without being so limited, nucleic acid encoding SLAMF7 protein, protein, peptide, small molecule, antibodies, etc. More particularly, it includes SLAMF7, elotuzumab, etc. Candidate compounds are tested using a variety of methods and assays.

SLAMF7 can be used as an agonist to target SLAMF7 negative tumor cells using e.g., adenoviruses or other gene/protein delivery (see e.g., instant FIGS. 5D-G) and thereby force SLAMF7 expression on the tumor cells. Such tumor cells may thereafter benefit from treatments described herein for SLAMF7 positive tumors.

As used herein, “increase” of SLAMF7 expression and/or activity refers to an increase in SLAMF7 expression level or activity level of at least 5% as compared to reference SLAMF7 expression and/or activity (e.g., a measurement of SLAMF7 expression and/or activity in the subject before treatment with a SLAMF7 stimulator). In an embodiment, the increase in SLAMF7 expression level or activity level is of at least 10% higher, in a further embodiment, at least 15% higher, in a further embodiment, at least 20% higher, in a further embodiment of at least 30% higher, in a further embodiment of at least 40% higher, in a further embodiment of at least 50% higher, in a further embodiment of at least 60% higher, in a further embodiment of at least 70% higher, in a further embodiment of at least 80% lower, in a further embodiment of at least 90% lower, in a further embodiment of 100% lower.

Results reported herein at e.g., Examples 2-6, show that SLAMF7 presence on tumor cells, increases macrophage phagocytose of such tumor cells in the presence of a SIRPalpha-CD47 blocker (inhibitor).

Without being so limited, SLAMF7 agonists include a SLAMF7 gene, RNA or protein such as that shown in FIGS. 13A-T, etc.

Screening Assays

Given the correlation between SLAMF7 expression/activity on a tumor and susceptibility of the tumor to SIRPalpha-CD47 checkpoint blockade induced phagocytosis, compounds which are capable of (i) inhibiting the SIRPalpha-CD47 checkpoint and/or compounds; or (ii) increasing SLAMF7 activity and/or expression may be used for the prevention and/or treatment of SLAMF7 positive cancer. Similarly, compounds which are capable of decreasing SLAMF7 activity and/or expression may be used for the prevention and/or treatment of SLAMF7 negative cancers.

Screening for SIRPalpha-CD47 Checkpoint Inhibitors

Therefore, the disclosure further relates to screening methods using SLAMF7 positive cells for the identification and characterization of compounds capable of inhibiting SIRPalpha-CD47 checkpoint which may be used for the prevention and/or treatment of SLAMF7 positive tumors.

The present disclosure also provides a method (e.g., an in vitro method) for determining whether a test compound is useful for the prevention and/or treatment of SLAMF7 positive tumors, said method comprising: (a) contacting said test compound with a (tumor) cell expressing SLAMF7 and CD47 and macrophages (e.g., Mouse bone marrow-derived macrophages (BMDMs) or peritoneal macrophages); and (b) determining the phagocytosis of the cell by the macrophages, in the presence or absence of said test compound; wherein an increase in the phagocytosis in the presence of said test compound relative to the absence thereof is indicative that said test compound may be used for the prevention and/or treatment of SLAMF7 positive cancer.

Screening for SLAMF7 Modulators

The present disclosure also provides a method (e.g., an in vitro method) for determining whether a test compound is useful for the prevention and/or treatment of cancer, said method comprising: (a) contacting said test compound with a SLAMF7 polypeptide, or a fragment thereof or variant thereof having SLAMF7 activity; and (b) determining the expression and/or activity of the SLAMF7 polypeptide, fragment or variant thereof, in the presence or absence of said test compound; wherein said modulation in the expression and/or activity of SLAMF7 in the presence of said test compound relative to the absence thereof is indicative that said test compound may be used for the prevention and/or treatment of cancer.

The present disclosure also provides a method (e.g., an in vitro method) for determining whether a test compound is useful for the prevention and/or treatment of cancer, said method comprising: (a) contacting said test compound with a cell comprising a first nucleic acid comprising a transcriptionally regulatory element normally associated with a SLAMF7 gene, operably linked to a second nucleic acid comprising a reporter gene encoding a reporter protein; and (b) determining whether the reporter gene expression and/or reporter protein activity is modulated in the presence of said test compound; wherein said modulation in reporter gene expression and/or reporter protein activity is indicative that said test compound may be used for prevention and/or treatment of cancer.

The present disclosure also provides a method (e.g., an in vitro method) for determining whether a test compound is useful for the prevention and/or treatment of cancer, said method comprising: (a) contacting said test compound with a cell comprising a first nucleic acid comprising a transcriptionally regulatory element normally associated with a gene whose expression is modulated by SLAMF7 activity, operably linked to a second nucleic acid comprising a reporter gene encoding a reporter protein; and (b) determining whether the reporter gene expression and/or reporter protein activity is modulated in the presence of said test compound; wherein said modulation in reporter gene expression and/or reporter protein activity is indicative that said test compound may be used for prevention and/or treatment of cancer.

More particularly, an increase in the expression and/or activity of SLAMF7 in the presence of said test compound relative to the absence thereof, is indicative that said test compound may be used in combination with a SIRPalpha-CD47 checkpoint inhibitor for the prevention and/or treatment of cancers characterized by SLAMF7 positive tumor(s). A decrease in the expression and/or activity of SLAMF7 in the presence of said test compound relative to the absence thereof, is indicative that said test compound may be used for the prevention and/or treatment of cancers characterized by SLAMF7 negative tumor(s).

The above-mentioned methods may be employed either with a single test compound or a plurality or library (e.g., a combinatorial library) of test compounds. In the latter case, synergistic effects provided by combinations of compounds may also be identified and characterized. The above-mentioned compounds may be used for prevention and/or treatment of cancer, or may be used as lead compounds for the development and testing of additional compounds having improved specificity, efficacy and/or pharmacological (e.g., pharmacokinetic) properties. In an embodiment, the compound may be a prodrug which is altered into its active form at the appropriate site of action, (e.g., a cell, tissue or organ affected by cancer. In certain embodiments, one or a plurality of the steps of the screening/testing methods of the disclosure may be automated.

Such assay systems may comprise a variety of means to enable and optimize useful assay conditions. Such means may include but are not limited to: suitable buffer solutions, for example, for the control of pH and ionic strength and to provide any necessary components for optimal SLAMF7 activity and stability, temperature control means for SLAMF7 activity and or stability, and detection means to enable the detection of a SLAMF7 activity reaction product. A variety of such detection means may be used, including but not limited to one or a combination of the following: radiolabelling (e.g., ³²P, ¹⁴O, ³H), antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/(strept)avidin), and others.

The assay may be carried out in vitro utilizing a source of SLAMF7 which may comprise naturally isolated or recombinantly produced SLAMF7, in preparations ranging from crude to pure. Recombinant SLAMF7 may be produced in a number of prokaryotic or eukaryotic expression systems, which are well known in the art. Such assays may be performed in an array format.

As noted above, the disclosure further relates to methods for the identification and characterization of compounds capable of modulating SLAMF7 gene expression. Such a method may comprise assaying SLAMF7 gene expression in the presence versus the absence of a test compound. Such gene expression may be measured by detection of the corresponding RNA or protein, or via the use of a suitable reporter construct comprising one or more transcriptional regulatory element(s) normally associated with a SLAMF7 gene, operably-linked to a reporter gene.

A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences.

Generally, operably-linked DNA sequences are contiguous and, where necessary to join two protein coding regions, in reading frame. However, since, for example, enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals which induce or control transcription of protein coding sequences with which they are operably-linked. The expression of such a reporter gene may be measured on the transcriptional or translational level, e.g., by the amount of RNA or protein produced. RNA may be detected by for example Northern analysis or by the reverse transcriptase-polymerase chain reaction (RT-PCR) method (see for example Sambrook et al. (1989) Molecular Cloning: A Laboratory Manual (2^{nd} edition), Cold Spring Harbor Laboratory Press, Cold Spring Harbor, N.Y., USA).

Protein levels may be detected either directly using affinity reagents (e.g., an antibody or fragment thereof (for methods, see for example Harlow, E. and Lane, D (1988) Antibodies: A Laboratory Manual, Cold Spring Harbor Laboratory Press, Cold Spring Harbor, N.Y.); a ligand which binds the protein) or by other properties (e.g., fluorescence in the case of green fluorescent protein) or by measurement of the protein's activity, which may entail enzymatic activity to produce a detectable product (e.g., with altered spectroscopic properties) or a detectable phenotype (e.g., alterations in cell growth/function). Suitable reporter genes include but are not limited to chloramphenicol acetyltransferase, beta-D galactosidase, luciferase, or green fluorescent protein (GFP).

SLAMF7 expression levels could be determined using any standard methods known in the art. Non-limiting examples of such methods include Western blot, immunoblot, enzyme-linked immunosorbant assay (ELISA), radioimmunoassay (RIA), immunoprecipitation, immunocytochemistry, immunohistochemistry, as well as methods to determine mRNA levels such as RT-PCR and northern analysis, real-time PCR, PCR, in situ hybridization and so on.

For example, a test compound may be added to a reaction mixture containing a purified SLAMF7 and a SLAMF7 ligand or a peptide fragment of a SLAMF7 ligand (e.g., Anti-SLAMF7, CD11), and the binding between SLAMF7 and the SLAMF7 ligand is determined and compared to the binding when the mixture is incubated under similar conditions but without the test compound. A lower binding in the presence of the test compound is indicative that the test compound may be useful for inhibiting SLAMF7 activity and in turn for the prevention and/or treatment of cancer. The detection step (i.e. determination of the binding) could be monitored by any number of means including, but not limited to binding-dependent optical spectroscopy, fluorimetry, and radioactive label variation and could use various techniques such as Surface Plasmon resonance, FRET, yeast two hybrids, and alpha-screen.

In another aspect, the present disclosure provides an agent that modulates SLAMF7 expression or activity identified by the above-noted screening method.

Neoplastic Diseases

The terminology “neoplastic disease” or “invasive disease” is meant herein to refer to a disease associated with new growth of any body tissue. A neoplastic tissue according to the disclosure is derived from a pre-neoplastic tissue and may retain some characteristics of the tissue from which it arises but has biochemical characteristics that are distinct from those of the parent tissue. The tissue formed due to neoplastic growth is a mutant version of the original tissue and appears to serve no physiologic function in the same sense as did the original tissue. It may be benign or malignant (e.g., cancer).

Cancer is defined herein as a disease characterized by the presence of cancer cells which possess two heritable properties: they and their progeny are able (1) to reproduce unrestrained in defiance of the normal restraints (i.e., they are neoplastic) and (2) invade and colonize territories normally reserved for other cells (i.e., they are malignant). Invasiveness of cancer cells usually implies an ability to break loose, enter the bloodstream or lymphatic vessels, and form secondary tumors, or metastases at the other distant sites in the body. The term “cancer cells” refers herein to a cluster of cancer or tumor cells showing over proliferation by non-coordination of the growth and proliferation of cells due to the loss of the differentiation ability of cells. The terms “cancer cell” and “tumor cell” are used interchangeably herein.

As used herein, the term “hematopoietic tumor” is meant to refer to leukemia (e.g., chronic lymphocytic leukemia), lymphoma (e.g., diffuse large B cell lymphoma), multiple myeloma, plasmacytoma, pre-leukemia, myelodysplastic syndrome and mastocytoma.

As used herein, the terms “B-cell derived tumors” include B-cell derived leukemia, B-cell derived lymphoma (including Burkitt's lymphoma).

As used herein, the term “solid tumor” or “solid tumor cancer” is meant to refer to cancers such as colon, bile duct, breast, chondrosarcoma, colorectal, endometrium, esophagus, Ewings sarcoma, glioma, kidney, liver, non-small cell lung, small cell lung, medulloblastoma, melanoma, mesothelioma, neuroblastoma, osteosarcoma, ovary, pancreas, prostate, soft tissue, stomach, thyroid, upper aerodigestive, urinary tract cancers, etc.

Neoplastic Markers

SLAMF7 is not systematically expressed in all cancers. For example, SLAMF7 expression variations were observed between hematopoietic and non-hematopoietic tumors, whereas hematopoietic tumors generally express SLAMF7 while non-hematopoietic tumors generally do not express SLAMF7.

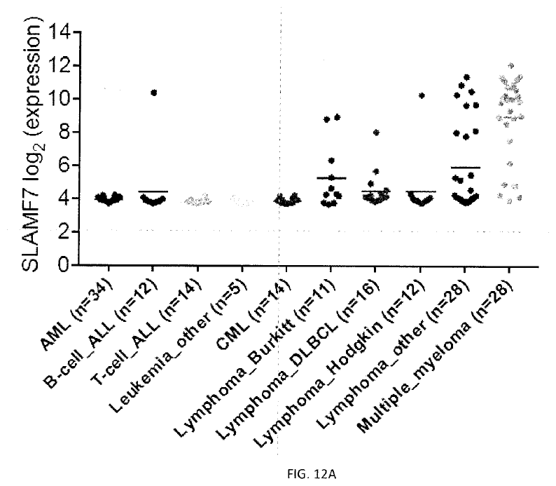

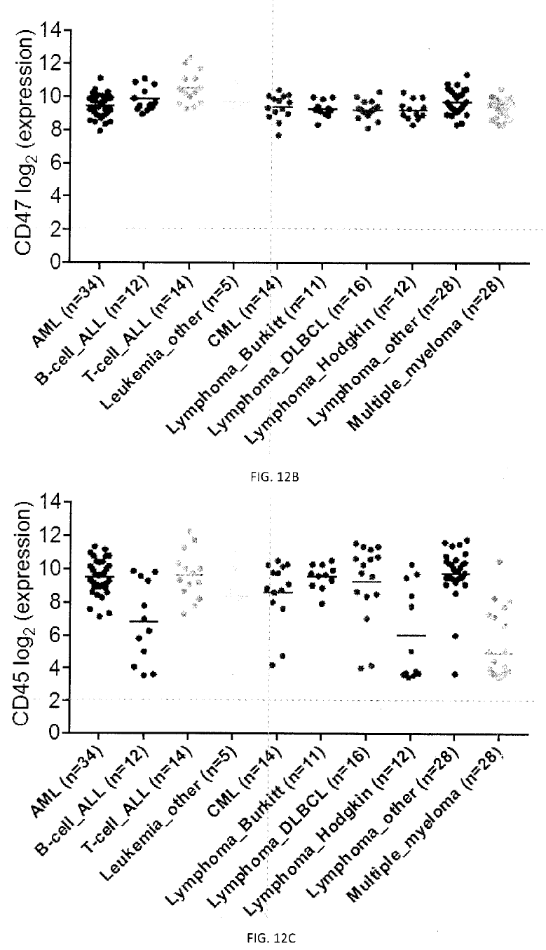

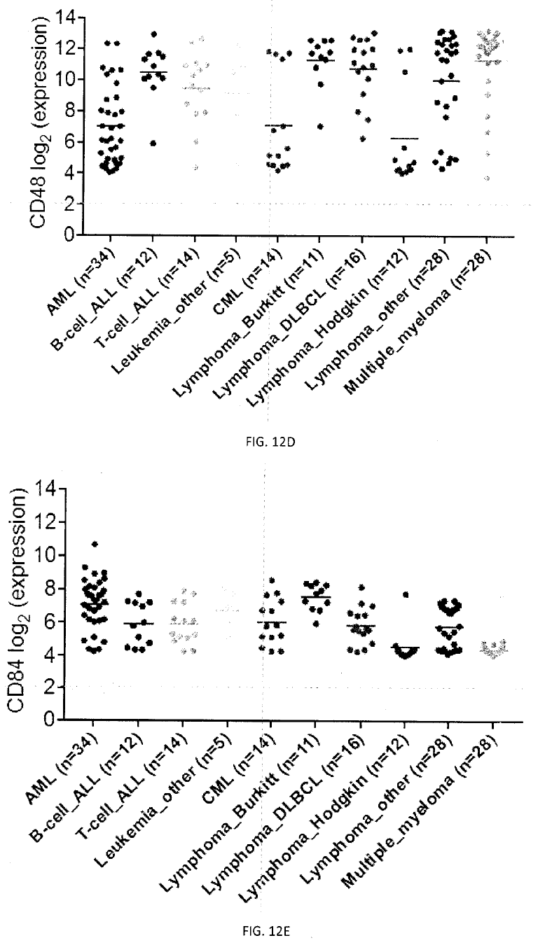

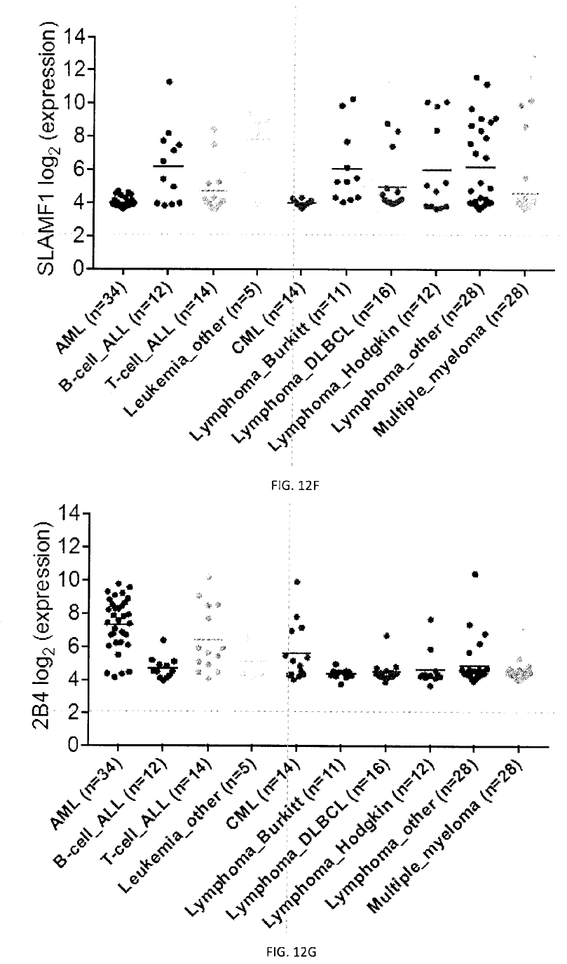

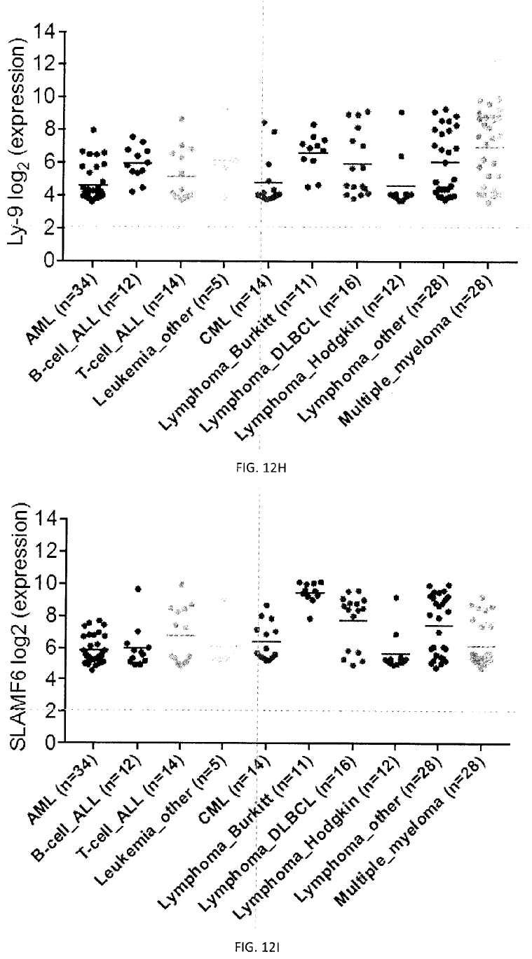

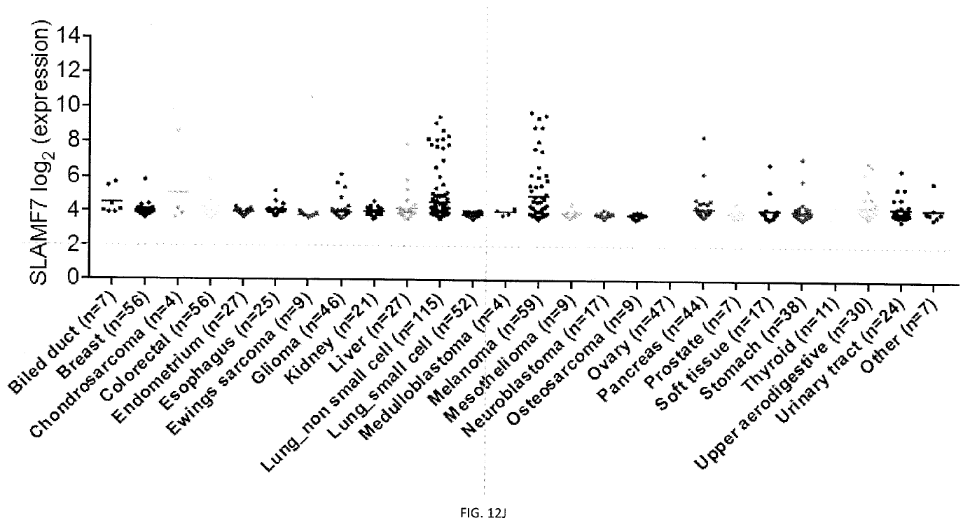

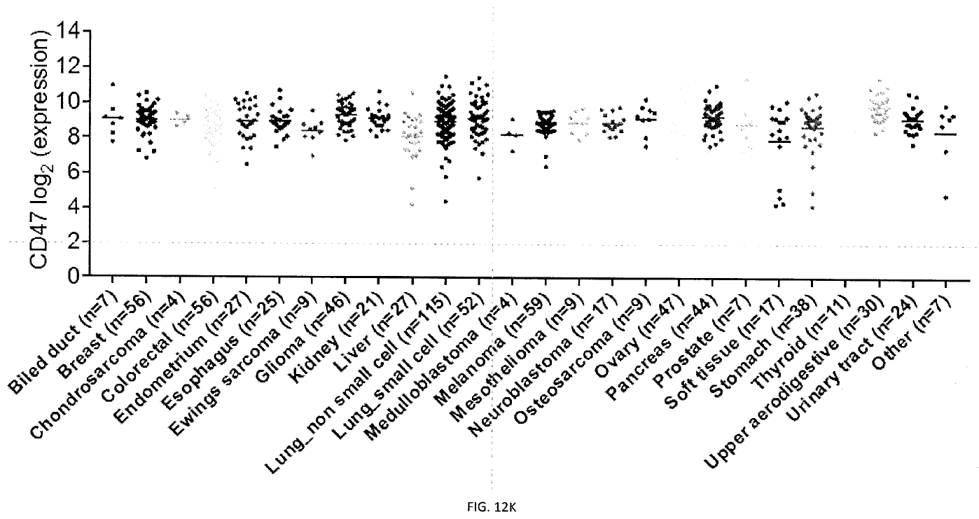

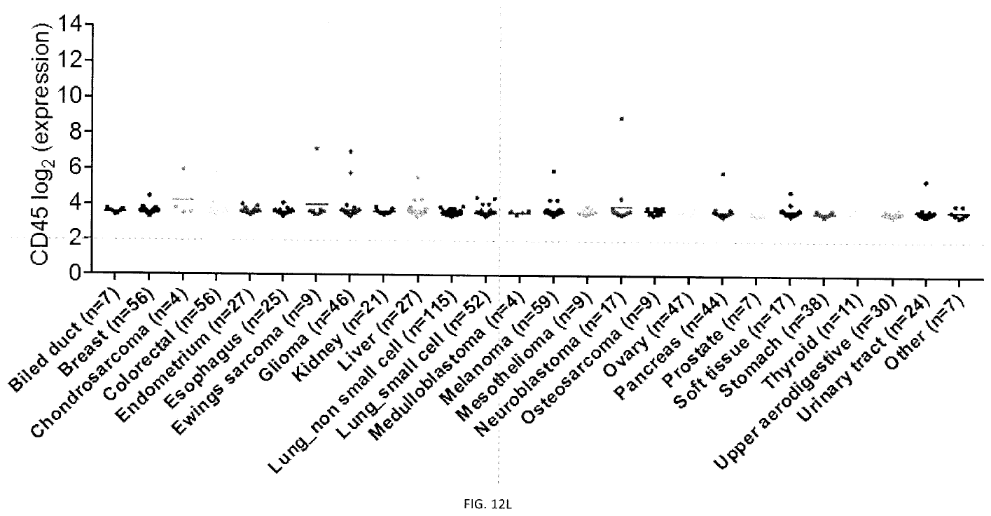

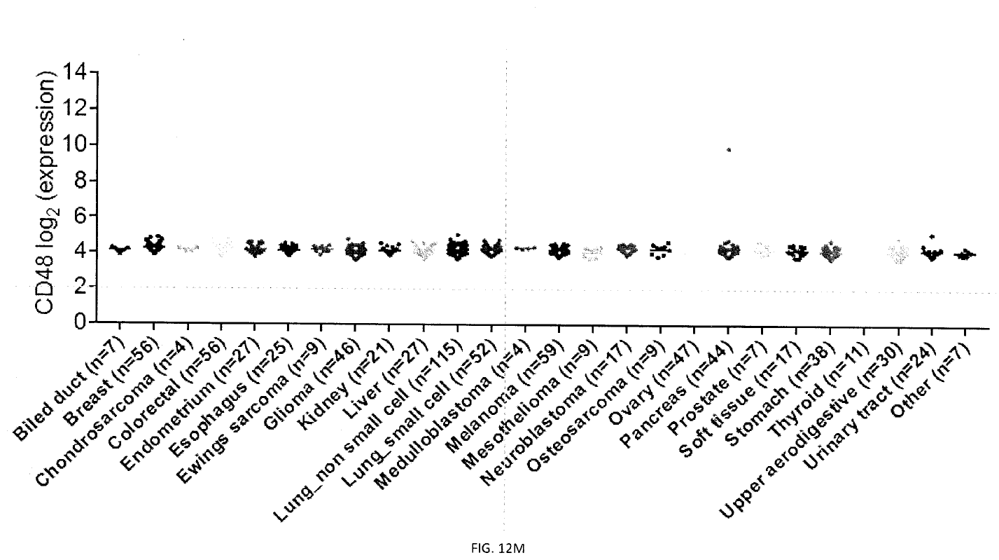

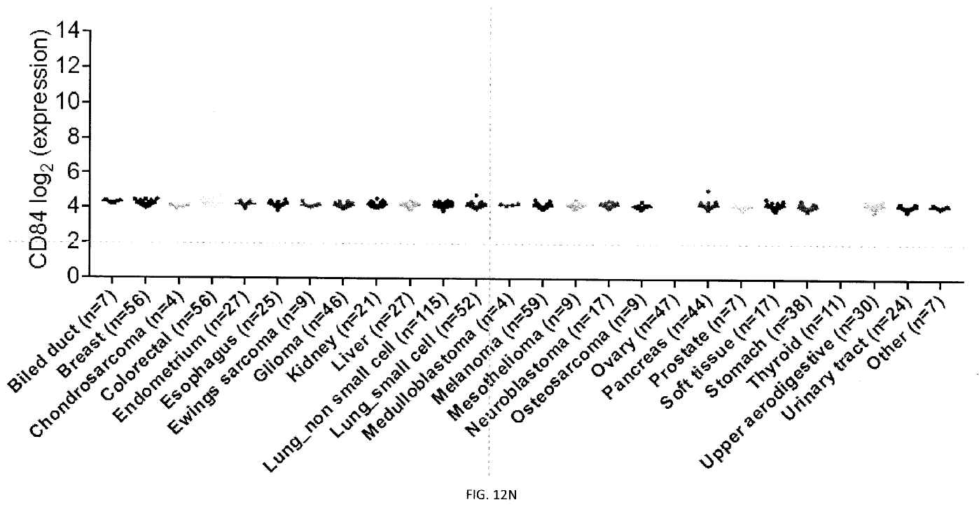

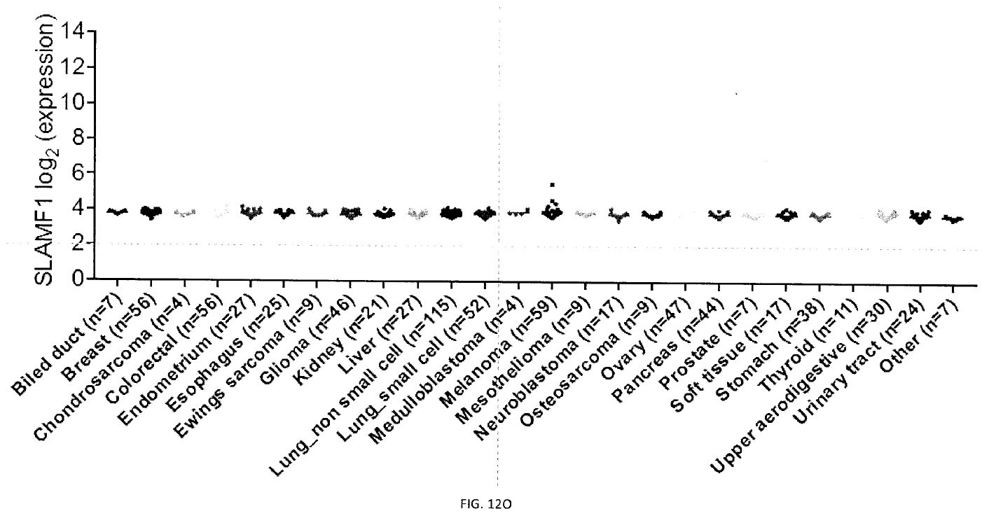

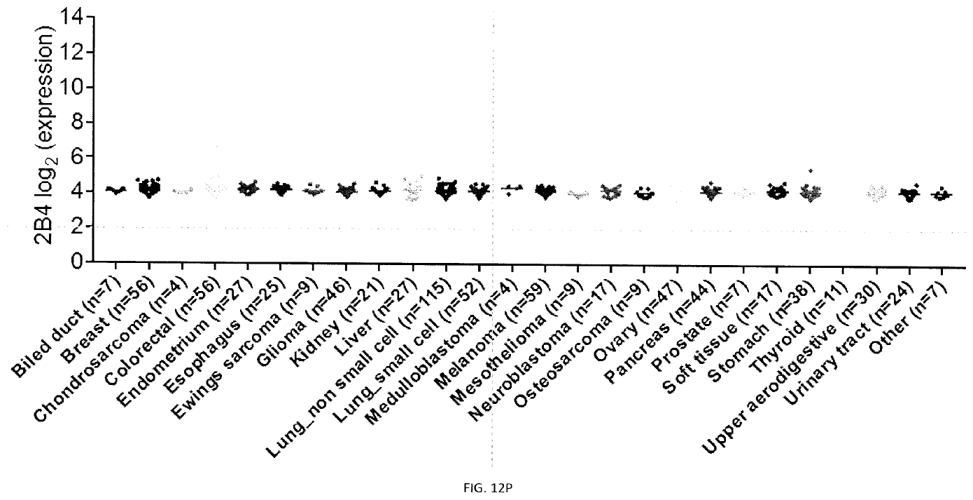

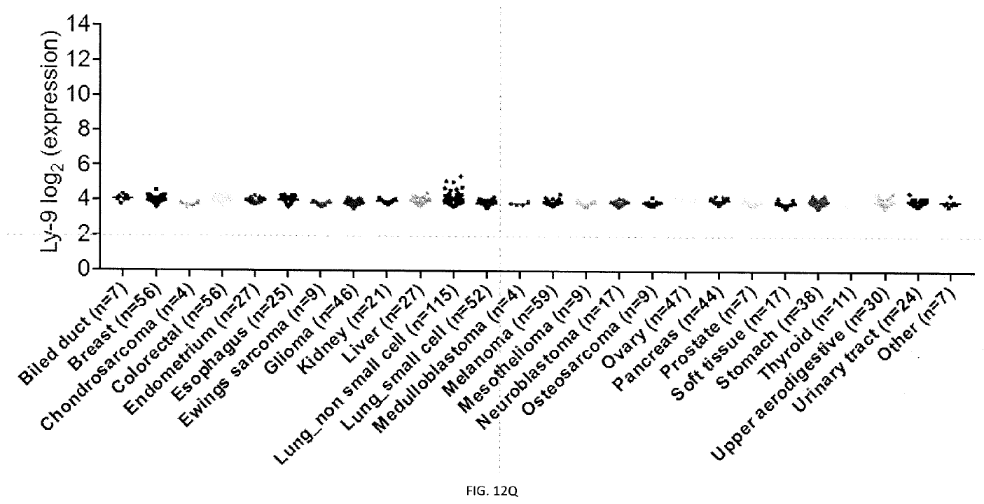

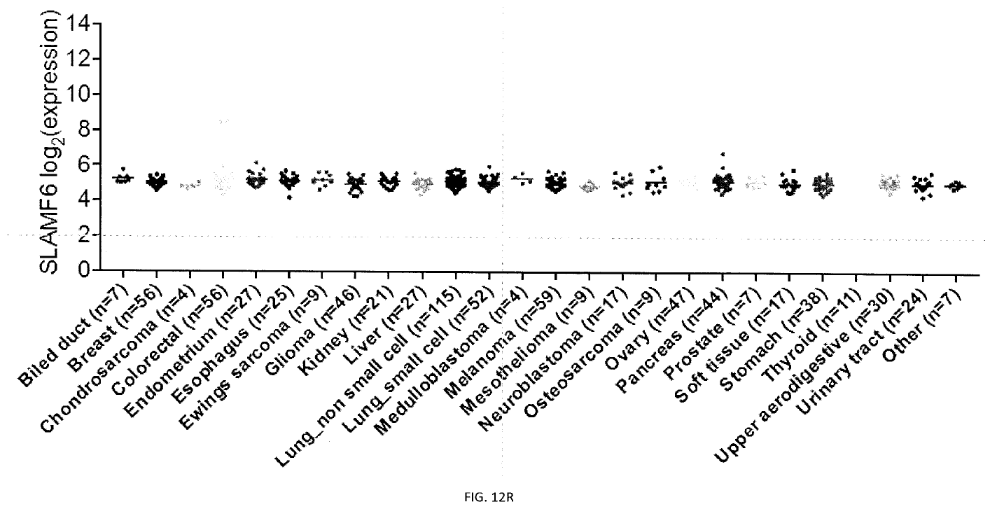

However, certain hematopoietic tumors may be SLAMF7 negative and certain non-hematopoietic (solid tumors) may be SLAMF7 positive. For example, FIG. 12J shows that a certain percentage of bile duct, breast, colorectal, esophagus, glioma, liver, non-small cell lung, melanoma, ovary, pancreas, soft tissue, stomach, upper aerodigestive and urinary tract tumors are SLAMF7 positive. More particularly, this Figure shows that about 20-25% and 35-45% of non-small cell lung tumors and melanoma are SLAMF7 positive, respectively.

The presence of SLAMF7 on a tumor in a subject is an indication that a prevention and/or treatment of the subject with a signal regulatory protein alpha (SIRPalpha)-cluster of differentiation 47 (CD47) checkpoint inhibitor will be effective. It is also an indication that the further use of a SLAMF7 agonist for preventing and/or treating such tumors will be effective.

In contrast, the absence of SLAMF7 on a tumor in a subject is an indication that a prevention and/or treatment of the subject with a signal regulatory protein alpha (SIRPalpha)-cluster of differentiation 47 (CD47) checkpoint inhibitor will not be effective. Then a SLAMF7 inhibitor can be used to treat these tumors to activate T cells, along optionally with another agent that activates T cells such as anti-PD-1, anti-PD-L1 or anti-CTLA-4. Alternatively, a SIRPalpha-CD47 checkpoint inhibitor could be used with an Fc receptor-binding antibody or fragment thereof targeting an antigen (e.g., CD20) expressed at the surface of tumor cells. (e.g., rituximab). An “Fc receptor binding antibody or fragment thereof” as used herein is an intact antibody or a fragment thereof that comprises a functional Fc domain (e.g. intact antibody or heavy chain antibody). See list of potential antibodies and antibody fragments herein.

In one embodiment, the present disclosure relates to benign neoplastic disease. In another embodiment, the present disclosure relates to malignant neoplastic disease. In specific embodiments, the malignant neoplastic disease is cancer.

In an embodiment, the above-mentioned cancer/tumor is associated with SLAMF7 expression and/or activity (e.g., presence of SLAMF7 expression and/or activity, also referred to as SLAMF7-expressing or SLAMF7-positive tumor). In another embodiment, the above-mentioned cancer/tumor is associated with absence of SLAMF7 expression and/or activity (e.g., absence of SLAMF7 expression and/or activity, also referred to as SLAMF7-negative tumor).

Treatment and Prevention

The terms “treat/treating/treatment” and “prevent/preventing/prevention” as used herein, refers to eliciting the desired biological response, i.e., a therapeutic and prophylactic effect, respectively. In accordance with the subject disclosure, the therapeutic effect comprises one or more of a decrease/reduction in the severity of a human disease (e.g., a reduction or inhibition of cancer progression and/or metastasis development), a decrease/reduction in at least one symptom or disease-related effect, an amelioration of at least one symptom or disease-related effect, a decrease/reduction of the development of the cancer resistance to a drug treatment, and an increased survival time of the affected host animal, following administration of the at least one inhibitor (e.g., CD47 inhibitor, SIRPalpha inhibitor, SLAMF7 inhibitor) or agonist (e.g., SLAMF7 agonist), or of a composition comprising the inhibitor or agonist. In accordance with the disclosure, a prophylactic effect may comprise a complete or partial avoidance/inhibition of cancer or a delay of cancer (e.g., a complete or partial avoidance/inhibition of metastasis development or a delay of metastasis development), of drug resistance, and an increased survival time of the affected host animal, following administration of the at least one inhibitor (e.g., CD47 inhibitor, SIRPalpha inhibitor, SLAMF7 inhibitor) or agonist (e.g., SLAMF7 agonist) or of a composition comprising the inhibitor.

As such, a “therapeutically effective” or “prophylactically effective” amount of inhibitor (e.g., CD47 inhibitor, SIRPalpha inhibitor, SLAMF7 inhibitor) or agonist (e.g., SLAMF7 agonist) affecting CD47, SIRPalpha, or SLAMF7 expression and/or activity, or a combination of such inhibitors/agonist, may be administered to an animal, in the context of the methods of treatment and prevention, respectively, described herein.

Types of Samples from the Subject and of Control Samples

As used herein, the term “organism” refers to a living thing which, in at least some form, is capable of responding to stimuli, reproduction, growth or development, or maintenance of homeostasis as a stable whole (e.g., an animal). The organism may be composed of many cells which may be grouped into specialized tissues or organs.

“Sample” or “biological sample” refers to any solid or liquid sample isolated from a live being. In a particular embodiment, it refers to any solid (e.g., tissue sample) or liquid sample isolated from an animal (e.g., human), such as a biopsy material (e.g., solid tissue sample), blood (e.g., plasma, serum or whole blood), saliva, synovial fluid, urine, amniotic fluid and cerebrospinal fluid. Such sample may be, for example, fresh, fixed (e.g., formalin-, alcohol- or acetone-fixed), paraffin-embedded or frozen prior to analysis of CD47, SIRPalpha, or SLAMF7 expression level. In an embodiment, the above-mentioned sample is obtained from a tumor.

As used herein, the term “tissue” or “tissue sample” refers to a group of cells, not necessarily identical, but from the same origin, that together carry out a specific function. A tissue is a cellular organizational level intermediate between cells and a complete organism. Organs are formed by the functional grouping together of multiple tissues. Examples of tissues include dermal, adipose, connective tissue, epithelial, muscle, nervous tissues. Other examples of biological tissues include blood cells populations (e.g., B or T lymphocytes populations), breast, skin, lung or colon tissues.

Similarly, the expression “reference gene expression and/or activity of a gene” refers to the expression and/or activity of that gene used as a control for the measure performed in a sample from a subject. “Reference gene sample” as used herein refers to a sample comprising a reference expression and/or activity of a gene.

More particularly, the expression “reference SLAMF7 expression and/or activity” and “reference CD47 expression and/or activity” and “reference SIRPalpha expression and/or activity” refers to the SLAMF7, CD47 or SIRPalpha expression and/or activity, respectively, used as a control for the measure performed in a sample from a subject. “Reference SLAMF7 sample” or “reference CD47 sample” or “reference SIRPalpha sample” as used herein refer to a sample comprising a “reference SLAMF7 expression and/or activity” and “reference CD47 expression and/or activity” and “reference SIRPalpha expression and/or activity”, respectively.

Depending on the type of assay performed, the reference SLAMF7 expression and/or activity and reference CD47 expression and/or activity and reference SIRPalpha expression and/or activity can be selected from an established standard, a corresponding SLAMF7, CD47 or SIRPalpha expression and/or activity, respectively, determined in the subject (in a sample from the subject) at an earlier time; a corresponding SLAMF7, CD47 or SIRPalpha expression and/or activity, respectively, determined in one or more control subject(s) known to not being predisposed to a neoplastic disease, known to not having an hematopoietic derived tumor (in specific embodiments, a B-cell derived tumor, a myeloid cell derived tumor, a multiple myeloma or a mastocytoma), known to not having a solid tumor cancer (e.g., colon, breast, lung or skin cancer (melanoma)) or known to have a good prognosis; known to have a predisposition to an neoplastic disease or known to have an neoplastic disease (e.g., a specific tumor subtype) or known to have a poor prognosis. In another embodiment, reference SLAMF7 expression and/or activity and reference CD47 expression and/or activity and reference SIRPalpha expression and/or activity is the average or median value obtained following determination of SLAMF7, CD47 or SIRPalpha expression or activity, respectively, in a plurality of samples (e.g., samples obtained from several healthy subjects or samples obtained from several subjects having a neoplastic disease (e.g., cancer)).

“Corresponding normal tissue” or “corresponding tissue” as used herein refers to a reference sample obtained from the same tissue as that obtained from a subject. Corresponding tissues between organisms (e.g., human subjects) are thus tissues derived from the same origin (e.g., two B lymphocyte populations).

Measurement of SLAMF7, CD47 or SIRPalpha in a Sample

The present disclosure encompasses methods comprising detecting the presence of SLAMF7, CD47 or SIRPalpha activity and/or expression in a subject sample. In a specific embodiment, the present disclosure encompasses detecting the presence of SLAMF7 and CD47 activity and/or expression in a subject sample. In another specific embodiment, the present disclosure encompasses detecting the presence of SLAMF7, CD47, and SIRPalpha activity and/or expression in a subject sample. In another specific embodiment, the present disclosure encompasses detecting the presence of SLAMF7 activity and/or expression in a subject sample.

In another embodiment, the present disclosure encompasses methods comprising determining whether SLAMF7, CD47 or SIRPalpha activity and/or expression in a subject sample is substantially similar to that in a reference expression and/or activity. In a specific embodiment, the present disclosure encompasses determining whether SLAMF7 and CD47 activity and/or expression activity and/or expression in a subject sample is substantially similar to that in a reference expression and/or activity. In a specific embodiment, the present disclosure encompasses determining whether SLAMF7, CD47, and SIRPalpha activity and/or expression in a subject sample is substantially similar to that in a reference expression and/or activity. In a specific embodiment, the present disclosure encompasses determining whether SLAMF7 activity and/or expression in a subject sample is substantially similar to that in a reference expression and/or activity

In another embodiment, the present disclosure encompasses methods comprising determining whether SLAMF7, CD47 or SIRPalpha activity and/or expression in a subject sample is higher than a reference expression and/or activity. In a specific embodiment, the present disclosure encompasses determining whether SLAMF7 and CD47 activity and/or expression in a subject sample is higher than that in a reference expression and/or activity. In a specific embodiment, the present disclosure encompasses determining whether SLAMF7, CD47, and SIRPalpha activity and/or expression in a subject sample is higher than that in a reference expression and/or activity. In a specific embodiment, the present disclosure encompasses determining whether SLAMF7 activity and/or expression in a subject sample is higher than that in a reference expression and/or activity

In cases where the reference SLAMF7, CD47 or SIRPalpha sample is from the subject at an earlier time; from subject(s) known not to being predisposed to an neoplastic disease, known not to have an neoplastic disease, or known to have a good prognosis, (1) an increased/higher SLAMF7 and (i) CD47 and/or (ii) SIRPalpha, expression and/or activity, respectively in the sample from the subject relative to the reference SLAMF7 and (i) CD47 and/or (ii) SIRPalpha expression and/or activity, respectively, is indicative that the subject would likely benefit from a SIRPalpha-CD47 blocker (inhibitor) and potentially from a SLAMF7 agonist, while a comparable or lower expression or activity in a sample from the subject relative to the reference expression and/or activity is indicative that the subject would likely not benefit from a SIRPalpha-CD47 blocker (inhibitor) or from a SLAMF7 agonist. In such a case, then a SLAMF7 inhibitor can be used to treat these tumors to activate T cells, along optionally with another agent that activates T cells.

In cases where the reference SLAMF7, CD47 or SIRPalpha sample is from subject(s) known to have a predisposition to an neoplastic disease, known to have an neoplastic disease or known to have a poor prognosis, (1) a comparable or an increased/higher SLAMF7 and (i) CD47 and/or (ii) SIRPalpha, expression and/or activity, respectively in the sample from the subject relative to the reference SLAMF7 and (i) CD47 and/or (ii) SIRPalpha expression and/or activity, respectively, is indicative that the subject would likely benefit from a SIRPalpha-CD47 blocker (inhibitor) and potentially from a SLAMF7 agonist, while a lower expression or activity in a sample from the subject relative to the reference expression and/or activity is indicative that the subject would likely not benefit from a SIRPalpha-CD47 blocker (inhibitor), or from a SLAMF7 agonist. Then a SLAMF7 inhibitor can be used to treat these tumors to activate T cells, along optionally with another agent that activates T cells.

As used herein, a “higher” or “increased” level refers to levels of expression or activity in a sample (i.e. sample from the subject) which exceeds with statistical significance that in the reference sample (e.g., an average corresponding level of expression or activity a healthy subject or of a population of healthy subjects, or when available, the normal counterpart of the affected or pathological tissue) measured through direct (e.g., Anti-SLAMF7 antibody, Anti-CD47 antibody, or anti-SIRPalpha, quantitative PCR) or indirect methods. The increased level of expression and/or activity refers to level of expression and/or activity in a sample (i.e. sample from the subject) which is at least 10% higher, in another embodiment at least 15% higher, in another embodiment at least 20% higher, in another embodiment at least 25%, in another embodiment at least 30% higher, in a further embodiment at least 40% higher; in a further embodiment at least 50% higher, in a further embodiment at least 60% higher, in a further embodiment at least 100% higher (i.e. 2-fold), in a further embodiment at least 200% higher (i.e. 3-fold), in a further embodiment at least 300% higher (i.e. 4-fold), relative to the reference expression and/or activity (e.g., in corresponding normal adjacent tissue or alternatively, in a define group of subject).

As used herein, a “substantially similar level” refers to a difference in the level of expression or activity between the level determined in a first sample (e.g., sample from the subject) and the reference expression and/or activity which is less than about 10%; in a further embodiment, 5% or less, in a further embodiment, 2% or less.

Methods for measuring SLAMF7, CD47 or SIRPalpha expression and/or activity are well known. See in particular under title “Protein expression” above and Examples herein.

Subjects Stratification Methods

The methods of the present disclosure may also be used for classifying or stratifying a subject into subgroups based on SLAMF7, and optionally CD47 and/or SIRPalpha (and/or protein(s) of one or more other inhibitory checkpoint) expression and/or activity enabling a better characterization of the subject disease and a better selection of treatment and/or to determine whether a subject should be included in a clinical trial testing SIRPalpha-CD47 inhibitors, depending on the subgroup to which the subject belongs. If a subject belongs to the subgroup of subjects having SLAMF7 positive tumors, he would likely be a good candidate for inclusion in a clinical trial testing a SIRPalpha-CD47 inhibitor (i.e. likely responsive to such inhibitor). If a subject belongs to the subgroup of subjects having SLAMF7 negative tumors, he would likely not be a good candidate for inclusion in a clinical trial testing a SIRPalpha-CD47 inhibitor (i.e. likely not responsive to such inhibitor).

In one aspect, the present disclosure provides a method for stratifying a subject, said method comprising: (a) detecting/determining the expression and/or activity of SLAMF7 in a sample from the subject, and optionally (b) comparing said expression and/or activity to a reference expression and/or activity; and (c) stratifying said subject based on said detection and/or said comparison. In a specific embodiment, the method further comprises detecting/determining the expression and/or activity of CD47 and/or SIRPalpha and/or protein(s) of one or more other inhibitory checkpoints.

The disclosure provides a method for stratifying a subject based on the expression and/or activity of such biomarkers as determined in a tissue sample (e.g., a biopsy) from the subject using the assays/methods described herein.

Combination of Therapies

In an embodiment, the above-mentioned prevention/treatment comprises the use/administration of more than one (i.e. a combination of) therapies (e.g., active/therapeutic agent (e.g., an agent capable of inhibiting SIRPalpha-checkpoint expression and/or activity; or an agent capable of activating T cells). The combination of prophylactic/therapeutic agents and/or compositions of the present disclosure may be administered or co-administered (e.g., consecutively, simultaneously, at different times) in any conventional dosage form. Co-administration in the context of the present disclosure refers to the administration of more than one prophylactic or therapeutic agent in the course of a coordinated treatment to achieve an improved clinical outcome. Such co-administration may also be coextensive, that is, occurring during overlapping periods of time. For example, a first agent may be administered to a subject before, concomitantly, before and after, or after a second active agent is administered. The agents may in an embodiment be combined/formulated in a single composition and thus administered at the same time. In an embodiment, the one or more active agent(s) of the present disclosure is used/administered in combination with one or more agent(s) currently used to prevent or treat the disorder in question (e.g., an antineoplastic agent).

Currently used combined therapies for treating cancer include the administration of radiation therapy with therapeutic antineoplastic agents.

Inhibitor/Agonists of the Present Disclosure Combined Treatment in SLAMF7-Positive or SLAMF7-Negative Cells

In one embodiment, the treatment of SLAMF7-positive neoplastic cells with a compound reducing the expression and/or activity of SIRPalpha-CD47 checkpoint is combined with at least one other active agent (e.g., antineoplastic agent in order to increase macrophage phagocytose of tumor cells).

In an embodiment, the SIRPalpha-CD47 checkpoint inhibitor is used in combined therapy with a SLAMF7 agonist (e.g., elotuzumab).

In another embodiment, in SLAMF7-positive (e.g., solid tumor cancers), the SIRPalpha-CD47 checkpoint inhibitor is used in combined therapy with is used in combined therapy with another immune checkpoint inhibitor such as the anti-PD-1 or anti-PDL1 inhibitor).

In another embodiment, the treatment of SLAMF7-negative neoplastic cells with a compound reducing the expression and/or activity of SLAMF7 is combined with at least one other active agent (e.g., another agent that activates T cells and/or antineoplastic agent in order to increase macrophage phagocytose of tumor cells).

In an embodiment, the SLAMF7 inhibitor is used in combined therapy with another agent that activates T cells.

In other embodiments of the above, the least one SIRPalpha-CD47 checkpoint inhibitor (and eventually SLAMF7 agonist (e.g., elotuzumab)) or the at least one SLAMF7 inhibitor (and eventually agent that activates T cells) is used with another antineoplastic agent known for the treatment of the specific SLAMF7-positive cancer (e.g., B-cell lymphomas, leukemias, multiple myeloma, plasmacytoma, mastocytoma, solid tumor cancers such as bile duct, breast, colorectal, esophagus, glioma, liver, non-small cell lung, melanoma, ovary, pancreas, soft tissue, stomach, upper aerodigestive or urinary tract cancer, preferably glioma, liver, non-small cell lung, melanoma, upper aerodigestive or urinary tract tumor, and more preferably non-small cell lung cancer or melanoma), or the specific SLAMF7-negative cancer (e.g., leukemias and solid tumor cancers such as breast cancer, colon cancer, lung cancer, melanoma), respectively. In specific aspects of the present disclosure, in SLAMF7-positive cancers the SIRPalpha-CD47 checkpoint inhibitor (and eventually SLAMF7 agonist) is combined to at least one of chemotherapy, radiotherapy, surgery, immunomodulatory drugs or other treatments, as indicated by ongoing medical practices. In specific aspects of the present disclosure, in SLAMF7-negative cancers, the SLAMF7 inhibitor (and eventually another agent that activates T cells e.g., PD-1 inhibitor) is combined to at least one of chemotherapy, radiotherapy, surgery, immunomodulatory drugs or other treatments, as indicated by ongoing medical practices.

In other specific aspects of the present disclosure, in SLAMF7-negative cancers, a SLAMF7 protein or nucleic acid can first be used to force SLAMF7 expression on the tumor(s). Then the treatment described herein for SLAMF7 positive tumors can be applied.

In other embodiments, where the target cells are generally less susceptible to macrophage phagocytosis, the treatment described above can be administered with an inflammatory stimulant enhancing the ability of macrophage to phagocytose.

Nucleic Acids and Host Cells

The present disclosure also relates to nucleic acids comprising nucleotide sequences encoding the above-mentioned inhibitors or agonists (e.g., SLAMF7). The nucleic acid may be codon-optimized. The nucleic acid can be a DNA or an RNA. The nucleic acid sequence can be deduced by the skilled artisan on the basis of the disclosed amino acid sequences.

The present disclosure also encompasses vectors (plasmids) comprising the above-mentioned nucleic acids. The vectors can be of any type suitable, e.g., for expression of said polypeptides or propagation of genes encoding said polypeptides in a particular organism. The organism may be of eukaryotic or prokaryotic origin. The specific choice of vector depends on the host organism and is known to a person skilled in the art. In an embodiment, the vector comprises transcriptional regulatory sequences or a promoter operably-linked to a nucleic acid comprising a sequence encoding one or more of the above-mentioned inhibitors or agonists (e.g., SLAMF7) of the disclosure. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequence. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous. “Transcriptional regulatory sequences” or “transcriptional regulatory elements” are generic terms that refer to DNA sequences, such as initiation and termination signals, enhancers, and promoters, splicing signals, polyadenylation signals, etc., which induce or control transcription of protein coding sequences with which they are operably-linked.

A recombinant expression vector comprising a nucleic acid sequence of the present disclosure may be introduced into a cell, e.g., a host cell (such as a tumor cell), which may include a living cell capable of expressing the protein coding region from the defined recombinant expression vector. Accordingly, the present disclosure also relates to cells (host cells) comprising the nucleic acid and/or vector as described above. The suitable host cell may be any cell of eukaryotic or prokaryotic (bacterial) origin that is suitable, e.g., for expression of or propagation of genes/nucleic acids encoding said above-mentioned inhibitors or agonists (e.g., SLAMF7). The eukaryotic cell line may be of mammalian, of yeast, or invertebrate origin. The specific choice of cell line is known to a person skilled in the art. Choice of bacterial strain will depend on the task at hand and is known to a person skilled in the art. The terms “host cell” and “recombinant host cell” are used interchangeably herein. Such terms refer not only to the particular subject cell, but also to the progeny or potential progeny of such a cell. Because certain modifications may occur in succeeding generations due to either mutation or environmental influences, such progeny may not, in fact, be identical to the parent cell, but are still included within the scope of the term as used herein. Vectors can be introduced into cells via conventional transformation or transfection techniques. The terms “transformation” and “transfection” refer to techniques for introducing foreign nucleic acid into a host cell (such as a tumor cell), including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), Sambrook and Russell (supra) and other laboratory manuals. Methods for introducing nucleic acids into mammalian cells in vivo are also known, and may be used to deliver the vector DNA of the disclosure to a subject for gene therapy.

The above-mentioned nucleic acid or vector may be delivered to cells in vivo (to induce the expression of the above-mentioned inhibitors or agonists (e.g., SLAMF7)) using methods well known in the art such as direct injection of DNA, receptor-mediated DNA uptake, viral-mediated transfection or non-viral transfection and lipid-based transfection, all of which may involve the use of gene therapy vectors. Direct injection has been used to introduce naked DNA into cells in vivo. A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor. Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes.

Defective retroviruses are well characterized for use as gene therapy vectors (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include psiCrip, psiCre, psi2 and psiAm. Retroviruses have been used to introduce a variety of genes into many different cell types, including epithelial cells, endothelial cells, lymphocytes, myoblasts, hepatocytes, bone marrow cells, in vitro and/or in vivo.

For use as a gene therapy vector, the genome of an adenovirus may be manipulated so that it encodes and expresses a nucleic acid of the disclosure (e.g., a nucleic acid encoding one of the above-mentioned inhibitors or agonists (e.g., SLAMF7)), but is inactivated in terms of its ability to replicate in a normal lytic viral life cycle. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium, endothelial cells, hepatocytes, and muscle cells.

Adeno-associated virus (AAV) may be used as a gene therapy vector for delivery of DNA for gene therapy purposes. AAV is a naturally occurring defective virus that requires another virus, such as an adenovirus or a herpes virus, as a helper virus for efficient replication and a productive life cycle. AAV may be used to integrate DNA into non-dividing cells. Lentiviral gene therapy vectors may also be adapted for use in the disclosure.

Dosage

The amount of the agent or pharmaceutical composition which is effective in the prevention and/or treatment of a particular disease, disorder or condition (e.g., neoplastic disease) will depend on the nature and severity of the disease, the chosen prophylactic/therapeutic regimen (i.e., compound, DNA construct, protein, cells), systemic administration versus localized delivery, the target site of action, the patient's body weight, patient's general health, patient's sex, special diets being followed by the patient, concurrent medications being used (drug interaction), the administration route, time of administration, and other factors that will be recognized and will be ascertainable with routine experimentation by those skilled in the art. The dosage will be adapted by the clinician in accordance with conventional factors such as the extent of the disease and different parameters from the patient. Typically, 0.001 to 1000 mg/kg of body weight/of subject per day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a subject, in the context of the present disclosure should be sufficient to produce a beneficial prophylactic and/or therapeutic response in the patient over time. The size of the dose will also be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

Adjustment of Dose of Inhibitors/Agonist of the Present Disclosure

In one embodiment of the present disclosure, the dose of the at least one SLAMF7, CD47, SIRPalpha or SLAMF7 agonist administered to inhibit SLAMF7, CD47, or SIRPalpha expression and/or activity or increase SLAMF7 expression and/or activity, is adjusted to the level of SLAMF7, CD47 or SIRPalpha in the sample (e.g., tumor tissue).

In another aspect, the present disclosure provides a method for adjusting a treatment, for example the dose of an inhibitor to administer to a subject. Such method comprising: (a) determining the expression and/or activity of SLAMF7, CD47, or SIRPalpha in a sample from said patient; (b) comparing said expression and/or activity to a reference expression and/or activity of SLAMF7, CD47, or SIRPalpha determined in a biological sample obtained from said patient at an earlier time (e.g., at the start of treatment); wherein a decrease in said expression and/or activity relative to a corresponding expression and/or activity of SLAMF7, CD47, or SIRPalpha determined in a biological sample obtained from said patient at an earlier time (at the start of treatment) is indicative that the dose of the at least one SLAMF7, CD47, or SIRPalpha inhibitor administered is appropriate whereas a similar level or an increase of SLAMF7, CD47, or SIRPalpha expression and/or activity over time is indicative that the dose of the at least one SLAMF7, CD47, or SIRPalpha inhibitor administered to the subject should be increased, Similarly, an increase in said expression and/or activity relative to a corresponding expression and/or activity of SLAMF7 determined in a biological sample obtained from said patient at an earlier time (at the start of treatment) is indicative that the dose of the at least one SLAMF7 agonist administered is appropriate whereas a similar level or a decrease of SLAMF7 expression and/or activity over time is indicative that the dose of the at least one SLAMF7 agonist administered to the subject should be increased.

Pharmaceutical Composition

The disclosure also provides a pharmaceutical composition (medicament) comprising at least one agent of the disclosure (e.g., a SLAMF7, CD47, or SIRPalpha inhibitor or SLAMF7 agonist) (alone or in combination with another agent—see combined treatment above), and a pharmaceutically acceptable carrier (e.g., diluent, solvent, excipient, salt or adjuvant). Such carriers include, for example, saline, buffered saline, dextrose, water, glycerol, ethanol, and combinations thereof. In a specific embodiment, the pharmaceutically acceptable carrier is appropriate for targeting neoplastic cells. The pharmaceutical composition may be adapted for the desired route of administration (e.g., oral, sublingual, nasal, parenteral, intravenous, intramuscular, intra-peritoneal, aerosol). In some embodiments, gene therapy is utilized to deliver therapeutic molecules (e.g., SLAMF7) to the patient. In an embodiment, SLAMF7 negative tumors may be rendered SLAMF7 positive as described e.g., in Example 5, FIGS. 5B-A to B-F, 5C-A to 5C-P, 5D, 5E. See also section on nucleic acids and hosts above. The tumor cells may then be subjected to treatments as described herein for SLAMF7 positive tumors.

Kit or Package

The present disclosure also provides a kit or package comprising the above-mentioned agent (inhibitor or agonist) or pharmaceutical compositions. Such kit may further comprise, for example, instructions for the prevention and/or treatment of a neoplastic disease (e.g., hematopoietic cancer such as B-cell lymphomas, leukemia, non-hematopoietic cancer such as non-small cell lung cancer or melanoma), containers, devices for administering the agent/composition, etc.

The present disclosure also provides a kit or package comprising a reagent useful for determining SLAMF7, CD47, or SIRPalpha expression and/or activity (e.g., a ligand that specifically binds SLAMF7, CD47, or SIRPalpha polypeptide such as an anti-SLAMF7 or anti-CD47 or anti-SIRPalpha antibody, or a ligand that specifically binds a SLAMF7, CD47, or SIRPalpha nucleic acid such as an oligonucleotide). Such kit may further comprise, for example, instructions for the prognosis and/or diagnosis of cancer, control samples, containers, reagents useful for performing the methods (e.g., buffers, enzymes), etc.

As used herein the term “subject” is meant to refer to any animal, such as a mammal including human, mice, rat, dog, cat, pig, cow, monkey, horse, etc. In a particular embodiment, it refers to a human.

A “subject in need thereof” or a “patient” in the context of the present disclosure is intended to include any subject that will benefit or that is likely to benefit from the decrease in the expression or activity of SLAMF7, CD47, or SIRPalpha; or increase of the expression of SLAMF7. In an embodiment, the subject in need thereof is a subject diagnosed as expressing SLAMF7 in tumor cells. In another embodiment, the subject in need thereof is a subject diagnosed as not expressing SLAMF7 in tumor cells.

As used herein, the term “a” or “the” means “at least one”.

Although the present disclosure has been described hereinabove by way of specific embodiments thereof, it can be modified, without departing from the spirit and nature of the subject disclosure as defined in the appended claims.

The present disclosure is illustrated in further details by the following non-limiting examples.

### Example 1: Material and Methods

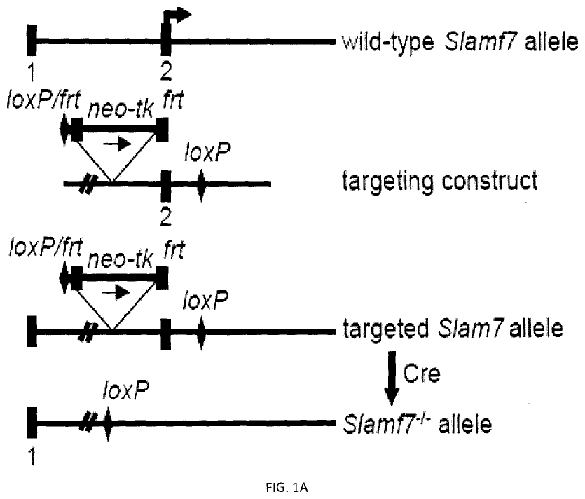

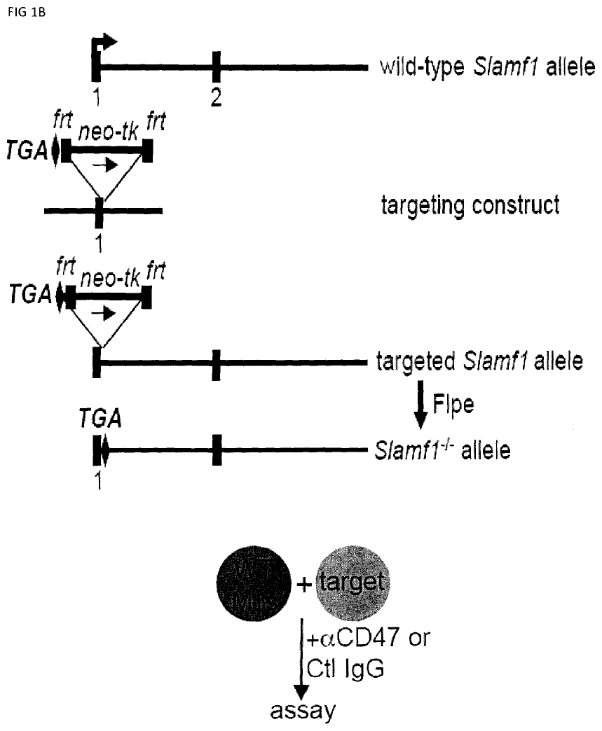

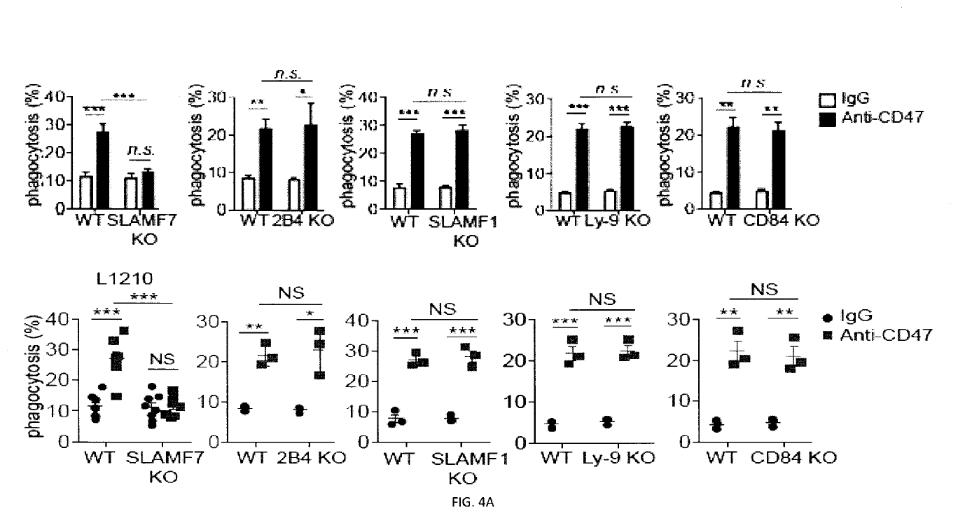

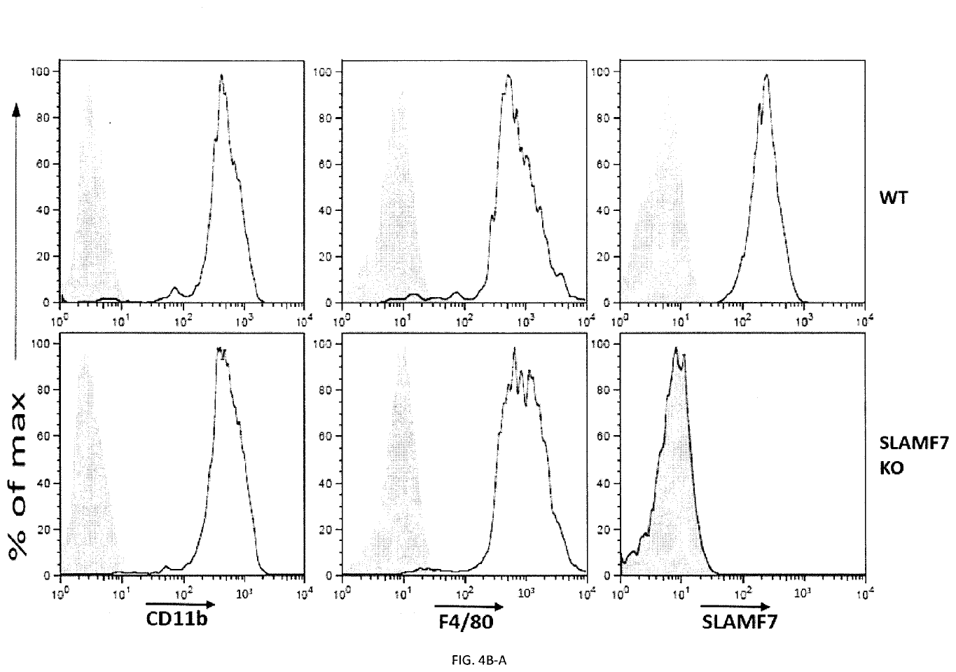

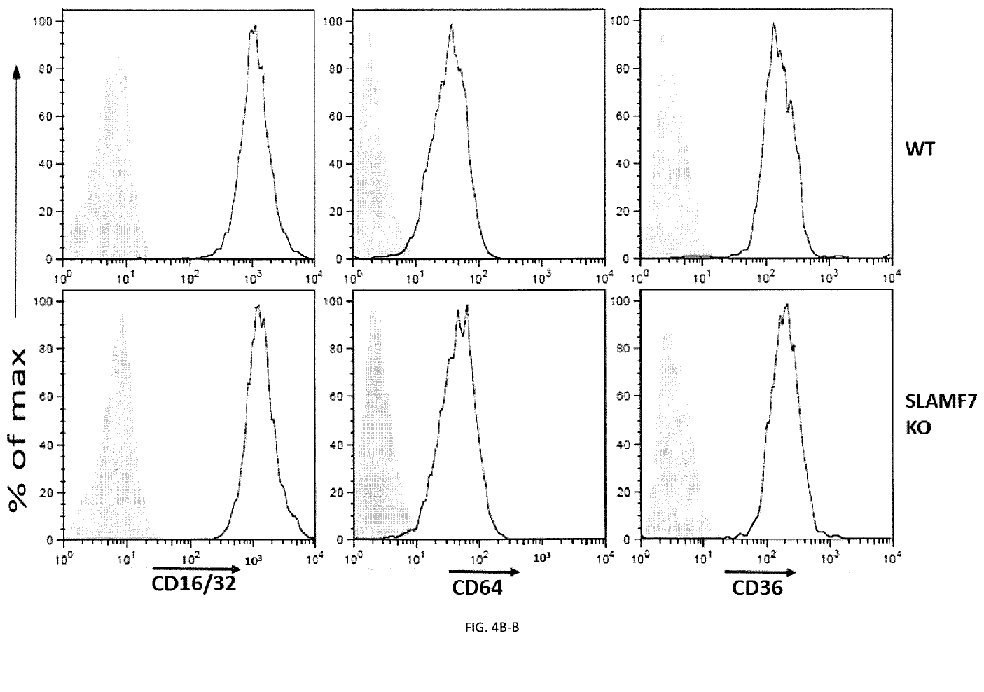

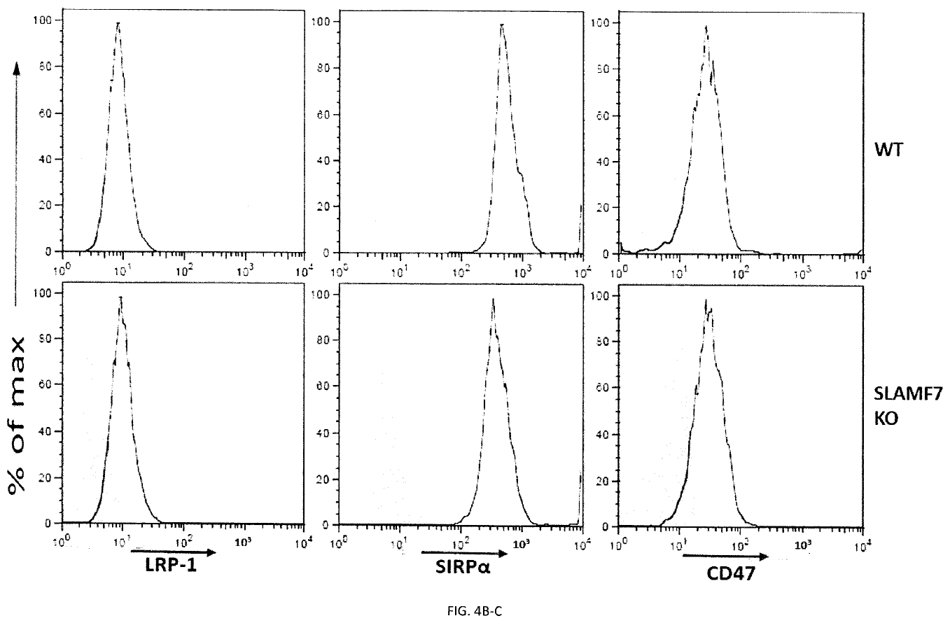

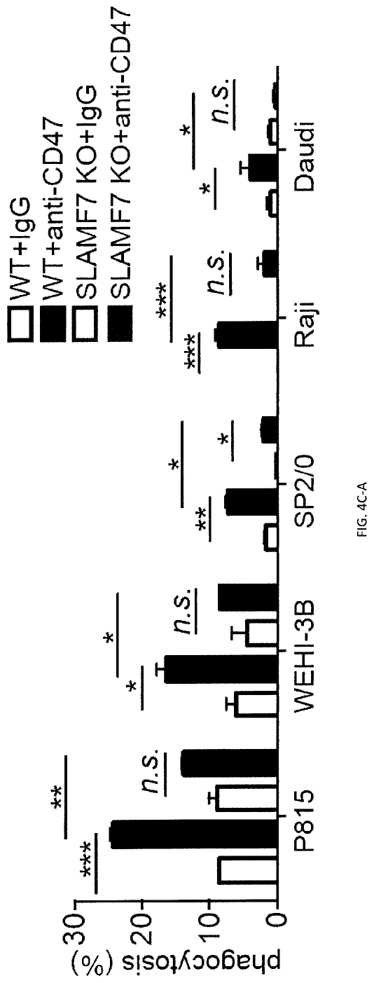

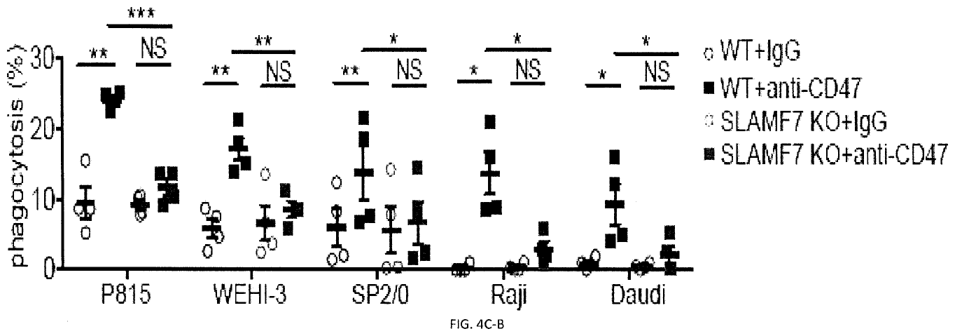

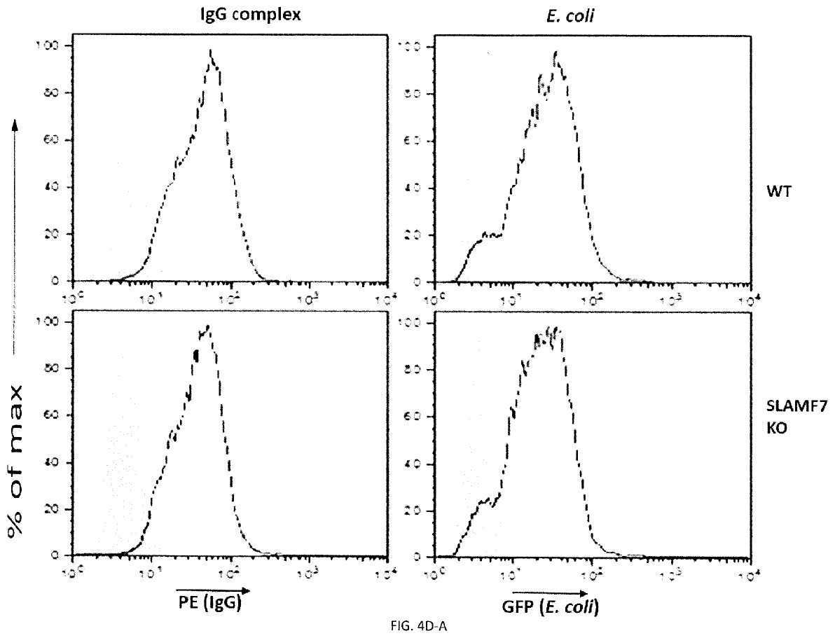

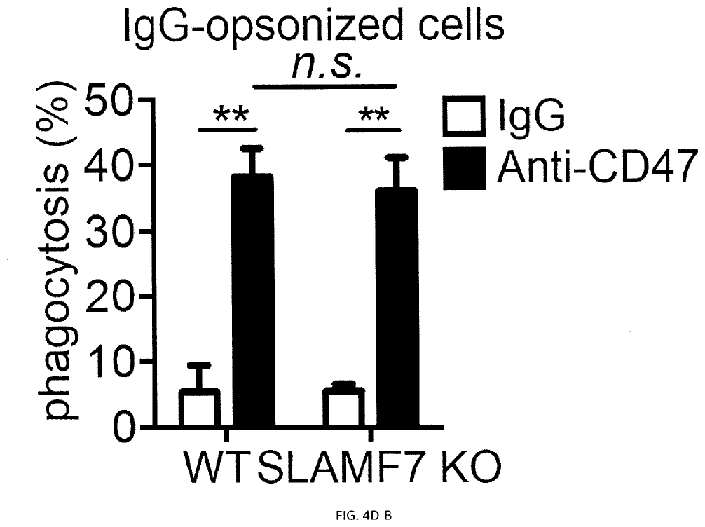

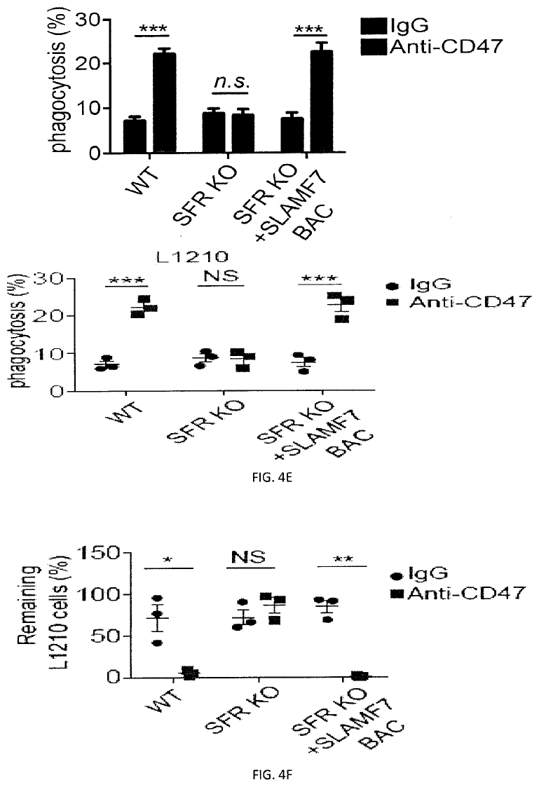

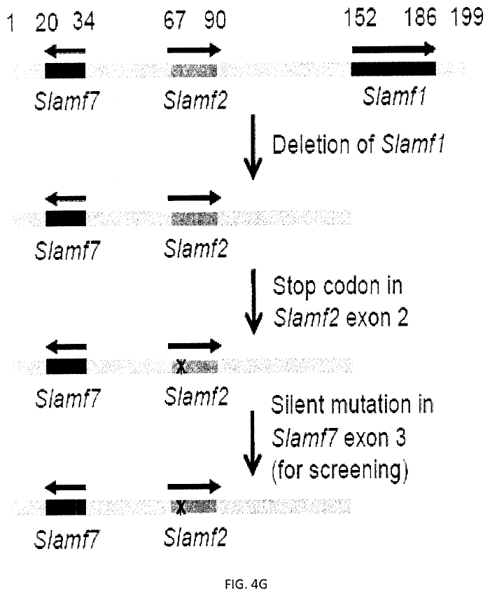

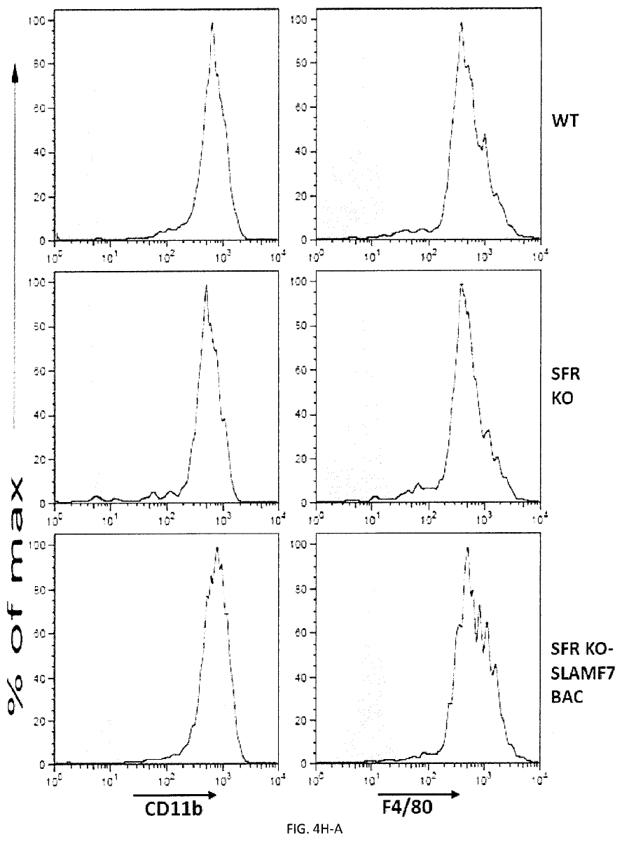

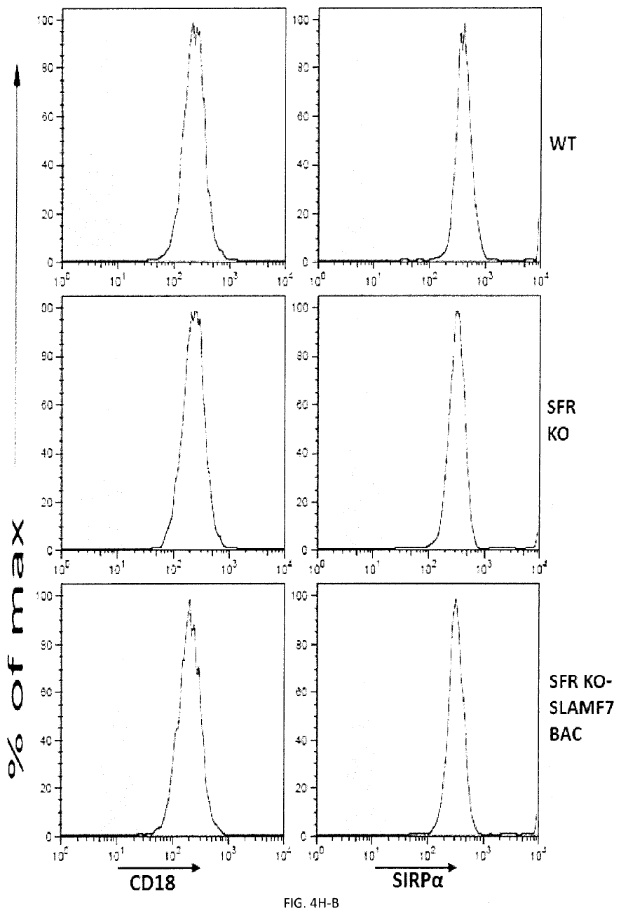

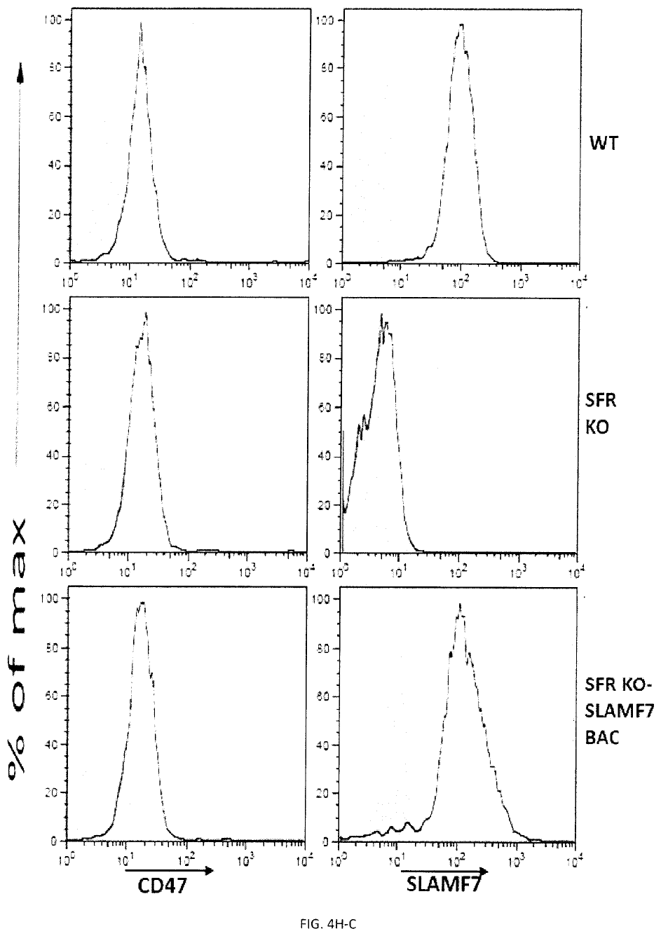

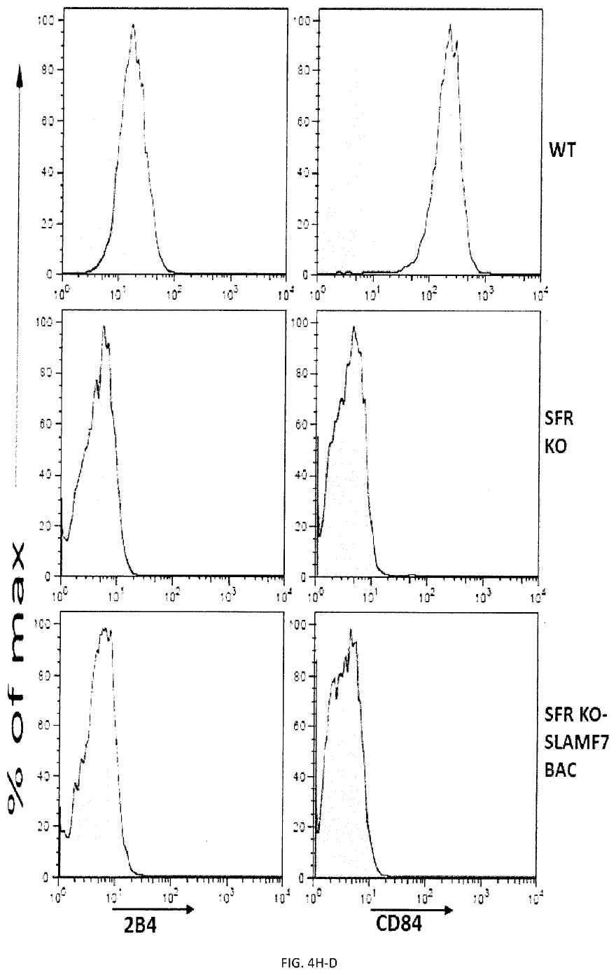

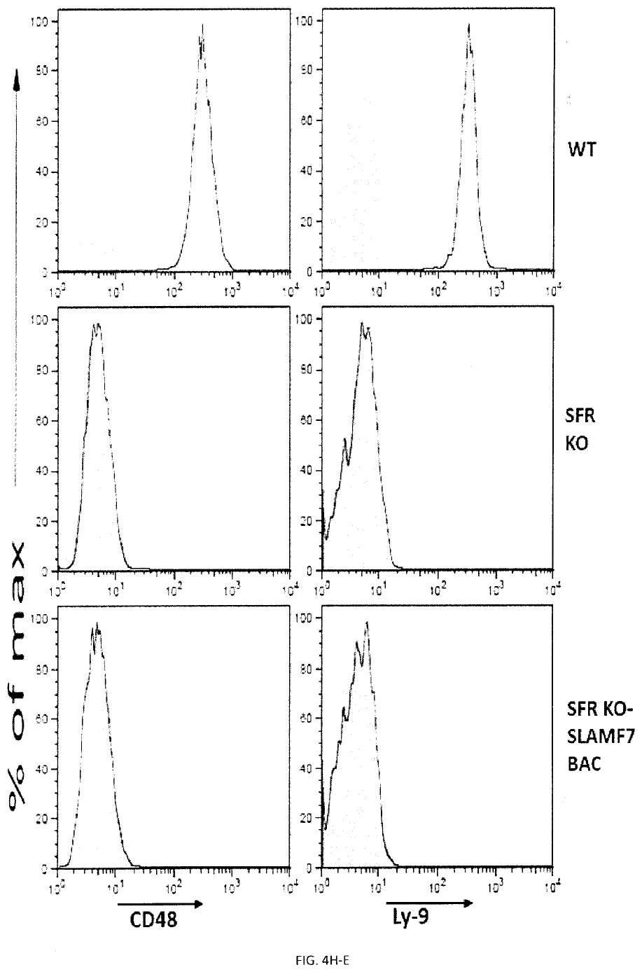

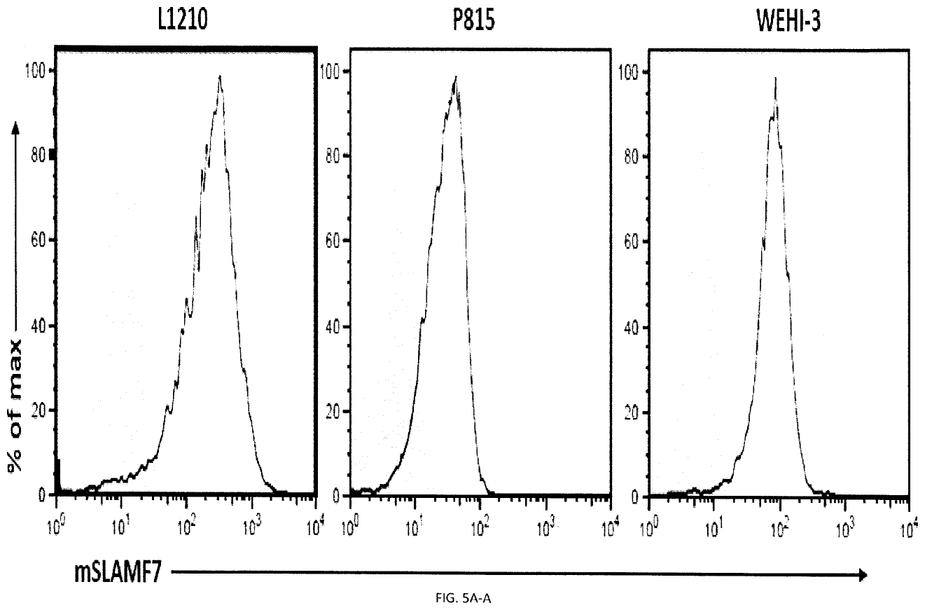

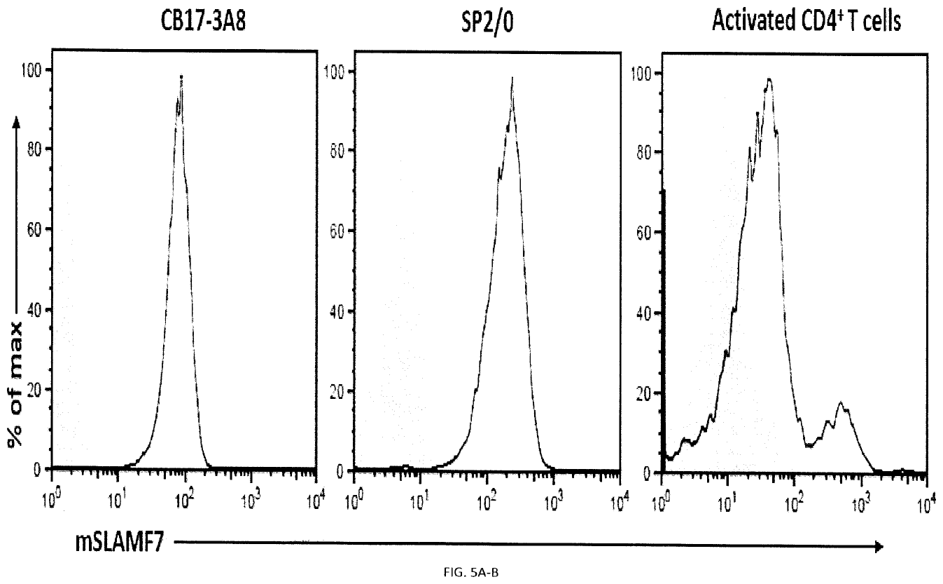

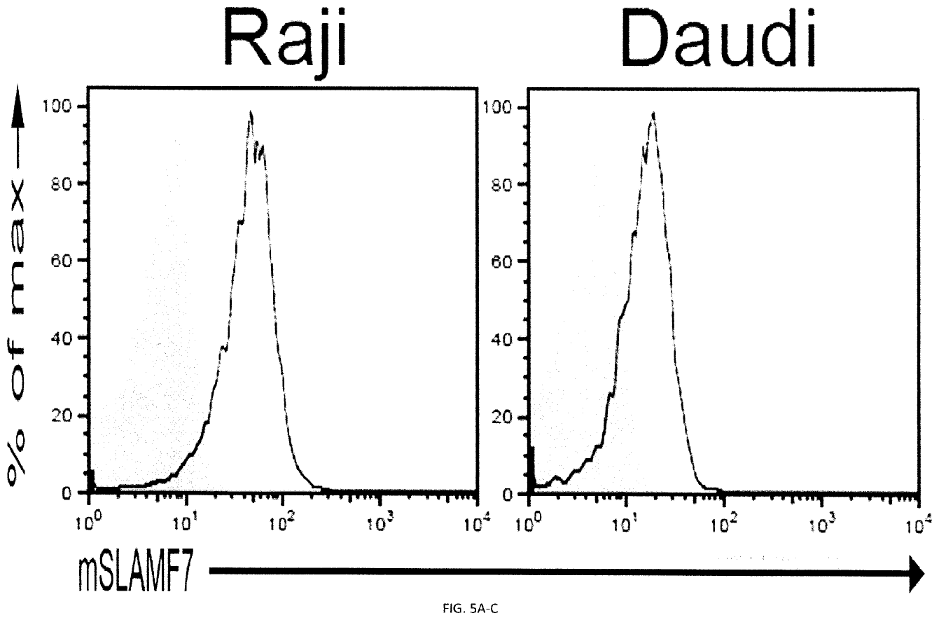

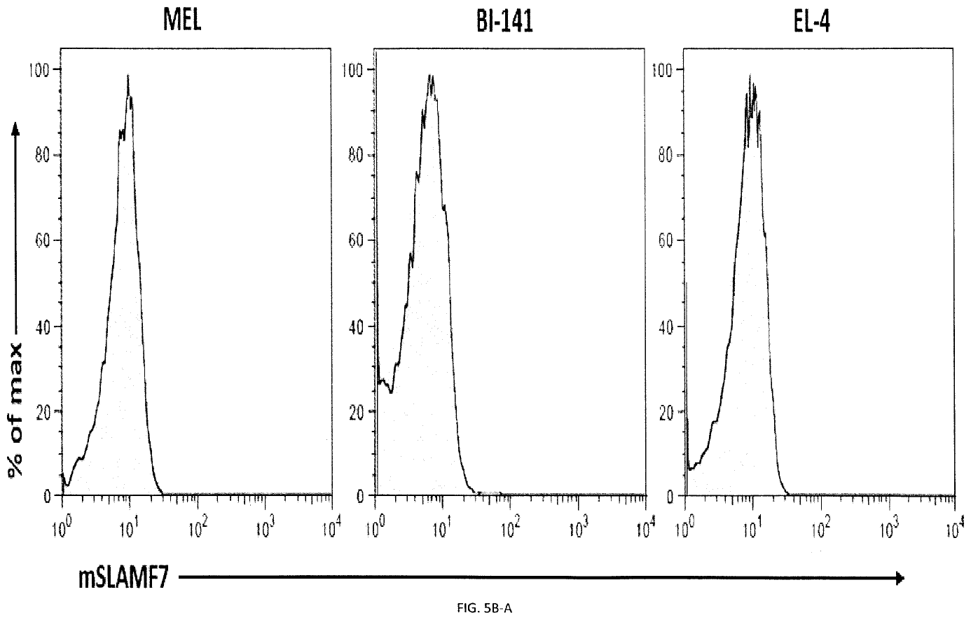

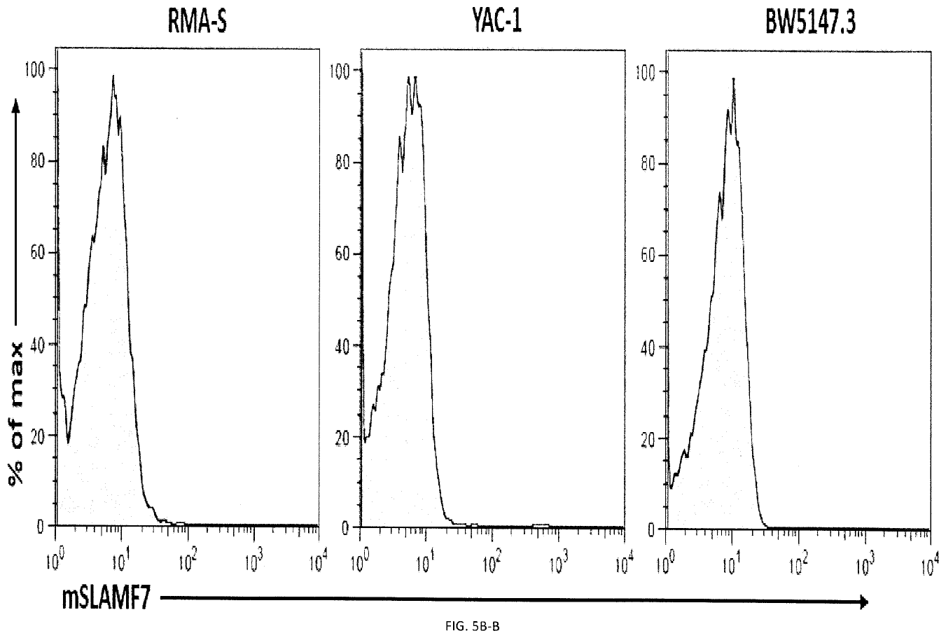

Mice. Mice lacking all SFRs (SFR KO), SLAMF7 (Slamf7^{-/-}) or 2B4 (Slamf4^{-/-}) were described elsewhere⁵⁰. In essence, SFR KO mice were created by deletion of the entire 400 kilobase (kb)-Slam locus in Bruce 4 C57BL/6 embryonic stem (ES) cells. Mice were subsequently backcrossed to the C57BL/6 background for 6-10 generations. Mice lacking SLAMF7 (Slamf7^{-/-}) were created using the strategy and construct depicted in FIG. 1A. After linearizing the construct, the DNA was electroporated into the Bruce 4 C57BL/6 ES cell line, and transfected cells were selected with G418. Clones showing homologous recombination were injected in blastocysts and germ line transmission of the “floxed” allele (Slamf7^{fl/fl}) was achieved. Then, mice were bred with a transgenic mouse expressing the Cre recombinase to delete the neo cassette and exon 2, thereby generating the Slamf7^{-/-} mouse. To produce mice lacking SLAMF1 (Slamf1^{-/-}), DNA fragments encoding SLAMF1 were amplified by PCR from a bacterial artificial chromosome (BAC) clone derived from 129S1/Sv mice. The 5′ and 3′ genomic fragments were then cloned on either side of neo in the vector pJA1617 (FIG. 1B). DNA was then transfected into the 129 mouse embryonic stem cell line R1, and cells were selected with G418. After removal of the neo cassette by transient transfection the Flpe recombinase, positive clones were injected into blastocysts. The resulting chimeric mice were crossed with 12951/Sv mice. Slamf7 BAC transgenic mice (SLAMF7 BAC Tg) were generated using the BAC clone RP23-145F9. The BAC clone was modified using DNA recombineering, to eliminate the Slamf1 gene and introduce a stop codon in Slamf2, the gene coding for CD48 (FIG. 4G). It was then injected into B6-C3H F1 fertilized oocytes to generate SLAMF7 BAC Tg mice. Mice were then bred with SFR KO mice to create SFR KO-SLAMF7 BAC Tg mice. Mice lacking Ly-9 (Slamf3^{-/-}) in the 12951/Sv background, and mice lacking EAT-2 (Sh2d1b1^{-/-}) in the C57BL/6 background, were described previously^{30,31.} The following mice were obtained from The Jackson Laboratory (Bar Harbor, Me.): CD84 KO (Slamf5^{-/-}); CD11b KO (Itgam^{-/-}); CD11a KO (Itgal^{-/-}), CD47 KO (Cd47^{-/-}); LRP-1 conditional KO (Lrp1^{fl/fl}); Lyz2-Cre; RAG-1 KO (Rag1^{-/-}); NRG (NOD;Rag1^{-/-}IL2Ryc^{-/-}), which are NOD congenic mice lacking T cells, B cells and NK cells; and X-linked immunodeficiency (XID) mice (in the CBA/CaHN background), which carry a loss-of-function point mutation in Btk²⁴; and T cell-deficient mice (B6.129P2-Tcrb^{tmlMom} Tcrd^{tmlMom}/J). Mice lacking Syk in bone marrow cells were generated by transplantation of fetal liver from Syk^{-/-} mice into irradiated RAG-deficient mice³². Mice lacking FcR gamma (Fcerlg^{-/-}) were obtained from Taconics (Hudson, N.Y.)³³. Mice lacking DAP12 (Tyrobp^{-/-}) were kindly provided by Dr. Toshiyuki Takai (Sendai, Japan). Mice devoid of FcR gamma and DAP12 (Fcer1g^{-/-}; Tyrobp^{-/-}) were reported elsewhere³⁴. All mice were maintained in the C57BL/6 background, unless specified. They were also kept in a specific-pathogen free (SPF) environment. Either males or females were used, typically 8- to 12-weeks of age. Littermates were used as controls in all experiments, except for the studies involving Syk KO, FcRgamma KO, FcR gamma-DAP12 dKO and XID mice, where wild-type syngeneic mice were used. Animal experimentation was approved by the Animal Care Committee of IRCM and performed as defined by the Canadian Council of Animal Care (A.V.), or by the Institutional Animal Care and Use Committee (IACUC) of the University of California at San Francisco, in accordance with the recommendations in the Guide for the Care and Use of Laboratory Animals of the National Institutes of Health (C.A.L.). For experiments with mice, sample size was chosen based on previous studies in this field and to achieve statistical significance. No randomization or blinding was performed. No animals were excluded from the analyses.

Cells and retroviral infection. Mouse BMDMs were produced as described elsewhere³⁵. In brief, femora and tibiae were flushed with tissue culture medium and propagated in bacterial petri dishes for ˜7 days, in medium supplemented with 30% (vol/vol) L929 cell-conditioned medium as a source of colony-stimulating factor 1 (CSF-1). In some experiments, BMDMs were treated with IFN-gamma (100 ng/ml; Miltenyi Biotec, Bergisch Gladbach, Germany) or LPS (100 μg/ml; Sigma-Aldrich, St. Louis, Mo.) prior to experimentation. Thioglycollate-elicited peritoneal macrophages were generated as outlined elsewhere³⁵. In a first set of experiments, L1210 (B cell lymphocytic leukemia), P815 (mastocytoma), SP2/0 (multiple myeloma), the v-Abl-induced B cell line CB17-3A8 (v-Abl-transformed B cell leukemia), WEHI-3 (myelomonocytic leukemia), the BW5147-derived T cell hybridoma BI-141, EL-4 (T cell lymphoma), RMA-S (T cell lymphoma), YAC-1 (thymoma), B16 (melanoma), CMT-93 (rectal carcinoma), RAW264.7 (v-Abl-transformed monocyte/macrophage) and L929 (immortalized fibroblast) from stocks described previously^{30,35-38} were used. Moreover, Raji (B cell lymphoma) and Daudi (B cell lymphoma) were provided by Dr. Javier Di Noia (Montreal, QC, Canada). MEL, CB17-3A8 and WEHI-3B were provided by Dr. Chris Paige (Toronto, ON, Canada). Colo205 (colon carcinoma), SW480 (colon carcinoma) and SW620 (colon carcinoma) were provided by Dr. Nathalie Rivard (Sherbrooke, QC, Canada). Cells were sporadically tested for Mycoplasma and found to be negative. Hematopoietic cells were authenticated by flow cytometry (The foregoing cell lines are designated herein “non-authenticated cells”). Results of experiments (e.g., phagocytosis) performed with these cells were typically expressed as histograms. In subsequent experiments, L1210 (CCL-219), P815 (TIB-64), WEHI-3 (TIB-68), SP2/0 (CRL-1581), BW5147.3 (TIB-47), EL-4 (TIB-39), YAC-1 (TIB-160), B16 (CRL-6475), CMT-93 (CCL-223), L929 (CCL-1), Raji (CCL-86), Daudi (CCL-213), Colo205 (CCL-222), SW480 (CCL-228), SW620 (CCL-227), HCC827; Lung Carcinoma; Human (Homo sapiens) (CRL-2868); NCI-H1838; Lung Carcinoma; Human (Homo sapiens) (CRL-5899); NCI-H1373; Lung Adenocarcinoma; Human (Homo sapiens) (CRL-5866) (negative control); SK-MEL-1; Malignant Melanoma; Human (Homo sapiens) (HTB-67); SK-MEL-28; Melanoma; Human (Homo sapiens) (HTB-72) were freshly obtained from American Type Culture Collection (ATCC; Manassas, Va.), while MEL (mouse erythroleukemia; #96121718) and RAW264.7 (#91062702) were freshly obtained from the European Collection of Authenticated Cell Cultures (ECACC; Sigma-Aldrich, St. Louis, Mo.). ATCC and ECACC authenticated these cell lines and showed that they were negative for Mycoplasma. RMA-S was obtained from the institute that initially generated this cell line (provided through Benedict Chambers, Karolinska Institute, Stockholm, Norway). They were Mycoplasma-negative. (The foregoing cells obtained from the ATCC, ECAC and Karolinska institute are designated herein “authenticated cells”). Results of experiments (e.g., phagocytosis) performed with these authenticated cells were typically expressed in graphs wherein each symbol represents one mouse (or healthy donor in certain cases, as specified). CD47 KO L1210 cells were generated using CRISPR-Cas-mediated gene editing, using the guide RNA sequences CACCGAGCAACAGCGCCGCCGCCAA (SEQ ID NO: 24) and CACCG TTGGCGGCGGCGCTGTTGCT (SEQ ID NO: 25). Activated CD4⁺ T cells were produced as detailed previously³⁶ or were obtained by stimulating purified splenic CD4⁺ T cells with concanavalin A (ConA; 4 Ng/ml; Sigma-Aldrich) for 2 days, followed by IL-2 (50 U/ml) for 3 days. Unstimulated B cells were obtained by isolating total splenocytes from T cell-deficient mice, whereas activated B cells were obtained by stimulating purified B cells with lipopolysaccharide (LPS; 5 Ng/ml) for 5 days. Purity of T cells and B cells was greater than 90%. In some experiments, BMDMs, MEL cells or RAW264.7 cells were infected with retroviruses (using the vector pFB-GFP) encoding various SLAMF7 proteins, in combination with green fluorescent protein (GFP). Retroviruses encoding GFP alone were used as control. For BMDMs, GFP-positive cells were purified by cell sorting 48 hours after infection and propagated for an additional 48 hours in growth medium, prior to experimentation. For MEL and RAW264.7, GFP-positive cells were isolated by cell sorting, when sufficient numbers of cells were available. The constructs encoding mouse SLAMF7 Y→F mutant (Y261 F;Y266F;Y281 F) and FLAG-tagged mouse SLAMF7 were created by PCR. L1210 derivatives expressing Tac (CD25) were generated by transfection, using a plasmid (pSRalpha-puro) encoding a cytoplasmic domain-deleted version of CD25 fused to the transmembrane domain of 2B4. Transfected cells were selected in medium containing puromycin and purified by cell sorting. Human blood samples were collected from healthy donors following informed consent for the McGill University Health Centre (MUHC) institutional review board-approved research protocol GEN10-256. Peripheral blood mononuclear cells (PBMCs) were then isolated using Ficoll-Paque™ PLUS (GE Healthcare, Burlington, ON, Canada), according to the manufacturer's protocol. PBMCs were seeded for 1-3 hours at 37° C. in tissue culture dishes containing serum-free RPMI medium. After gentle washes, adherent cells (which mostly represent monocytes) were cultured in RPMI medium supplemented with 10% human serum (Valley Biomedical). Medium was changed on days 3 and 6. Cells were used for experimentation on day 7.

Antibodies. For flow cytometry or blocking, the following monoclonal antibodies (MAbs) were used. Anti-CD11b (M1/70), anti-F4/80 (BM8), anti-CD18 (M18/2), anti-CD11a (M17/4), anti-CD29 (HMb1-1), anti-CD11c (N418), anti-CD61 (2C9.G3), anti-CD16/32 (93), anti-CD36 (72-1), anti-mouse CD47 (Miap301) and anti-human CD47 (B6H12) were from eBioscience (San Diego, Calif.). Anti-mouse CD47 (Miap301) was from Biolegend (San Diego, Calif.). Anti-CD64 (MAb X54-5/7.1), anti-human CD47 (MAb CC2C6), anti-human SLAMF7 (MAb 162.1), anti-human SLAMF1 (A12 (7D4)), anti-human Ly-9 (HLy-9.1.25), anti-human NTB-A (NT-7), anti-human CD84 (CD84.1.21) and anti-human CD48 (BJ40) were also from Biolegend (San Diego, Calif.). Anti-LRP-1 (MAb 5A6) was from Novus Biologicals (Littleton, Colo.). Anti-CD11b (MAb 5C6) was from AbD Serotec (Kidlington, UK). Anti-SIRPalpha (MAb P84) was from BD Biosciences (Mississauga, ON, Canada). Antibodies directed against mouse SFRs and CD48 were described previously³⁶. Anti-human SLAMF7 MAb 162 was reported elsewhere³⁹. F(ab′)₂ fragments of anti-mouse CD47 MAb (Miap301), anti-human CD47 MAb (B6H12), anti-human SLAMF7 MAb (162) and control IgG were generated using pepsin (Sigma-Aldrich), according to standard protocols. Purity and integrity of F(ab′)₂ fragments were confirmed by protein gel electrophoresis. For immunoprecipitations and immunoblots, the following antibodies were used: anti-Syk and anti-SLAMF7 rabbit antisera (generated in the inventors' Iaboratory^{40,41}), anti-beta-actin (C4; Santa Cruz Biotechnology, Santa Cruz, Calif.), anti-DAP12 (D7G1X; New England Biolabs, Ipswich, Mass.), anti-FcR gamma (PM068; MBL International, Woburn, Mass.), anti-CD11b (EPR1344; Abcam, Toronto, ON) and anti-LRP-1 (MAb 5A6; Abcam). For immunofluorescence, the following antibodies were used: anti-CD18 (MAb M18/2), anti-FLAG (MAb M2; Sigma-Aldrich), anti-CD11 b (MAb EPR1344; Abcam) and anti-beta-actin (MAb AC-74, Sigma-Aldrich).

In vitro phagocytosis assays. For the microscopy-based assay, 5×10⁴ macrophages were seeded overnight in a 24-well tissue culture plate. On the next day, target cells were washed and labeled with 2.5 μM of carboxyfluorescein succinimidyl ester (CFSE), using the CFSE Cell Proliferation Kit (C34554; Life Technologies, Burlington, ON, Canada). After incubating macrophages in serum-free medium for 2 hours, 2×10⁵ CFSE-labeled target cells were added to the macrophages, in the presence of anti-CD47 Ab or control IgG (10 μg/mL). After incubation for 2 hours at 37° C., macrophages were extensively washed and imaged with an inverted microscope (Carl Zeiss Axiovert™ S100 TV). The phagocytosis efficiency was calculated as the number of macrophages containing CFSE⁺ target cells per 100 macrophages. For the flow cytometry-based assay, macrophages were prepared and then incubated with Tac-expressing L1210 cells as targets, as detailed for the microscopy assay, except that targets were labeled with 0.2 μM of CFSE. Once the phagocytosis period was completed, all cells in the well were harvested in the presence of Accutase™. Cells were then stained on ice for 30 minutes with APC conjugated-anti-human Tac (CD25) and PE-conjugated MAb F4/80, and analyzed by flow cytometry. After gating on F4/80⁺Tac⁻ cells (which include macrophages but exclude non-phagocytosed Tac-positive L1210 cells), phagocytosis efficiency was determined as the percentage of F4/80⁺Tac⁻ cells containing CFSE-derived green fluorescence (detected in FL1 channel). For the pHrodo™⁴-based assay, macrophages were prepared and incubated with targets as detailed for the microscopy assay, except that targets were labeled with 100 ng/ml of pHrodo™ Green AM Intracellular pH Indicator (Thermo Fisher Scientific, Waltham, Mass.), according to the manufacturer's protocol. pHrodo™ dyes are non-fluorescent at neutral pH and become fluorescent in acidic environments such as phagolysosome. Once the phagocytosis period was completed, all cells in the well were harvested in the presence of Accutase™. They were then stained with APC-conjugated anti-F4/80 and analyzed by flow cytometry. Phagocytosis efficiency was determined as the percentage of F4/80⁺ cells containing pHrodo™-derived green fluorescence (detected in FL1 channel). For phagocytosis of IgG-opsonized tumor cells, L1210 cells expressing Tac (CD25) were opsonized with anti-Tac MAb 7G7 (a mouse IgG2ₐ) for 1 hour at 37° C., prior to the microscopy-based phagocytosis assay. For phagocytosis of C3b;-opsonized tumor cells, L1210 cells were incubated with C5-deficient human serum (Sigma-Aldrich) for 1 hour at 37° C., prior to the microscopy-based phagocytosis assay. For phagocytosis of apoptotic thymocytes, thymocytes (2×10⁷ cells/ml) from 4- to 8-week-old C57BL/6 mice were treated at 37° C. for 10 hours with 1 μM of dexamethasone (Sigma-Aldrich), which caused about 70% of cells to become apoptotic (annexin V-positive; data not shown). Cells were then labeled with 2.5 μM of CFSE and incubated with macrophages at a ratio of 20:1 for 30 minutes at 37° C. Phagocytosis was monitored by microscopy. For endocytosis of immune complexes, biotinylated mouse IgG (eBioscience) was inactivated at 65° C. for 30 minutes and mixed with PE-coupled streptavidin (eBioscience) on ice for 30 minutes, to form immune complexes. Then, IgG immune complexes were incubated with macrophages at 37° C. for 30 minutes. A solution of PBS (pH 2.5) was added to the cell mixture for 1 minute at 4° C., to remove non-endocytosed immune complexes. Cells were subsequently washed, fixed with 4% paraformaldehyde (PFA) and analyzed by flow cytometry. For phagocytosis of bacteria, GFP-expressing E. coli (DHSalpha) were cultured overnight at 37° C. They were then mixed with macrophages at a ratio of 100:1 and incubated at 37° C. for 15 minutes or 30 minutes. After washing, the mixture was digested with lysozyme at 37° C. for 30 minutes, to remove non-phagocytosed bacteria. Then, cells were washed, fixed with 4% PFA and analyzed by flow cytometry. For phagocytosis of opsonized sheep red blood cells (sRBCs), sRBCs (MP Biomedicals, Santa Ana, Calif.) were washed with cold PBS and opsonized with rabbit anti-sRBC IgG (MP Biomedicals) for 1.5 hour at 37° C. sRBCs were then labeled with PKH26 (Sigma-Aldrich), according to the manufacturer's protocol. Macrophages and sRBCs were incubated at a ratio of 1:10 at 37° C. for 30 minutes. After lysing non-phagocytosed sRBCs with RBC lysis buffer (Sigma-Aldrich), macrophages were washed, fixed with 4% PFA and analyzed by flow cytometry. For phagocytosis of mouse RBCs, freshly isolated mouse RBCs from wild-type or CD47 KO mice were labeled with PKH26. After incubating macrophages in serum-free medium for 2 hours, 1×10⁶ mouse RBCs were added to the macrophages, as detailed for the fluorescence microscopy assay. After incubation for 30 minutes at 37° C., non-phagocytosed RBCs were lysed with RBC lysis buffer and macrophages were imaged by fluorescence microscopy. For experiments with pharmacological inhibitors, phagocytosis was monitored using the microscopy-based assay, except that macrophages were pre-incubated for 1 hour with the following pharmacological inhibitors: Btk family kinase inhibitor, ibrutinib (10 nM; Selleckchem, Burlington, ON, Canada); Syk kinase inhibitor, R406 (750 nM; Calbiochem, Burlington, ON, Canada); or Src kinase inhibitor, SU6656 (100 nM; 572635; Calbiochem). Inhibitors were added to macrophages one hour prior to and during the phagocytosis assay. They had no deleterious impact on cell viability, as verified by staining cells with propidium iodide (PI) and annexin V (data not shown).

Intra-peritoneal tumor clearance assay. Mice (6-8-week-old) were injected intra-peritoneally with 1.5 ml of 4% (w/v) thioglycollate medium (BD Biosciences). After 4 days, 5×10⁶ CFSE-labeled tumor cells (L1210 or MEL; in 200 μl of PBS) were injected intra-peritoneally (I.P.), in the presence of anti-CD47 or control IgG. After 24 hours, cells in the peritoneal cavity were collected using cold PBS washing buffer containing 2% fetal bovine serum and 1 mM EDTA. Numbers of remaining CFSE-positive target cells were quantified by flow cytometry. Numbers of macrophages were also determined, by staining with anti-F4/80. A fixed number of fluorescent beads (1×10⁴; 7.58 μm in diameter; Flow Cytometry Absolute Count Standard, Full Spectrum; Bangs Laboratories, Fishers, Ind.) was added to one twentieth of the cell suspension before flow cytometry. Equivalent numbers of fluorescent beads (2×10²) were acquired for standardization of cell numbers. In some experiments, mice were injected I.P. on days −1 and 3 with 200 μl of liposomes containing clodronate or PBS (ClodronateLiposomes.com; Amsterdam, The Netherlands), to deplete macrophages.

Intra-peritoneal tumor clearance assay for non-hematopoietic tumor cells. Mice (6-8-week-old) are injected intra-peritoneally with 1.5 ml of 4% (w/v) thioglycollate medium (BD Biosciences). After 4 days, 5×10⁶ CFSE-labeled tumor cells (parental and CD47-deficient solid tumor cells) are injected intra-peritoneally (I.P.). After 24 hours, cells in the peritoneal cavity are collected using cold PBS washing buffer containing 2% fetal bovine serum and 1 mM EDTA. Numbers of remaining CFSE-positive target cells are quantified by flow cytometry. Numbers of macrophages are also determined, by staining with anti-F4/80. A fixed number of fluorescent beads (1×10⁴; 7.58 μm in diameter; Flow Cytometry Absolute Count Standard, Full Spectrum; Bangs Laboratories, Fishers, Ind.) is added to one twentieth of the cell suspension before flow cytometry. Equivalent numbers of fluorescent beads (2×10²) are acquired for standardization of cell numbers. In some experiments, mice are injected I.P. on days −1 and 3 with 200 μl of liposomes containing clodronate or PBS (ClodronateLiposomes.com; Amsterdam, The Netherlands), to deplete macrophages.

Sub-cutaneous tumor transplantation assay. 1×10⁶ L1210 cells (in some cases, expressing GFP) were injected sub-cutaneously into the right flank of 6-10-week old RAG-1 KO or RAG-1 SFR dKO mice. RAG-1 KO mice were used in order to avoid T cell-mediated rejection of L1210 cells, which are derived from a mouse strain (DBA) different from that of SFR KO mice (C57BL/6). Starting on day 4, mice were injected daily I.P. with 200 μg of control rat IgG2a or rat anti-mouse CD47 (Miap301). Tumor volume was measured every day using a caliper and the formula (lengthxwidth²)/2. Antibody treatment was stopped at day 11 and mice were immediately sacrificed. Tumor size limit allowed was 1.5 cm in diameter. Experiments were terminated when or before this size was reached. Tumors were then dissected and weighed. Volumes were also assessed. Then, tumors were sliced into small pieces and pressed through a strainer using the plunger end of a syringe. Cells were washed twice with cold PBS with 2% fetal bovine serum. Total cell numbers were determined, while live cells were enumerated by staining with trypan blue to exclude dead cells. Tumor cells were detected by flow cytometry, using the marker GFP, while immune cells were detected by staining with the relevant antibodies and flow cytometry.

Sub-cutaneous solid tumor transplantation assay. 1×10⁶ solid tumor cells (parental and CD47-negative; GFP-positive) are injected sub-cutaneously into the right flank of 6-10-week-old RAG-1 KO or RAG-1 SFR dKO mice. RAG-1 KO mice are used in order to avoid T cell-mediated rejection of solid tumor cells, which are of human origin. Tumor volume is measured every day using a caliper and the formula (lengthxwidth²)/2. Tumor size limit allowed is 1.5 cm in diameter. Experiments are terminated when or before this size is reached. Tumors are then dissected and weighed. Volumes are also assessed. Then, tumors are sliced into small pieces and pressed through a strainer using the plunger end of a syringe. Cells are washed twice with cold PBS with 2% fetal bovine serum. Total cell numbers are determined, while live cells are enumerated by staining with trypan blue to exclude dead cells. Tumor cells are detected by flow cytometry, using the marker GFP, while immune cells were detected by staining with the relevant antibodies and flow cytometry.

T cell adoptive transfer. ConA-activated wild-type and SLAMF7 KO CD4⁺ T cells were labeled with 5 μM of CFSE or Cell Trace Violet (CTV; Life Technologies), respectively. Then, a 1:1 mixture of wild-type and SLAMF7 KO T cells was injected intravenously into wild-type mice, along with control IgG or anti-CD47 Ab. After 24 hours, PBMCs were isolated and the presence of CFSE-positive or CTV-positive cells was detected by flow cytometry.

Adhesion assays. For the microscopy-based assay, BMDMs (2×10⁵) were labeled with CTV and plated overnight onto cover slips. The next day, target cells (L1210) were labeled with CFSE. Macrophages and target cells were then mixed at a 1:4 ratio in serum-free culture medium and incubated for 30 minutes at 37° C. to allow conjugate formation. Cells were subsequently washed extensively to remove unconjugated cells. Slides were then analyzed by LSM 710 laser scanning confocal microscope (Carl Zeiss Canada Ltd., Toronto, ON, Canada). Conjugates between BMDMs and target cells were counted. For the flow cytometry-based assay, macrophages were labeled on ice with APC-conjugated anti-F4/80 MAb BM8, while target cells were labeled in a similar way using 0.5 μM of CFSE. After washing, cells were resuspended at a concentration of 2×10⁶ cells per ml. Conjugate formation was analyzed by mixing macrophages with targets, and by incubating the mixture for various periods of time at 37° C., as described³¹ Conjugates were detected by flow cytometry.

Stimulation of target cells with anti-CD47 antibodies. To detect apoptosis, 2×10⁵ L1210 or P815 cells were incubated in 24 well-plate in the presence of 10 μg/ml of control rat IgG or rat anti-CD47 Ab. Cells were collected the next day and cell death was examined by staining with annexin V and propidium iodide (PI), using the Annexin V Apoptosis Detection Kit APC (eBioscience). To measure proliferation, 2×10⁵ L1210 or P815 cells were labeled with 5 μM of CFSE and incubated overnight in cell culture medium containing 10 μg/ml of control rat IgG or rat anti-CD47 Ab. Cells were collected the next day and CFSE intensity was analyzed by flow cytometry. To probe calcium flux, L1210 or P815 cells (2×10⁶ cells/sample) were loaded with the calcium indicator dye Indo-1, and then stimulated or not with rat anti-CD47 Ab and rabbit anti-rat Ab. Calcium flux was assessed by flow cytometry, using the FL4/FL5 fluorescence ratio. Ionomycin served as positive control. To study protein tyrosine phosphorylation, 2×10⁶ L1210 or P815 cells were stimulated or not with rat anti-CD47 Ab plus rabbit anti-rat Ab. Protein tyrosine phosphorylation was detected by immunoblotting of total cell lysates with anti-phosphotyrosine antibodies (MAb 4G10).

Immunoprecipitations, mass spectrometry, immunoblots. Immunoprecipitations and mass spectrometry were performed as described^{42, 51}, using Brij99 as detergent in lysis buffer. Mass spectrometry was performed by the IRCM Proteomics Core Facility, as outlined elsewhere⁴⁹. In brief, immunoprecipitated proteins were digested with trypsin (Promega, Madison, Wis.) and analyzed by LC-MS/MS on a LTQ Orbitrap™ Velos (ThermoFisher Scientific, Bremen, Germany) equipped with a Proxeon Nanoelectrospray™ ion source. A 100 minutes' gradient was used for LC separation and standard proteomics parameters were used for the mass spectrometers. Protein database searching was performed with Mascot 2.5 (Matrix Science) and data analysis was conducted using Scaffold (version 3.6). The following criteria were used to select potentially relevant SLAMF7 interactors: 1) to be present in SLAMF7 immunoprecipitates from WT, but not from SFR KO, macrophages; 2) to be observed in a minimum of 4 of the 5 independent SLAMF7 immunoprecipitates from WT macrophages; and 3) to be a receptor known to regulate macrophage activation. The following criteria were used to select potentially relevant CD11b interactors: 1) to be present in CD11b immunoprecipitates from WT, but not from CD11b KO, macrophages; 2) to be observed in a minimum of 5 of the 6 independent CD11b immunoprecipitates from WT macrophages; and 3) to be a receptor known to regulate macrophage function. Immunoblots were performed as reported elsewhere⁴³

Immunofluorescence. RAW264.7 cells expressing FLAG-SLAMF7 or GFP alone (3×10⁵) were seeded onto glass cover slips in 6 well-plates. On the next day, cells were fixed in phosphate-buffered saline (PBS) containing 4% formaldehyde. Subsequently, they were washed twice with PBS and permeabilized for 15 min at 4° C. in PBS containing 0.1% Triton X-100. After additional washes, non-specific staining was prevented by blocking for 30 minutes in PBS supplemented with 5% bovine serum albumin and supernatants (25% v/v) of mouse IgG2a MAb 7G7 and rat anti-CD16/32 MAb 2.4G2, which block Fc receptors. Cells were washed again and incubated for 1 hour with anti-CD11b rabbit MAb EPR1344, anti-CD18 rat MAb M18/2 and anti-FLAG mouse MAb M2, as specified in the Figure legends. After further washing, cells were incubated for 1 hour with Alexa Fluor 594-coupled goat anti-rabbit IgG, Alexa Fluor 488-coupled goat anti-rat IgG and Alexa Fluor-488coupled goat anti-mouse IgG (Thermo Fisher Scientific). Following three additional washes, cover slips were mounted over glass slides using fluorescent mounting medium (Dako, Markham, ON, Canada). Data were acquired using a laser-scanning confocal microscope LSM-710 (Carl Zeiss Canada Ltd., Toronto, ON, Canada). To study actin polarization, BMDMs (2×10⁵) were stained with Cell Trace Violet (CTV) dye (Life Technologies) and seeded onto cover slips. On the next day, target cells (L1210) were stained with CFSE, following the instructions of the manufacturer. Macrophages and targets were then mixed at a 1:4 ratio in serum-free culture medium and incubated for 30 minutes at 37° C. to allow conjugate formation. Cells were subsequently fixed, washed and permeabilized, and non-specific staining was blocked, as detailed above for RAW264.7 cells. Then, cells were washed and incubated for 1 hour with anti-actin mouse MAb AC-74 (Sigma-Aldrich). After further washing, they were incubated for 1 hour with Alexa Fluor 594-coupled goat anti-mouse IgG (Thermo Fisher Scientific). Slides were then processed and analyzed by confocal microscopy, as detailed above for RAW264.7 cells. Conjugates with full polarization of actin at the area of contact between the macrophage and the target cell were quantitated.

Gene expression dataset analyses. Expression of SLAMF7 and CD47 RNA was analyzed using public datasets of human hematologic tumors. Normalized data for samples of leukemia were extracted from the MILE Study⁵², whereas normalized data for AML were obtained from The Cancer Genome Atlas (TCGA). In both cases, data were downloaded through Bloodspot⁴⁷ (www.bloodspot.eu). Data for various samples of multiple myeloma were extracted from GSE26760⁴⁸, using the “GEOquery”™ R package. Lastly, data for various samples of AML and DLBCL were directly extracted from TCGA, using the “TCGAretriever”™ R package. Data were plotted and statistical analyses (unpaired Student's t-tests (two-tailed)) were performed using the R software.

Statistical analyses. Except for the gene expression dataset analyses, unpaired Student's t-tests (two-tailed) were performed using Prism 6 (GraphPad™ Software). Sample sizes in experiments were chosen based on sizes reported in this field and to achieve statistical significance.

### Example 2: Susceptibility of Hematopoietic Tumor Cells to Enhanced Phagocytosis in Response to SIRPalpha-CD47 Checkpoint Blockade

The inventors sought to identify the pro-phagocytic receptor(s) enabling macrophages to engulf tumor cells following disruption of the SIRPalpha-CD47 checkpoint. Mouse bone marrow-derived macrophages (BMDMs, also designated MΦs) were tested for phagocytosis of various target cells, in the presence of blocking anti-CD47 antibodies (Ab) or control IgG using several assays. Phagocytosis was monitored using a fluorescence-based microscopy assay, which was validated by confocal microscopy (FIG. 2A-B). Data were further corroborated using a flow cytometry-based assay (FIG. 2C) and a pH-sensitive pHrodo™-based assay (FIG. 2D). Hematopoietic and non-hematopoietic target cells, of either mouse or human origin, were analyzed. An augmentation of phagocytosis was seen with the mouse hematopoietic B cell lineage and myeloid tumor cell lines L1210 (B cell lymphocytic leukemia), CB17-3A8 (B cell leukemia), SP210 (multiple myeloma), P815 (mastocytoma) and WEHI-3B (myelomonocytic leukemia), treated with anti-CD47 Ab compared to control IgG (FIG. 2E). This was seen with intact Ab or F(ab′)₂ fragments of Ab, implying that it was Fc receptor (FcR)-independent (FIG. 2F). Similar results were obtained with thioglycollate (TG)-elicited peritoneal macrophages and interferon (IFN)-gamma-stimulated BMDMs (FIG. 2G-H). Increased phagocytosis during CD47 blockade was also observed using normal activated CD4⁺ T cells as targets (FIG. 2I). Anti-CD47 Ab had no direct effect on tumor cells (FIGS. 2J-A, 2J-B, 2K, 2L and 2M).

No such anti-CD47 Ab-dependent increase in phagocytosis was seen with several T cell-derived tumor cell lines, the MEL erythroleukemia cell line and various non-hematopoietic cell lines, even though they expressed CD47 (FIGS. 2N, 20-A to 20-G). In contrast to parental L1210 cells, L1210 cells rendered CD47-deficient (CD47 knock-out (KO)) by CRISPR-Cas-mediated gene editing had enhanced phagocytosis in the absence of anti-CD47 Ab (FIGS. 2P-A, 2P-B). Increased phagocytosis in response to anti-CD47 Ab (F(ab′)₂ was also seen with the human B cell lymphoma lines Raji and Daudi, but not with several non-hematopoietic human tumor cells also showing the Fc receptor (FcR)-independent impact of the observed phagocytosis (FIG. 2Q). As the anti-human CD47 Ab is a mouse Ab, F(ab′)₂ fragments of mouse-originated anti-human CD47 Ab were used to avoid triggering mouse Fc receptor-mediated phagocytosis of human targets. However, phagocytosis of non-hematopoietic targets was seen when BMDMs were treated with bacterial lipopolysaccharide (LPS), a strong inflammatory stimulus (FIG. 2R).

Non-transformed mouse activated CD4⁺ T cells, also displayed increased phagocytosis in response to anti-CD47 Ab while thymocytes, freshly isolated CD4⁺ T cells and B cells, and activated B cells did not (FIG. 2S). These findings implied that some, but not all, hematopoietic tumor cells and normal cells displayed enhanced phagocytosis in response to SIRPalpha-CD47 checkpoint blockade. Engulfment of non-hematopoietic cells was also possible, when macrophages were exposed to strong inflammatory stimuli.

### Example 3: Impact of Absence of SLAM Family Receptors on Targeted Tumor Cells on Phagocytosis in Response to SIRPalpha-CD47 Checkpoint Blockade

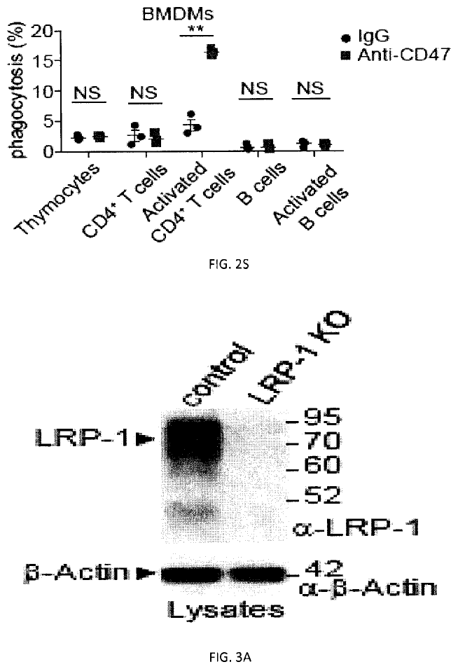

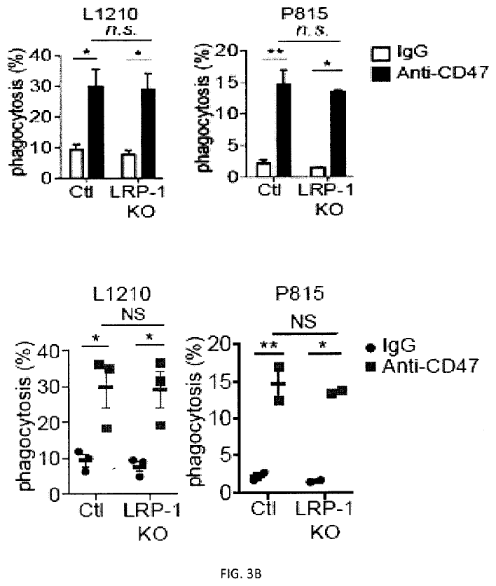

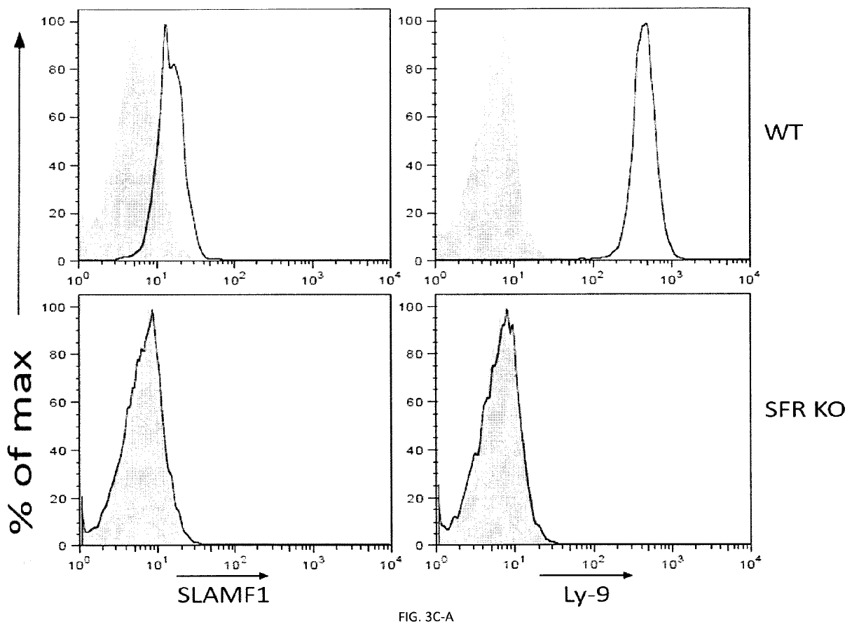

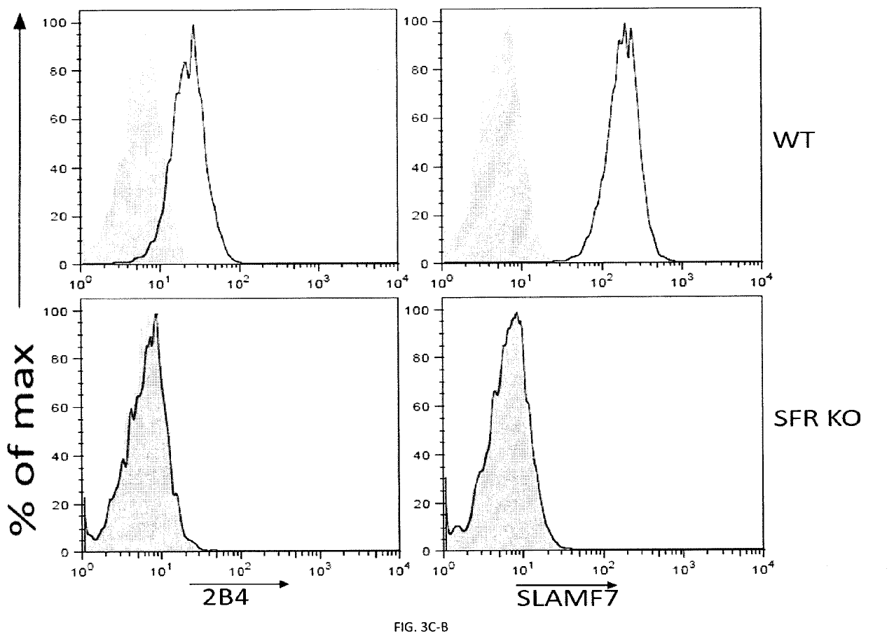

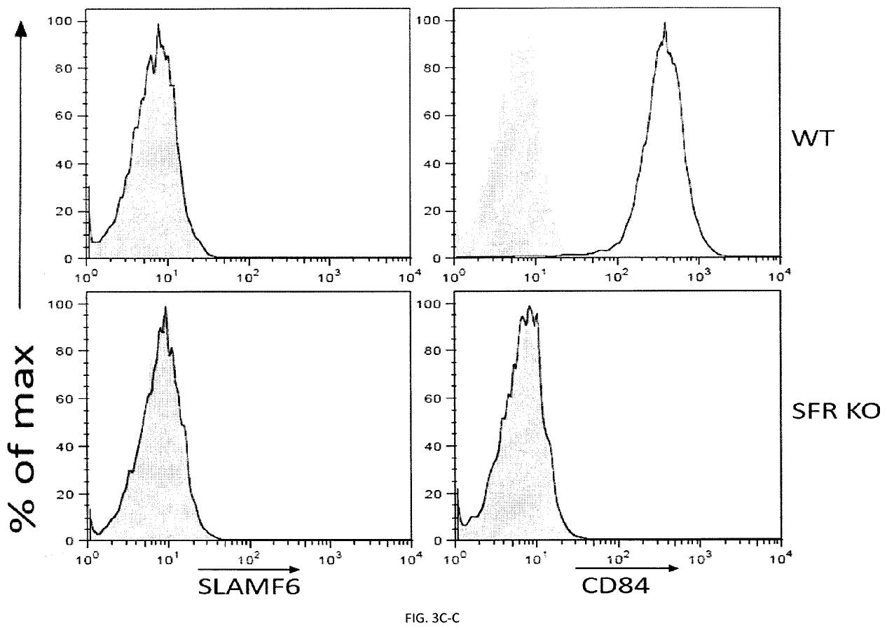

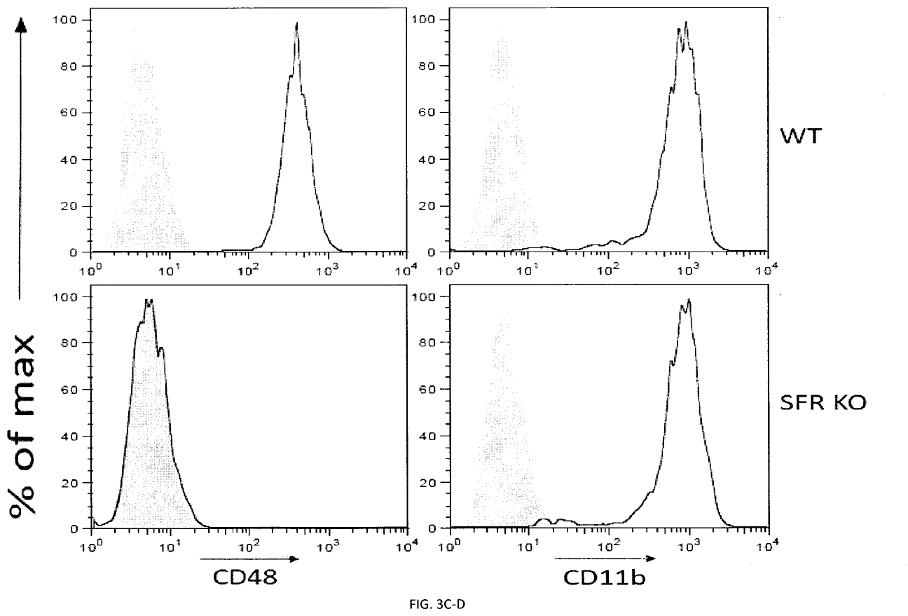

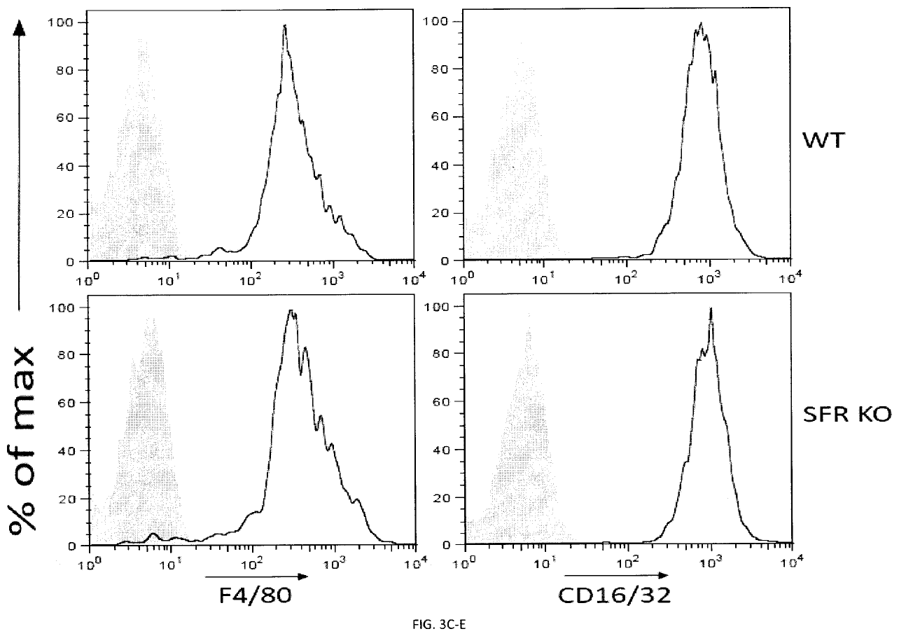

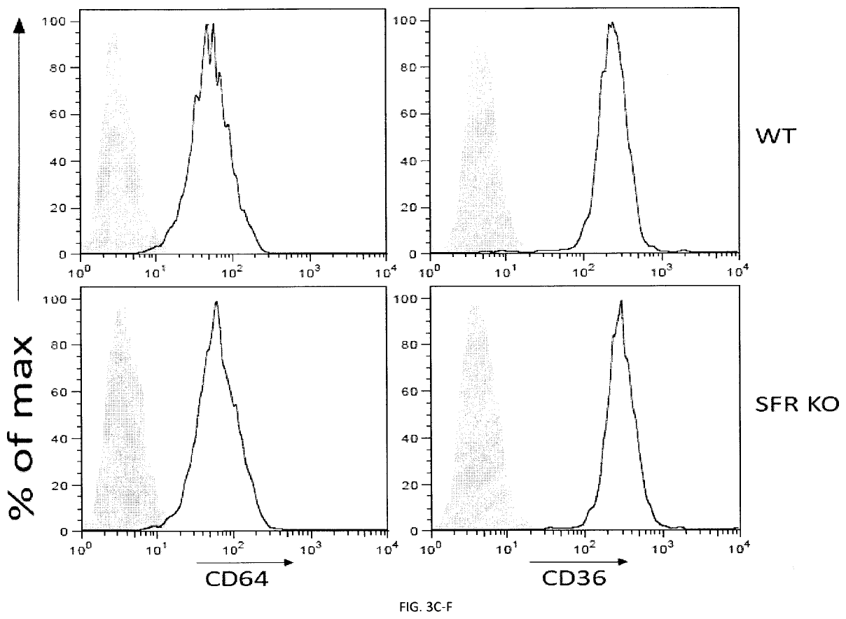

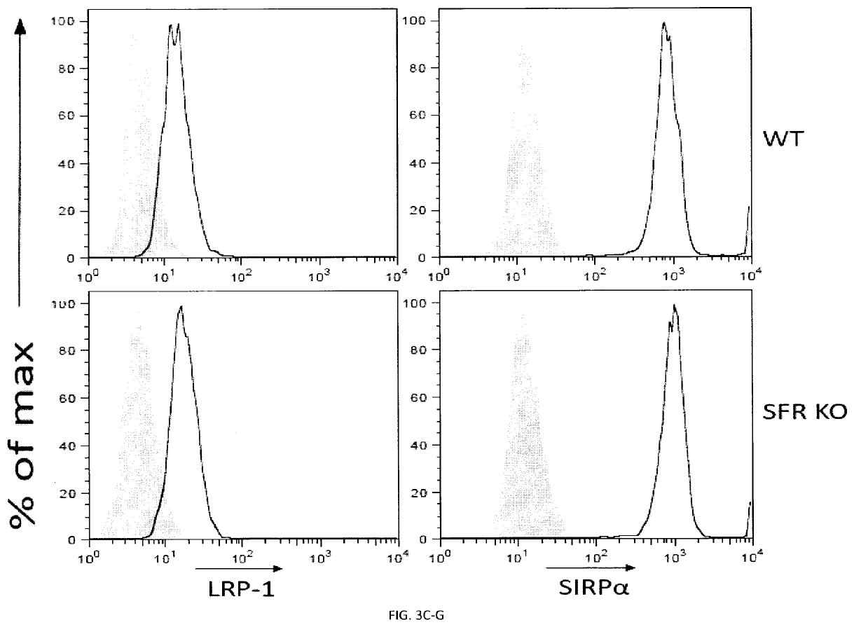

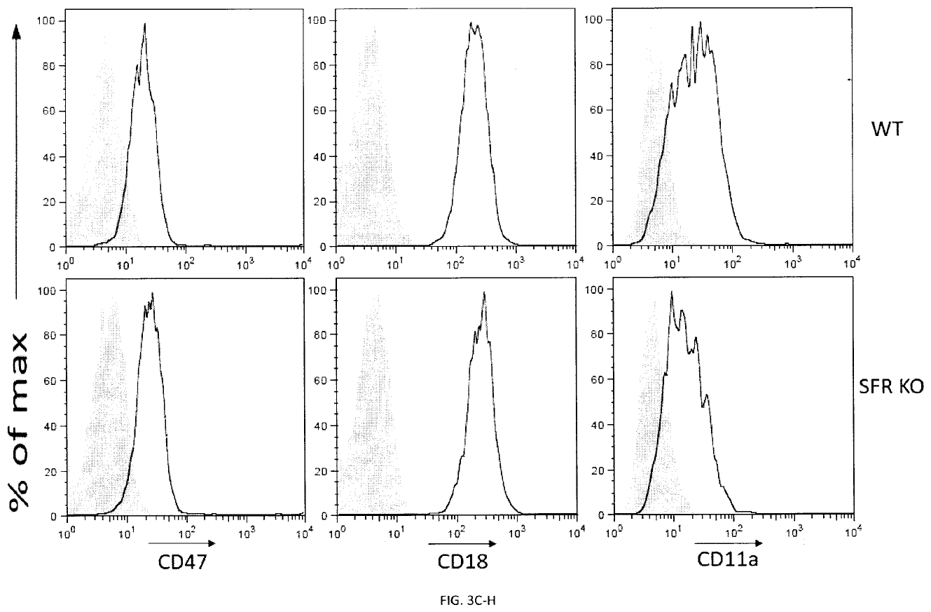

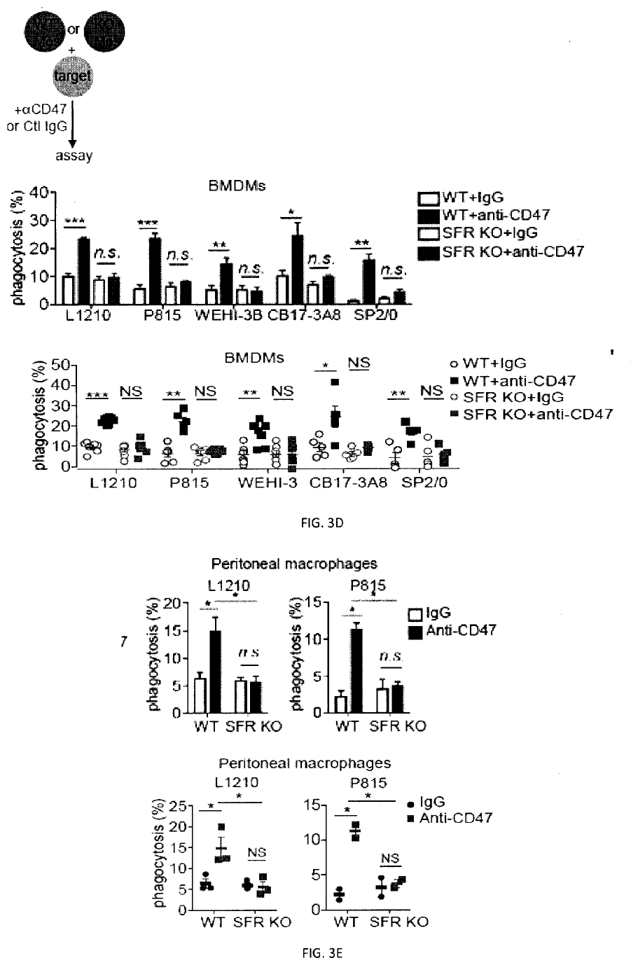

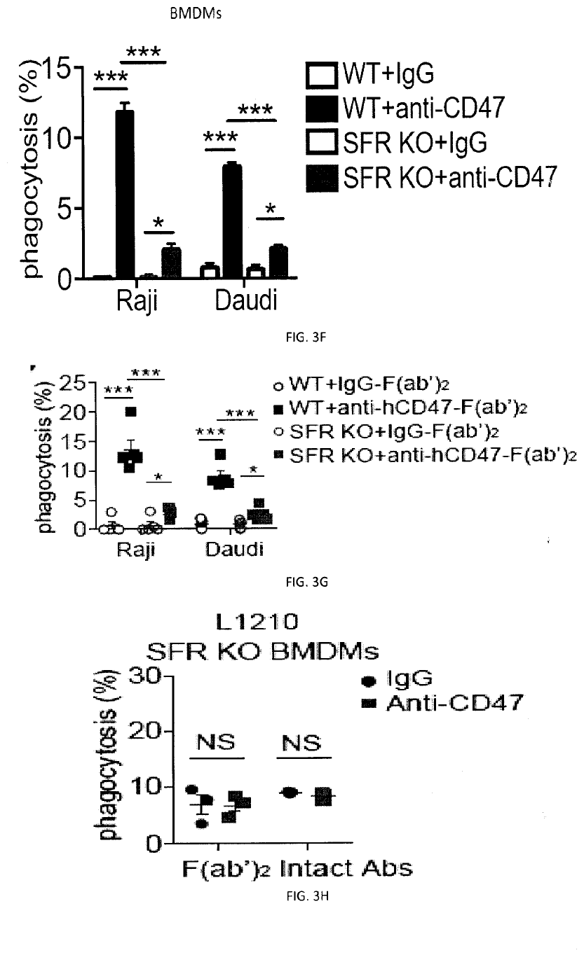

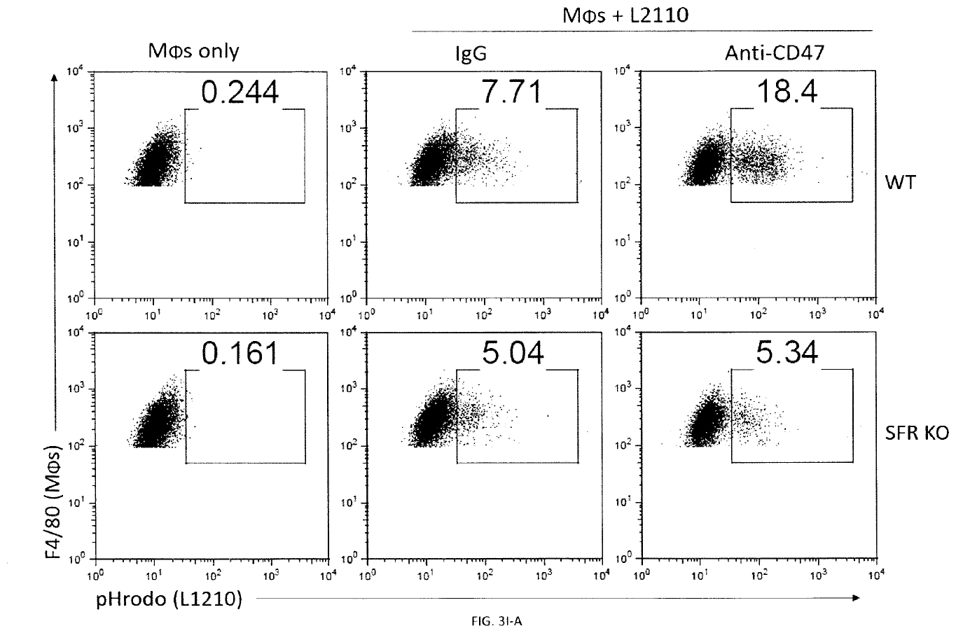

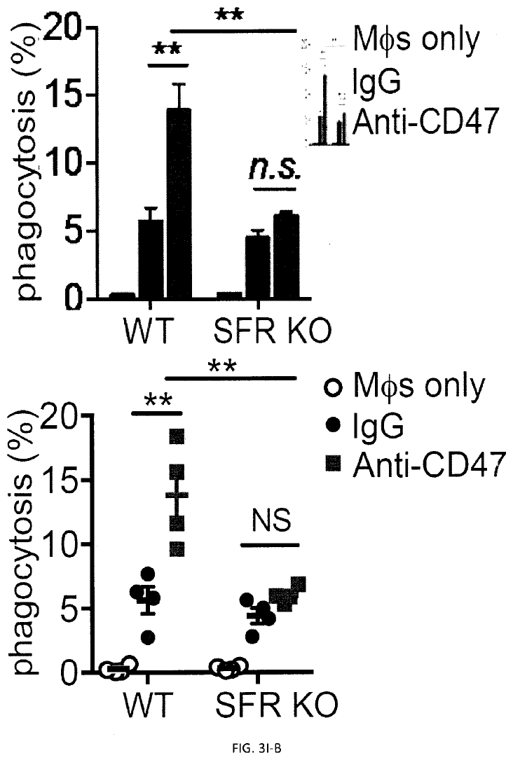

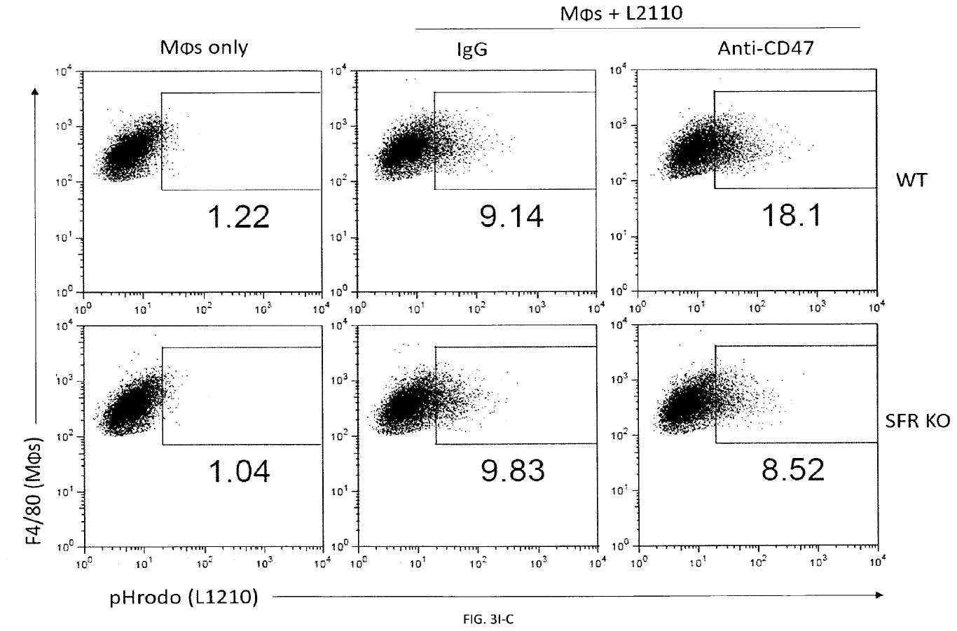

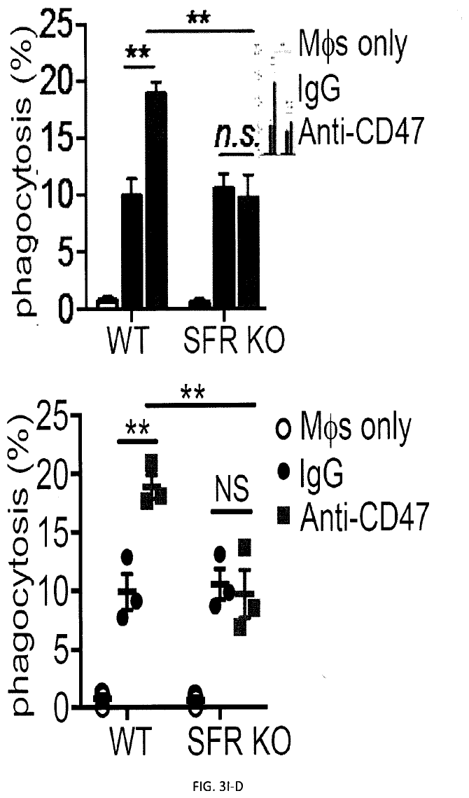

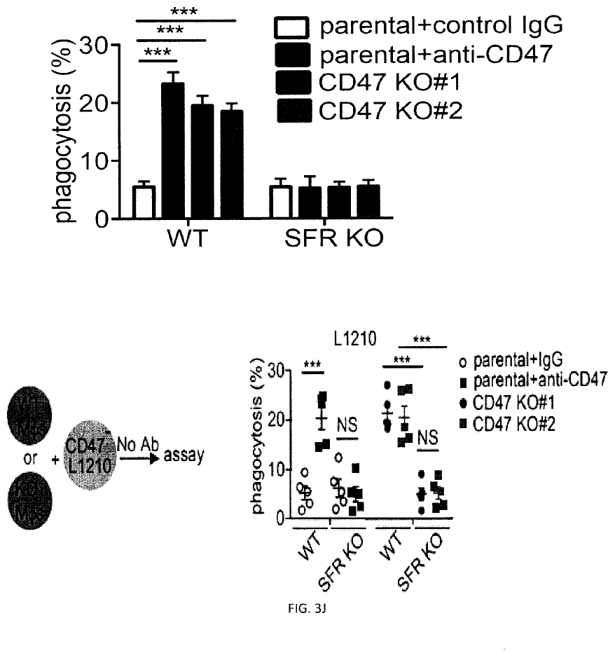

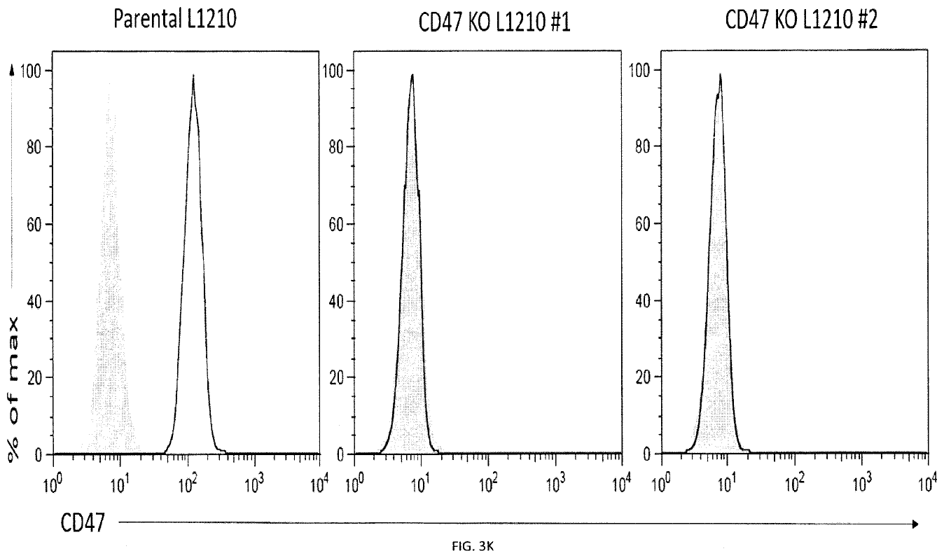

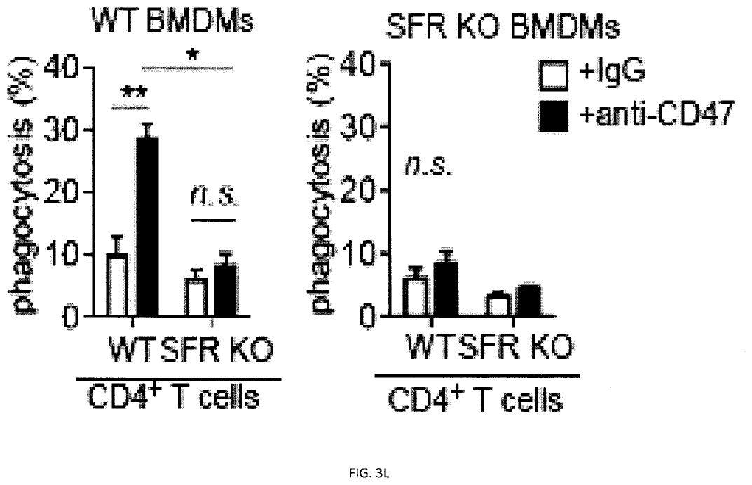

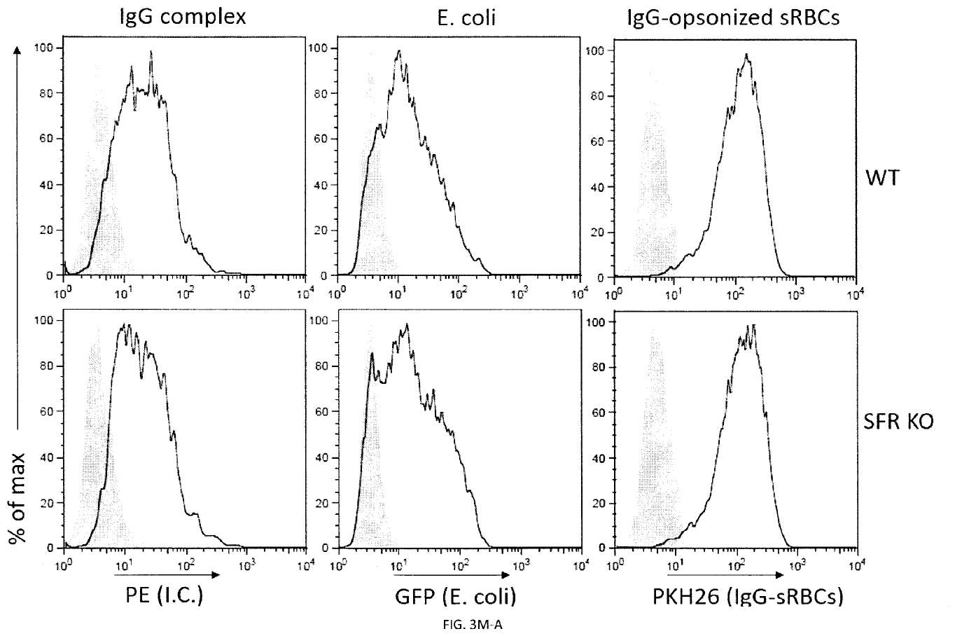

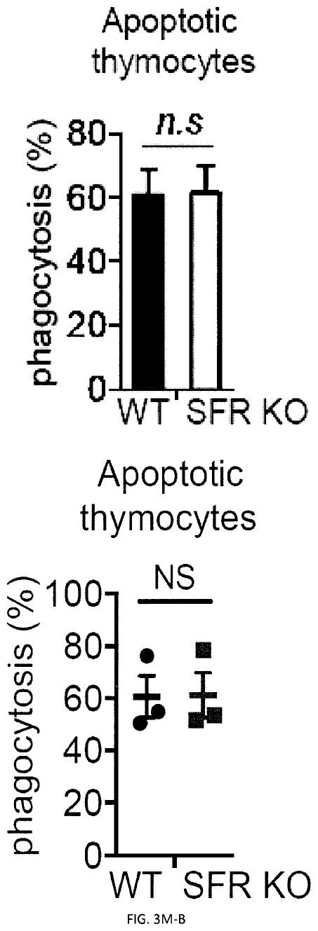

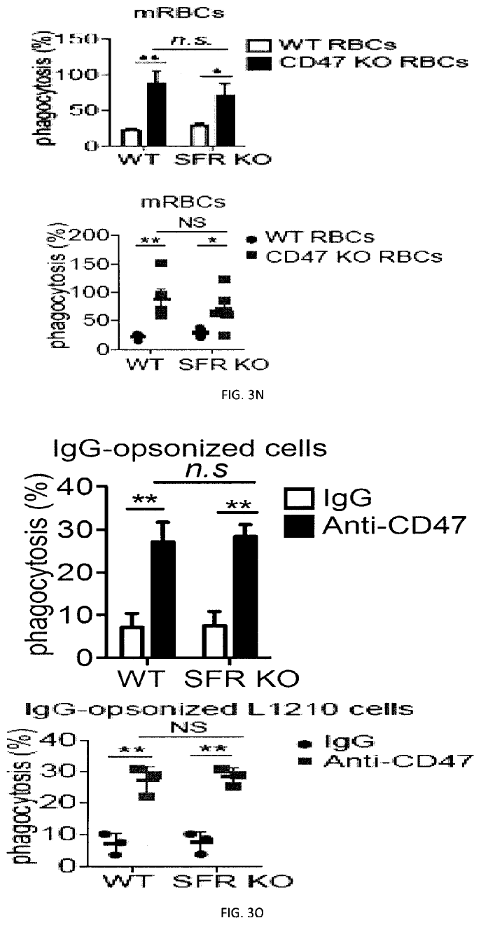

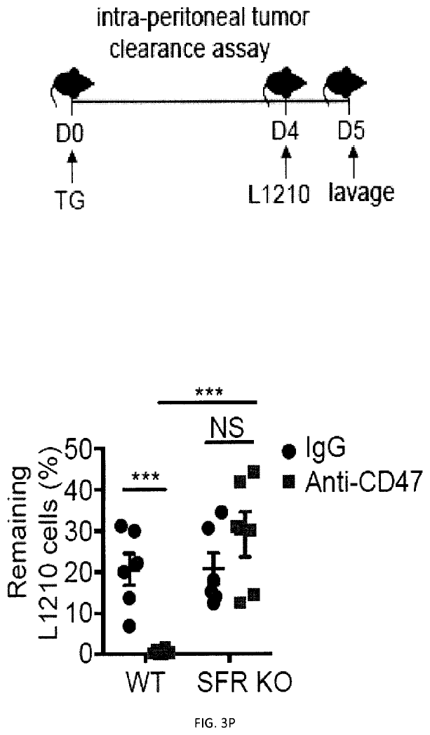

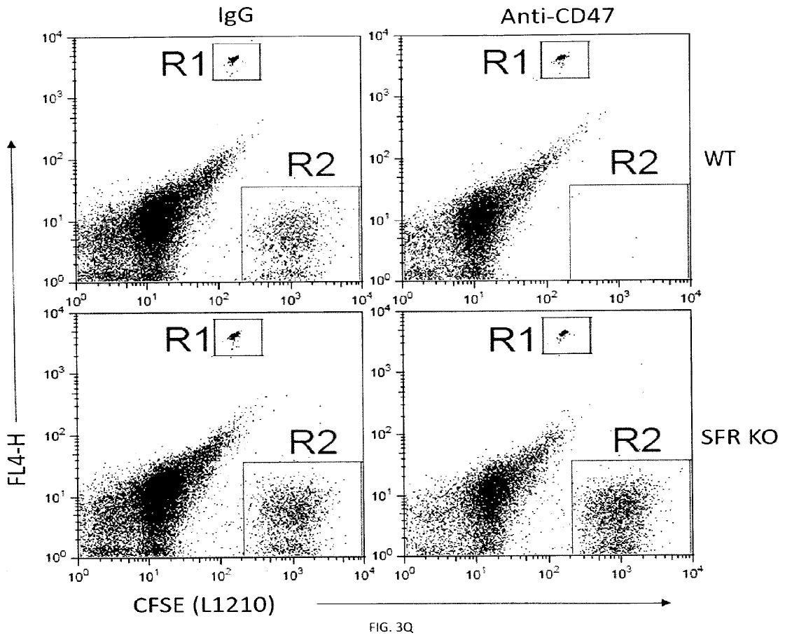

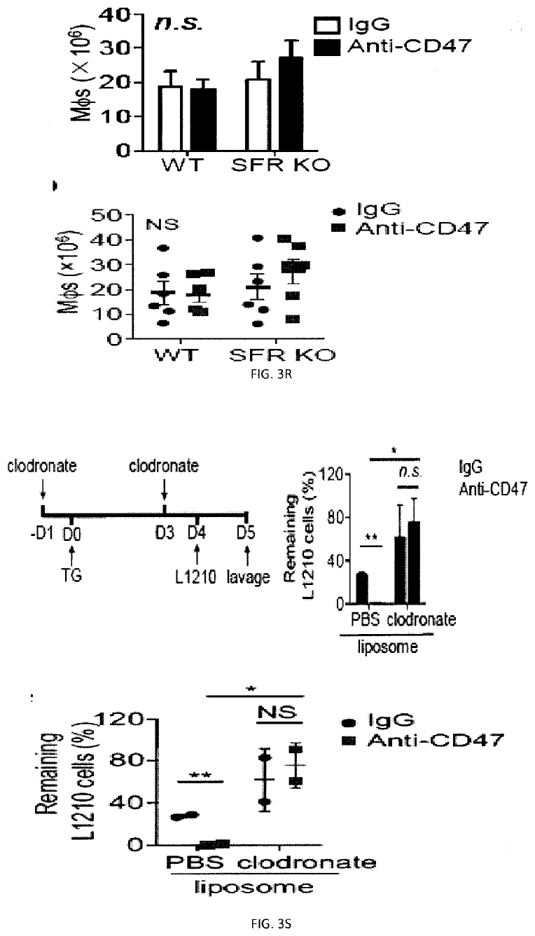

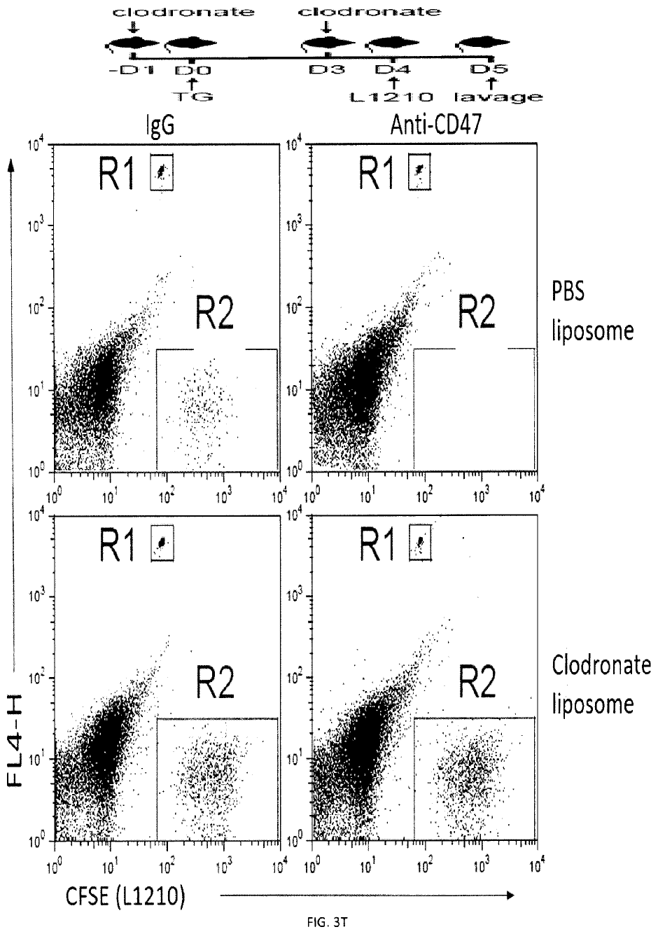

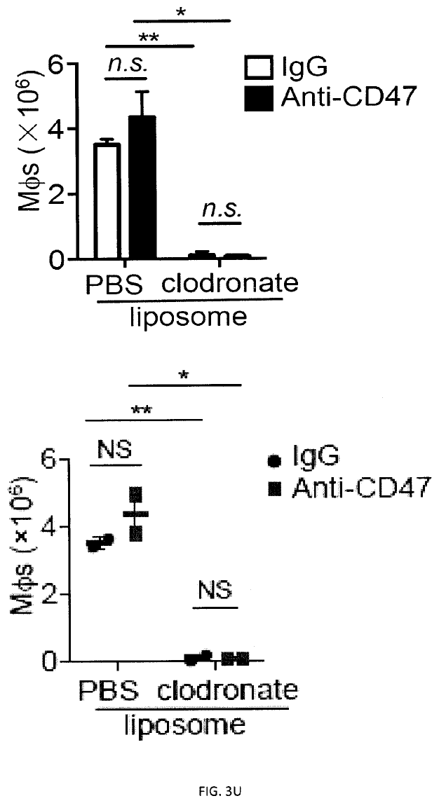

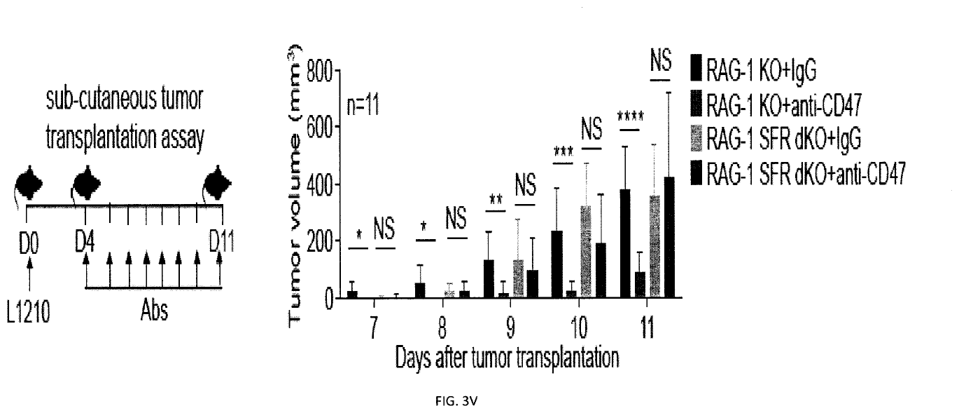

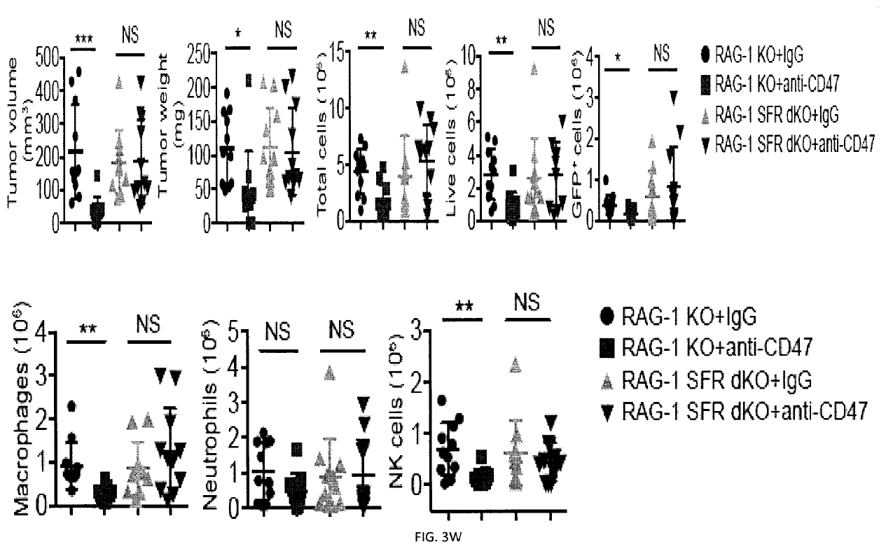

Previous studies suggested that phagocytosis of tumor cells is mediated by the LRP-1 receptor, which can recognize calreticulin on tumor cells²¹. However, the instant inventors observed that phagocytosis of L1210 and P815 cells was equivalent in control and LRP-1 KO macrophages, implying an alternative mechanism (FIGS. 3A-B; data not shown). Since CD47 Ab blockade had the greatest effect on macrophage engulfment of hematopoietic targets, the instant inventors tested the possible involvement of SLAM family receptors (SFRs), a group of homotypic receptors expressed on hematopoietic cells¹³⁻¹⁵, particularly on a subset of human B cell and T cell lymphomas, and on nearly all cases of multiple myeloma². Analyses of a mouse lacking all SFRs (SFR KO mouse) revealed macrophages with normal differentiation markers (i.e. equivalent to wild-type cells) (FIGS. 3C-A to 3C-H). However, in contrast to cells from wild-type mice, SFR KO macrophages did not display an increase in phagocytosis of hematopoietic cells in the presence of anti-CD47 Ab (FIGS. 3D-F), either intact or F(ab′)₂ fragments (FIGS. 3G-I). This defect was seen both with mouse and human targets, and with BMDMs and peritoneal macrophages. The inventors confirmed these results with the flow cytometry-based and the pHrodo™ dye-based assays (FIGS. 3I-A to 3I-D). SFR KO macrophages also displayed a marked defect in phagocytosis of CD47 KO L1210 cells, compared to wild-type macrophages, in the absence of added anti-CD47 Ab (FIGS. 3J-K). A defect in phagocytosis in response to anti-CD47 Ab was also seen when wild-type macrophages were incubated with SFR KO CD4⁺ T cells, compared to wild-type CD4⁺ T cells (FIG. 3L). SFR KO macrophages exhibited normal phagocytosis of several other types of targets i.e. IgG immune complexes, E. coli, IgG-opsonized sheep red blood cells (RBCs), CD47 KO mouse RBCs, apoptotic thymocytes and IgG-opsonized L1210, when compared to wild-type macrophages (FIGS. 3M-A, 3M-B, 3N and 3O).

To show that SLAM receptors were required for enhanced phagocytosis in response to SIRPalpha-CD47 blockade in vivo, the inventors developed a peritoneal tumor clearance assay. In wild-type mice, co-injection of anti-CD47 Ab, but not control IgG, resulted in clearance of L1210 cells introduced in the peritoneal cavity (FIGS. 3P-Q). However, in SFR KO mice, L1210 cells were not cleared despite normal numbers of peritoneal macrophages in these mice (FIGS. 3P-R). Clearance of L1210 cells from wild-type mice was abrogated by pre-treatment with clodronate, implying that their elimination was macrophage-mediated (FIGS. 35-U). Additionally, when L1210 cells were inoculated sub cutaneously in RAG-1 KO mice, anti-CD47 Ab significantly reduced tumor growth, compared to control IgG (FIGS. 3V-W). The anti-CD47 Ab-dependent effects were absent in RAG-1 SFR double (d) KO mice (FIGS. 3V-W). Hence, SFRs were necessary for the macrophages to eliminate tumor cells in response to SIRPalpha-CD47 blockade in vitro and in vivo. Anti-CD47 Ab delayed, but did not abolish, tumor growth in mice (FIG. 3V), suggesting that as single agent, it cannot completely control tumor growth in vivo.

### Example 4: Determining which SLAM Receptor(s) Disruption Affected for Phagocytosis of Hematopoietic Cells

The SLAM family, which comprises six bona fide members¹³⁻¹⁵, is completely absent in SFR KO mice. Five of the six SFRs, namely SLAMF1 (CD150), 2B4 (CD244), SLAMF7 (CRACC, CS1), Ly-9 and CD84, are expressed on macrophages (FIG. 3C). Several approaches were used to identify which SLAM receptor(s) was(were) responsible for phagocytosis of hematopoietic cells. First, mice lacking individual SFRs were tested. SLAMF7 single KO macrophages, but not SLAMF1, 2B4, Ly-9 or CD84 single KO macrophages, had a defect in phagocytosis in response to anti-CD47 Ab, that was nearly comparable to that of SFR KO macrophages (FIGS. 4A, 4B-A to 4B-C, 4C-A, 4C-B). SLAMF7 KO macrophages did not display any defect in phagocytosis of IgG complex, E, coil or IgG-opsonized L1210 cells, and had normal expression of macrophage markers except for loss of SLAMF7 (FIGS. 4B-A to 4B-C, 4D-A and 4D-B).

Moreover, the defect in phagocytosis observed in SFR KO macrophages was rescued by re-expression of mouse Slamf7, using bacterial artificial chromosome-mediated transgenesis (FIGS. 4E, 4F, 4G, 4H-A to 4H-E).

### Example 5: Determining Whether Hematopoietic Target Cells Susceptible to Enhanced Phagocytosis in Response to Anti-CD47 Ab Express SLAMF7

The inventors then tested whether hematopoietic target cells susceptible to enhanced phagocytosis in response to anti-CD47 Ab expressed SLAMF7. All susceptible target cells, mouse or human, expressed SLAMF7 (FIGS. 5A-A to 5A-C). In contrast, none of the non-susceptible target cells, except normal B cells, did (FIGS. 2N and Q; FIGS. 5B-A, 5B-F). Susceptible targets also expressed other SFR ligands, but macrophages lacking these SFRs showed no defect in phagocytosis (FIGS. 5C-A to 5C-P; FIG. 4A).

Then, ectopic expression of SLAMF7 was enforced on the SLAMF7-negative MEL cell line and its susceptibility to phagocytosis was tested (FIGS. 5B-A, 5B-F). Expression of SLAMF7 rendered MEL cells prone to enhanced phagocytosis by wild-type macrophages, but not by SFR KO or SLAMF7 KO macrophages, in the presence of anti-CD47 Ab (FIGS. 5C-A to 5C-P). Expression of SLAMF7 also promoted anti-CD47 Ab-evoked elimination of MEL cells in vivo, as shown by their peritoneal clearance in NOD-RAG-1 KO-common gamma chain KO (NRG) mice (FIGS. 5D-E).

The inventors also found that, unlike wild-type activated CD4⁺ T cells, SLAMF7 KO activated CD4⁺ T cells did not display increased phagocytosis by wild-type macrophages with anti-CD47 Ab (FIGS. 5H-A, 5H-B). Likewise, when injected intravenously in wild-type mice, SLAMF7 KO CD4⁺ T cells were less efficiently cleared from the blood in response to anti-CD47 Ab, compared to wild-type CD4⁺ T cells (FIG. 5I). Then, as human target cells were also susceptible to augmented phagocytosis by mouse macrophages in the presence of anti-CD47 Ab (FIG. 2Q), human SLAMF7 were re-expressed on SFR KO mouse macrophages, using retrovirus-mediated gene transfer (FIGS. 5J-A to 5J-C). Expression of human SLAMF7, like mouse SLAMF7, recreated the enhanced phagocytosis response during CD47 Ab blockade (FIGS. 5J A to 5J-C). Lastly, anti-mouse SLAMF7 Ab 4G2, but not a control Ab, interfered with the enhanced ability of wild-type macrophages, from either C57BL/6 mice or NRG mice, to engulf L1210 cells in the presence of anti-CD47 Ab (FIG. 5K). Similarly, anti-human SLAMF7 Ab 162 blocked the augmented capacity of human blood-derived macrophages to engulf Raji cells in response to anti-CD47 Ab (F(ab′)₂) (FIG. 5L). Therefore, SLAMF7 expression on macrophages and tumor cells was required to endow mouse and human macrophages with the capacity to phagocytose hematopoietic tumor cells in the presence of anti-CD47 Ab.

### Example 6: Determining Mechanism of Anti-CD47-Dependent Enhancement of Phagocytosis of SLAM Positive Cells

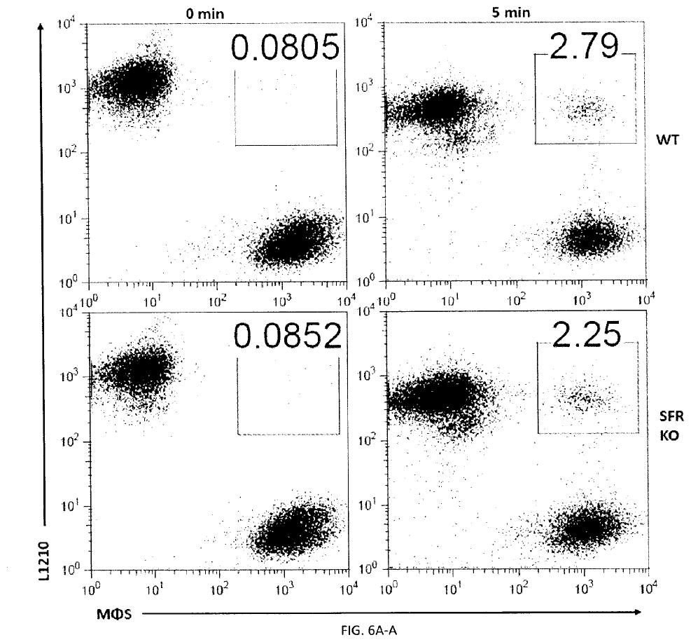

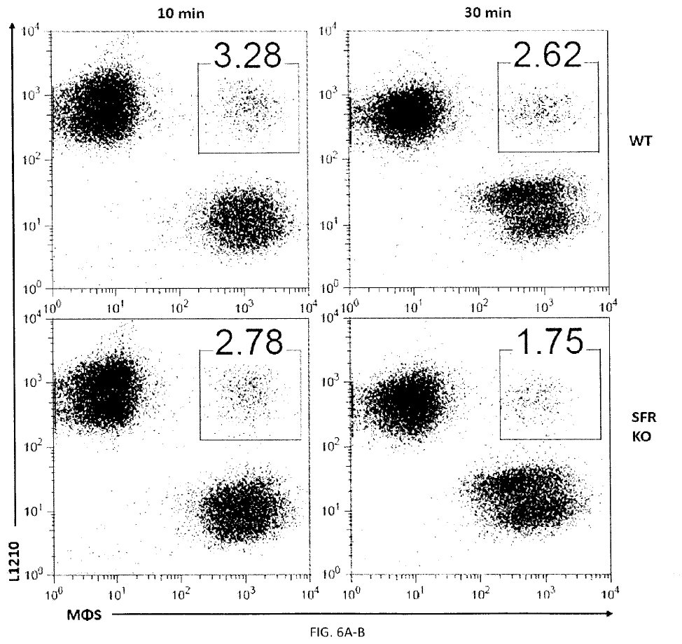

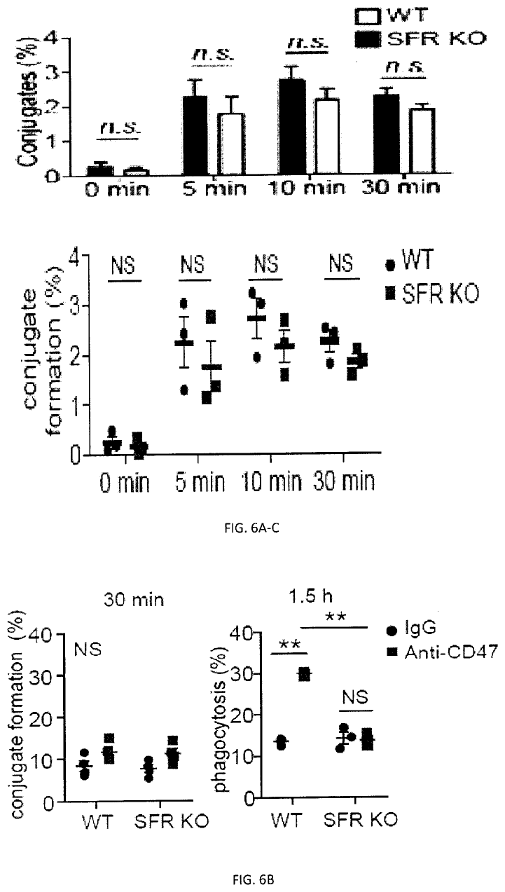

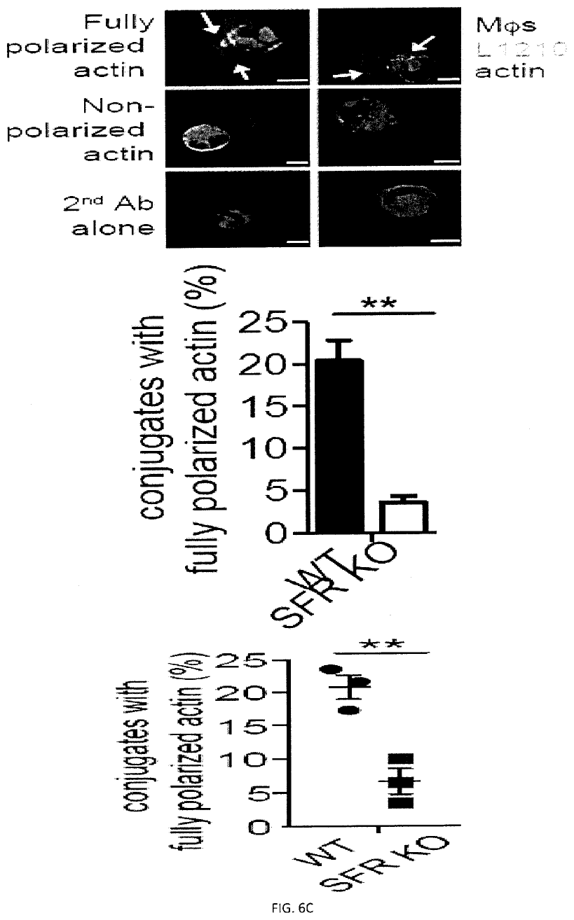

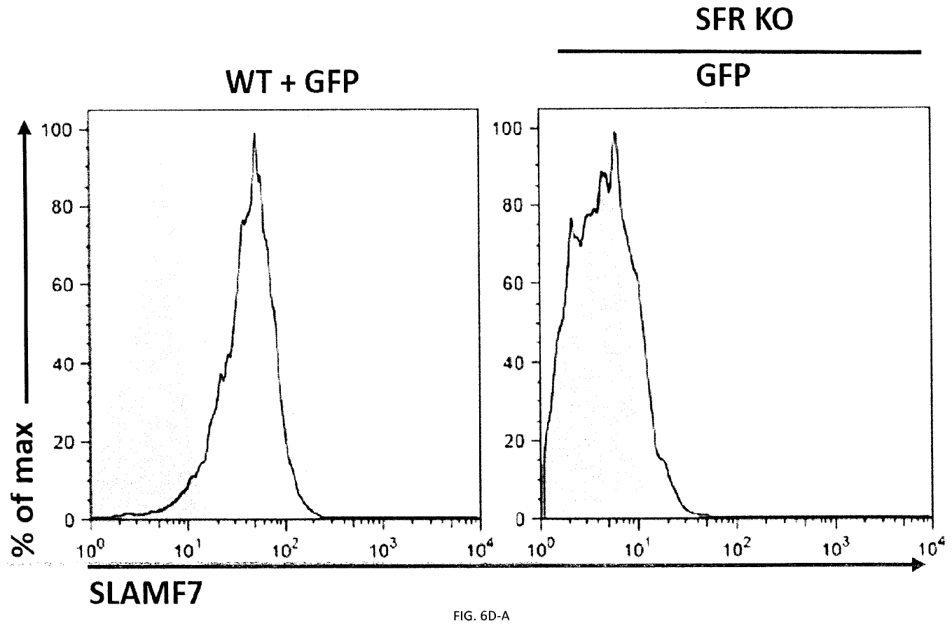

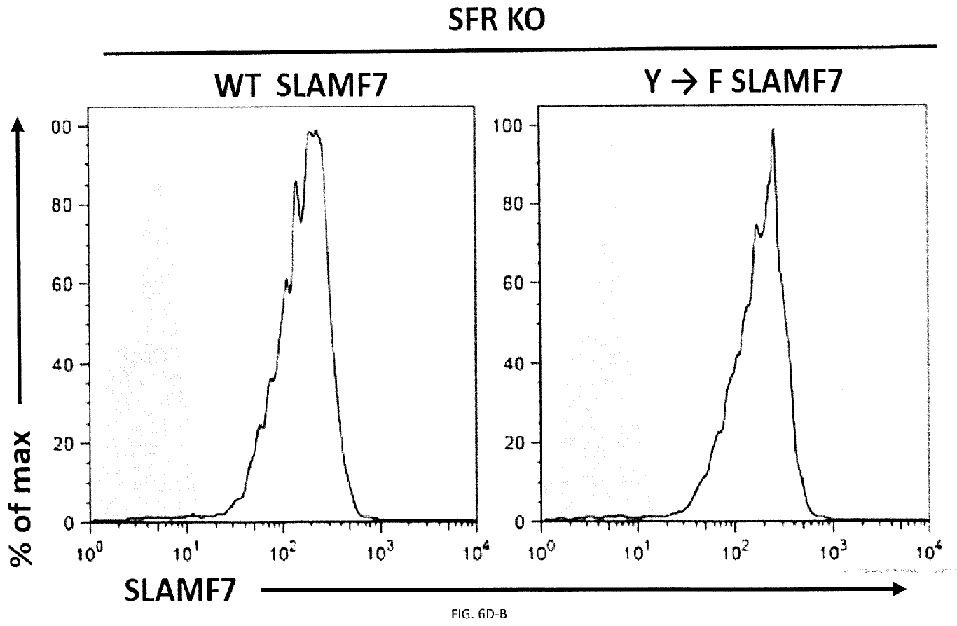

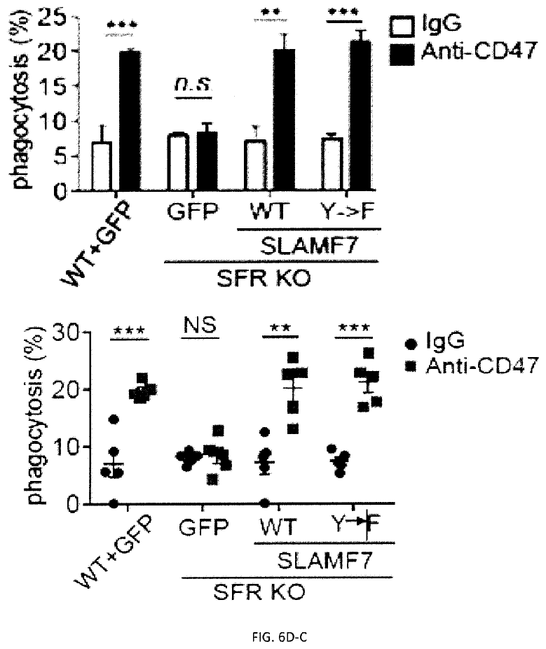

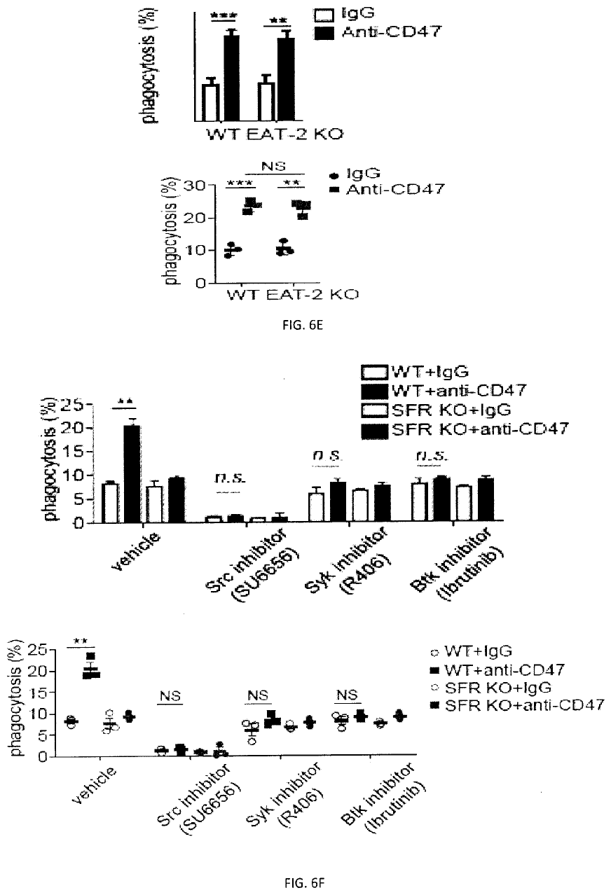

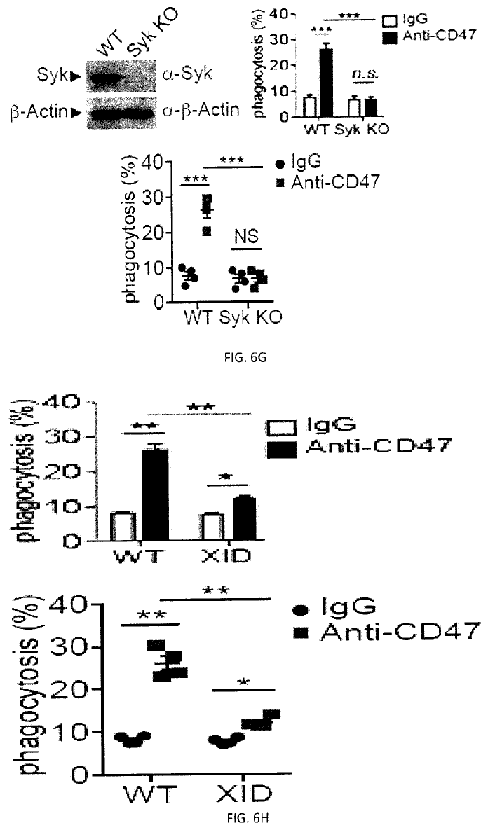

The inventors tested the possibility that SLAMF7 acted as an adhesion molecule augmenting physical contacts between macrophages and target cells. However, conjugate formation assays revealed little or no defect in the frequency of conjugates or conjugate formation between SFR KO macrophages and L1210 cells in the presence of anti-CD47 Ab, compared to wild-type macrophages (FIGS. 6A-A to 6A-C), although a phagocytosis defect was seen at a later time point (FIG. 6B). The inventors also tested the possibility that SLAMF7 promotes signals triggering phagocytosis. Confocal microscopy studies of conjugates formed between macrophages and L1210 cells in the presence of anti-CD47 Ab showed that actin polarization towards target cells, a key step during phagocytosis, was markedly reduced in SFR KO macrophages, compared to wild-type macrophages (FIG. 6C). Hence, SLAMF7 did not detectably enhance adhesion to targets, but did stimulate cytoskeletal reorganization required for phagocytosis.

To elucidate how SLAMF7 mediated these effects, a SLAMF7 variant in which three intra-cytoplasmic tyrosine residues (Y) were mutated to phenylalanines (F) (Y-F mutations) were first expressed in SFR KO macrophages (FIG. 6D). These tyrosines couple SLAMF7 to various effectors, including the SAP adaptor EAT-2^{13-15,23}. The Y→F mutations had no impact on phagocytosis (FIGS. 6D-A to 6D-C). Similarly, and in agreement with this finding, phagocytosis was also unaffected in EAT-2 KO macrophages (FIG. 6E). As SLAM receptors typically mediate their function through intra-cytoplasmic tyrosines that bind SAP adaptors¹³⁻¹⁵ these observations implied that SLAMF7 promoted phagocytosis by a novel SAP adaptor-independent mechanism.

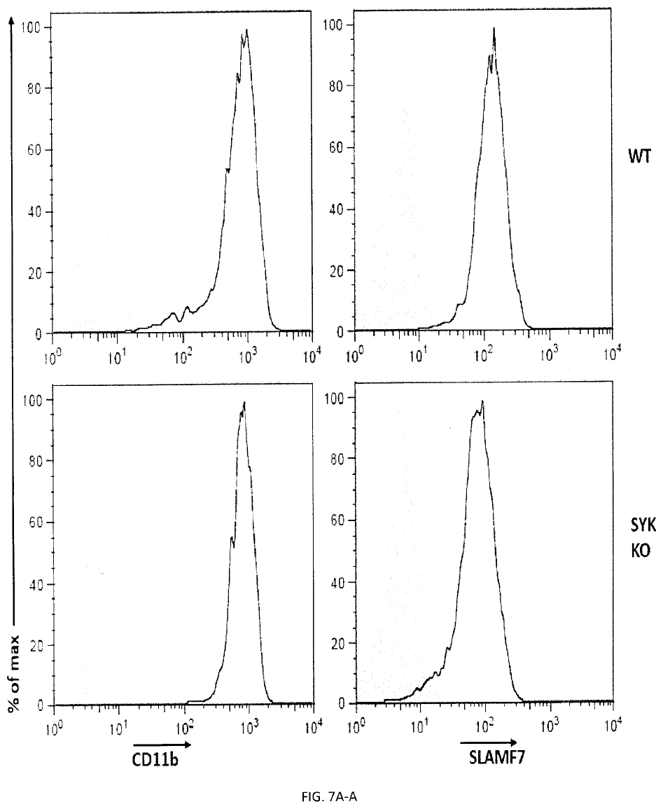

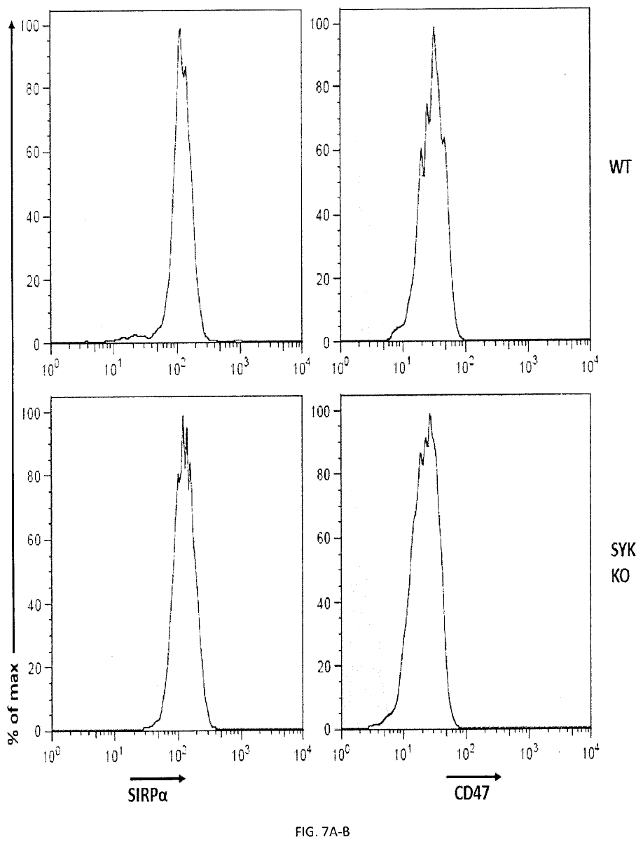

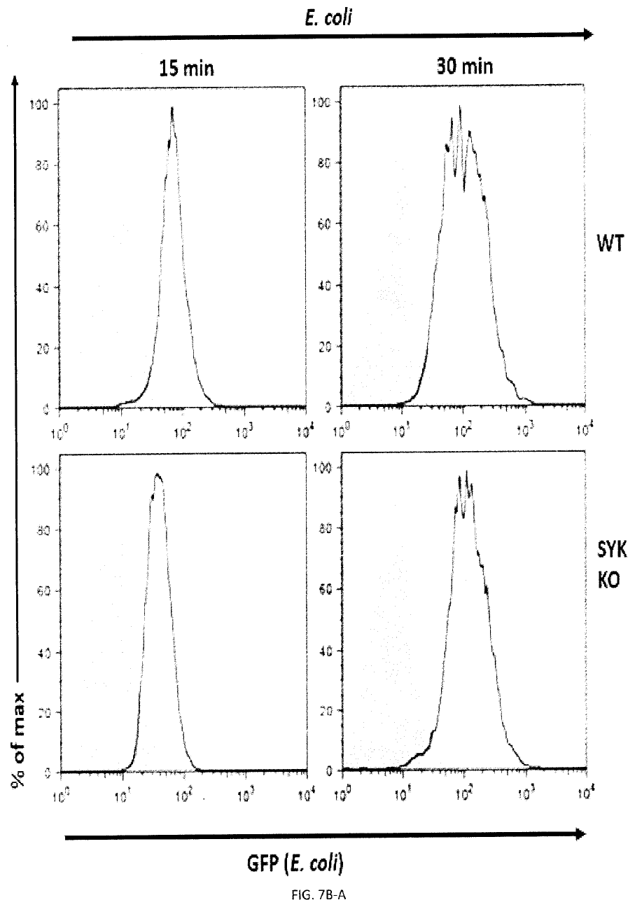

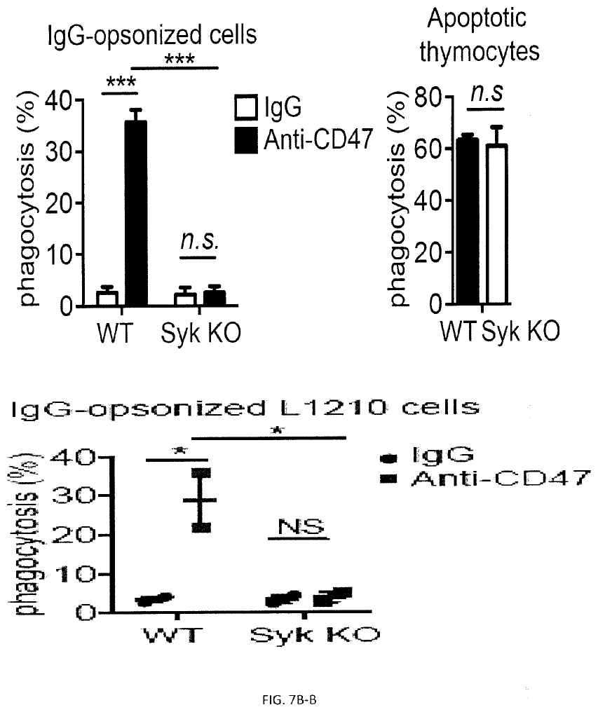

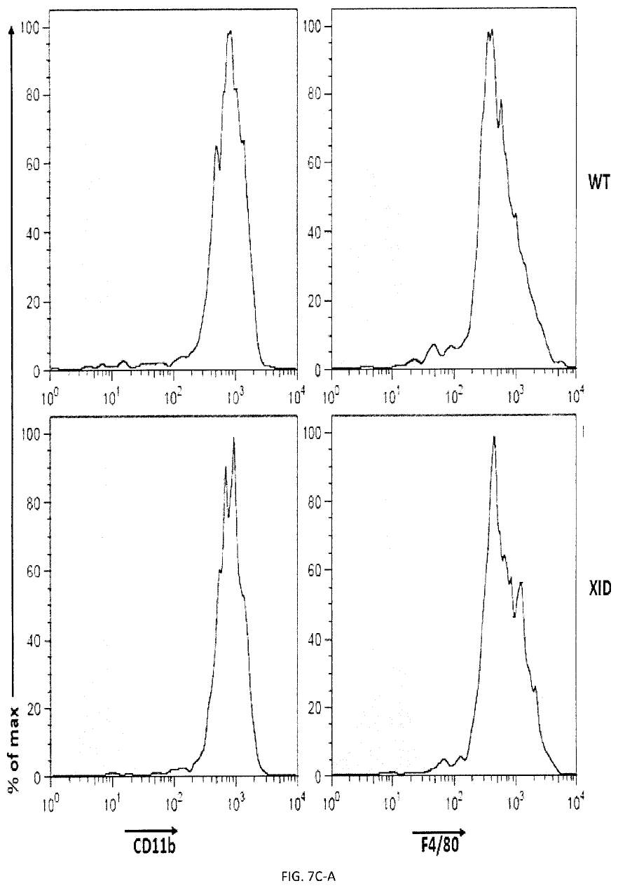

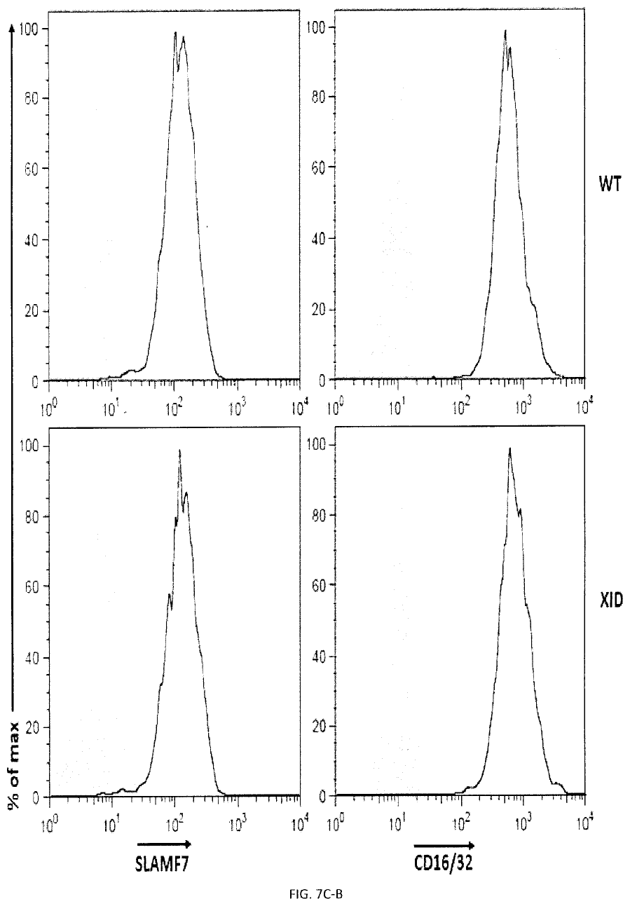

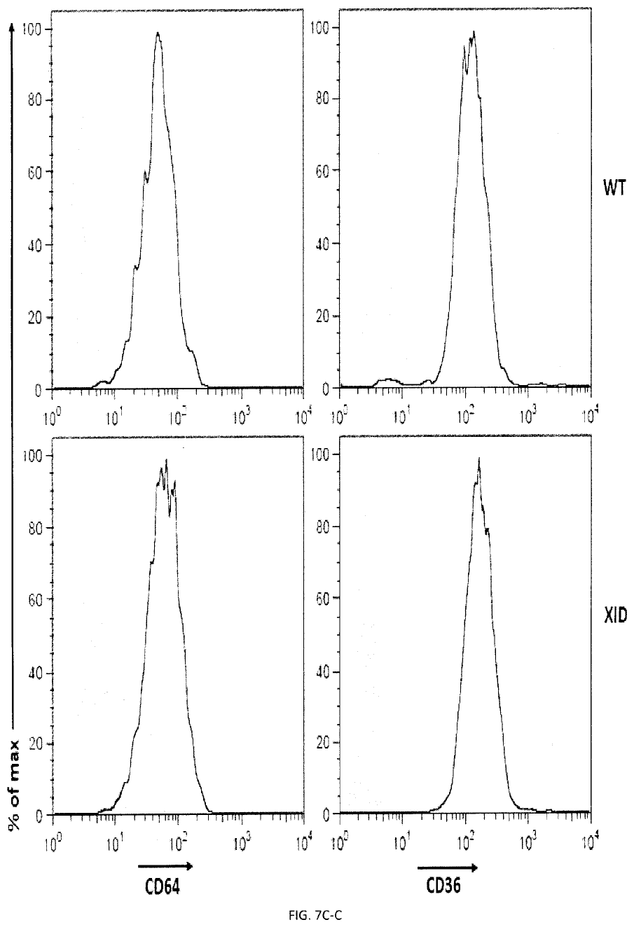

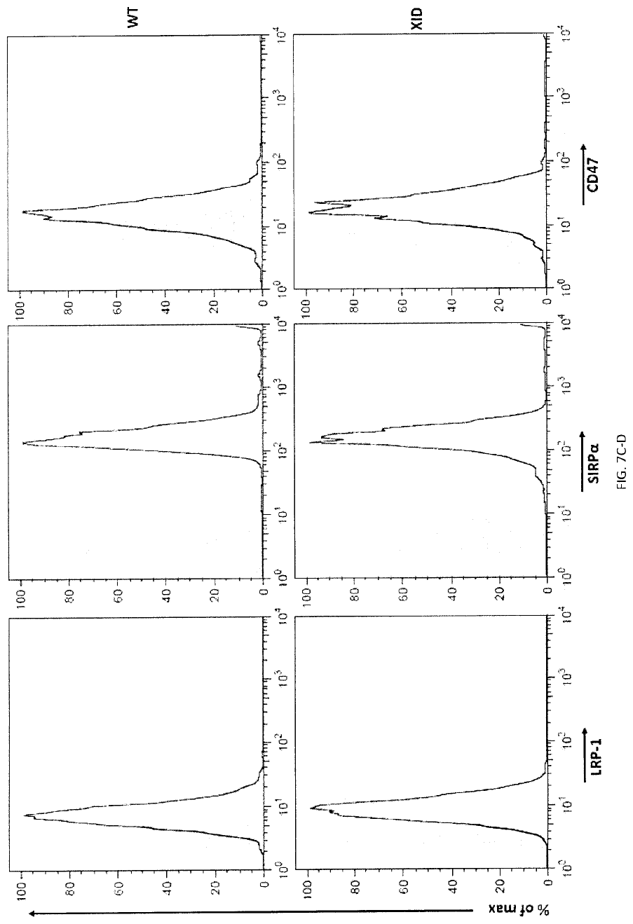

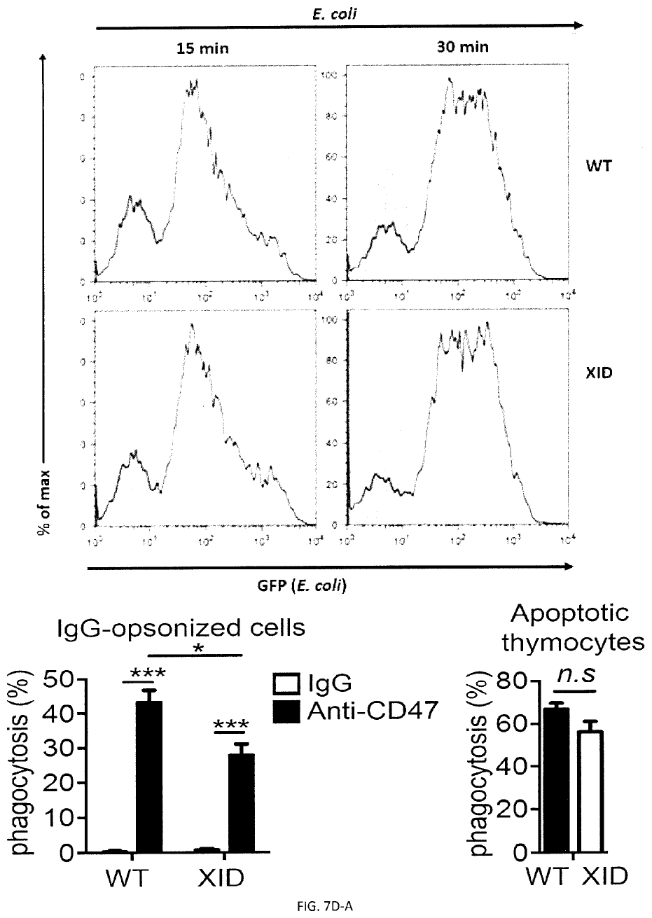

To identify this mechanism, the involvement of various signaling effectors was analyzed in SLAMF7-dependent phagocytosis. Pharmacological inhibitors of three classes of protein tyrosine kinases involved in phagocytosis by other receptors^{4,19}, the Src family, Syk and Btk kinases, abrogated the enhanced phagocytosis in response to anti-CD47 Ab (FIG. 6F). Moreover, the enhanced phagocytosis of L1210 cells in response to anti-CD47 Ab was abrogated in Syk KO macrophages (FIG. 6G), and reduced in macrophages from X-linked immunodeficiency (XID) mice²⁴, which carry a loss-of-function mutation of Btk (FIG. 6H). Loss of Syk or the XID mutation had no impact on expression of SLAMF7 or other macrophage markers (FIGS. 7A-A, 7A-B, 7B-A, 7B-B, 7C-A to 7C-D, 7D-A and 7D-B). However, Syk KO and XID macrophages had defects in phagocytosis of IgG-opsonized targets, as expected from the involvement of Syk and Btk in Fc receptor (FcR)-mediated phagocytosis²⁰, but had no defect in phagocytosis of apoptotic thymocytes or E. coli (FIG. 7A A, 7A-B, 7B-A, 7B-B, 7C-A to 7C-D, 7D-A and 7D-B). Hence, SLAMF7-dependent phagocytosis required Src, Syk and Btk kinases, and this effect was macrophage-intrinsic for at least Syk and Btk.

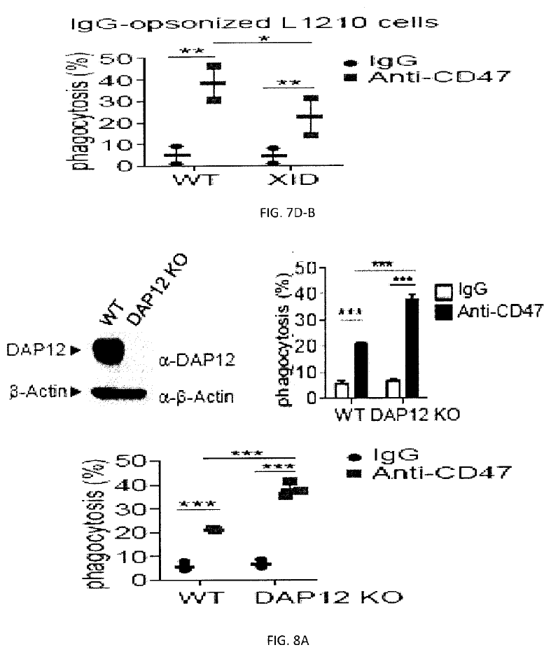

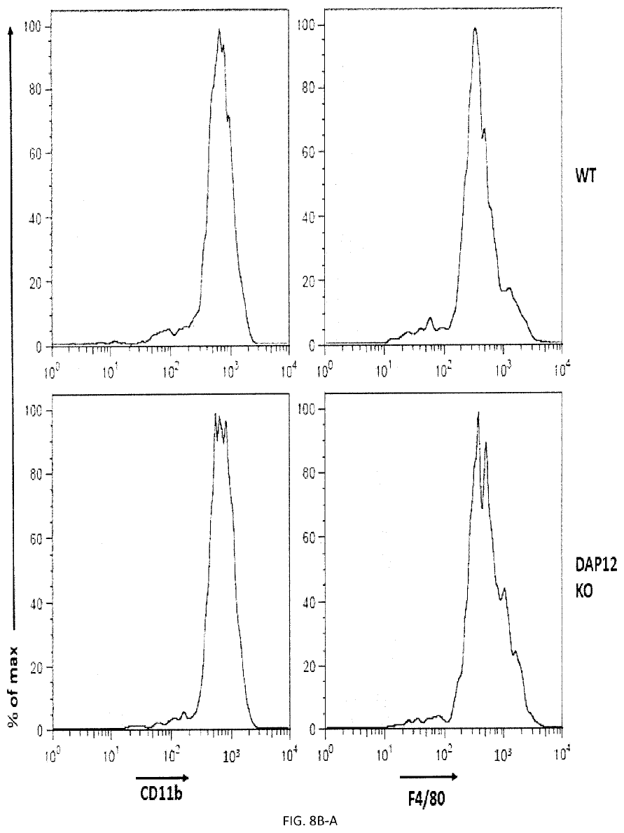

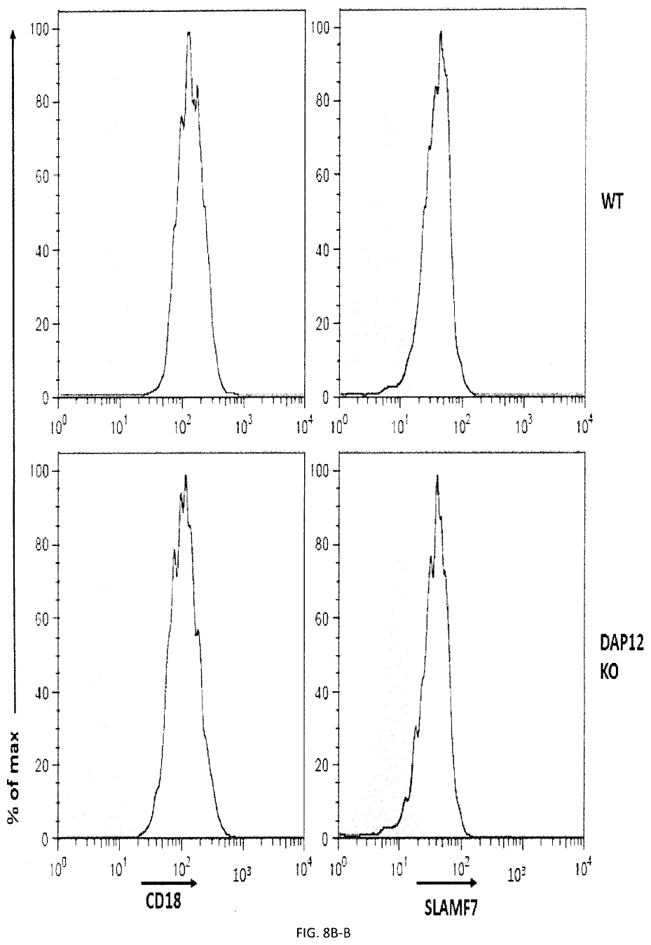

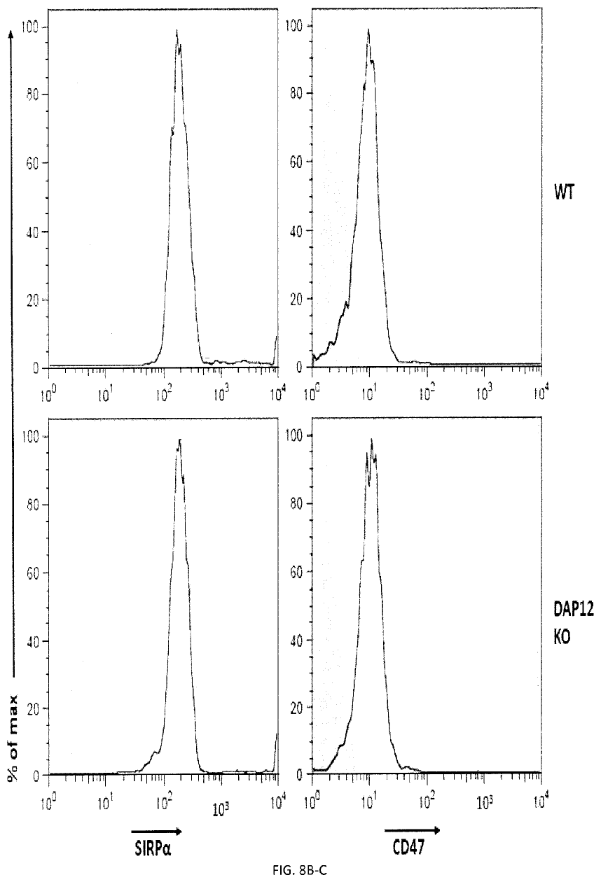

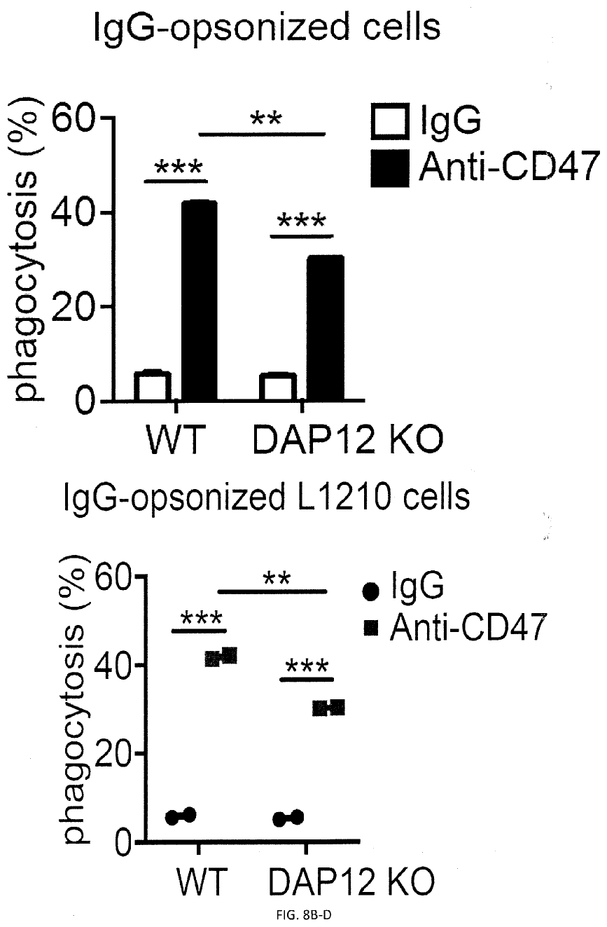

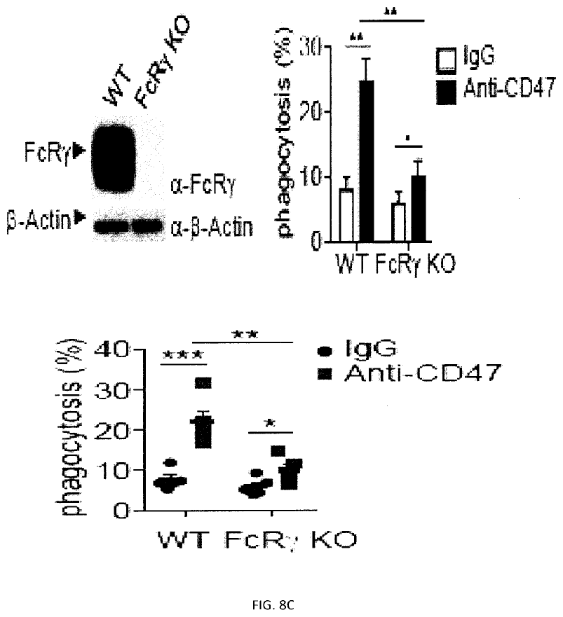

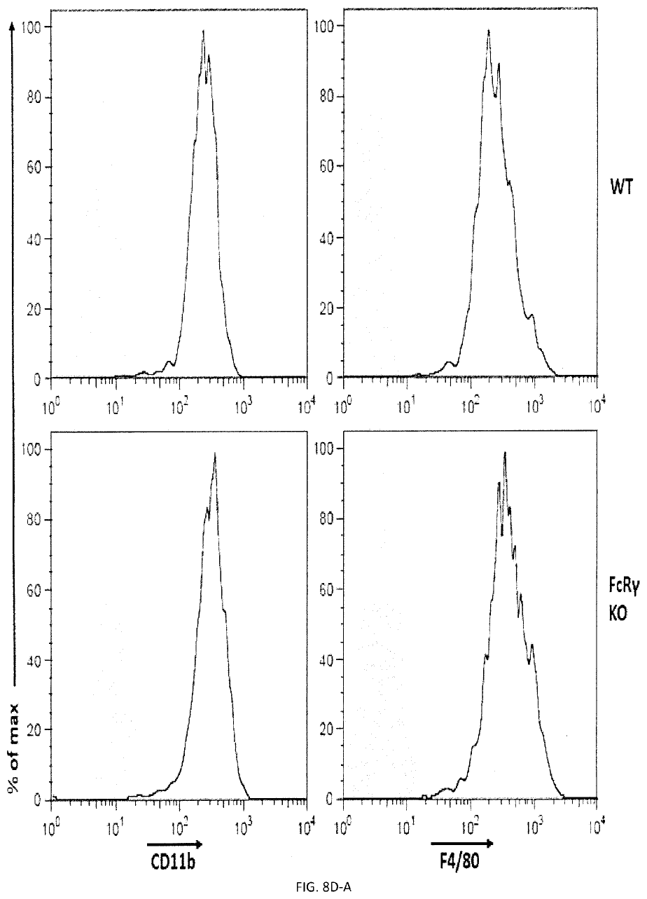

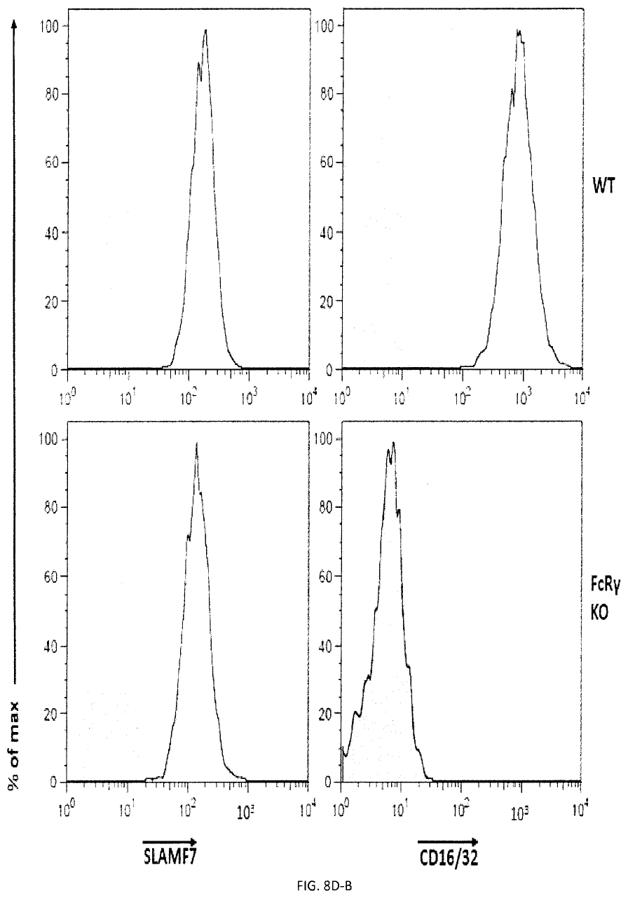

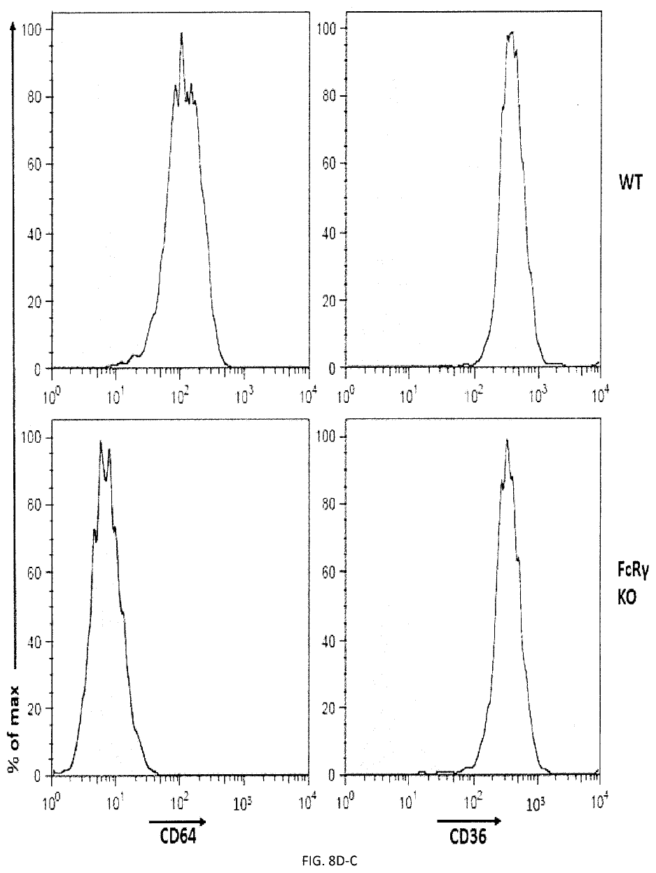

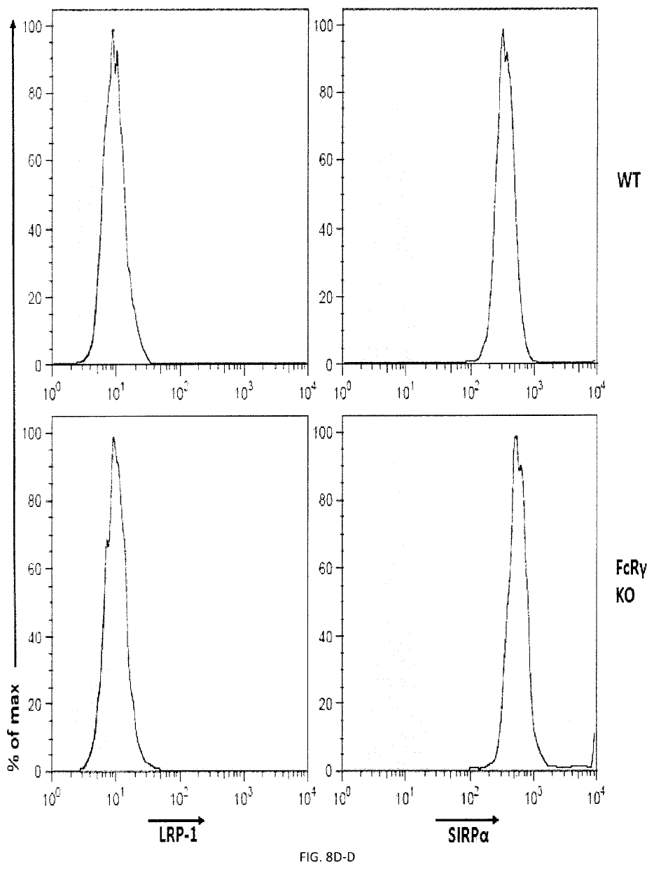

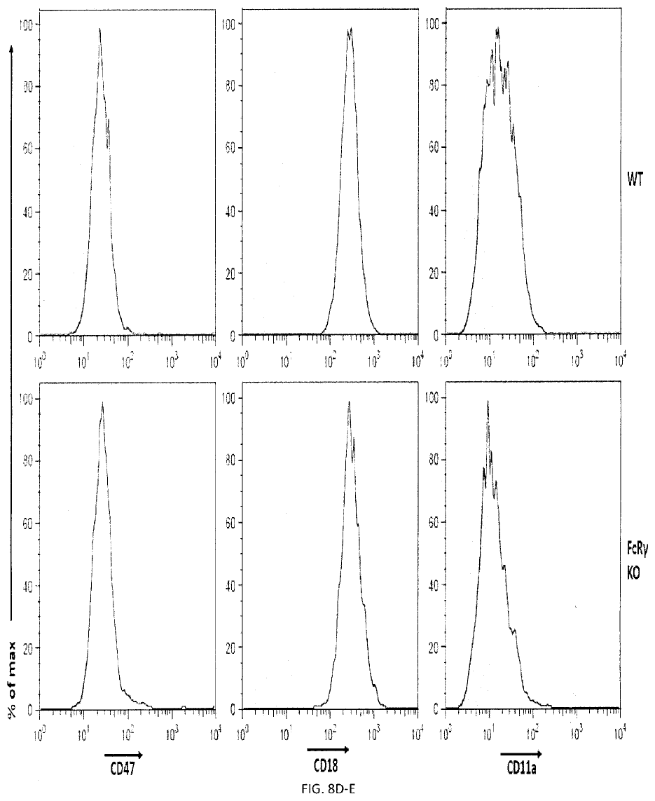

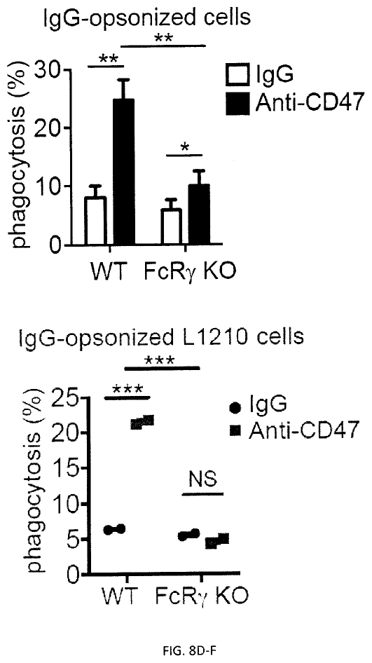

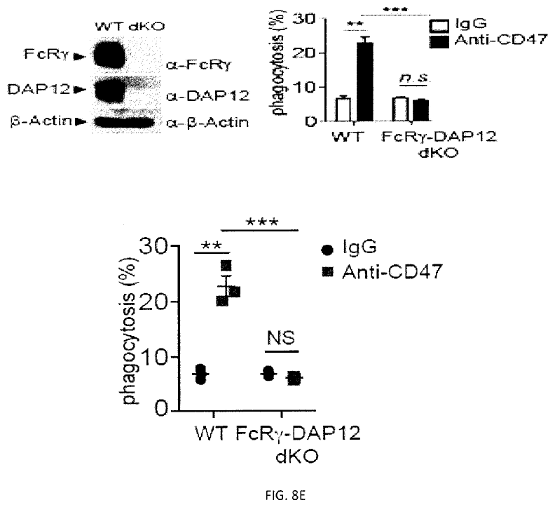

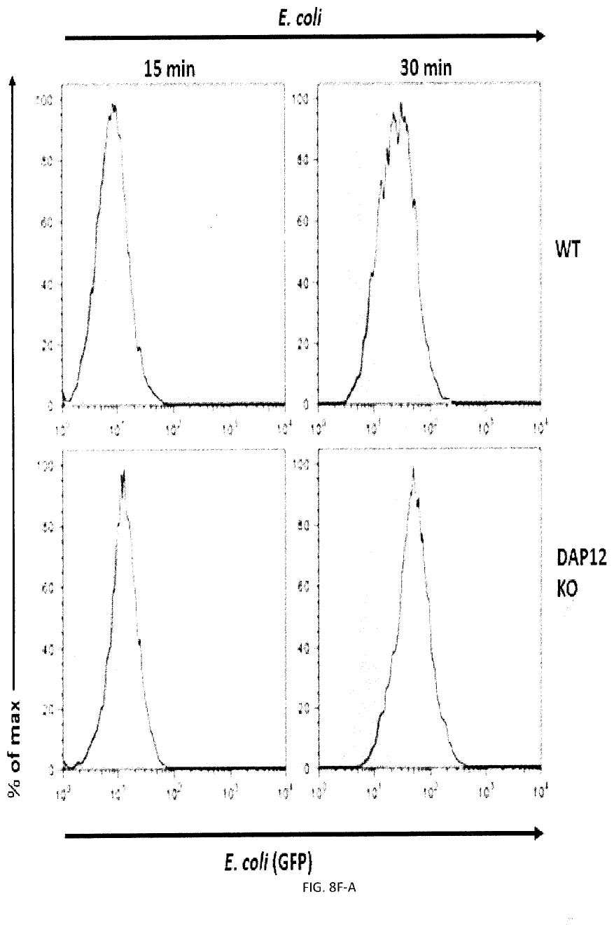

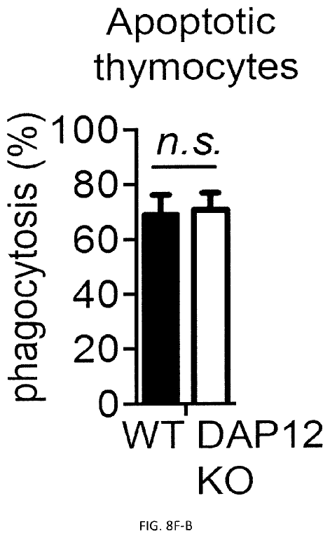

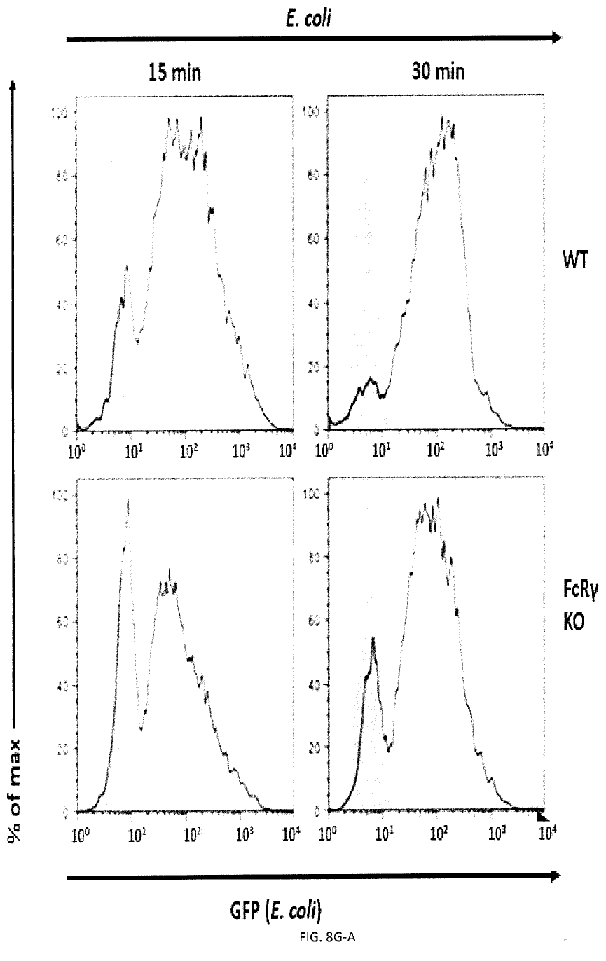

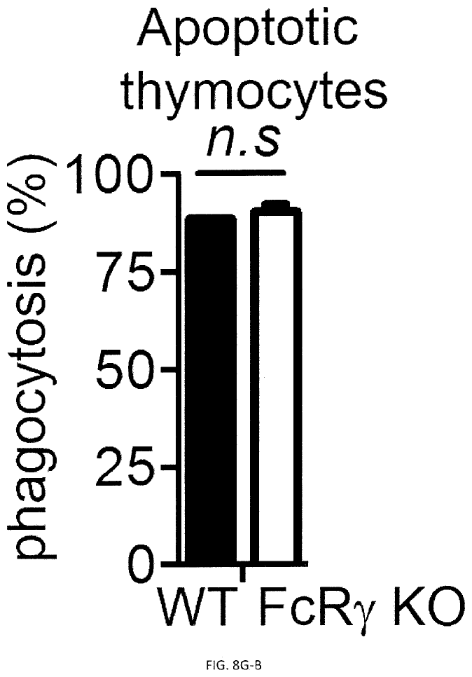

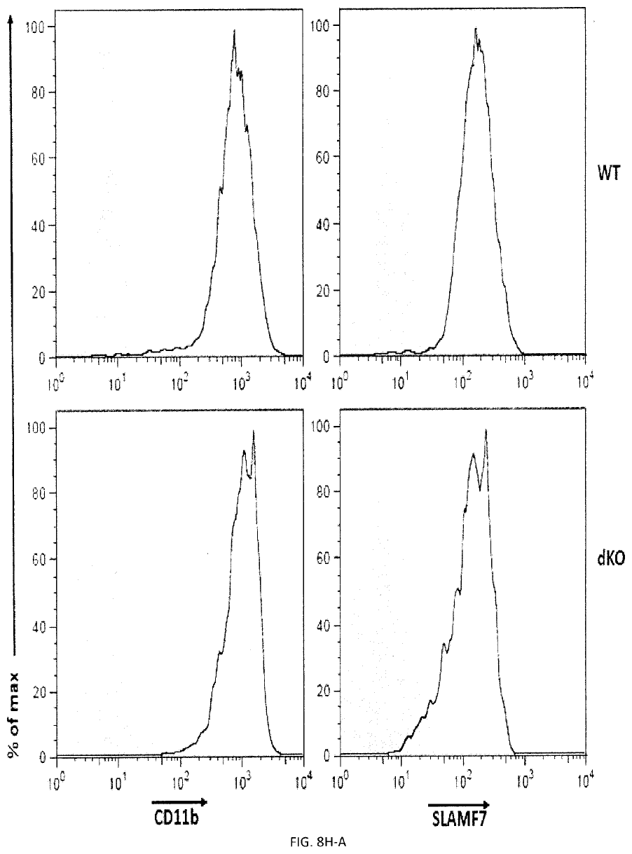

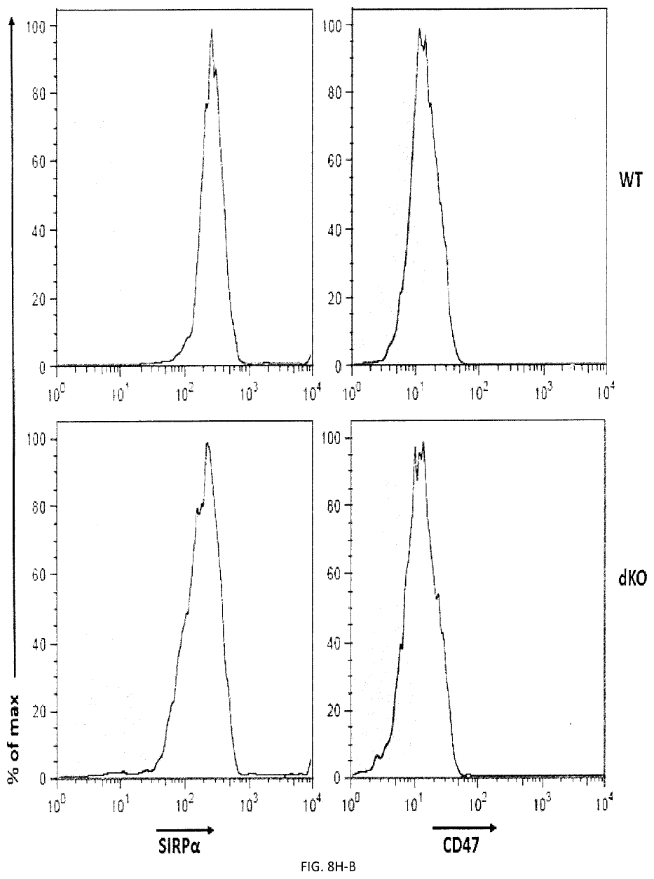

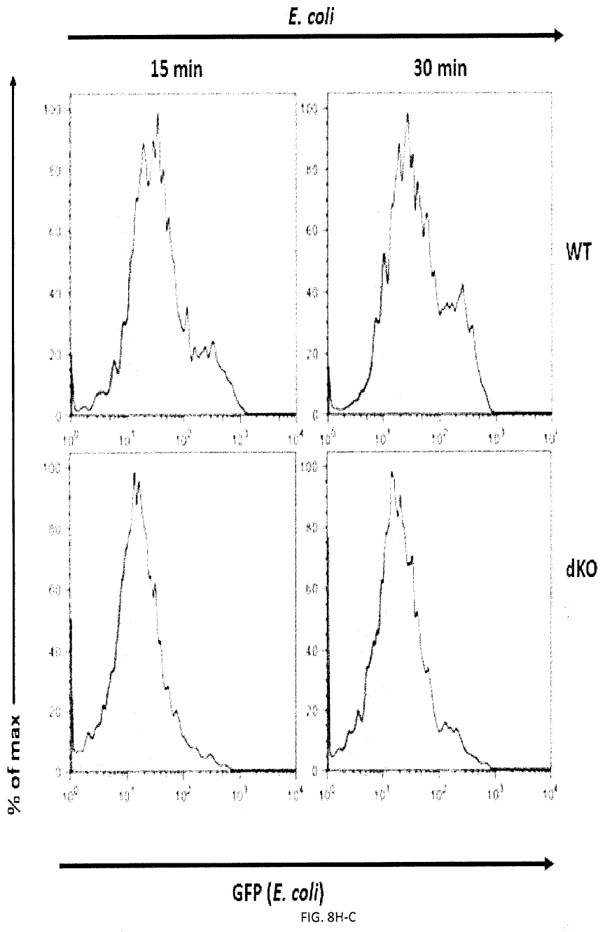

These findings suggested that SLAMF7 might promote phagocytosis by utilizing ITAM-containing proteins, which mediate immune cell activation via sequential involvement of the Src, Syk and Btk kinases^{19,20}. Macrophages express two ITAM-containing proteins, DAP12 and Fc receptor-associated gamma (FcR gamma) chain²⁰. No defect in anti-CD47-dependent enhancement of phagocytosis of L1210 was seen in DAP12 KO macrophages (FIGS. 8A, 8B-A to 8B-D). However, a partial defect in FcR gamma KO macrophages was observed (FIGS. 8C, 8D-A to 8D-F). Macrophages lacking both FcR gamma and DAP12 displayed a complete defect (FIG. 8E). Absence of DAP12, FcR gamma or both had no impact on expression of SLAMF7 or other macrophage markers, with the exception of the Fc receptors CD16/32 and CD64, which were absent in FcR gamma KO macrophages, as described²⁵ (FIGS. 8B-A to 8B-D, D-A to 8D-F, 8F-A, 8F-B, 8G-A, 8G-B, 8H-A to 8H-E). Defects in FcR-mediated phagocytosis were also seen in FcR gamma KO macrophages, as expected²⁰ (FIGS. 8D-A to 8D-F, 8F-A, 8F-B, 8G-A, 8G-B, 8-A to 8H-E). Thus, SLAMF7-dependent induction of phagocytosis was dependent on contributions from FcR gamma and DAP12.

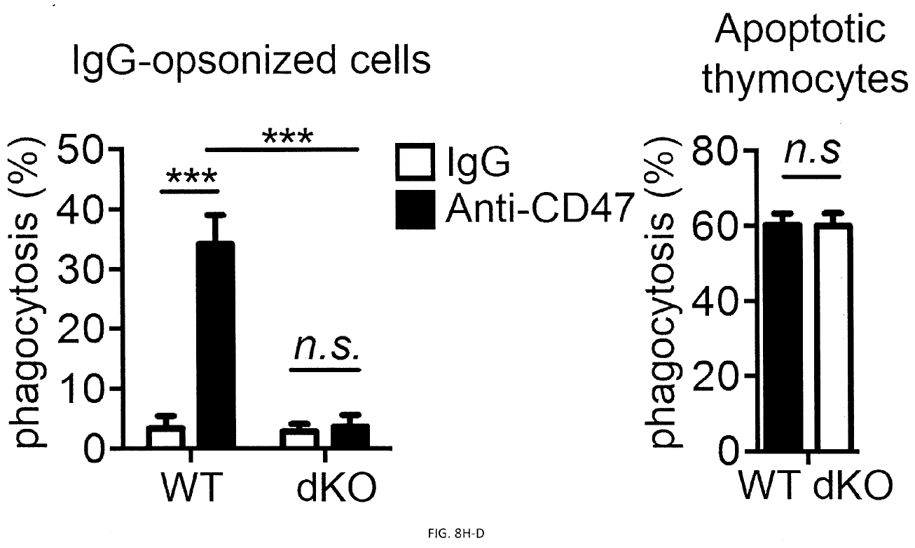

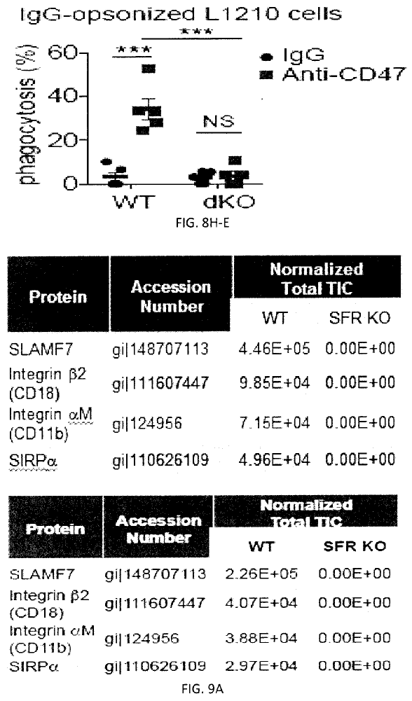

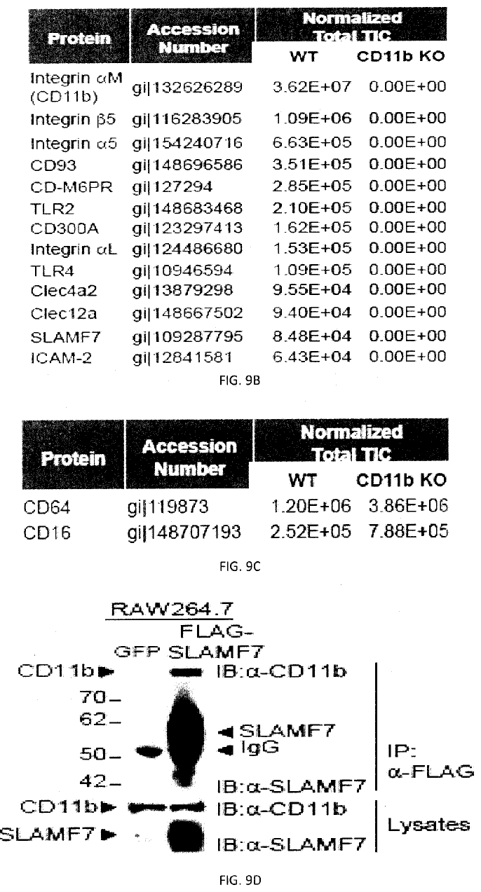

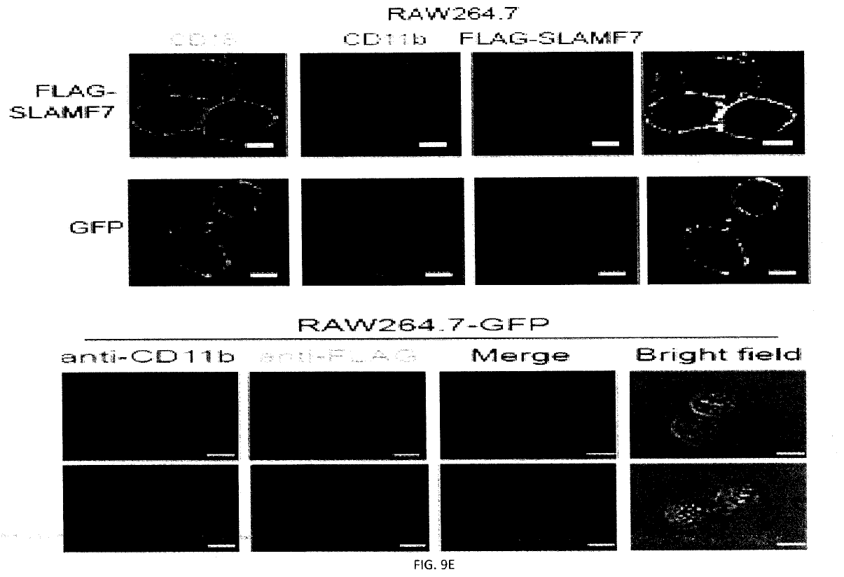

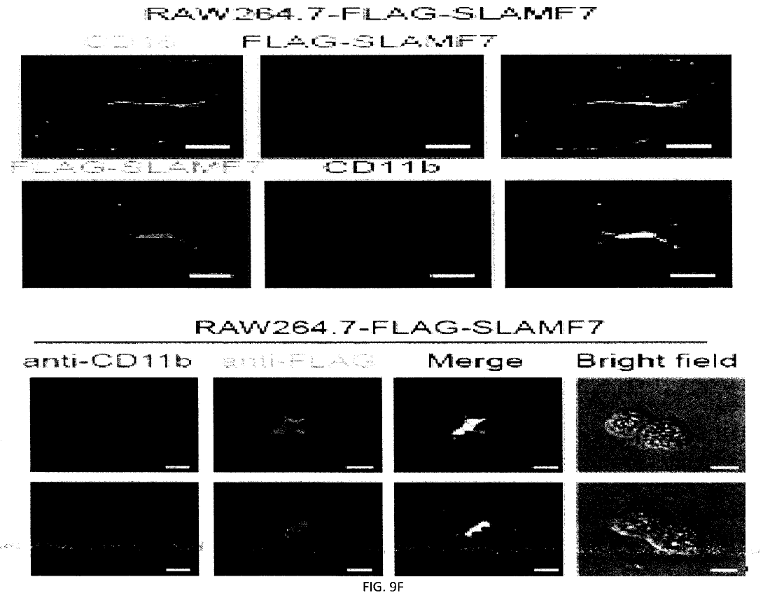

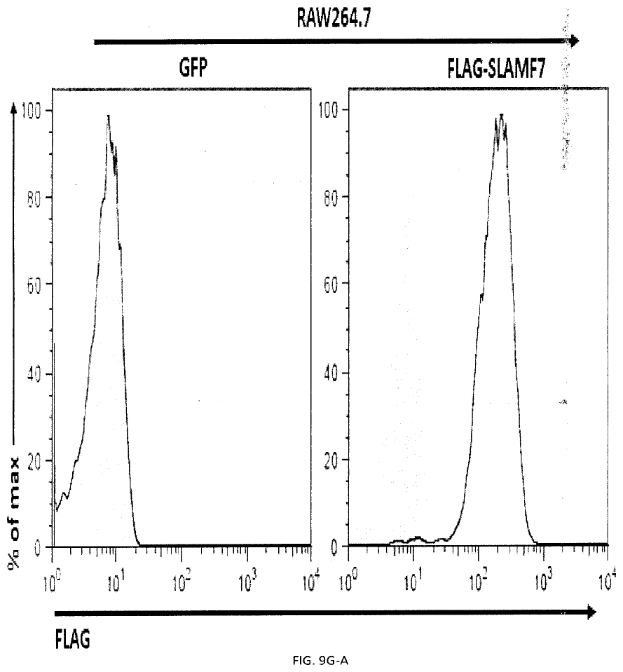

ITAM-containing proteins are typically associated with transmembrane receptors, which recognize the extracellular ligands that trigger ITAM-dependent cell activation^{19,20}. SLAMF7 lacks a charged transmembrane domain residue that is needed to bind ITAM-containing subunits. Hence, to assess if SLAMF7 might interact with FcR gamma and DAP12 through other receptors, SLAMF7 was immunoprecipitated from wild-type macrophages, and associated proteins were analyzed by mass spectrometry. In addition to SLAMF7, these SLAMF7 immunoprecipitates contained two integrin proteins, the alpha subunit CD11 b (alphaM) and the beta subunit CD18 (beta2), which constitute Mac-1¹⁶ 18 (FIG. 9A). SIRPalpha was also identified as a SLAMF7-associated protein. Mac-1 and SIRPalpha were absent from anti-SLAMF7 immunoprecipitates prepared from SFR KO macrophages. Conversely, SLAMF7 was identified in anti-CD11b immunoprecipitates from wild-type, but not CD11b KO, macrophages (FIG. 9B). Anti-CD11b immunoprecipitates from wild-type macrophages also contained other receptors, but no other SFRs. As the other receptors found in CD11b immunoprecipitates were not seen in SLAMF7 immunoprecipitates, the complexes of CD11 b with SLAMF7 or these other receptors were presumably independent. CD64 and CD16 were equally present in anti-CD11 b immunoprecipitates from WT and CD11 b KO macrophages, implying that these co-immunoprecipitations were non-specific (FIG. 9C).

Mac-1 is known to interact with FcR gamma and DAP12^{19,20}. It has multiple broadly expressed ligands such as ICAM-1 and promotes phagocytosis of various types of targets, including pathogens. It is also known as complement receptor 3 (CR3), due to its ability to bind targets opsonized by inactive C3b complement (C3bᵢ)^{16-18,26.} The association between SLAMF7 and Mac-1 (CD11b) and their co-localization on the cell surface were confirmed by immunoblot analyses (FIG. 9D) and confocal microscopy studies (FIGS. 9E-F), respectively, using derivatives of the macrophage cell line RAW264.7 expressing a FLAG-tagged version of SLAMF7 (FIGS. 9GA and 9G-B). Co-localization of SLAMF7 with Mac-1 (CD11b and CD18) was especially dense at areas of cell-cell contact and was associated with foci of SLAMF7 accumulation (FIGS. 9E-F).

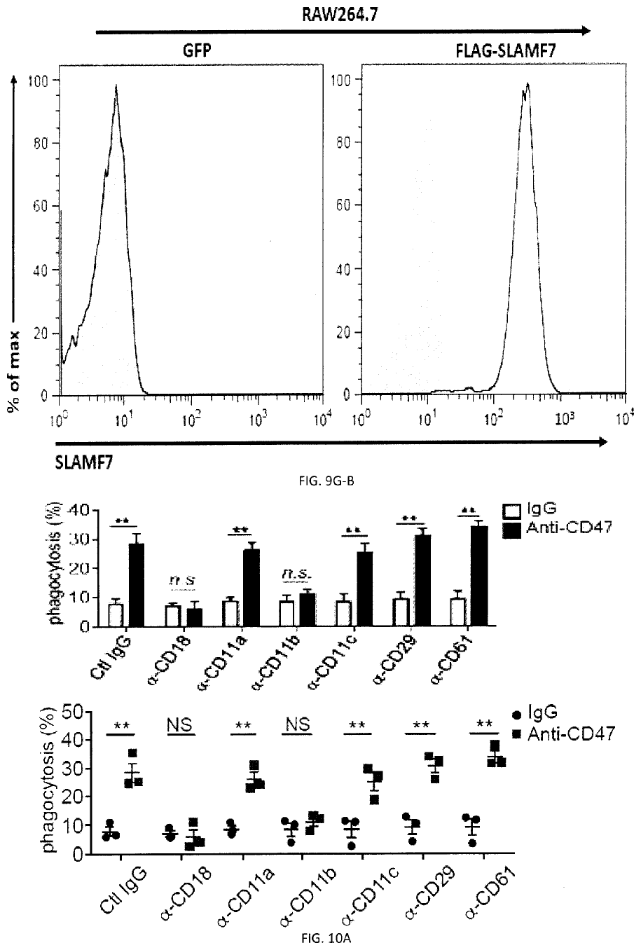

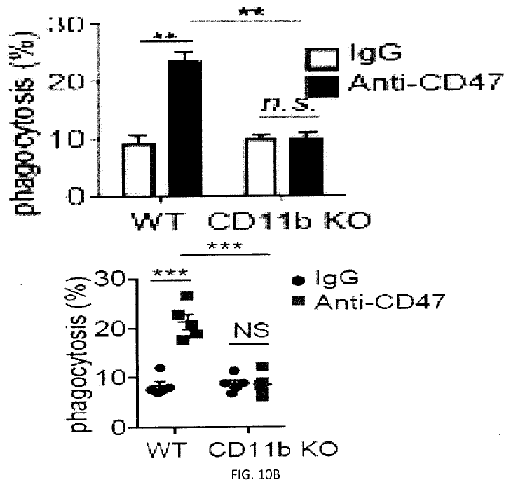

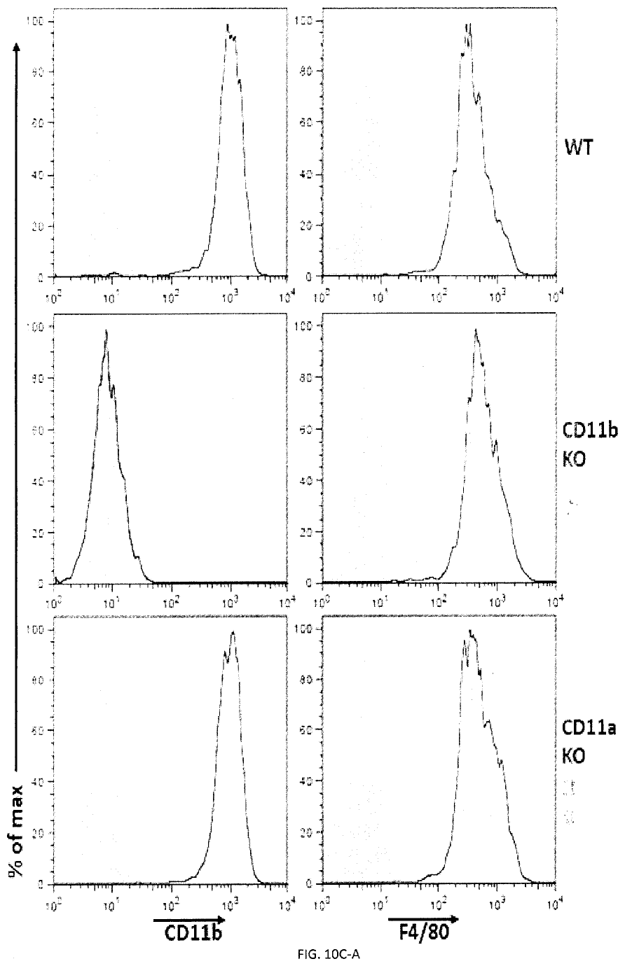

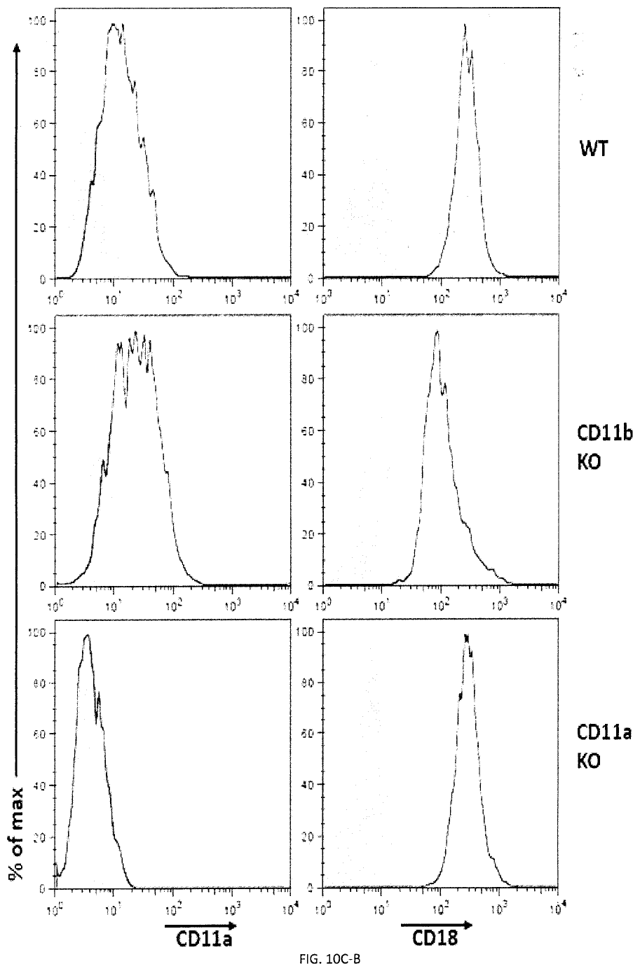

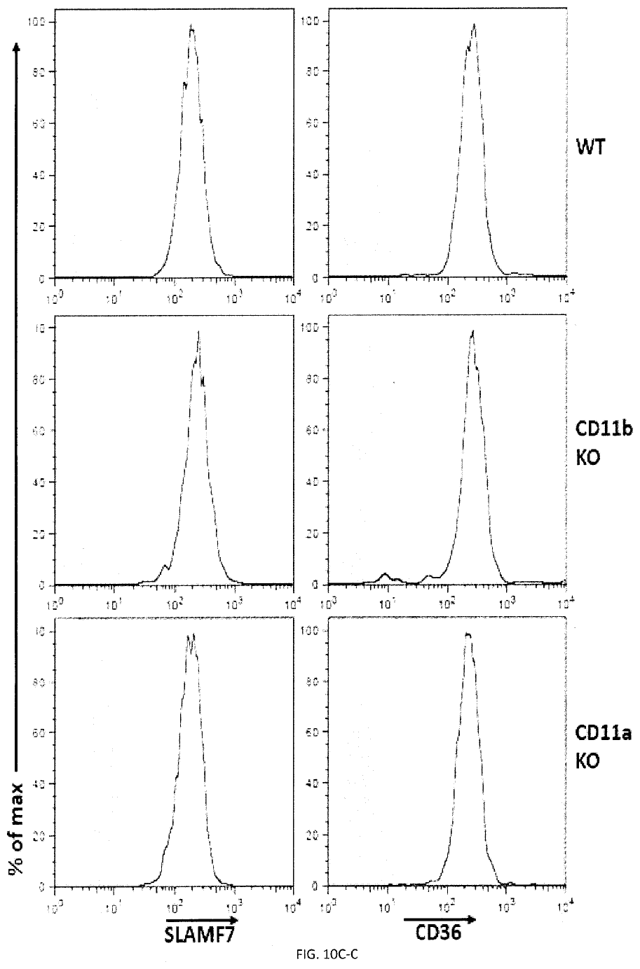

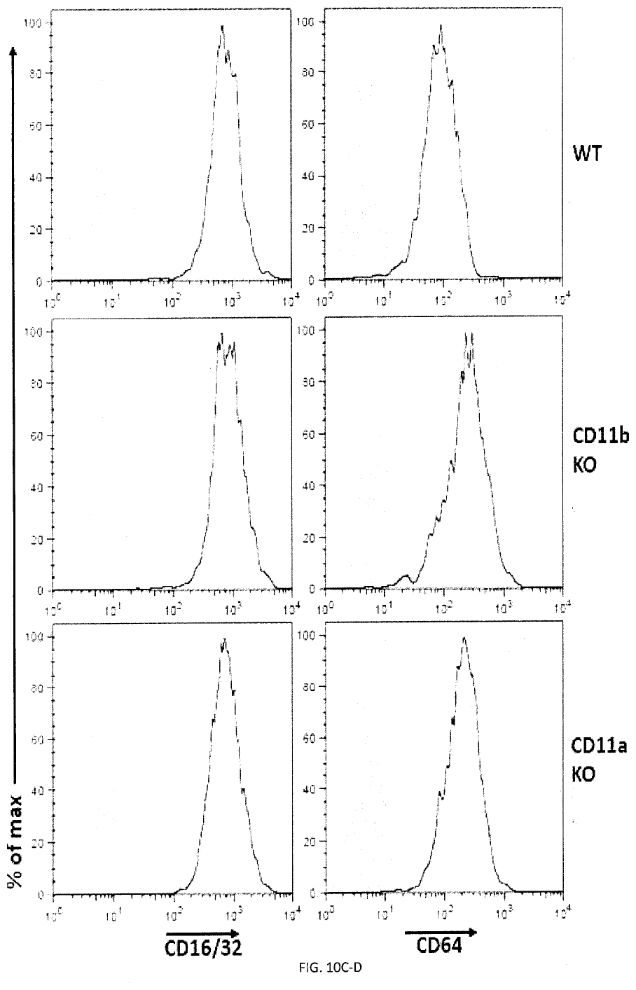

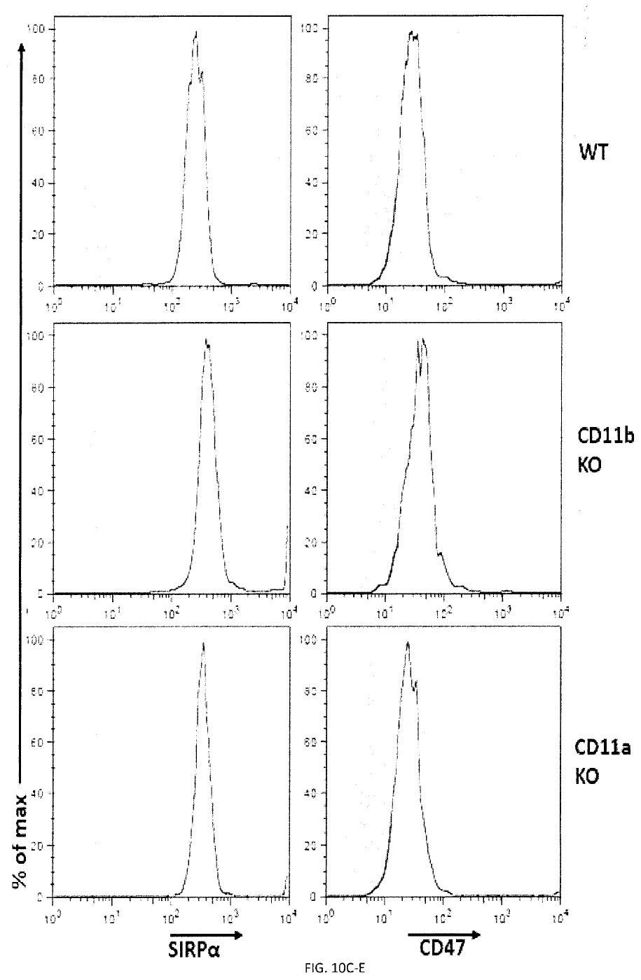

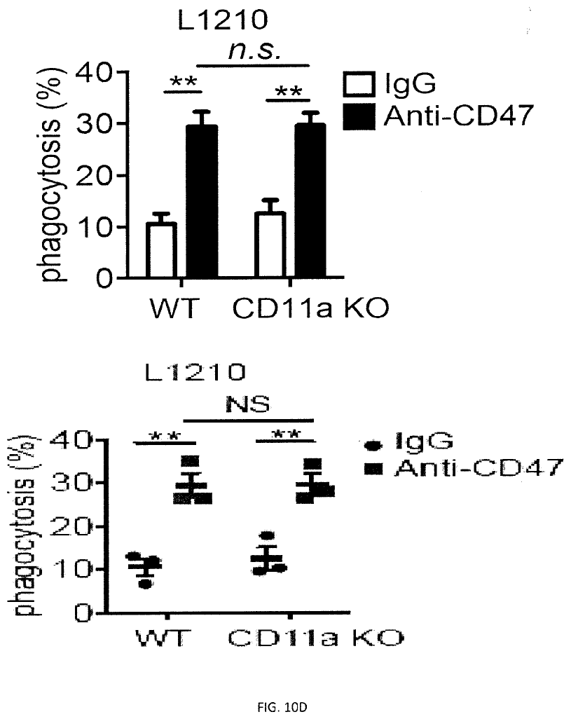

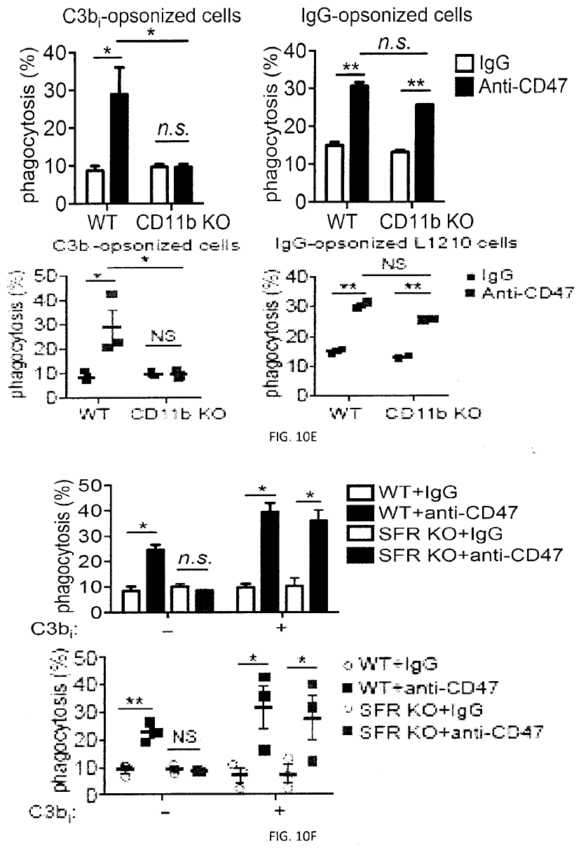

To test if Mac-1 was also required for phagocytosis of SLAMF7-dependent targets, Ab-blocking experiments were performed. Ab blockade of CD11b or CD18, but not of integrins CD11a (alphaL; LFA-1), CD11c (alpha_{X}), CD29 (beta1) or CD61 (beta3), significantly attenuated the enhancement of L1210 phagocytosis in the presence of anti-CD47 Ab (FIG. 10A). Anti-CD47 Ab-induction of L1210 cell phagocytosis was also severely compromised in CD11b KO macrophages, but not in CD11a KO macrophages (FIGS. 10B, 10C-A to 10C-E, 10D). In keeping with the known ability of Mac-1 to bind C3b²⁶, CD11 b KO macrophages displayed reduced anti-CD47 Ab-induced phagocytosis of C3b;-opsonized, but not of IgG-opsonized, L1210 cells (FIG. 10E). To determine whether SLAMF7 might be reciprocally required for the capacity of Mac-1 to initiate phagocytosis of C3b;-opsonized targets, the inventors assessed the ability of SFR KO macrophages to phagocytose C3b; opsonised L1210. SFR KO macrophages did not display a defect in phagocytosis of C3b;-opsonised L1210 cells in the presence of anti-CD47, compared to non-opsonized L1210 (FIG. 10F). Therefore, Mac-1 expression on macrophages was required for SLAMF7-dependent phagocytosis of tumor cells, but Mac-1 did not display a reciprocal requirement of SLAMF7 for phagocytosis of C3b1-opsonized targets.

### Example 7: Determining which Human Hematologic Tumors are Susceptible to SLAMF7-Dependent Phagocytosis During SIRPalpha-CD47 Blockade Therapy

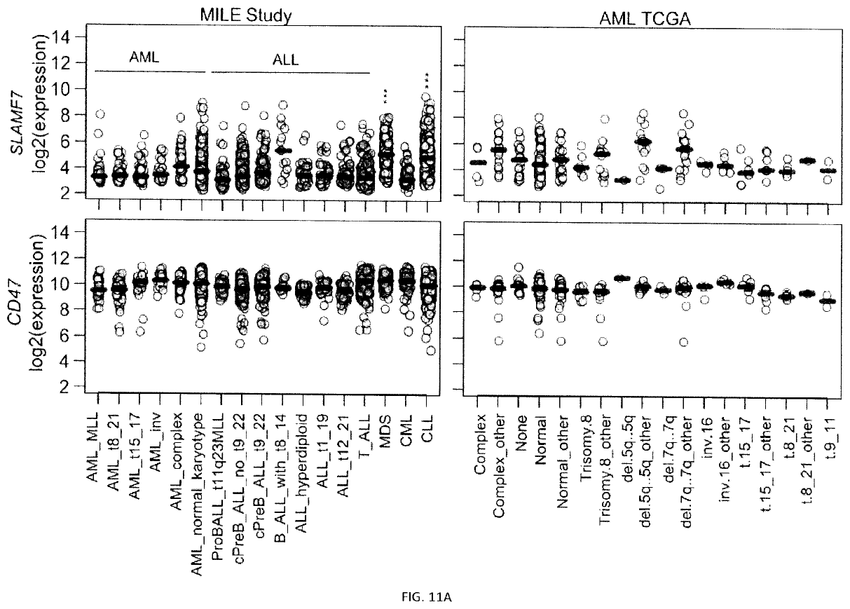

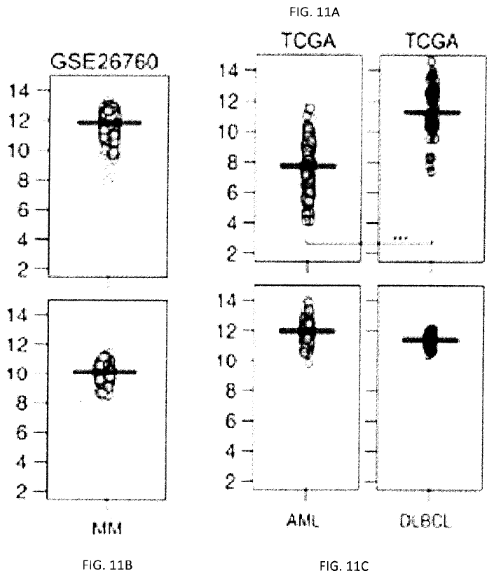

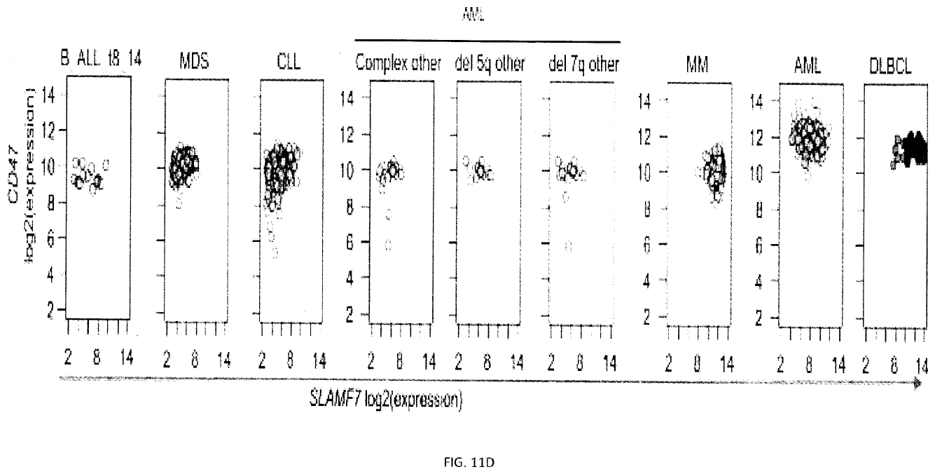

To ascertain which human hematologic tumors may be susceptible to SLAMF7-dependent phagocytosis during SIRPalpha-CD47 blockade therapy, expression datasets of primary human hematologic malignancies were extracted for SLAMF7 and CD47. Acute myelogenous leukemia (AML), acute lymphocytic leukemia (ALL) and chronic myelogenous leukemia (CML) expressed lower median levels of SLAMF7, although some individual samples had higher levels (FIGS. 11A-B). In contrast, chronic lymphocytic leukemia (CLL), myelodysplastic syndrome (MDS), multiple myeloma (MM) and diffuse large B cell lymphoma (DLBCL) had higher levels of SLAMF7 (FIGS. 11A-C). CD47 was uniformly expressed at high levels in all tumor types, including samples displaying higher levels of SLAMF7 (FIGS. 11A-D). Thus, some hematologic cancer, in particular CLL, MDS, MM and DLBCL, frequently co-expressed high levels of SLAMF7 and CD47.

### Example 8: Expression of SLAMF7 in Non-Hematopoietic Human Tumor Cells

To examine if SLAMF7 is expressed in non-hematopoietic tumors, expression of SLAMF7 was analyzed using a public dataset of 1019 human non-hematopoietic and hematopoietic tumor cell lines (Cancer Cell Line Encyclopedia)⁵⁴. Expression of CD47, CD45 (a marker of hematopoietic cells) and other SLAM family receptors (CD48, CD84, SLAMF1, 2B4, Ly-9 and SLAMF6) was also analyzed in parallel. Normalized data were extracted from The Cancer Genome Atlas (TCGA) and were initially published⁵⁴.

The inventors' analyses showed that, similar to hematopoietic tumor cells (FIG. 12A), several non-hematopoietic tumor cells expressed SLAMF7 (FIG. 12J). This was particularly the case for melanoma and non-small cell lung cancer. Expression of SLAMF7 was also seen in other types of solid tumors, including glioma, liver cancer, upper aerodigestive cancer and urinary tract cancer. All tumors expressed CD47 (FIGS. 12B and K). Hematopoietic tumor cells also expressed CD45 and other SLAM family receptors (FIGS. 12C-I), as expected²². However, non-hematopoietic tumor cells did not express CD45 and other SLAM family receptors (FIGS. 12L-R).

Some non-hematopoietic tumor cells, in particular, melanoma and non-small cell lung cancer, express SLAMF7, in addition to CD47.

### Example 9: Susceptibility of SLAMF7 Positive Non-Hematopoietic Tumor Cells to Enhanced Phagocytosis in Response to SIRPalpha-CD47 Checkpoint Blockade

BMDMs are tested for phagocytosis of various non-hematopoietic target cells (HCC827; Lung Carcinoma; Human (Homo sapiens) (CRL-2868); NCI-H1838; Lung Carcinoma; Human (Homo sapiens) (CRL-5899); NCI-H1373; Lung Adenocarcinoma; Human (Homo sapiens) (CRL-5866) (negative control); SK-MEL-1; Malignant Melanoma; Human (Homo sapiens) (HTB-67); SK-MEL-28; Melanoma; Human (Homo sapiens) (HTB-72)), in the presence of blocking anti-CD47 antibodies (Ab) or control IgG using several assays. Phagocytosis is monitored using a fluorescence-based microscopy assay.

### Example 10: Determining which Human Non-Hematologic Tumors are Susceptible to SLAMF7-Dependent Phagocytosis During SIRPalpha-CD47 Blockade Therapy

To ascertain which human non-hematologic tumors are susceptible to SLAMF7-dependent phagocytosis during SIRPalpha-CD47 blockade therapy, expression of SLAMF7 is analyzed using several public datasets of primary non-hematopoietic human tumors (TCGA)⁵⁴. Expression of CD47, CD45 (a marker of hematopoietic cells) and other SLAM family receptors (CD48, CD84, SLAMF1, 2B4, Ly-9, SLAMF6) is also analyzed in parallel.

### Example 11: SIRPalpha-CD47 Checkpoint Blockade in Solid Tumor Mice Models

To show that SLAM receptors are required for enhanced phagocytosis of solid tumor cells in response to SIRPalpha-CD47 blockade in vivo, the above mentioned intra-peritoneal tumor clearance assay for non-hematopoietic tumor cells is used. The impact of SIRPalpha-CD47 blockade in SLAM positive tumor cells is further assessed in vivo in a sub-cutaneous transplantation assay showing the impact of on tumor growth. The present shows that macrophages selectively phagocytose SLAMF7 positive tumor cells in response to SIRPalpha-CD47 checkpoint blockade, in an FcR-independent manner. Phagocytosis was mediated by the homotypic receptor SLAMF7^{15-17,24,25} . It also required expression of integrin Mac-1, and of ITAM-containing subunits FcRgamma and DAP12^{21,22.} The instant finding that Mac-1 blocking antibodies prevented phagocytosis of hematopoietic tumor cells implies that, like SLAMF7, Mac-1 plays a direct role in target cell recognition.

Without being so limited, the above results suggest that a dual cooperative mechanism between SLAMF7 and Mac-1 promotes phagocytosis during SIRPalpha-CD47 checkpoint blockade. SLAMF7 synergizes with Mac-1 both to recognize ligands expressed on target cells, and to generate signals leading to actin polarization and phagocytosis. Normal B cells, which highly express SLAMF7 and CD47, are not susceptible to enhanced phagocytosis during SIRPalpha-CD47 blockade. This is possibly due to lack of relevant ligands for Mac-1, or expression of ligands for inhibitory receptors other than SIRPalpha.

Since the expression pattern of SLAMF7 on tumors is more restricted than that of ICAM-1 (and other Mac-1 ligands), and without being limited by this hypothesis, the above results suggest that SLAMF7 expression determines which tumor cells are engulfed by phagocytes in response to SIRPalpha-CD47 checkpoint blockade.

The scope of the claims should not be limited by the preferred embodiments set forth in the examples but should be given the broadest interpretation consistent with the description as a whole.

### REFERENCES

1 Arandjelovic, S. & Ravichandran, K. S. Phagocytosis of apoptotic cells in homeostasis. Nat Immunol 16, 907-917, doi:10.1038/ni.3253 (2015).
2 Gordon, S., Pluddemann, A. & Martinez Estrada, F. Macrophage heterogeneity in tissues: phenotypic diversity and functions. Immunol Rev 262, 36-55, doi:10.1111/imr.12223 (2014).
3 Schultze, J. L. & Schmidt, S. V. Molecular features of macrophage activation. Semin Immunol, doi:10.1016/j.smim.2016.03.009 (2016).
4 Freeman, S. A. & Grinstein, S. Phagocytosis: receptors, signal integration, and the cytoskeleton. Immunol Rev 262, 193-215, doi:10.1111/imr.12212 (2014).
5 Chao, M. P. et al. Anti-CD47 antibody synergizes with rituximab to promote phagocytosis and eradicate non-Hodgkin lymphoma. Cell 142, 699-713, doi:10.1016/j.cell.2010.07.044 (2010).
6 Majeti, R. et al. CD47 is an adverse prognostic factor and therapeutic antibody target on human acute myeloid leukemia stem cells. Cell 138, 286-299, doi:10.1016/j.cel.2009.05.045 (2009).
7 Jaiswal, S. et al. CD47 is upregulated on circulating hematopoietic stem cells and leukemia cells to avoid phagocytosis. Cell 138, 271-285, doi: 10.1016/j.cell.2009.05.046 (2009).
8 Theocharides, A. P. et al. Disruption of SIRPalpha signaling in macrophages eliminates human acute myeloid leukemia stem cells in xenografts. J Exp Med 209, 1883-1899, doi:10.1084/jem.20120502 (2012).
9 Murata, Y., Kotani, T., Ohnishi, H. & Matozaki, T. The CD47-SIRPalpha signalling system: its physiological roles and therapeutic application. J Biochem 155, 335-344, doi:10.1093/jb/mvu017 (2014).
10 Barclay, A. N. & Van den Berg, T. K. The interaction between signal regulatory protein alpha (SIRPalpha) and CD47: structure, function, and therapeutic target. Annu Rev Immunol 32, 25-50, doi:10.1146/annurev-immunol-032713-120142 (2014).
11 Chao, M. P., Weissman, 1. L. & Majeti, R. The CD47-SIRPalpha pathway in cancer immune evasion and potential therapeutic implications. Curr Opin Immunol 24, 225-232, doi: 10.1016/j.coi.2012.01.010 (2012).
12 Ho, J. M., Danska, J. S. & Wang, J. C. Targeting SIRPalpha in cancer. Oncoimmunology 2, e23081, doi:10.4161/onci.23081 (2013).
13 Wu, N. & Veillette, A. SLAM family receptors in normal immunity and immune pathologies. Curr Opin Immunol 38, 45-51, doi:10.1016/j.coi.2015.11.003 (2016).
14 Cannons, J. L., Tangye, S. G. & Schwartzberg, P. L. SLAM family receptors and SAP adaptors in immunity. Annu Rev Immunol 29, 665-705, doi:10.1146/annurev-immunol-030409-101302 (2011).
15 Calpe, S. et al. The SLAM and SAP gene families control innate and adaptive immune responses. Adv Immunol 97, 177-250, doi:10.1016/50065-2776(08)00004-7 (2008).
16 ffrench-Constant, C. & Colognato, H. Integrins: versatile integrators of extracellular signals. Trends Cell Biol 14, 678-686, doi: 10.1016/j.tcb.2004.10.005 (2004).
17 Miranti, C. K. & Brugge, J. S. Sensing the environment: a historical perspective on integrin signal transduction. Nat Cell Biol 4, E83-90, doi:10.1038/ncb0402-e83 (2002).
18 Arnaout, M. A., Mahalingam, B. & Xiong, J. P. Integrin structure, allostery, and bidirectional signaling. Annu Rev Cell Dev Biol 21, 381-410, doi:10.1146/annurev.cellbio.21.090704.151217 (2005).
19 Jakus, Z., Fodor, S., Abram, C. L., Lowell, C. A. & Mocsai, A. Immunoreceptor-like signaling by beta 2 and beta 3 integrins. Trends Cell Biol 17, 493-501, doi:10.1016/j.tcb.2007.09.001 (2007).
20 Hamerman, J. A., Ni, M., Killebrew, J. R., Chu, C. L. & Lowell, C. A. The expanding roles of ITAM adapters FcRgamma and DAP12 in myeloid cells. Immunol Rev 232, 42-58, doi:10.1111/j.1600-065X.2009.00841.x (2009).
21 Chao, M. P. et al. Calreticulin is the dominant pro-phagocytic signal on multiple human cancers and is counterbalanced by CD47. Sci Transl Med 2, 63ra94, doi:10.1126/scitranslmed.3001375 (2010).
22 Veillette, A. & Guo, H. CS1, a SLAM family receptor involved in immune regulation, is a therapeutic target in multiple myeloma. Crit Rev Oncol Hematol 88, 168-177, doi:10.1016/j.critrevonc.2013.04.003 (2013).
23 Guo, H., Cruz-Munoz, M. E., Wu, N., Robbins, M. & Veillette, A. Immune cell inhibition by SLAMF7 is mediated by a mechanism requiring src kinases, CD45, and SHIP-1 that is defective in multiple myeloma cells. Mol Cell Biol 35, 41-51, doi:10.1128/MCB.01107-14 (2015).
24 Rawlings, D. J. et al. Mutation of unique region of Bruton's tyrosine kinase in immunodeficient XID mice. Science 261, 358-361 (1993).
25 Takai, T., Li, M., Sylvestre, D., Clynes, R. & Ravetch, J. V. FcR gamma chain deletion results in pleiotrophic effector cell defects. Cell 76, 519-529 (1994).
26 Todd, R. F., 3rd. The continuing saga of complement receptor type 3 (CR3). J Clin Invest 98, 1-2, doi:10.1172/JC1118752 (1996).
27 Sharma, P. & Allison, J. P. Immune checkpoint targeting in cancer therapy: toward combination strategies with curative potential. Cell 161, 205-214, doi:10.1016/j.cell.2015.03.030 (2015).
28 Korman, A. J., Peggs, K. S. & Allison, J. P. Checkpoint blockade in cancer immunotherapy. Adv Immunol 90, 297-339, doi:10.1016/50065-2776(06)90008-X (2006).
29 Hsi, E. D. et al. CS1, a potential new therapeutic antibody target for the treatment of multiple myeloma. Clin Cancer Res14, 2775-2784, doi:10.1158/1078-0432.CCR-07-4246 (2008).
30 Wu, N. et al. A hematopoietic cell-driven mechanism involving SLAMF6 receptor, SAP adaptors and SHP-1 phosphatase regulates NK cell education. Nat Immunol 17, 387-396, doi:10.1038/ni.3369 (2016).
31 Dong, Z. et al. The adaptor SAP controls NK cell activation by regulating the enzymes Vav-1 and SHIP-1 and by enhancing conjugates with target cells. Immunity 36, 974-985, doi: 10.1016/j.immuni.2012.03.023 (2012).
32 Elliott, E. R. et al. Deletion of Syk in neutrophils prevents immune complex arthritis. J Immunol 187, 4319-4330, doi:10.4049/jimmunol.1100341 (2011).
33 Kaifu, T. et al. Osteopetrosis and thalamic hypomyelinosis with synaptic degeneration in DAP12-deficient mice. J Clin Invest 111, 323-332, doi:10.1172/JC116923 (2003).
34 Mocsai, A. et al. Integrin signaling in neutrophils and macrophages uses adaptors containing immunoreceptor tyrosine-based activation motifs. Nat Immunol 7, 1326-1333, doi:10.1038/ni1407 (2006).
35 Rhee, 1., Davidson, D., Souza, C. M., Vacher, J. & Veillette, A. Macrophage fusion is controlled by the cytoplasmic protein tyrosine phosphatase PTP-PEST/PTPN12. Mol Cell Biol 33, 2458-2469, doi:10.1128/MCB.00197-13 (2013).
36 Dong, Z. et al. Essential function for SAP family adaptors in the surveillance of hematopoietic cells by natural killer cells. Nat Immunol 10, 973-980, doi:10.1038/ni.1763 (2009).
37 Chow, L. M. et al. Two distinct protein isoforms are encoded by ntk, a csk-related tyrosine protein kinase gene. Oncogene 9, 3437-3448 (1994).
38 Abraham, N., Miceli, M. C., Parnes, J. R. & Veillette, A. Enhancement of T-cell responsiveness by the lymphocyte-specific tyrosine protein kinase p56lck. Nature 350, 62-66, doi:10.1038/350062a0 (1991).
39 Bouchon, A., Cella, M., Grierson, H. L., Cohen, J. 1. & Colonna, M. Activation of NK cell-mediated cytotoxicity by a SAP-independent receptor of the CD2 family. J Immunol 167, 5517-5521 (2001).
40 Latour, S., Chow, L. M. & Veillette, A. Differential intrinsic enzymatic activity of Syk and Zap-70 protein-tyrosine kinases. J Biol Chem 271, 22782-22790 (1996).
41 Cruz-Munoz, M. E., Dong, Z., Shi, X., Zhang, S. & Veillette, A. Influence of CRACC, a SLAM family receptor coupled to the adaptor EAT-2, on natural killer cell function. Nat Immunol 10, 297-305, doi:10.1038/ni.1693 (2009).
42 Zhang, Z. et al. DNAM-1 controls NK cell activation via an ITT-like motif. J Exp Med212, 2165-2182, doi:10.1084/jem.20150792 (2015).
43 Veillette, A., Bookman, M. A., Horak, E. M. & Bolen, J. B. The CD4 and CD8 T cell surface antigens are associated with the internal membrane tyrosine-protein kinase p56lck. Cell 55, 301-308 (1988).
44 Jaiswal S1, Jamieson C H, Pang W W, Park C Y, Chao M P, Majeti R, Traver D, van Rooijen N, Weissman I L. CD47 is upregulated on circulating hematopoietic stem cells and leukemia cells to avoid phagocytosis. Cell, 138:271-85 (2009).
45 Pardoll D M, The blockade of immune checkpoints in cancer immunotherapy Nature Reviews. Cancer 12 (4): 252-64 (2012).
46 Karwacz K et al. PD-L1 co-stimulation contributes to ligand-induced T cell receptor down-modulation on CD8⁺ T cells, EMBO Molecular Medicine 3 (10): 581-592 (2011).
47 Bagger, F. O. et al. BloodSpot: a database of gene expression profiles and transcriptional programs for healthy and malignant haematopoiesis. Nucleic Acids Res 44, D917-924, doi:10.1093/nar/gkv1101 (2016).
48 Chapman, M. A. et al. Initial genome sequencing and analysis of multiple myeloma. Nature 471, 467-472, doi:10.1038/nature09837 (2011).
49 Cloutier, P., Lavallee-Adam, M., Faubert, D., Blanchette, M. & Coulombe, B. Methylation of the DNA/RNA-binding protein Kin17 by METTL22 affects its association with chromatin. J Proteomics 100, 115-124, doi:10.1016/j.jprot.2013.10.008 (2014).
50 Guo, H. et al. Deletion of Slam locus in mice reveals inhibitory role of SLAM family in NK cell responses regulated by cytokines and LFA-1. J Exp Med 213, 2187-2207, doi:10.1084/jem.20160552 (2016).
51 Heit, B. et al. Multimolecular signaling complexes enable Syk-mediated signaling of CD36 internalization. Dev Cell 24, 372-383, doi:10.1016/j.device1.2013.01.007 (2013).
52 Kohlmann, A. et al. An international standardization programme towards the application of gene expression profiling in routine leukaemia diagnostics: the Microarray Innovations in LEukemia study prephase. Br J Haematol 142, 802-807, doi:10.1111/j.1365-2141.2008.07261.x (2008).
53 Sockolosky, J. T. et al. Durable antitumor responses to CD47 blockade require adaptive immune stimulation Proc Natl Acad Sci USA. 113(19): E2646-E2654. (2016).
54 Barretina, J. et al. The Cancer Cell Line Encyclopedia enables predictive modelling of anticancer drug sensitivity. Nature 483 (7391), 603-607, doi:10.1038/nature11003 (2012).

1. A method for the prevention and/or treatment of a neoplastic disease comprising a solid tumor in a subject in need thereof, said method comprising administering an effective amount of (i) a signal regulatory protein alpha (SIRPalpha)-cluster of differentiation 47 (CD47) checkpoint inhibitor; or (ii) a composition comprising the inhibitor, and a pharmaceutically acceptable carrier; to a subject having solid tumor cells expressing signaling lymphocytic activation molecule family member 7 (SLAMF7) and CD47.

2. The method of claim 1, wherein the solid tumor is (i) a bile duct, breast, colorectal, esophagus, glioma, liver, non-small cell lung, melanoma, ovary, pancreas, soft tissue, stomach, upper aerodigestive or urinary tract tumor; or (iii) a glioma, liver, non-small cell lung, melanoma, upper aerodigestive or urinary tract tumor; or (iii) a non-small cell lung tumor or a melanoma.

3. (canceled)

4. (canceled)

5. The method of claim 1, further comprising detecting (i) SLAMF7 expression and/or activity; (ii) CD47 expression and/or activity; or (iii) a combination of at (i) and (ii) in the tumor cells.

6. The method of claim 1, wherein the SIRPalpha-CD47 checkpoint inhibitor is (i) a non-Fc receptor binding inhibitor; (ii) an antibody or antibody fragment that specifically binds to CD47 and/or an antibody or an antibody fragment that specifically binds to SIRPalpha; (iii) a non-Fc receptor binding antibody fragment.

7. (canceled)

8. (canceled)

9. The method of claim 1, further comprising administering at least one further therapeutic agent to the subject, preferably wherein the at least one further therapeutic agent comprises a SLAMF7 agonist such as elotuzumab.

10. (canceled)

11. (canceled)

12. A method for stratifying a subject having a neoplastic disease comprising a solid tumor comprising detecting signaling lymphocytic activation molecule family member 7 (SLAMF7) expression and/or activity in the subject's tumor cells, wherein said detecting enables the stratification of the subject, preferably wherein when SLAMF7 expression and/or activity is detected the subject's tumor cells, the subject is included in a clinical trial for a SIRPalpha-CD47 checkpoint inhibitor.

13. The method of claim 12, wherein when SLAMF7 expression and/or activity is detected, the method further comprises administering an effective amount of (i) a signal regulatory protein alpha (SIRPalpha)-cluster of differentiation 47 (CD47) checkpoint inhibitor; or (ii) a composition comprising the inhibitor, and a pharmaceutically acceptable carrier, to the subject.

14. The method of claim 13, wherein the SIRPalpha-CD47 checkpoint inhibitor is (i) as non-Fc receptor binding inhibitor; (ii) an antibody or an antibody fragment that specifically binds to CD47 and/or an antibody or an antibody fragment that specifically binds to SIRPalpha; or (iii) a non-Fc receptor binding antibody fragment.

15. (canceled)

16. (canceled)

17. The method of claim 13, further comprising administering at least one further therapeutic agent to the subject, preferably wherein the at least one further therapeutic agent comprises a SLAMF7 agonist such as elotuzumab.

18. (canceled)

19. (canceled)

20. The method of claim 12, wherein when SLAMF7 expression and/or activity is not detected, the method further comprises administering (a) an effective amount of (i) a SLAMF7 inhibitor; (ii) an SIRPalpha-CD47 checkpoint inhibitor and of an Fc receptor-binding antibody or fragment thereof targeting an antigen expressed at the surface of the subject's tumor cells; or (iii) a combination of (i) and (ii); or (b) a composition comprising (a), and a pharmaceutically acceptable carrier, to the subject, preferably further comprising administering at least one further therapeutic agent to the subject, most preferably wherein the at least one further therapeutic agent comprises another agent that activates T cells.

21. (canceled)

22. (canceled)

23. A kit for preventing and/or treating a neoplastic disease comprising a solid tumor in a subject, comprising (A) (a) a signal regulatory protein alpha (SIRPalpha)-cluster of differentiation 47 (CD47) checkpoint inhibitor; and (b) (i) a pharmaceutically acceptable carrier; (ii) at least one further therapeutic agent; or (iii) a combination of (i) and (ii); or (B) (a) a signaling lymphocytic activation molecule family member 7 (SLAMF7) inhibitor; and (b) (i) a pharmaceutically acceptable carrier; (ii) at least one further therapeutic agent; or (iii) a combination of (i) and (ii).

24. The kit of claim 23 (A), wherein the SIRPalpha-CD47 checkpoint inhibitor is (i) non-Fc receptor binding inhibitor; (ii) an antibody or an antibody fragment that specifically binds to CD47 and/or an antibody or an antibody fragment that specifically binds to SIRPalpha; or (iii) a non-Fc receptor binding antibody fragment.

25. (canceled)

26. (canceled)

27. The kit of claim 23 (A), wherein the at least one further therapeutic agent comprises a SLAMF7 agonist, preferably wherein the SLAMF7 agonist is elotuzumab.

28. (canceled)

29. A method for the prevention and/or treatment of a neoplastic disease comprising a solid tumor in a subject in need thereof, said method comprising administering an effective amount of (A) (i) a signaling lymphocytic activation molecule family member 7 (SLAMF7) inhibitor; or (ii) a composition comprising the inhibitor, and a pharmaceutically acceptable carrier; or (B) (i) (a) a signaling lymphocytic activation molecule family member 7 (SLAMF7) protein or nucleic acid; or (b) a composition comprising the protein or nucleic acid, and a pharmaceutically acceptable carrier; and (ii) (a) a signal regulatory protein alpha (SIRPalpha)-cluster of differentiation 47 (CD47) checkpoint inhibitor; or (b) a composition comprising the SIRPalpha-CD47 checkpoint inhibitor, and a pharmaceutically acceptable carrier,
to a subject having solid tumor cells that do not express signaling lymphocytic activation molecule family member 7 (SLAMF7).

30. The method of claim 29, wherein the solid tumor is (i) bile duct, breast, colorectal, esophagus, glioma, liver, non-small cell lung, melanoma, ovary, pancreas, soft tissue, stomach, upper aerodigestive or urinary tract tumor; (ii) a glioma, liver, non-small cell lung, melanoma, upper aerodigestive or urinary tract tumor; or (iii) a non-small cell lung tumor or a melanoma.

31. (canceled)

32. (canceled)

33. The method of claim 29, further comprising determining SLAMF7 expression and/or activity in the tumor cells.

34. The method of claim 29, further comprising administering at least one further therapeutic agent to the subject, preferably wherein the at least one further therapeutic agent comprises another agent that activates T cells.

35. (canceled)

36. (canceled)

37. The kit of claim 23(B), wherein the at least one further therapeutic agent comprises another agent that activates T cells.

38. (canceled)

39. The method of claim 29 (B), wherein the administrations of (i) and (ii) are performed sequentially.

40. A kit for stratifying a subject having a neoplastic disease comprising a solid tumor, comprising (a) a signaling lymphocytic activation molecule family member 7 (SLAMF7) ligand; and (b) (i) a cluster of differentiation 47 (CD47) ligand; (ii) signal regulatory protein alpha (SIRPalpha) ligand; or (iii) a combination of (i) and (ii), wherein preferably (i) the SLAMF7 ligand is an antibody that specifically binds to SLAMF7; (ii) the CD47 ligand is an antibody that specifically binds to CD47; (iii) the SIRPalpha ligand is an antibody that specifically binds to SIRPalpha; or (iv) any combination of at least two of (i) to (iii).

41. (canceled)


SEQUENCE LISTING (25 sequences)

SEQ ID NO: 1 - protein, 335 aa, homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 2 - DNA, 2908 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 3 - protein, 165 aa, homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYENNPKGRSSKYGLLHCGNTEKDGKSPLTAHDARHTKAICL

SEQ ID NO: 4 - DNA, 2411 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 5 - protein, 204 aa, homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEYIEEKKRVDICRETPNICPHSGENTEYDTIPHTNRTILKEDPANTVYSTVEIPKKMENPHSLLTMPDTPRLFAYENVI

SEQ ID NO: 6 - DNA, 2515 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 7 - protein, 228 aa, homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 8 - DNA, 2587 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 9 - protein, 188 aa, homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEEYIEEKKRVDICRETPNICPHSGENTEYDTIPHTNRTILKEDPANTVYSTVEIPKKMENPHSLLTMPDTPRLFAYENVI

SEQ ID NO: 10 - DNA, 2467 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 11 - protein, 296 aa, homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEENNPKGRSSKYGLLHCGNTEKDGKSPLTAHDARHTKAICL

SEQ ID NO: 12 - DNA, 2804 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 13 - protein, 149 aa, homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEENNPKGRSSKYGLLHCGNTEKDGKSPLTAHDARHTKAICL

SEQ ID NO: 14 - DNA, 2363 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaagagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 15 - protein, 241 aa, homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEGDCLSPLHRRLCPGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 16 - DNA, 2626 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgactgcctctcccctctccacaggagactctgcccaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 17 - protein, 201 aa, homo sapiens
MGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 18 - DNA, 2369 bp, homo sapiens
agctaaaatataaaatgggaatataccaaatgctgatgaagatggggagcaaatagatctctcatagattgctggtggcaagagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 19 - protein, 189 aa, homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEENNPKGRSSKYGLLHCGNTEKDGKSPLTAHDARHTKAICL

SEQ ID NO: 20 - DNA, 2483 bp, homo sapiens
agctaaaatataaaatgggaatataccaaatgctgatgaagatggggagcaaatagatctctcatagattgctggtggcaaggtaaaatgctctattcactctgaaaataatttagcaattactcaatctcacatgtctgcggcgtgacccctcctgcttctttaaatatcagctggggaagaggtctgagtaatacctaagagggaagtggcttcatttcagtggctgacttccagagagcaatatggctggttccccaacatgcctcaccctcatctatatcctttggcagctcacagagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaaaaaaaaaaaaa

SEQ ID NO: 21 - protein, 305 aa, homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 22 - protein, 323 aa, homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 23 - protein, 305 aa, homo sapiens; identical to SEQ ID NO: 21

SEQ ID NO: 24 - DNA, 25 bp, Artificial sequence
  synthetic sequence
caccgagcaacagcgccgccgccaa

SEQ ID NO: 25 - DNA, 25 bp, Artificial sequence
  synthetic sequence
caccgttggcggcggcgctgttgct